SEQUENCE LISTING (45 sequences)

SEQ ID NO: 1 - DNA, 47 bp, Artificial Sequence
  5′UTR-001 (Upstream UTR)
gggaaataagagagaaaagaagagtaagaagaaatataagagccacc

SEQ ID NO: 2 - DNA, 47 bp, Artificial Sequence
  5′UTR-002 (Upstream UTR)
gggagatcagagagaaaagaagagtaagaagaaatataagagccacc

SEQ ID NO: 3 - DNA, 145 bp, Artificial Sequence
  5′UTR-003 (Upstream UTR)
ggaataaaagtctcaacacaacatatacaaaacaaacgaatctcaagcaatcaagcattctacttctattgcagcaatttaaatcatttcttttaaagcaaaagcaattttctgaaaattttcaccatttacgaacgatagcaac

SEQ ID NO: 4 - RNA, 42 nt, Artificial Sequence
  5′UTR-004 (Upstream UTR)
gggagacaagcuuggcauuccgguacuguugguaaagccacc

SEQ ID NO: 5 - DNA, 47 bp, Artificial Sequence; identical to SEQ ID NO: 2
  5′UTR-005 (Upstream UTR)

SEQ ID NO: 6 - DNA, 145 bp, Artificial Sequence; identical to SEQ ID NO: 3
  5′UTR-006 (Upstream UTR)

SEQ ID NO: 7 - RNA, 42 nt, Artificial Sequence; identical to SEQ ID NO: 4
  5′UTR-007 (Upstream UTR)

SEQ ID NO: 8 - DNA, 47 bp, Artificial Sequence
  5′UTR-008 (Upstream UTR)
gggaattaacagagaaaagaagagtaagaagaaatataagagccacc

SEQ ID NO: 9 - DNA, 47 bp, Artificial Sequence
  5′UTR-009 (Upstream UTR)
gggaaattagacagaaaagaagagtaagaagaaatataagagccacc

SEQ ID NO: 10 - DNA, 47 bp, Artificial Sequence
  5′UTR-010 (Upstream UTR)
gggaaataagagagtaaagaacagtaagaagaaatataagagccacc

SEQ ID NO: 11 - DNA, 47 bp, Artificial Sequence
  5′UTR-011 (Upstream UTR)
gggaaaaaagagagaaaagaagactaagaagaaatataagagccacc

SEQ ID NO: 12 - DNA, 47 bp, Artificial Sequence
  5′UTR-012 (Upstream UTR)
gggaaataagagagaaaagaagagtaagaagatatataagagccacc

SEQ ID NO: 13 - DNA, 47 bp, Artificial Sequence
  5′UTR-013 (Upstream UTR)
gggaaataagagacaaaacaagagtaagaagaaatataagagccacc

SEQ ID NO: 14 - DNA, 47 bp, Artificial Sequence
  5′UTR-014 (Upstream UTR)
gggaaattagagagtaaagaacagtaagtagaattaaaagagccacc

SEQ ID NO: 15 - DNA, 47 bp, Artificial Sequence
  5′UTR-015 (Upstream UTR)
gggaaataagagagaatagaagagtaagaagaaatataagagccacc

SEQ ID NO: 16 - DNA, 47 bp, Artificial Sequence
  5′UTR-016 (Upstream UTR)
gggaaataagagagaaaagaagagtaagaagaaaattaagagccacc

SEQ ID NO: 17 - DNA, 47 bp, Artificial Sequence
  5′UTR-017 (Upstream UTR)
gggaaataagagagaaaagaagagtaagaagaaatttaagagccacc

SEQ ID NO: 18 - DNA, 92 bp, Artificial Sequence
  5′UTR-018 (Upstream UTR)
tcaagcttttggaccctcgtacagaagctaatacgactcactatagggaaataagagagaaaagaagagtaagaagaaatataagagccacc

SEQ ID NO: 19 - DNA, 142 bp, Artificial Sequence
  142-3p 5′UTR-001 (Upstream UTR including miR142-3p)
tgataatagtccataaagtaggaaacactacagctggagcctcggtggccatgcttcttgccccttgggcctccccccagcccctcctccccttcctgcacccgtacccccgtggtctttgaataaagtctgagtgggcggc

SEQ ID NO: 20 - DNA, 142 bp, Artificial Sequence
  142-3p 5′UTR-002 (Upstream UTR including miR142-3p)
tgataataggctggagcctcggtggctccataaagtaggaaacactacacatgcttcttgccccttgggcctccccccagcccctcctccccttcctgcacccgtacccccgtggtctttgaataaagtctgagtgggcggc

SEQ ID NO: 21 - DNA, 142 bp, Artificial Sequence
  142-3p 5′UTR-003 (Upstream UTR including miR142-3p)
tgataataggctggagcctcggtggccatgcttcttgccccttccataaagtaggaaacactacatgggcctccccccagcccctcctccccttcctgcacccgtacccccgtggtctttgaataaagtctgagtgggcggc

SEQ ID NO: 22 - DNA, 142 bp, Artificial Sequence
  142-3p 5′UTR-004 (Upstream UTR including miR142-3p)
tgataataggctggagcctcggtggccatgcttcttgccccttgggcctccccccagtccataaagtaggaaacactacacccctcctccccttcctgcacccgtacccccgtggtctttgaataaagtctgagtgggcggc

SEQ ID NO: 23 - DNA, 142 bp, Artificial Sequence
  142-3p 5′UTR-005 (Upstream UTR including miR142-3p)
tgataataggctggagcctcggtggccatgcttcttgccccttgggcctccccccagcccctcctccccttctccataaagtaggaaacactacactgcacccgtacccccgtggtctttgaataaagtctgagtgggcggc

SEQ ID NO: 24 - DNA, 142 bp, Artificial Sequence
  142-3p 5′UTR-006 (Upstream UTR including miR142-3p)
tgataataggctggagcctcggtggccatgcttcttgccccttgggcctccccccagcccctcctccccttcctgcacccgtaccccctccataaagtaggaaacactacagtggtctttgaataaagtctgagtgggcggc

SEQ ID NO: 25 - DNA, 142 bp, Artificial Sequence
  142-3p 5′UTR-007 (Upstream UTR including miR142-3p)
tgataataggctggagcctcggtggccatgcttcttgccccttgggcctccccccagcccctcctccccttcctgcacccgtacccccgtggtctttgaataaagttccataaagtaggaaacactacactgagtgggcggc

SEQ ID NO: 26 - DNA, 371 bp, Artificial Sequence
  3′UTR-001 (Creatine Kinase UTR)
gcgcctgcccacctgccaccgactgctggaacccagccagtgggagggcctggcccaccagagtcctgctccctcactcctcgccccgccccctgtcccagagtcccacctgggggctctctccacccttctcagagttccagtttcaaccagagttccaaccaatgggctccatcctctggattctggccaatgaaatatctccctggcagggtcctcttcttttcccagagctccaccccaaccaggagctctagttaatggagagctcccagcacactcggagcttgtgctttgtctccacgcaaagcgataaataaaagcattggtggcctttggtctttgaataaagcctgagtaggaagtctaga

SEQ ID NO: 27 - DNA, 568 bp, Artificial Sequence
  3′UTR-002 (Myoglobin UTR)
gcccctgccgctcccacccccacccatctgggccccgggttcaagagagagcggggtctgatctcgtgtagccatatagagtttgcttctgagtgtctgctttgtttagtagaggtgggcaggaggagctgaggggctggggctggggtgttgaagttggctttgcatgcccagcgatgcgcctccctgtgggatgtcatcaccctgggaaccgggagtggcccttggctcactgtgttctgcatggtttggatctgaattaattgtcctttcttctaaatcccaaccgaacttcttccaacctccaaactggctgtaaccccaaatccaagccattaactacacctgacagtagcaattgtctgattaatcactggccccttgaagacagcagaatgtccctttgcaatgaggaggagatctgggctgggcgggccagctggggaagcatttgactatctggaacttgtgtgtgcctcctcaggtatggcagtgactcacctggttttaataaaacaacctgcaacatctcatggtctttgaataaagcctgagtaggaagtctaga

SEQ ID NO: 28 - DNA, 289 bp, Artificial Sequence
  3′UTR-003 (alpha-actin UTR)
acacactccacctccagcacgcgacttctcaggacgacgaatcttctcaatgggggggcggctgagctccagccaccccgcagtcactttctttgtaacaacttccgttgctgccatcgtaaactgacacagtgtttataacgtgtacatacattaacttattacctcattttgttatttttcgaaacaaagccctgtggaagaaaatggaaaacttgaagaagcattaaagtcattctgttaagctgcgtaaatggtctttgaataaagcctgagtaggaagtctaga

SEQ ID NO: 29 - DNA, 379 bp, Artificial Sequence
  3′UTR-004 (Albumin UTR)
catcacatttaaaagcatctcagcctaccatgagaataagagaaagaaaatgaagatcaaaagcttattcatctgtttttctttttcgttggtgtaaagccaacaccctgtctaaaaaacataaatttctttaatcattttgcctcttttctctgtgcttcaattaataaaaaatggaaagaatctaatagagtggtacagcactgttatttttcaaagatgtgttgctatcctgaaaattctgtaggttctgtggaagttccagtgttctctcttattccacttcggtagaggatttctagtttcttgtgggctaattaaataaatcattaatactcttctaatggtctttgaataaagcctgagtaggaagtctaga

SEQ ID NO: 30 - DNA, 118 bp, Artificial Sequence
  3′UTR-005 (alpha-globin UTR)
gctgccttctgcggggcttgccttctggccatgcccttcttctctcccttgcacctgtacctcttggtctttgaataaagcctgagtaggaaggcggccgctcgagcatgcatctaga

SEQ ID NO: 31 - DNA, 908 bp, Artificial Sequence
  3′UTR-006 (G-CSF UTR)
gccaagccctccccatcccatgtatttatctctatttaatatttatgtctatttaagcctcatatttaaagacagggaagagcagaacggagccccaggcctctgtgtccttccctgcatttctgagtttcattctcctgcctgtagcagtgagaaaaagctcctgtcctcccatcccctggactgggaggtagataggtaaataccaagtatttattactatgactgctccccagccctggctctgcaatgggcactgggatgagccgctgtgagcccctggtcctgagggtccccacctgggacccttgagagtatcaggtctcccacgtgggagacaagaaatccctgtttaatatttaaacagcagtgttccccatctgggtccttgcacccctcactctggcctcagccgactgcacagcggcccctgcatccccttggctgtgaggcccctggacaagcagaggtggccagagctgggaggcatggccctggggtcccacgaatttgctggggaatctcgtttttcttcttaagacttttgggacatggtttgactcccgaacatcaccgacgcgtctcctgtttttctgggtggcctcgggacacctgccctgcccccacgagggtcaggactgtgactctttttagggccaggcaggtgcctggacatttgccttgctggacggggactggggatgtgggagggagcagacaggaggaatcatgtcaggcctgtgtgtgaaaggaagctccactgtcaccctccacctcttcaccccccactcaccagtgtcccctccactgtcacattgtaactgaacttcaggataataaagtgtttgcctccatggtctttgaataaagcctgagtaggaaggcggccgctcgagcatgcatctaga

SEQ ID NO: 32 - DNA, 835 bp, Artificial Sequence
  3′UTR-007 (Col1a2; collagen, type I, alpha 2 UTR)
actcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 33 - DNA, 297 bp, Artificial Sequence
  3′UTR-008 (Col6a2; collagen, type VI, alpha 2 UTR)
cgccgccgcccgggccccgcagtcgagggtcgtgagcccaccccgtccatggtgctaagcgggcccgggtcccacacggccagcaccgctgctcactcggacgacgccctgggcctgcacctctccagctcctcccacggggtccccgtagccccggcccccgcccagccccaggtctccccaggccctccgcaggctgcccggcctccctccccctgcagccatcccaaggctcctgacctacctggcccctgagctctggagcaagccctgacccaataaaggctttgaacccat

SEQ ID NO: 34 - DNA, 602 bp, Artificial Sequence
  3′UTR-009 (RPN1; ribophorin I UTR)
ggggctagagccctctccgcacagcgtggagacggggcaaggaggggggttattaggattggtggttttgttttgctttgtttaaagccgtgggaaaatggcacaactttacctctgtgggagatgcaacactgagagccaaggggtgggagttgggataatttttatataaaagaagtttttccactttgaattgctaaaagtggcatttttcctatgtgcagtcactcctctcatttctaaaatagggacgtggccaggcacggtggctcatgcctgtaatcccagcactttgggaggccgaggcaggcggctcacgaggtcaggagatcgagactatcctggctaacacggtaaaaccctgtctctactaaaagtacaaaaaattagctgggcgtggtggtgggcacctgtagtcccagctactcgggaggctgaggcaggagaaaggcatgaatccaagaggcagagcttgcagtgagctgagatcacgccattgcactccagcctgggcaacagtgttaagactctgtctcaaatataaataaataaataaataaataaataaataaataaaaataaagcgagatgttgccctcaaa

SEQ ID NO: 35 - DNA, 785 bp, Artificial Sequence
  3′UTR-010 (LRP1; low density lipoprotein receptor-related protein 1 UTR)
ggccctgccccgtcggactgcccccagaaagcctcctgccccctgccagtgaagtccttcagtgagcccctccccagccagcccttccctggccccgccggatgtataaatgtaaaaatgaaggaattacattttatatgtgagcgagcaagccggcaagcgagcacagtattatttctccatcccctccctgcctgctccttggcacccccatgctgccttcagggagacaggcagggagggcttggggctgcacctcctaccctcccaccagaacgcaccccactgggagagctggtggtgcagccttcccctccctgtataagacactttgccaaggctctcccctctcgccccatccctgcttgcccgctcccacagcttcctgagggctaattctgggaagggagagttctttgctgcccctgtctggaagacgtggctctgggtgaggtaggcgggaaaggatggagtgttttagttcttgggggaggccaccccaaaccccagccccaactccaggggcacctatgagatggccatgctcaacccccctcccagacaggccctccctgtctccagggcccccaccgaggttcccagggctggagacttcctctggtaaacattcctccagcctcccctcccctggggacgccaaggaggtgggccacacccaggaagggaaagcgggcagccccgttttggggacgtgaacgttttaataatttttgctgaattcctttacaactaaataacacagatattgttataaataaaattgt

SEQ ID NO: 36 - DNA, 3001 bp, Artificial Sequence
  3′UTR-011 (Nnt1; cardiotrophin-like cytokine factor 1 UTR)
atattaaggatcaagctgttagctaataatgccacctctgcagttttgggaacaggcaaataaagtatcagtatacatggtgatgtacatctgtagcaaagctcttggagaaaatgaagactgaagaaagcaaagcaaaaactgtatagagagatttttcaaaagcagtaatccctcaattttaaaaaaggattgaaaattctaaatgtctttctgtgcatattttttgtgttaggaatcaaaagtattttataaaaggagaaagaacagcctcattttagatgtagtcctgttggattttttatgcctcctcagtaaccagaaatgttttaaaaaactaagtgtttaggatttcaagacaacattatacatggctctgaaatatctgacacaatgtaaacattgcaggcacctgcattttatgttttttttttcaacaaatgtgactaatttgaaacttttatgaacttctgagctgtccccttgcaattcaaccgcagtttgaattaatcatatcaaatcagttttaattttttaaattgtacttcagagtctatatttcaagggcacattttctcactactattttaatacattaaaggactaaataatctttcagagatgctggaaacaaatcatttgctttatatgtttcattagaataccaatgaaacatacaacttgaaaattagtaatagtatttttgaagatcccatttctaattggagatctctttaatttcgatcaacttataatgtgtagtactatattaagtgcacttgagtggaattcaacatttgactaataaaatgagttcatcatgttggcaagtgatgtggcaattatctctggtgacaaaagagtaaaatcaaatatttctgcctgttacaaatatcaaggaagacctgctactatgaaatagatgacattaatctgtcttcactgtttataatacggatggattttttttcaaatcagtgtgtgttttgaggtcttatgtaattgatgacatttgagagaaatggtggctttttttagctacctctttgttcatttaagcaccagtaaagatcatgtctttttatagaagtgtagattttctttgtgactttgctatcgtgcctaaagctctaaatataggtgaatgtgtgatgaatactcagattatttgtctctctatataattagtttggtactaagtttctcaaaaaattattaacacatgaaagacaatctctaaaccagaaaaagaagtagtacaaattttgttactgtaatgctcgcgtttagtgagtttaaaacacacagtatcttttggttttataatcagtttctattttgctgtgcctgagattaagatctgtgtatgtgtgtgtgtgtgtgtgtgcgtttgtgtgttaaagcagaaaagacttttttaaaagttttaagtgataaatgcaatttgttaattgatcttagatcactagtaaactcagggctgaattataccatgtatattctattagaagaaagtaaacaccatctttattcctgccctttttcttctctcaaagtagttgtagttatatctagaaagaagcaattttgatttcttgaaaaggtagttcctgcactcagtttaaactaaaaataatcatacttggattttatttatttttgtcatagtaaaaattttaatttatatatatttttatttagtattatcttattctttgctatttgccaatcctttgtcatcaattgtgttaaatgaattgaaaattcatgccctgttcattttattttactttattggttaggatatttaaaggatttttgtatatataatttcttaaattaatattccaaaaggttagtggacttagattataaattatggcaaaaatctaaaaacaacaaaaatgatttttatacattctatttcattattcctctttttccaataagtcatacaattggtagatatgacttattttatttttgtattattcactatatctttatgatatttaagtataaataattaaaaaaatttattgtaccttatagtctgtcaccaaaaaaaaaaaattatctgtaggtagtgaaatgctaatgttgatttgtctttaagggcttgttaactatcctttattttctcatttgtcttaaattaggagtttgtgtttaaattactcatctaagcaaaaaatgtatataaatcccattactgggtatatacccaaaggattataaatcatgctgctataaagacacatgcacacgtatgtttattgcagcactattcacaatagcaaagacttggaaccaacccaaatgtccatcaatgatagacttgattaagaaaatgtgcacatatacaccatggaatactatgcagccataaaaaaggatgagttcatgtcctttgtagggacatggataaagctggaaaccatcattctgagcaaactattgcaaggacagaaaaccaaacactgcatgttctcactcataggtgggaattgaacaatgagaacacttggacacaaggtggggaacaccacacaccagggcctgtcatggggtggggggagtggggagggatagcattaggagatatacctaatgtaaatgatgagttaatgggtgcagcacaccaacatggcacatgtatacatatgtagcaaacctgcacgttgtgcacatgtaccctagaacttaaagtataattaaaaaaaaaaagaaaacagaagctatttataaagaagttatttgctgaaataaatgtgatctttcccattaaaaaaataaagaaattttggggtaaaaaaacacaatatattgtattcttgaaaaattctaagagagtggatgtgaagtgttctcaccacaaaagtgataactaattgaggtaatgcacatattaattagaaagattttgtcattccacaatgtatatatacttaaaaatatgttatacacaataaatacatacattaaaaaataagtaaatgta

SEQ ID NO: 37 - DNA, 1037 bp, Artificial Sequence
  3′UTR-012 (Col6a1; collagen, type VI, alpha 1 UTR)
cccaccctgcacgccggcaccaaaccctgtcctcccacccctccccactcatcactaaacagagtaaaatgtgatgcgaattttcccgaccaacctgattcgctagattttttttaaggaaaagcttggaaagccaggacacaacgctgctgcctgctttgtgcagggtcctccggggctcagccctgagttggcatcacctgcgcagggccctctggggctcagccctgagctagtgtcacctgcacagggccctctgaggctcagccctgagctggcgtcacctgtgcagggccctctggggctcagccctgagctggcctcacctgggttccccaccccgggctctcctgccctgccctcctgcccgccctccctcctgcctgcgcagctccttccctaggcacctctgtgctgcatcccaccagcctgagcaagacgccctctcggggcctgtgccgcactagcctccctctcctctgtccccatagctggtttttcccaccaatcctcacctaacagttactttacaattaaactcaaagcaagctcttctcctcagcttggggcagccattggcctctgtctcgttttgggaaaccaaggtcaggaggccgttgcagacataaatctcggcgactcggccccgtctcctgagggtcctgctggtgaccggcctggaccttggccctacagccctggaggccgctgctgaccagcactgaccccgacctcagagagtactcgcaggggcgctggctgcactcaagaccctcgagattaacggtgctaaccccgtctgctcctccctcccgcagagactggggcctggactggacatgagagccccttggtgccacagagggctgtgtcttactagaaacaacgcaaacctctccttcctcagaatagtgatgtgttcgacgttttatcaaaggccccctttctatgttcatgttagttttgctccttctgtgtttttttctgaaccatatccatgttgctgacttttccaaataaaggttttcactcctctc

SEQ ID NO: 38 - DNA, 577 bp, Artificial Sequence
  3′UTR-013 (Calr; calreticulin UTR)
agaggcctgcctccagggctggactgaggcctgagcgctcctgccgcagagctggccgcgccaaataatgtctctgtgagactcgagaactttcatttttttccaggctggttcggatttggggtggattttggttttgttcccctcctccactctcccccaccccctccccgcccttttttttttttttttttaaactggtattttatctttgattctccttcagccctcacccctggttctcatctttcttgatcaacatcttttcttgcctctgtccccttctctcatctcttagctcccctccaacctggggggcagtggtgtggagaagccacaggcctgagatttcatctgctctccttcctggagcccagaggagggcagcagaagggggtggtgtctccaaccccccagcactgaggaagaacggggctcttctcatttcacccctccctttctcccctgcccccaggactgggccacttctgggtggggcagtgggtcccagattggctcacactgagaatgtaagaactacaaacaaaatttctattaaattaaattttgtgtctcc

SEQ ID NO: 39 - DNA, 2212 bp, Artificial Sequence
  3′UTR-014 (Col1a1; collagen, type I, alpha 1 UTR)
ctccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaacataatccgcagtggcctcctaatttccttctttggagttgggggaggggtagacatggggaaggggctttggggtgatgggcttgccttccattcctgccctttccctccccactattctcttctagatccctccataaccccactcccctttctctcacccttcttataccgcaaacctttctacttcctctttcattttctattcttgcaatttccttgcaccttttccaaatcctcttctcccctgcaataccatacaggcaatccacgtgcacaacacacacacacactcttcacatctggggttgtccaaacctcatacccactccccttcaagcccatccactctccaccccctggatgccctgcacttggtggcggtgggatgctcatggatactgggagggtgaggggagtggaacccgtgaggaggacctgggggcctctccttgaactgacatgaagggtcatctggcctctgctcccttctcacccacgctgacctcctgccgaaggagcaacgcaacaggagaggggtctgctgagcctggcgagggtctgggagggaccaggaggaaggcgtgctccctgctcgctgtcctggccctgggggagtgagggagacagacacctgggagagctgtggggaaggcactcgcaccgtgctcttgggaaggaaggagacctggccctgctcaccacggactgggtgcctcgacctcctgaatccccagaacacaacccccctgggctggggtggtctggggaaccatcgtgcccccgcctcccgcctactcctttttaagctt

SEQ ID NO: 40 - DNA, 729 bp, Artificial Sequence
  3′UTR-015 (Plod1; procollagen-lysine, 2-oxoglutarate 5-dioxygenase 1 UTR)
ttggccaggcctgaccctcttggacctttcttctttgccgacaaccactgcccagcagcctctgggacctcggggtcccagggaacccagtccagcctcctggctgttgacttcccattgctcttggagccaccaatcaaagagattcaaagagattcctgcaggccagaggcggaacacacctttatggctggggctctccgtggtgttctggacccagcccctggagacaccattcacttttactgctttgtagtgactcgtgctctccaacctgtcttcctgaaaaaccaaggcccccttcccccacctcttccatggggtgagacttgagcagaacaggggcttccccaagttgcccagaaagactgtctgggtgagaagccatggccagagcttctcccaggcacaggtgttgcaccagggacttctgcttcaagttttggggtaaagacacctggatcagactccaagggctgccctgagtctgggacttctgcctccatggctggtcatgagagcaaaccgtagtcccctggagacagcgactccagagaacctcttgggagacagaagaggcatctgtgcacagctcgatcttctacttgcctgtggggaggggagtgacaggtccacacaccacactgggtcaccctgtcctggatgcctctgaagagagggacagaccgtcagaaactggagagtttctattaaaggtcatttaaacca

SEQ ID NO: 41 - DNA, 847 bp, Artificial Sequence
  3′UTR-016 (Nucb1; nucleobindin 1 UTR)
tcctccgggaccccagccctcaggattcctgatgctccaaggcgactgatgggcgctggatgaagtggcacagtcagcttccctgggggctggtgtcatgttgggctcctggggcgggggcacggcctggcatttcacgcattgctgccaccccaggtccacctgtctccactttcacagcctccaagtctgtggctcttcccttctgtcctccgaggggcttgccttctctcgtgtccagtgaggtgctcagtgatcggcttaacttagagaagcccgccccctccccttctccgtctgtcccaagagggtctgctctgagcctgcgttcctaggtggctcggcctcagctgcctgggttgtggccgccctagcatcctgtatgcccacagctactggaatccccgctgctgctccgggccaagcttctggttgattaatgagggcatggggtggtccctcaagaccttcccctaccttttgtggaaccagtgatgcctcaaagacagtgtcccctccacagctgggtgccaggggcaggggatcctcagtatagccggtgaaccctgataccaggagcctgggcctccctgaacccctggcttccagccatctcatcgccagcctcctcctggacctcttggcccccagccccttccccacacagccccagaagggtcccagagctgaccccactccaggacctaggcccagcccctcagcctcatctggagcccctgaagaccagtcccacccacctttctggcctcatctgacactgctccgcatcctgctgtgtgtcctgttccatgttccggttccatccaaatacactttctggaacaaa

SEQ ID NO: 42 - DNA, 110 bp, Artificial Sequence
  3′UTR-017 (alpha-globin)
gctggagcctcggtggccatgcttcttgccccttgggcctccccccagcccctcctccccttcctgcacccgtacccccgtggtctttgaataaagtctgagtgggcggc

SEQ ID NO: 43 - DNA, 119 bp, Artificial Sequence
  3′UTR-018
tgataataggctggagcctcggtggccatgcttcttgccccttgggcctccccccagcccctcctccccttcctgcacccgtacccccgtggtctttgaataaagtctgagtgggcggc

SEQ ID NO: 44 - protein, 239 aa, Artificial Sequence
  GFP Sequence
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 45 - RNA, 1838 nt, Artificial Sequence
  Firefly luciferase with 5′-UTR, 3′-UTR and miR-122 binding site
gggaaauaagagagaaaagaagaguaagaagaaauauaagagccaccauggaagaugcgaagaacaucaagaagggaccugccccguuuuacccuuuggaggacgguacagcaggagaacagcuccacaaggcgaugaaacgcuacgcccugguccccggaacgauugcguuuaccgaugcacauauugagguagacaucacauacgcagaauacuucgaaaugucggugaggcuggcggaagcgaugaagagauauggucuuaacacuaaucaccgcaucgugguguguucggagaacucauugcaguuuuucaugccgguccuuggagcacuuuucaucggggucgcagucgcgccagcgaacgacaucuacaaugagcgggaacucuugaauagcaugggaaucucccagccgacggucguguuugucuccaaaaaggggcugcagaaaauccucaacgugcagaagaagcuccccauuauucaaaagaucaucauuauggauagcaagacagauuaccaaggguuccagucgauguauaccuuugugacaucgcauuugccgccaggguuuaacgaguaugacuucguccccgagucauuugacagagauaaaaccaucgcgcugauuaugaauuccucggguagcaccgguuugccaaaggggguggcguugccccaccgcacugcuugugugcgguucucgcacgcuagggauccuaucuuugguaaucagaucauucccgacacagcaauccuguccgugguaccuuuucaucacgguuuuggcauguucacgacucucggcuauuugauuugcgguuucagggucguacuuauguaucgguucgaggaagaacuguuuuugagauccuugcaagauuacaagauccagucggcccuccuugugccaacgcuuuucucauucuuugcgaaaucgacacuuauugauaaguaugaccuuuccaaucugcaugagauugccucagggggagcgccgcuuagcaaggaagucggggaggcaguggccaagcgcuuccaccuucccggaauucggcagggauacgggcucacggagacaacauccgcgauccuuaucacgcccgagggugacgauaagccgggagccgucggaaaagugguccccuucuuugaagccaaggucguagaccucgacacgggaaaaacccucggagugaaccagaggggcgagcucugcgugagagggccgaugaucaugucagguuacgugaauaacccugaagcgacgaaugcgcugaucgacaaggaugggugguugcauucgggagacauugccuauugggaugaggaugagcacuucuuuaucguagaucgacuuaagagcuugaucaaauacaaaggcuaucagguagcgccugccgagcucgagucaauccugcuccagcaccccaacauuuucgacgccggaguggccggguugcccgaugacgacgcgggugagcugccagcggccgugguaguccucgaacaugggaaaacaaugaccgaaaaggagaucguggacuacguagcaucacaagugacgacugcgaagaaacugaggggagggguagucuuuguggacgaggucccgaaaggcuugacugggaagcuugacgcucgcaaaauccgggaaauccugauuaaggcaaagaaaggcgggaaaaucgcugucugauaauaggcuggagccucgguggccaugcuucuugccccuugggccuccccccagccccuccuccccuuccugcacccguaccccccaaacaccauugucacacuccaguggucuuugaauaaagucugagugggcggc

(19) United States
(12) Patent Application Publication
(10) Pub. No.: US 2019/0167811 A1
(43) Pub. Date: Jun. 6, 2019

(54) LIPID COMPOSITIONS AND THEIR USES FOR INTRATUMORAL POLYNUCLEOTIDE DELIVERY

(71) Applicant: ModernaTX, Inc., Cambridge, MA (US)

(72) Inventors: Kerry BENENATO, Sudbury, MA (US); Bo YING, Arlington, MA (US); Luis BRITO, Concord, MA (US)

(21) Appl. No.: 16/092,612

(22) PCT Filed: Apr. 13, 2017
(86) PCT No.: PCT/US2017/027492

§ 371 Date: Oct. 10, 2018

Related U.S. Application Data
(60) Provisional application No. 62/415,395, filed on Oct. 31, 2016.
(60) Provisional application No. 62/338,139, filed on May 18, 2016.
(60) Provisional application No. 62/338,126, filed on May 18, 2016.
(60) Provisional application No. 62/321,933, filed on Apr. 13, 2016.

(51) Int. Cl. A61K 48/00; A61P 35/00; A61K 9/00; A61K 47/24; A61K 47/28; A61K 9/127; A61K 47/44

(52) U.S. Cl. CPC A61K 48/0033; A61P 35/00; A61K 9/0019; A61K 47/44; A61K 47/28; A61K 9/1272; A61K 47/24

(57) ABSTRACT

The present application provides a composition comprising (1) a lipid composition comprising an ionizable amino lipid and a quaternary amine compound and (2) a polynucleotide. The present application also provides a composition comprising (1) a lipid composition comprising an asymmetric phospholipid, an ionizable amino lipid, and optionally a quaternary amine compound and (2) a polynucleotide, wherein the composition is formulated for intratumoral delivery of the polynucleotide. The present application further provides pharmaceutical compositions for intratumoral delivery comprising (1) a lipid composition comprising a compound of formula (I) and (2) therapeutic agent or a polynucleotide encoding the therapeutic agent, e.g., an mRNA encoding a therapeutic protein or a fragment thereof. Further provided is a method of increasing retention of a polynucleotide in a tumor tissue by using such a composition.

### BACKGROUND

Lipid-based nanoparticles have been used to deliver therapeutic agents such as siRNA and mRNA to the target cells in a subject. Lipid nanoparticles are multiple components systems, typically comprising a lipid composition containing one or more lipids, e.g., phospholipids, sterol, PEG-lipid conjugates, etc. There is, however, limited success in delivering a therapeutic agent to target tissues specifically and efficiently. The effective targeted delivery of biologically active substances such as small molecule drugs, proteins, and nucleic acids represents a continuing medical challenge.

Some of the problems with the known lipid nanoparticles include lack of stability, specificity, and low activity. In particular, the delivery of nucleic acids to cells is made difficult by the relative instability and low cell permeability of such species. Thus, there exists a need to develop compositions and methods to facilitate the delivery of therapeutic and/or prophylactics such as nucleic acids to cells.

Intratumoral delivery is an attractive alternative to systemic administration.

However, when lipid nanoparticles are administered intratumorally, the nucleic acids or other therapeutic agents encapsulated in the lipid nanoparticles can leak to peritumoral tissue or to off-target tissue (e.g., liver). Accordingly, there is a need to develop compositions and methods to facilitate the intratumoral delivery of therapeutic agents wherein the expression and retention of the therapeutic agent in the tumoral tissue is increased, and wherein the leakage of the therapeutic agent to surround tissue or to other organs such as liver is decreased.

Lipid nanoparticles generally include one or more ionizable lipids, phospholipids, structural lipids (e.g., sterols), PEG-lipids, and other components. Though a variety of such lipid-containing nanoparticle compositions have been demonstrated, improvements in safety, efficacy, and specificity are still lacking.

### BRIEF SUMMARY

The present application provides a composition comprising (1) a lipid composition comprising an ionizable amino lipid and a quaternary amine compound and (2) a polynucleotide. In one embodiment, the amount of the quaternary amine compound ranges from about 0.01 mole % to about 20 mole % in the lipid composition. In another embodiment, the mole ratio of the ionizable amino lipid to the quaternary amine compound is about 100:1 to about 2.5:1.

The present application also provides a composition comprising (1) a lipid composition comprising an asymmetric phospholipid, an ionizable amino lipid, and optionally a quaternary amine compound and (2) a polynucleotide, wherein the composition is formulated for intratumoral delivery of the polynucleotide.

In another aspect, the present application provides a lipid composition (e.g., a lipid nanoparticle (LNP)) comprising (1) an ionizable amino lipid, (2) a quaternary amine compound, (3) optionally a helper lipid, (4) optionally a sterol, and (5) optionally a lipid conjugate.

In another aspect, the present application provides a lipid composition (e.g., a lipid nanoparticle (LNP)) comprising (1) an asymmetric phospholipid, (2) an ionizable amino lipid, (3) optionally a quaternary amine compound, (4) optionally a sterol, and (5) optionally a lipid conjugate.

In exemplary embodiments, the lipid composition (e.g., LNP) encapsulates a polynucleotide.

In some embodiments, a phospholipid is a glycerophospholipid, a phosphosphingolipid, or any combination thereof. A phospholipid can be symmetric or asymmetric.

Symmetric phospholipids can be selected from the non-limiting group consisting of 1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1,2-di-O-octadecenyl-sn-glycero-3-phosphocholine (18:0 Diether PC),
1,2-dilinolenoyl-sn-glycero-3-phosphocholine (DLnPC),
1,2-diarachidonoyl-sn-glycero-3-phosphocholine (DAPC),
1,2-didocosahexaenoyl-sn-glycero-3-phosphocholine (DHAPC),
1,2-dioleoyl-sn-glycero-3-phosphoethanolamine (DOPE),
1,2-diphytanoyl-sn-glycero-3-phosphoethanolamine (4ME 16:0 PE),
1,2-distearoyl-sn-glycero-3-phosphoethanolamine (DSPE),
1,2-dilinoleoyl-sn-glycero-3-phosphoethanolamine (DLPE),
1,2-dilinolenoyl-sn-glycero-3-phosphoethanolamine (DLnPE),
1,2-diarachidonoyl-sn-glycero-3-phosphoethanolamine (DAPE),
1,2-didocosahexaenoyl-sn-glycero-3-phosphoethanolamine (DHAPE),
1,2-dioleoyl-sn-glycero-3-phospho-rac-(1-glycerol)sodium salt (DOPG), and any combination thereof. In one embodiment, the symmetric phospholipid is DSPC.

Asymmetric phospholipids can be selected from the non-limiting group consisting of 1-myristoyl-2-palmitoyl-sn-glycero-3-phosphocholine (14:0-16:0 PC, MPPC),
1-myristoyl-2-stearoyl-sn-glycero-3-phosphocholine (14:0-18:0 PC, MSPC),
1-palmitoyl-2-myristoyl-sn-glycero-3-phosphocholine (16:0-14:0 PC, PMPC),
1-palmitoyl-2-stearoyl-sn-glycero-3-phosphocholine (16:0-18:0 PC, PSPC),
1-stearoyl-2-myristoyl-sn-glycero-3-phosphocholine (18:0-14:0 PC, SMPC),
1-stearoyl-2-palmitoyl-sn-glycero-3-phosphocholine (18:0-16:0 PC, SPPC), and any combination thereof. In one embodiment, the asymmetric phospholipid is MSPC.

In some embodiments, the ionizable amino lipid comprises two different tail groups. In one embodiment, the ionizable amino lipid comprises at least one tail group that is branched. In some embodiments, the ionizable amino lipid is selected from the group consisting of DLin-MC3-DMA (MC3), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof. In one embodiment, the ionizable amino lipid is MC3.

In some embodiments, the ionizable amino lipid is selected from the group consisting of (13Z,16Z)—N,N-dimethyl-3-nonyldocosa-13,16-dien-1-amine (L608), (20Z,23Z)—N,N-dimethylnonacosa-20,23-dien-10-amine, (17Z,20Z)—N,N-dimemylhexacosa-17,20-dien-9-amine, (16Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimethyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, (11E,20Z,23Z)—N,N-dimethylnonacosa-11,20,2-trien-10-amine, and any combination thereof.

In some embodiments, the ionizable amino lipid can be the compounds disclosed in International Publication No. WO 2015/199952 A1, hereby incorporated by reference in its entirety. In some embodiments, the ionizable amino lipid is

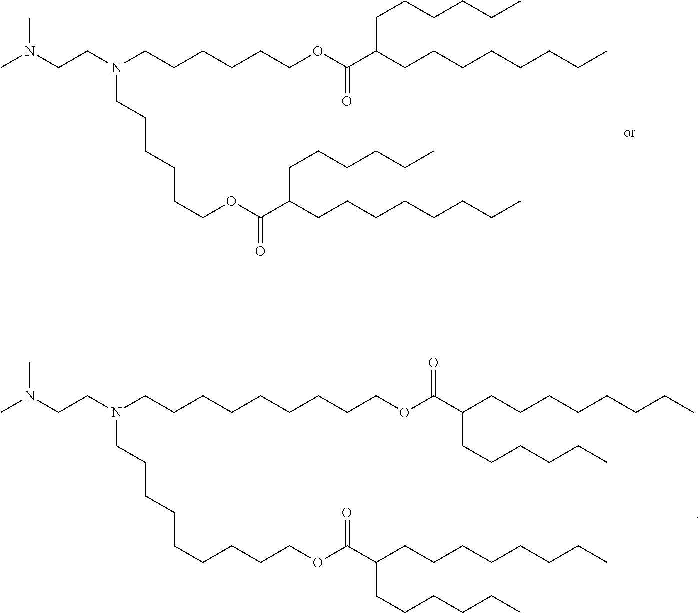

In some embodiment, the ionizable amino lipid is a compound having the formula (I)

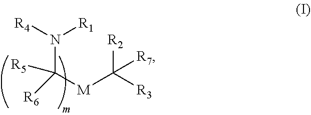

wherein

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof, wherein alkyl and alkenyl groups may be linear or branched,

provided when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In one example, the ionizable amino lipid is Compound 18:

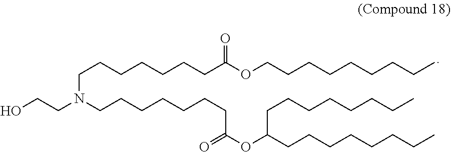 [SMILES: CCCCCCCCCOC(=O)CCCCCCCN(CCO)CCCCCCCC(=O)OC(CCCCCCCC)CCCCCCCC]

In one embodiment, the quaternary amine compound is selected from the group consisting of 1,2-dioleoyl-3-Trimethylammonium-propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), N,N-distearyl-N,N-dimethylammonium bromide (DDAB), N-(1,2-dimyristyloxyprop-3-yl)-N,N-dimethyl-N-hydroxyethyl ammonium bromide (DMRIE), N-(1,2-dioleoyloxyprop-3-yl)-N,N-dimethyl-N-hydroxyethyl ammonium bromide (DORIE), N,N-dioleyl-N,N-dimethylammonium chloride (DODAC), 1,2-dilauroyl-sn-glycero-3-ethylphosphocholine (DLePC), 1,2-distearoyl-3-trimethylammonium-propane (DSTAP), 1,2-dipalmitoyl-3-trimethylammonium-propane (DPTAP), 1,2-dilinoleoyl-3-trimethylammonium-propane (DLTAP), 1,2-dimyristoyl-3-trimethylammonium-propane (DMTAP), 1,2-distearoyl-sn-glycero-3-ethylphosphocholine (DSePC), 1,2-dipalmitoyl-sn-glycero-3-ethylphosphocholine (DPePC), 1,2-dimyristoyl-sn-glycero-3-ethylphosphocholine (DMePC), 1,2-dioleoyl-sn-glycero-3-ethylphosphocholine (DOePC), 1,2-di-(9Z-tetradecenoyl)-sn-glycero-3-ethylphosphocholine (14:1 EPC), 1-palmitoyl-2-oleoyl-sn-glycero-3-ethylphosphocholine (16:0-18:1 EPC), and any combination thereof. In one embodiment, the quaternary amine compound is DOTAP.

In one embodiment, the amount of the quaternary amine compound in the lipid composition ranges from about 0.5 to about 20.0 mole %, from about 0.5 to about 15.0 mole %, from about 0.5 to about 10.0 mole %, from about 1.0 to about 20.0 mole %, from about 1.0 to about 15.0 mole %, from about 1.0 to about 10.0 mole %, from about 2.0 to about 20.0 mole %, from about 2.0 to about 15.0 mole %, from about 2.0 to about 10.0 mole %, from about 3.0 to about 20.0 mole %, from about 3.0 to about 15.0 mole %, from about 3.0 to about 10.0 mole %, from about 4.0 to about 20.0 mole %, from about 4.0 to about 15.0 mole %, from about 4.0 to about 10.0 mole %, from about 5.0 to about 20.0 mole %, from about 5.0 to about 15.0 mole %, from about 5.0 to about 10.0 mole %, from about 6.0 to about 20.0 mole %, from about 6.0 to about 15.0 mole %, from about 6.0 to about 10.0 mole %, from about 7.0 to about 20.0 mole %, from about 7.0 to about 15.0 mole %, from about 7.0 to about 10.0 mole %, from about 8.0 to about 20.0 mole %, from about 8.0 to about 15.0 mole %, from about 8.0 to about 10.0 mole %, from about 9.0 to about 20.0 mole %, from about 9.0 to about 15.0 mole %, from about 9.0 to about 10.0 mole %, about 5.0 mole %, about 10.0 mole %, about 15.0 mole %, or about 20.0 mole %. In one embodiment, the amount of the quaternary amine compound in the lipid composition ranges from about 5 to about 10 mole %. In another embodiment, the amount of the quaternary amine compound in the lipid composition is about 5 mole %.

In some embodiments, the lipid composition further comprises a sterol. In one embodiment, the sterol is cholesterol.

In some embodiments, the lipid composition further comprises a PEG-lipid. In one embodiment, the PEG-lipid is PEG-1,2-dimyristoyl-sn-glycerol methoxypolyethylene glycol (PEG-DMG) or 1,2-distearoyl-sn-glycero-3-phosphoethanolamine-N-[amino(polyethylene glycol)] (PEG-DSPE).

In some embodiments, the net positive charge of the lipid composition is increased compared to the net positive charge of a corresponding lipid composition without the quaternary amine compound.

In one embodiment, the present application relates to a composition comprising (1) a lipid composition comprising MC3, L608, or Compound 18; a phospholipid; a sterol; a PEG-lipid; DOTAP; and (2) a polynucleotide. In one embodiment, the amount of DOTAP ranges from about 0.01 to about 20 mole % in the lipid composition. In another embodiment, the present application relates to a lipid composition comprising (i) MC3, L608, or Compound 18; (ii) a phospholipid; (iii) a sterol; (iv) a PEG-lipid; and (v) a quaternary amine compound which is DOTAP, DOTMA, DLePC, or DDAB.

In one embodiment, the amount of MC3, L608, or Compound 18 ranges from about 30 to about 70 mole % in the lipid composition. In one embodiment, the amount of the phospholipid ranges from about 1 to about 20 mole % in the lipid composition. In one embodiment, the amount of the sterol ranges from about 20 to about 60 mole % in the lipid composition. In one embodiment, the amount of PEG-lipid ranges from about 0.1 to about 5 mole % in the lipid composition.

In another embodiment, the present application relates to a composition comprising (1) a lipid composition comprising about 50 mole % of MC3; about 10 mole % of DSPC or MSPC; about 33.5 mole % of cholesterol; about 1.5 mole % of PEG-DMG (e.g., PEG₂ₖ-DMG); about 5 mole % of DOTAP; and (2) a polynucleotide.

In another embodiment, the present application relates to a composition comprising (1) a lipid composition comprising about 50 mole % of Compound 18; about 10 mole % of DSPC or MSPC; about 33.5 mole % of cholesterol; about 1.5 mole % of PEG₂ₖ-DMG; about 5 mole % of DOTAP; and (2) a polynucleotide.

In another embodiment, the present application relates to a composition comprising (1) a lipid composition comprising about 50 mole % of MC3; about 10 mole % of DSPC or MSPC; about 28.5 mole % of cholesterol; about 1.5 mole % of PEG-DMG (e.g., PEG₂ₖ-DMG); about 10 mole % of DOTAP; and (2) a polynucleotide.

In another embodiment, the present application relates to a composition comprising (1) a lipid composition comprising about 50 mole % of MC3; about 10 mole % of DSPC or MSPC; about 23.5 mole % of cholesterol; about 1.5 mole % of PEG-DMG (e.g., PEG₂ₖ-DMG); about 15 mole % of DOTAP; and (2) a polynucleotide.

In one embodiment, the present application relates to a lipid composition comprising about 50 mole % of MC3; about 10 mole % of DSPC or MSPC; about 33.5 mole % of cholesterol; about 1.5 mole % of PEG-DMG (e.g., PEG₂ₖ-DMG); and about 5 mole % of DOTAP.

In another embodiment, the present application relates to a lipid composition comprising about 50 mole % of Compound 18; about 10 mole % of DSPC or MSPC; about 33.5 mole % of cholesterol; about 1.5 mole % of PEG₂ₖ-DMG; and about 5 mole % of DOTAP.

In one embodiment, the present application relates to a composition comprising (1) a lipid composition comprising MC3, L608, or Compound 18; MSPC; a sterol; a PEG-lipid; and (2) a polynucleotide, wherein the composition is formulated for intratumoral delivery of the polynucleotide.

In one embodiment, the present application relates to a composition comprising (1) a lipid composition comprising about 50 mole % of MC3; about 10 mole % of MSPC; about 38.5 mole % of cholesterol; about 1.5 mole % of PEG-DMG (e.g., PEG₂ₖ-DMG); and (2) a polynucleotide.

In one embodiment, the present application relates to a composition comprising (1) a lipid composition comprising about 50 mole % of MC3; about 10 mole % of MSPC; about 39.5 mole % of cholesterol; about 0.5 mole % of PEG-DMG (e.g., PEG₂ₖ-DMG); and (2) a polynucleotide. In another embodiment, the present application relates to a composition comprising (1) a lipid composition comprising about 50 mole % of Compound 18; about 10 mole % of MSPC; about 38.5 mole % of cholesterol; about 1.5 mole % of PEG₂ₖ-DMG; and (2) a polynucleotide.

In some bodiments, the present application provides a lipid composition comprising Compound 18; an asymmetric phospholipid; a sterol; and a PEG-lipid. In one embodiment, the present application provides a lipid composition about 50 mole % of Compound 18; about 10 mole % of MSPC; about 38.5 mole % of cholesterol; and about 1.5 mole % of PEG₂ₖ-DMG.

In one embodiment, the present application relates to a composition comprising (1) a lipid composition comprising about 50 mole % of MC3; about 10 mole % of MSPC; about 35 mole % of cholesterol; about 5 mole % of PEG-DMG (e.g., PEG₂ₖ-DMG); and (2) a polynucleotide.

In one embodiment, the present application relates to a composition comprising (1) a lipid composition comprising about 50 mole % of Compound 18; about 10 mole % of MSPC; about 35 mole % of cholesterol; about 5 mole % of PEG-DMG (e.g., PEG₂ₖ-DMG); and (2) a polynucleotide.

In one embodiment, the present application relates to a composition comprising (1) a lipid composition comprising about 50 mole % of MC3; about 10 mole % of MSPC; about 30 mole % of cholesterol; about 10 mole % of PEG-DMG (e.g., PEG₂ₖ-DMG); and (2) a polynucleotide.

In one embodiment, the present application relates to a composition comprising (1) a lipid composition comprising about 50 mole % of Compound 18; about 10 mole % of MSPC; about 30 mole % of cholesterol; about 10 mole % of PEG-DMG (e.g., PEG₂ₖ-DMG); and (2) a polynucleotide.

The present disclosure also provides a pharmaceutical composition for intratumoral delivery comprising:

(a) a lipid composition comprising:
(i) a compound having the formula (I)

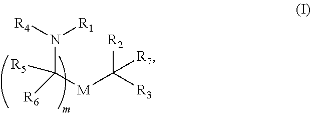

wherein

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof, wherein alkyl and alkenyl groups can be linear or branched.

In certain embodiments, a subset of compounds of formula (I) includes those of Formula (IA):

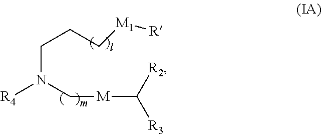

or a salt or stereoisomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which n is 1, 2, 3, 4, or 5 and Q is OH, —NHC(S)N(R)₂, or —NHC(O)N(R)₂; M and M′ are independently selected from —C(O)O—, —OC(O)—, —C(O)N(R′)—, —P(O)(OR′)O—, an aryl group, and a heteroaryl group; and

R₂ and R₃ are independently selected from the group consisting of H, C₁₋₁₄ alkyl, and C₂₋₁₄ alkenyl.

In certain embodiments, a subset of compounds of formula (I) includes those of Formula (II):

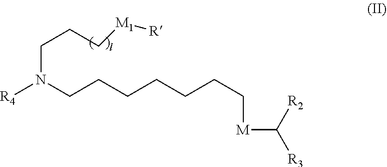

or a salt or stereoisomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, or —NHC(O)N(R)₂; M and M′ are independently selected from —C(O)O—, —OC(O)—, —C(O)N(R′)—, —P(O)(OR′)O—, an aryl group, and a heteroaryl group; and

R₂ and R₃ are independently selected from the group consisting of H, C₁₋₁₄ alkyl, and C₂₋₁₄ alkenyl.

In some embodiments, a subset of compounds of formula (I) is of the formula (IIa),

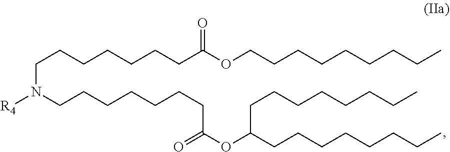

or a salt thereof, wherein R₄ is as described above.

In some embodiments, a subset of compounds of formula (I) is of the formula (IIb),

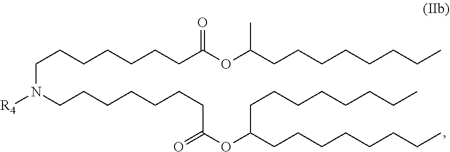

or a salt thereof, wherein R₄ is as described above.

In some embodiments, a subset of compounds of formula (I) is of the formula (IIc),

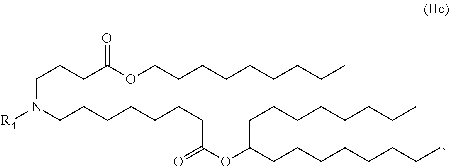

or a salt thereof, wherein R₄ is as described above.

In some embodiments, a subset of compounds of formula (I) is of the formula (IIe):

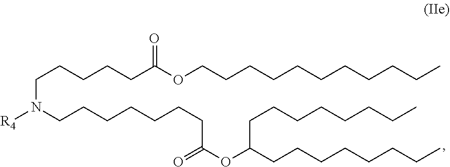

or a salt thereof, wherein R₄ is as described above.

In some embodiments, a subset of compounds of formula (I) is of the formula (IId),

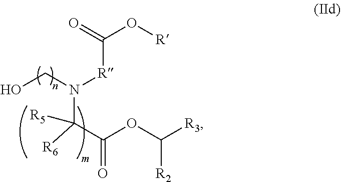

or a salt thereof, wherein R₂ and R₃ are independently selected from the group consisting of C₅₋₁₄ alkyl and C₅₋₁₄ alkenyl, n is selected from 2, 3, and 4, and R′, R″, R₅, R₆ and m are as defined above.

In some embodiments, the compound of formula (I) is Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, Compound 16, Compound 17, Compound 18, Compound 19, Compound 20, Compound 21, Compound 22, Compound 23, Compound 24, Compound 25, Compound 26, Compound 27, Compound 28, Compound 29, Compound 30, Compound 31, Compound 32, Compound 33, Compound 34, Compound 35, Compound 36, Compound 37, Compound 38, Compound 39, Compound 40, Compound 41, Compound 42, Compound 43, Compound 44, Compound 45, Compound 46, Compound 47, Compound 48, Compound 49, Compound 50, Compound 51, Compound 52, Compound 53, Compound 54, Compound 55, Compound 56, Compound 57, Compound 58, Compound 59, Compound 60, Compound 61, Compound 62, Compound 63, Compound 64, Compound 65, Compound 66, Compound 67, Compound 68, Compound 69, Compound 70, Compound 71, Compound 72, Compound 73, Compound 74, Compound 75, Compound 76, Compound 77, Compound 78, Compound 79, Compound 80, Compound 81, Compound 82, Compound 83, Compound 84, Compound 85, Compound 86, Compound 87, Compound 88, Compound 89, Compound 90, Compound 91, Compound 92, Compound 93, Compound 94, Compound 95, Compound 96, Compound 97, Compound 98, Compound 99, Compound 100, Compound 101, Compound 102, Compound 103, Compound 104, Compound 105, or Compound 106, Compound 107, Compound 108, Compound 109, Compound 110, Compound 111, Compound 112, Compound 113, Compound 114, Compound 115, Compound 116, Compound 117, Compound 118, Compound 119, Compound 120, Compound 121, Compound 122, Compound 123, Compound 124, Compound 125, Compound 126, Compound 127, Compound 128, Compound 129, Compound 130, Compound 131, Compound 132, Compound 133, Compound 134, Compound 135, Compound 136, Compound 137, Compound 138, Compound 139, Compound 140, Compound 141, Compound 142, Compound 143, Compound 144, Compound 145, Compound 146, or Compound 147, as defined below, or a combination thereof. In some embodiments, the compound of formula (I) is Compound 18.

In some embodiments, the lipid composition further comprises a phospholipid, which is a glycerophospholipid, a phosphosphingolipid, or any combination thereof. In some embodiments, the phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1,2-di-O-octadecenyl-sn-glycero-3-phosphocholine (18:0 Diether PC),
1,2-dilinolenoyl-sn-glycero-3-phosphocholine (DLnPC),
1,2-diarachidonoyl-sn-glycero-3-phosphocholine (DAPC),
1,2-didocosahexaenoyl-sn-glycero-3-phosphocholine (DHAPC),
1,2-dioleoyl-sn-glycero-3-phosphoethanolamine (DOPE),
1,2-diphytanoyl-sn-glycero-3-phosphoethanolamine (ME 16:0 PE),
1,2-distearoyl-sn-glycero-3-phosphoethanolamine (DSPE),
1,2-dilinoleoyl-sn-glycero-3-phosphoethanolamine (DLPE),
1,2-dilinolenoyl-sn-glycero-3-phosphoethanolamine (DLnPE),
1,2-diarachidonoyl-sn-glycero-3-phosphoethanolamine (DAPE),
1,2-didocosahexaenoyl-sn-glycero-3-phosphoethanolamine (DHAPE),
1,2-dioleoyl-sn-glycero-3-phospho-rac-(1-glycerol)sodium salt (DOPG), and any combination thereof.

In some the phospholipid is an asymmetric phospholipid. In some embodiments, the phospholipid is an asymmetric phospholipid selected from the group consisting of
1-myristoyl-2-palmitoyl-sn-glycero-3-phosphocholine (14:0-16:0 PC, MPPC),
1-myristoyl-2-stearoyl-sn-glycero-3-phosphocholine (14:0-18:0 PC, MSPC),
1-palmitoyl-2-myristoyl-sn-glycero-3-phosphocholine (16:0-14:0 PC, PMPC),
1-palmitoyl-2-stearoyl-sn-glycero-3-phosphocholine (16:0-18:0 PC, PSPC),
1-stearoyl-2-myristoyl-sn-glycero-3-phosphocholine (18:0-14:0 PC, SMPC),
1-stearoyl-2-palmitoyl-sn-glycero-3-phosphocholine (18:0-16:0 PC, SPPC), and any combination thereof.

In some embodiments, the asymmetric phospholipid is 1-myristoyl-2-stearoyl-sn-glycero-3-phosphocholine (MSPC).

In some embodiments, the lipid composition further comprises a quaternary amine compound. In some embodiments, the quaternary amine compound is selected from the group consisting of
1,2-dioleoyl-3-trimethylammonium-propane (DOTAP),
N-[1-(2,3-dioleoyloxy)propyl]-N,N,N-trimethylammonium chloride (DOTMA),
1-[2-(oleoyloxy)ethyl]-2-oleyl-3-(2-hydroxyethyl)imidazolinium chloride (DOTIM),
2,3-dioleyloxy-N-[2(sperminecarboxamido)ethyl]-N,N-dimethyl-1-propanaminium trifluoroacetate (DOSPA),
N,N-distearyl-N,N-dimethylammonium bromide (DDAB),
N-(1,2-dimyristyloxyprop-3-yl)-N,N-dimethyl-N-hydroxyethyl ammonium bromide (DMRIE),
N-(1,2-dioleoyloxyprop-3-yl)-N,N-dimethyl-N-hydroxyethyl ammonium bromide (DORIE),
N,N-dioleyl-N,N-dimethylammonium chloride (DODAC),
1,2-dilauroyl-sn-glycero-3-ethylphosphocholine (DLePC),
1,2-distearoyl-3-trimethylammonium-propane (DSTAP),
1,2-dipalmitoyl-3-trimethylammonium-propane (DPTAP),
1,2-dilinoleoyl-3-trimethylammonium-propane (DLTAP),
1,2-dimyristoyl-3-trimethylammonium-propane (DMTAP),
1,2-distearoyl-sn-glycero-3-ethylphosphocholine (DSePC),
1,2-dipalmitoyl-sn-glycero-3-ethylphosphocholine (DPePC),
1,2-dimyristoyl-sn-glycero-3-ethylphosphocholine (DMePC),
1,2-dioleoyl-sn-glycero-3-ethylphosphocholine (DOePC),
1,2-di-(9Z-tetradecenoyl)-sn-glycero-3-ethylphosphocholine (14:1 EPC),
1-palmitoyl-2-oleoyl-sn-glycero-3-ethylphosphocholine (16:0-18:1 EPC), and any combination thereof.

In some embodiments, the quaternary amine compound is 1,2-dioleoyl-3-trimethylammonium-propane (DOTAP).

In some embodiments, the lipid composition further comprises a structural lipid. In some embodiments, the structural lipid is a sterol. In some embodiments, the structural lipid is selected from the group consisting of cholesterol, fecosterol, sitosterol, ergosterol, campesterol, stigmasterol, brassicasterol, tomatidine, ursolic acid, alpha-tocopherol, and mixtures thereof. In some embodiments, the structural lipid is cholesterol.

In some embodiments, the lipid composition further comprises a polyethylene glycol (PEG)-lipid. In some embodiments, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the amount of compound of formula (I) in the lipid composition ranges from about 1 mol % to 99 mol % in the lipid composition. In some embodiments, the amount of compound of formula (I) ranges from about 30 mol % to about 70 mol % in the lipid composition. In some embodiments, the lipid composition comprises about 50 mol % of the compound of formula (I).

In some embodiments, the amount of the phospholipid ranges from about 1 mol % to about 20 mol % in the lipid composition. In some embodiments, the amount of the phospholipid is about 10 mol % in the lipid composition.

In some embodiments, the amount of the quaternary amine compound in the lipid composition ranges from about 5 mol % to about 10.0 mol %. In some embodiments, the amount of the quaternary amine compound in the lipid composition is about 5 mol %.

In some embodiments, the amount of the structural lipid ranges from about 20 mol % to about 60 mol % in the lipid composition. In some embodiments, the amount of the structural lipid in the composition is about 33.5 mol %.

In some embodiments, the amount of the PEG-lipid ranges from about 0.1 mol % to about 5.0 mol % in the lipid composition. In some embodiments, the amount of the PEG-lipid in the composition is about 1.5 mol %.

In some embodiments, the wt/wt ratio of the lipid composition to the polypeptide is from about 10:1 to about 60:1. In some embodiments, the wt/wt ratio of the lipid composition to the polypeptide is about 20:1. In some embodiments, the N:P ratio, i.e., the nitrogen to phosphorus ratio, is from about 2:1 to about 30:1. In some embodiments, the N:P ratio is about 5.67:1.

In one embodiment, the polynucleotide is selected from the group consisting of plasmid DNA, linear DNA selected from poly and oligo-nucleotides, chromosomal DNA, messenger RNA (mRNA), antisense DNA/RNA, siRNA, microRNA (miRNA), ribosomal RNA, oligonucleotide DNA (ODN) single and double strand, CpG imunostimulating sequence (ISS), locked nucleic acid (LNA), ribozyme, asymmetrical interfering RNA (aiRNA), dicer-substrate RNA (dsRNA), small hairpin RNA (shRNA), and any combination thereof.

In some embodiments, the mRNA comprises at least one chemically modified nucleobase.

In some embodiments, the at least one chemically modified nucleobase is selected from the group consisting of pseudouracil (ψ), 2-thiouracil (s2U), 4′-thiouracil, 5-methylcytosine, 2-thio-1-methyl-1-deaza-pseudouracil, 2-thio-1-methyl-pseudouracil, 2-thio-5-aza-uracil, 2-thio-dihydropseudouracil, 2-thio-dihydrouracil, 2-thio-pseudouracil, 4-methoxy-2-thio-pseudouracil, 4-methoxy-pseudouracil, 4-thio-1-methyl-pseudouracil, 4-thio-pseudouracil, 5-aza-uracil, dihydropseudouracil, 5-methyluracil, 5-methoxyuracil, 2′-O-methyl uracil, 1-methyl-pseudouracil (m1ψ), 1-ethyl-pseudouracil (e1ψ), 5-methoxy-uracil (mo5U), 5-methyl-cytosine (m5C), α-thio-guanine, α-thio-adenine, 5-cyano uracil, 4′-thio uracil, 7-deaza-adenine, 1-methyl-adenine (m1A), 2-methyl-adenine (m2A), N6-methyl-adenine (m6A), and 2,6-Diaminopurine, 1-methyl-inosine (m1I), wyosine (imG), methylwyosine (mimG), 7-deaza-guanine, 7-cyano-7-deaza-guanine (preQ0), 7-aminomethyl-7-deaza-guanine (preQ1), 7-methyl-guanine (m7G), 1-methyl-guanine (m1G), 8-oxo-guanine, 7-methyl-8-oxo-guanine, and two or more combinations thereof.

In some embodiments, the at least one chemically modified nucleobases is selected from the group consisting of pseudouracil (ψ), 1-methyl-pseudouracil (m1ψ), 1-ethyl-pseudouracil (e1ψ), 5-methylcytosine, 5-methoxyuracil, and any combination thereof.

In some embodiments, the nucleobases in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

In some embodiments, the chemically modified nucleobases are selected from the group consisting of uracil, adenine, cytosine, guanine, and any combination thereof.

In some embodiments, the uracils in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

In some embodiments, the adenines in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

In some embodiments, the cytosines in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

In some embodiments, the guanines in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

In some embodiments, the mRNA is sequence-optimized.

In one embodiment, the mRNA further comprises a 5′ UTR. In one embodiment, the 5′ UTR is sequence-optimized.

In one embodiment, the mRNA further comprises a 3′ UTR. In one embodiment, the 3′ UTR is sequence-optimized.

In one embodiment, the mRNA further comprises a 5′ terminal cap. In one embodiment, the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

In one embodiment, the mRNA further comprises a 3′ polyA tail.

In one embodiment, the mRNA is in vitro transcribed (IVT). In some embodiments, the mRNA is chimeric. In some embodiments, the mRNA is circular.

In some embodiments, wherein the polynucleotide encodes a polypeptide when administered intratumorally to a tumor tissue. In one embodiment, the polypeptide comprises a cytokine, a growth factor, a hormone, a cell surface receptor, an antibody or antigen binding portion thereof. In one embodiment, the polypeptide encodes a polypeptide which targets a cancer antigen.

In one embodiment, expression of the polypeptide in the tumor tissue is higher than expression of the polypeptide in a non-tumor tissue. In one embodiment, a ratio of the protein expression in the tumor tissue to that in the non-tumor tissue is at least about 200:1, at least about 250:1, at least about 300:1, at least about 350:1, at least about 400:1, at least about 450:1, at least about 500:1, at least about 600:1, at least about 700:1, at least about 800:1, at least about 900:1, or at least about 1000:1, when the protein expression is measured 24 hours post administration.

In one embodiment, a ratio of the protein expression in the tumor tissue to that in the non-tumor tissue is at least about 200:1, at least about 250:1, at least about 300:1, at least about 350:1, at least about 400:1, at least about 450:1, at least about 500:1, at least about 600:1, at least about 700:1, at least about 800:1, at least about 900:1, or at least about 1000:1, when the protein expression was measured 48 hours post administration.

In one embodiment, the composition of the present application, when administered intratumorally to a tumor tissue, increases retention of the polynucleotide in the tumor tissue as compared to a corresponding composition without the quaternary amine compound.

In one embodiment, the polynucleotide encodes a polypeptide when administered intratumorally to a tumor tissue, and the composition decreases expression of the polypeptide in a non-tumor tissue as compared to a corresponding composition without the quaternary amine compound.

In one embodiment, the present application provides a method of increasing retention of a polynucleotide in a tumor tissue of a subject, comprising administering intratumorally to the tumor tissue the composition disclosed herein compared to the retention of the polynucleotide in the tumor tissue by a corresponding composition without the quaternary amine compound. In one embodiment, the expression of the polypeptide in liver is decreased compared to the expression of the polypeptide in liver by a corresponding composition without the quaternary amine compound. In another embodiment, the polypeptide is expressed at the same level or at a higher level in the tumor tissue compared to the polypeptide expressed by a corresponding composition without the quaternary amine compound.

In one embodiment, the present application provides a method of increasing retention of a polynucleotide in a tumor tissue of a subject, comprising administering intratumorally to the tumor tissue the composition disclosed herein, wherein the retention of the polynucleotide in the tumor tissue is increased compared to the retention of the polynucleotide in the tumor tissue by a corresponding composition with a symmetric phospholipid.

In some embodiments, the expression of the polypeptide in a tumor and/or non-tumor tissue is measured at 6 hours, 8 hours, 10 hours, 12 hours, 14 hours, 16 hours, 18 hours, 20 hours, 22 hours, 24 hours, 36 hours, or 48 hours post administration.

In one embodiment, the present application provides a method of increasing expression of a polypeptide in a tumor tissue of a subject, comprising administering intratumorally to the tumor tissue the composition disclosed herein, wherein the expression level of the polypeptide in the tumor tissue is increased compared to the expression level of the polypeptide after administering a corresponding composition with a symmetric phospholipid.

The present application also relates to a method of producing a composition comprising a polynucleotide comprising formulating the polynucleotide in the lipid composition disclosed herein.

The present application further relates to a method of delivering a polynucleotide comprising formulating the polynucleotide in the lipid composition disclosed herein. In some embodiments, the present application provides a method of intratumorally delivering a polynucleotide by administering to a tumor tissue the composition disclosed herein.

In some embodiments, the polynucleotide formulated in a composition comprising a quaternary amine compound, e.g., DOTAP, encodes a polypeptide when administered intratumorally to a tumor tissue, and expression of the polypeptide is reduced in liver compared to a corresponding lipid composition without the quaternary amine compound, e.g., DOTAP.

### EMBODIMENTS

In addition to the various embodiments described herein, the present disclosure includes the following embodiments numbered E1 through E92. This list of embodiments is presented as an exemplary list and the application is not limited to these embodiments.

E1. A composition comprising:

(i) a lipid composition comprising
(1) an ionizable amino lipid; and
(2) a quaternary amine compound; and

(ii) a polynucleotide,

wherein the amount of the quaternary amine compound ranges from about 0.01 to about 20 mole % in the lipid composition.

E2. A composition comprising:

(i) a lipid composition comprising
(1) an ionizable amino lipid; and
(2) a quaternary amine compound; and

(ii) a polynucleotide,

wherein the mole ratio of the ionizable amino lipid to the quaternary amine compound is about 100:1 to about 2.5:1.

E3. The composition of E1 or E2, wherein the ionizable amino lipid is selected from the group consisting of DLin-MC3-DMA (MC3), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof.

E4. The composition of E1 or E2, wherein the ionizable amino lipid is selected from the group consisting of (13Z,16Z)—N,N-dimethyl-3-nonyldocosa-13,16-dien-1-amine (L608), (20Z,23Z)—N,N-dimethylnonacosa-20,23-dien-10-amine, (17Z,20Z)—N,N-dimemylhexacosa-17,20-dien-9-amine, (16Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, N,N-dimethyl-1-[(1S,2R)-2-octylcyclopropyl]eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, (11E,20Z,23Z)—N,N-dimethylnonacosa-11,20,2-trien-10-amine, and any combination thereof.

E5. The composition of any one of E1 or E2, wherein the ionizable amino lipid is selected from Compound 1 to Compound 147, and salts and stereoisomers thereof.

E6. The composition of any one of E1 or E2, wherein the ionizable amino lipid is selected from the group consisting of:

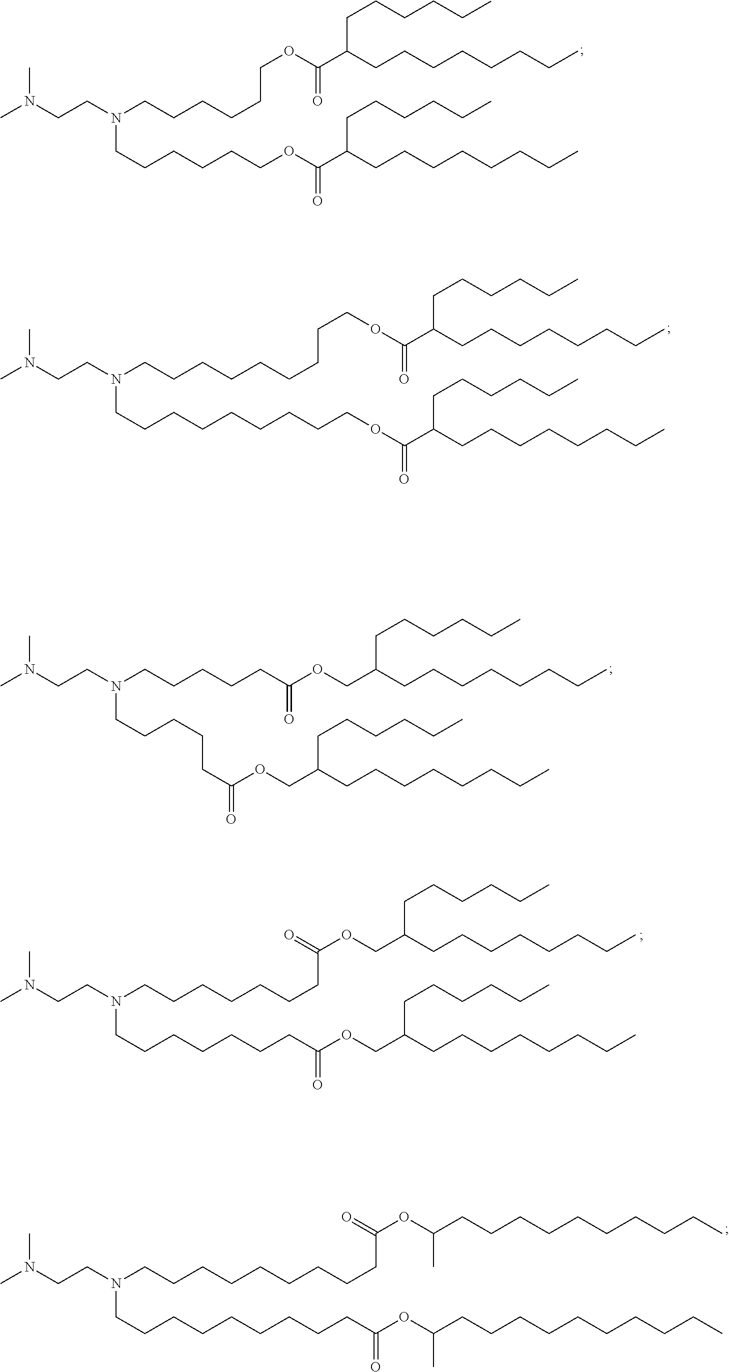

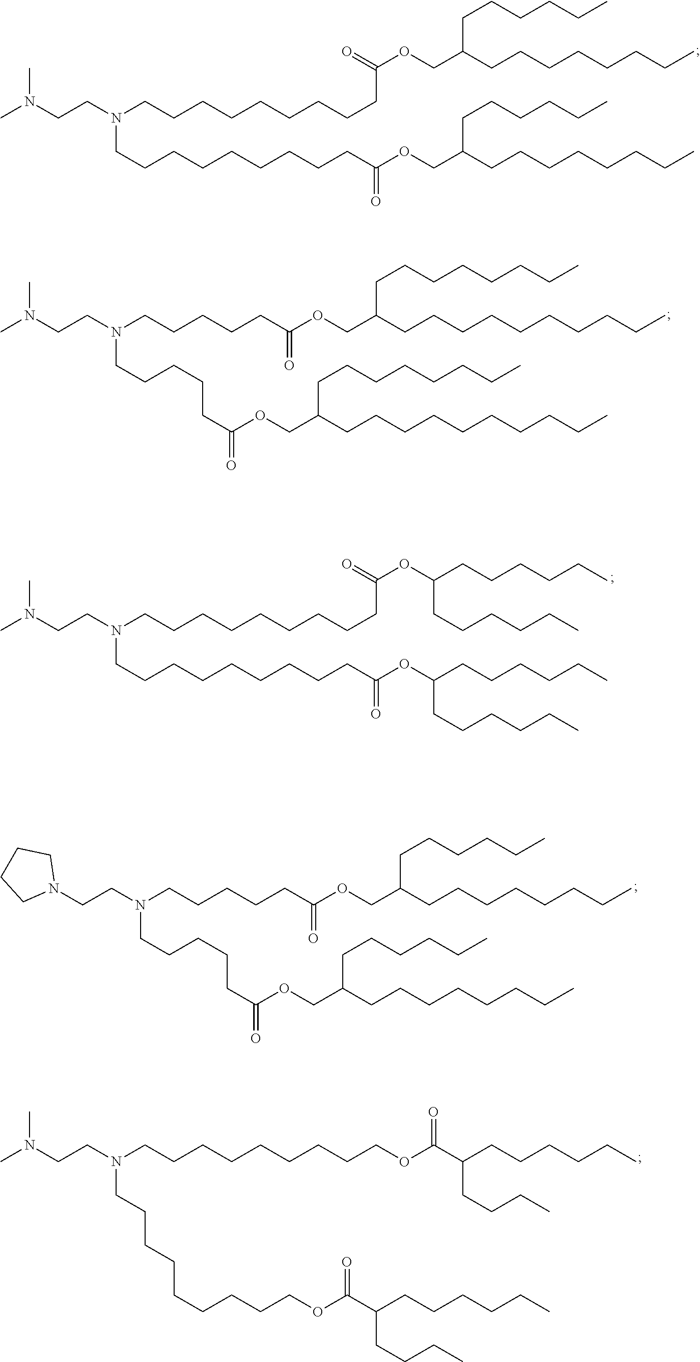

and any combination thereof.

E7. The composition of any one of E1 to E6, wherein the quaternary amine compound is selected from the group consisting of 1,2-dioleoyl-3-trimethylammonium-propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), N,N-distearyl-N,N-dimethylammonium bromide (DDAB), N-(1,2-dimyristyloxyprop-3-yl)-N,N-dimethyl-N-hydroxyethyl ammonium bromide (DMRIE), N-(1,2-dioleoyloxyprop-3-yl)-N,N-dimethyl-N-hydroxyethyl ammonium bromide (DORIE), N,N-dioleyl-N,N-dimethylammonium chloride (DODAC), 1,2-dilauroyl-sn-glycero-3-ethylphosphocholine (DLePC), 1,2-distearoyl-3-trimethylammonium-propane (DSTAP), 1,2-dipalmitoyl-3-trimethylammonium-propane (DPTAP), 1,2-dilinoleoyl-3-trimethylammonium-propane (DLTAP), 1,2-dimyristoyl-3-trimethylammonium-propane (DMTAP), 1,2-distearoyl-sn-glycero-3-ethylphosphocholine (DSePC), 1,2-dipalmitoyl-sn-glycero-3-ethylphosphocholine (DPePC), 1,2-dimyristoyl-sn-glycero-3-ethylphosphocholine (DMePC), 1,2-dioleoyl-sn-glycero-3-ethylphosphocholine (DOePC), 1,2-di-(9Z-tetradecenoyl)-sn-glycero-3-ethylphosphocholine (14:1 EPC), 1-palmitoyl-2-oleoyl-sn-glycero-3-ethylphosphocholine (16:0-18:1 EPC), and any combination thereof.

E8. The composition of any one of E1 to E7, wherein the amount of the quaternary amine compound in the lipid composition ranges from about 0.5 to about 20.0 mole %, from about 0.5 to about 15.0 mole %, from about 0.5 to about 10.0 mole %, from about 1.0 to about 20.0 mole %, from about 1.0 to about 15.0 mole %, from about 1.0 to about 10.0 mole %, from about 2.0 to about 20.0 mole %, from about 2.0 to about 15.0 mole %, from about 2.0 to about 10.0 mole %, from about 3.0 to about 20.0 mole %, from about 3.0 to about 15.0 mole %, from about 3.0 to about 10.0 mole %, from about 4.0 to about 20.0 mole %, from about 4.0 to about 15.0 mole %, from about 4.0 to about 10.0 mole %, from about 5.0 to about 20.0 mole %, from about 5.0 to about 15.0 mole %, from about 5.0 to about 10.0 mole %, from about 6.0 to about 20.0 mole %, from about 6.0 to about 15.0 mole %, from about 6.0 to about 10.0 mole %, from about 7.0 to about 20.0 mole %, from about 7.0 to about 15.0 mole %, from about 7.0 to about 10.0 mole %, from about 8.0 to about 20.0 mole %, from about 8.0 to about 15.0 mole %, from about 8.0 to about 10.0 mole %, from about 9.0 to about 20.0 mole %, from about 9.0 to about 15.0 mole %, from about 9.0 to about 10.0 mole %, about 5.0 mole %, about 10.0 mole %, about 15.0 mole %, or about 20.0 mole %.

E9. The composition of E8, wherein the amount of the quaternary amine compound in the lipid composition ranges from about 5 to about 10 mole %.

E10. The composition of E8, wherein the amount of the quaternary amine compound in the lipid composition is about 5 mole %.

E11. The composition of any one of E1 to E10, wherein the lipid composition further comprises a phospholipid.

E12. The composition of E11, wherein the phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1,2-di-O-octadecenyl-sn-glycero-3-phosphocholine (18:0 Diether PC),
1,2-dilinolenoyl-sn-glycero-3-phosphocholine (DLnPC),
1,2-diarachidonoyl-sn-glycero-3-phosphocholine (DAPC),
1,2-didocosahexaenoyl-sn-glycero-3-phosphocholine (DHAPC),
1,2-dioleoyl-sn-glycero-3-phosphoethanolamine (DOPE),
1,2-diphytanoyl-sn-glycero-3-phosphoethanolamine (4ME 16:0 PE),
1,2-distearoyl-sn-glycero-3-phosphoethanolamine (DSPE),
1,2-dilinoleoyl-sn-glycero-3-phosphoethanolamine (DLPE),
1,2-dilinolenoyl-sn-glycero-3-phosphoethanolamine (DLnPE),
1,2-diarachidonoyl-sn-glycero-3-phosphoethanolamine (DAPE),
1,2-didocosahexaenoyl-sn-glycero-3-phosphoethanolamine (DHAPE),
1,2-dioleoyl-sn-glycero-3-phospho-rac-(1-glycerol)sodium salt (DOPG), and any combination thereof.

E13. The composition of E11, wherein the phospholipid is selected from the group consisting of
1-myristoyl-2-palmitoyl-sn-glycero-3-phosphocholine (14:0-16:0 PC, MPPC),
1-myristoyl-2-stearoyl-sn-glycero-3-phosphocholine (14:0-18:0 PC, MSPC),
1-palmitoyl-2-myristoyl-sn-glycero-3-phosphocholine (16:0-14:0 PC, PMPC),
1-palmitoyl-2-stearoyl-sn-glycero-3-phosphocholine (16:0-18:0 PC, PSPC),
1-stearoyl-2-myristoyl-sn-glycero-3-phosphocholine (18:0-14:0 PC, SMPC),
1-stearoyl-2-palmitoyl-sn-glycero-3-phosphocholine (18:0-16:0 PC, SPPC), and any combination thereof.

E14. The composition of any one of E1 to E13, wherein the lipid composition further comprises a sterol.

E15. The composition of E11, wherein the sterol is cholesterol.

E16. The composition of any one of E1 to E15, wherein the lipid composition further comprises a PEG-lipid.

E17. The composition of any one of E1 to E16, wherein the net positive charge of the lipid composition is increased compared to the net positive charge of a corresponding lipid composition without the quaternary amine compound.

E18. A composition comprising:

(i) a lipid composition comprising
(1) MC3, L608, or

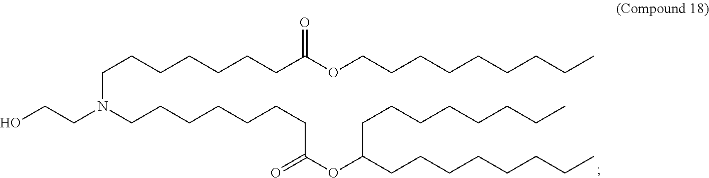 [SMILES: CCCCCCCCCOC(=O)CCCCCCCN(CCO)CCCCCCCC(=O)OC(CCCCCCCC)CCCCCCCC]

(2) a phospholipid;
(3) a sterol;
(4) a PEG-lipid;
(5) a quaternary amine compound which is DOTAP; and

(ii) a polynucleotide.

E19. The composition of E18, wherein the amount of the quaternary amine compound ranges from about 0.01 to about 20 mole % in the lipid composition.

E20. The composition of E18 or E19, wherein the amount of MC3, L608, or

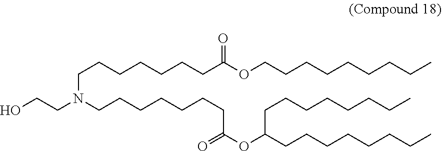 [SMILES: CCCCCCCCCOC(=O)CCCCCCCN(CCO)CCCCCCCC(=O)OC(CCCCCCCC)CCCCCCCC]

ranges from about 30 to about 70 mole % in the lipid composition.

E21. The composition of any one of E18 to E20, wherein the amount of the phospholipid ranges from about 1 to about 20 mole % in the lipid composition.

E22. The composition of any one of E18 to E21, wherein the amount of the sterol ranges from about 20 to about 60 mole % in the lipid composition.

E23. The composition of any one of E18 to E22, wherein the amount of the PEG-lipid ranges from about 0.1 to about 5 mole % in the lipid composition.

E24. The composition of E18 comprising:

(i) a lipid composition comprising
(1) about 50 mole % of MC3;
(2) about 10 mole % of DSPC or MSPC;
(3) about 33.5 mole % of cholesterol;
(4) about 1.5 mole % of PEG-DMG;
(5) about 5 mole % of DOTAP; and

(ii) a polynucleotide.

E25. The composition of E18 comprising:

(i) a lipid composition comprising
(1) about 50 mole % of

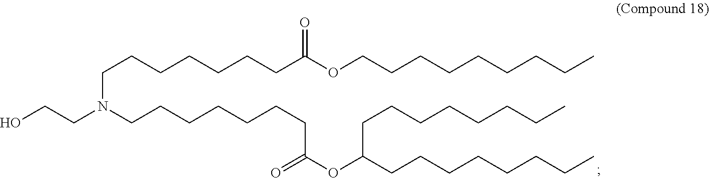 [SMILES: CCCCCCCCCOC(=O)CCCCCCCN(CCO)CCCCCCCC(=O)OC(CCCCCCCC)CCCCCCCC]

(2) about 10 mole % of DSPC or MSPC;
(3) about 33.5 mole % of cholesterol;
(4) about 1.5 mole % of PEG-DMG;
(5) about 5 mole % of DOTAP; and

(ii) a polynucleotide.

E26. A composition comprising:

(i) a lipid composition comprising
(1) an asymmetric phospholipid,
(2) an ionizable amino lipid, and
(3) optionally a quaternary amine compound; and

(ii) a polynucleotide,

wherein the composition is formulated for intratumoral delivery of the polynucleotide.

E27. The composition of E26, wherein the asymmetric phospholipid is selected from the group consisting of
1-myristoyl-2-palmitoyl-sn-glycero-3-phosphocholine (14:0-16:0 PC, MPPC),
1-myristoyl-2-stearoyl-sn-glycero-3-phosphocholine (14:0-18:0 PC, MSPC),
1-palmitoyl-2-myristoyl-sn-glycero-3-phosphocholine (16:0-14:0 PC, PMPC),
1-palmitoyl-2-stearoyl-sn-glycero-3-phosphocholine (16:0-18:0 PC, PSPC),
1-stearoyl-2-myristoyl-sn-glycero-3-phosphocholine (18:0-14:0 PC, SMPC),
1-stearoyl-2-palmitoyl-sn-glycero-3-phosphocholine (18:0-16:0 PC, SPPC), and any combination thereof.

E28. The composition of E26 or E27, wherein the lipid composition comprises a quaternary amine compound selected from the group consisting of 1,2-dioleoyl-3-trimethylammonium-propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), N,N-distearyl-N,N-dimethylammonium bromide (DDAB), N-(1,2-dimyristyloxyprop-3-yl)-N,N-dimethyl-N-hydroxyethyl ammonium bromide (DMRIE), N-(1,2-dioleoyloxyprop-3-yl)-N,N-dimethyl-N-hydroxyethyl ammonium bromide (DORIE), N,N-dioleyl-N,N-dimethylammonium chloride (DODAC), 1,2-dilauroyl-sn-glycero-3-ethylphosphocholine (DLePC), 1,2-di stearoyl-3-trimethylammonium-propane (DSTAP), 1,2-dipalmitoyl-3-trimethylammonium-propane (DPTAP), 1,2-dilinoleoyl-3-trimethylammonium-propane (DLTAP), 1,2-dimyristoyl-3-trimethylammonium-propane (DMTAP), 1,2-distearoyl-sn-glycero-3-ethylphosphocholine (DSePC), 1,2-dipalmitoyl-sn-glycero-3-ethylphosphocholine (DPePC), 1,2-dimyristoyl-sn-glycero-3-ethylphosphocholine (DMePC), 1,2-dioleoyl-sn-glycero-3-ethylphosphocholine (DOePC), 1,2-di-(9Z-tetradecenoyl)-sn-glycero-3-ethylphosphocholine (14:1 EPC), 1-palmitoyl-2-oleoyl-sn-glycero-3-ethylphosphocholine (16:0-18:1 EPC), and any combination thereof.

E29. The composition of any one of E26 to E28, wherein the ionizable amino lipid is selected from the group consisting of DLin-MC3-DMA (MC3), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof.

E30. The composition of any one of E26 to E28, wherein the ionizable amino lipid is selected from the group consisting of(13Z,16Z)—N,N-dimethyl-3-nonyldocosa-13,16-dien-1-amine (L608), (20Z,23Z)—N,N-dimethylnonacosa-20,23-dien-10-amine, (17Z,20Z)—N,N-dimemylhexacosa-17,20-dien-9-amine, (16Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)-N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, (11E,20Z,23Z)—N,N-dimethylnonacosa-11,20,2-trien-10-amine, and any combination thereof.

E31. The composition of any one of E26 to E28, wherein the ionizable amino lipid is selected from the group consisting of:

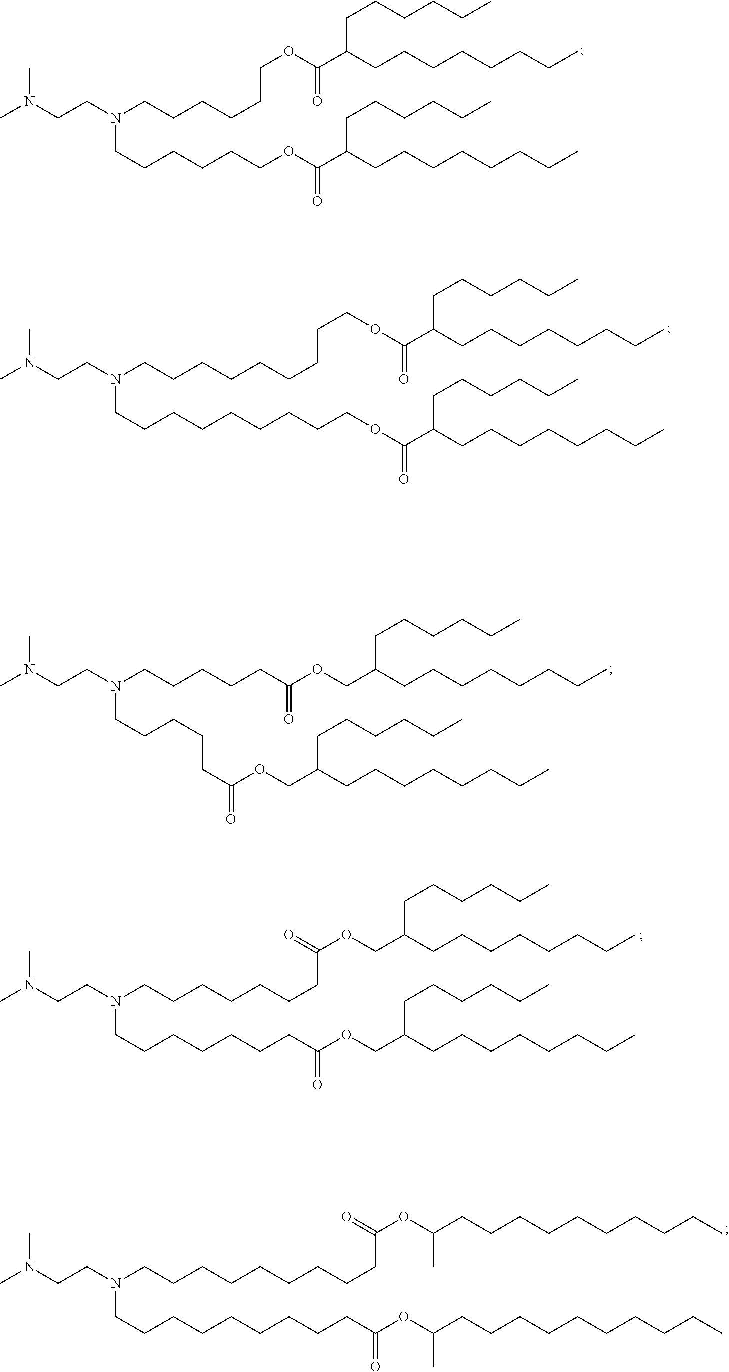

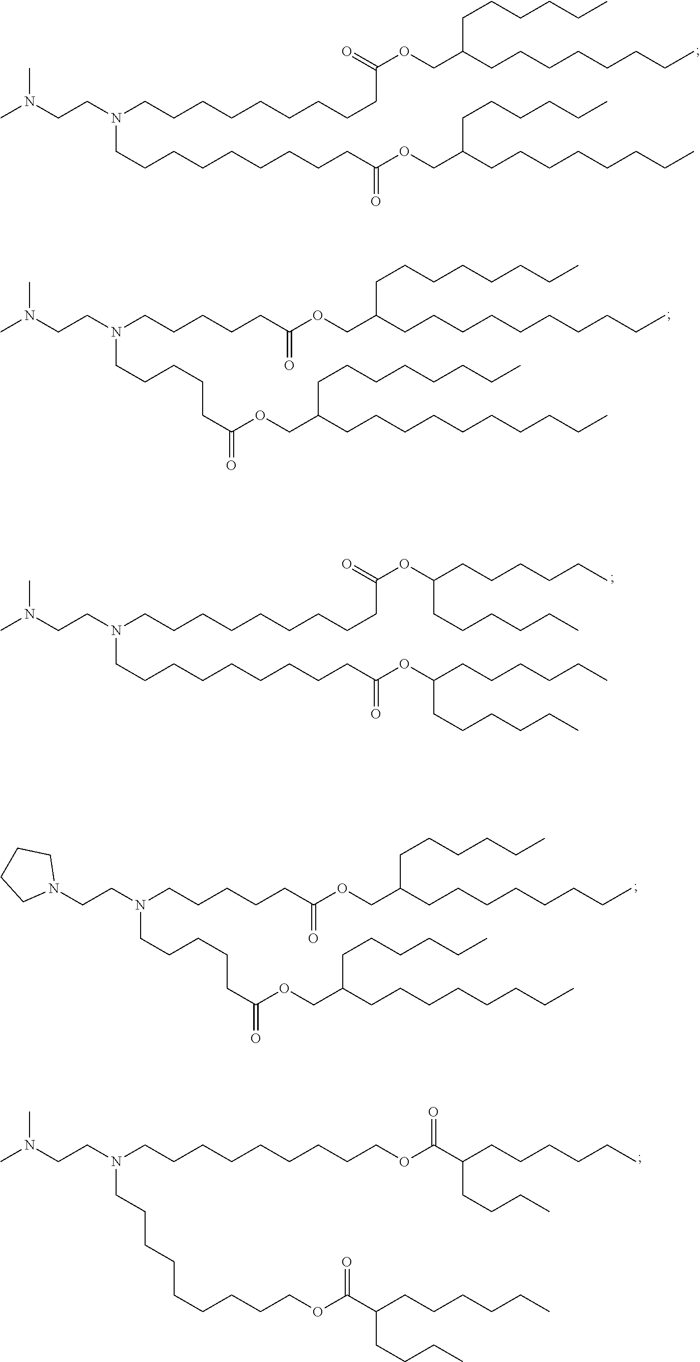

and any combination thereof.

E32. The composition of any one of E26 to E28, wherein wherein the ionizable amino lipid is selected from Compound 1 to Compound 147.

E33. The composition of any one of E26 to E32, wherein the lipid composition further comprises a sterol.

E34. The composition of E33, wherein the sterol is cholesterol.

E35. The composition of any one of E26 to E34, wherein the lipid composition further comprises a PEG-lipid.

E36. A composition comprising:

(i) a lipid composition comprising
(1) MC3, L608, or

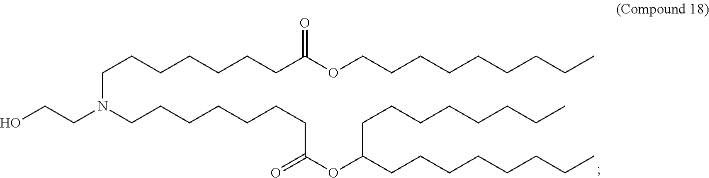 [SMILES: CCCCCCCCCOC(=O)CCCCCCCN(CCO)CCCCCCCC(=O)OC(CCCCCCCC)CCCCCCCC]

(2) an asymmetric phospholipid which is MSPC;
(3) a sterol;
(4) a PEG-lipid; and

(ii) a polynucleotide,

wherein the composition is formulated for intratumoral delivery of the polynucleotide.

E37. The composition of E36 comprising:

(i) a lipid composition comprising
(1) about 50 mole % of MC3 or

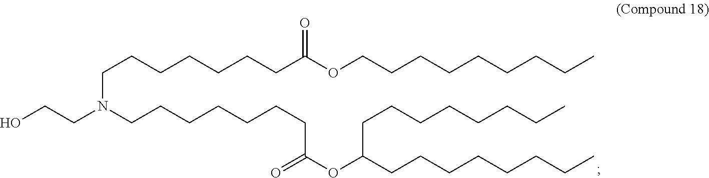 [SMILES: CCCCCCCCCOC(=O)CCCCCCCN(CCO)CCCCCCCC(=O)OC(CCCCCCCC)CCCCCCCC]

(2) about 10 mole % of MSPC;
(3) about 38.5 mole % of cholesterol;
(4) about 1.5 mole % of PEG-DMG; and

(ii) a polynucleotide.

E38. The composition of any one of E1 to E37, wherein the polynucleotide is selected from a group consisting of plasmid DNA, linear DNA selected from poly and oligo-nucleotides, chromosomal DNA, messenger RNA (mRNA), antisense DNA/RNA, siRNA, microRNA (miRNA), ribosomal RNA, oligonucleotide DNA (ODN) single and double strand, CpG imunostimulating sequence (ISS), locked nucleic acid (LNA), ribozyme, asymmetrical interfering RNA (aiRNA), Dicer-substrate RNA (dsRNA), small hairpin RNA (shRNA), and any combination thereof.

E39. The composition of E38, wherein the polynucleotide comprises mRNA.

E40. The composition of E39, wherein the mRNA comprises at least one chemically modified nucleobase.

E41. The composition of E40, wherein the at least one chemically modified nucleobase is selected from the group consisting of pseudouracil (ψ), 2-thiouracil (s2U), 4′-thiouracil, 5-methylcytosine, 2-thio-1-methyl-1-deaza-pseudouracil, 2-thio-1-methyl-pseudouracil, 2-thio-5-aza-uracil, 2-thio-dihydropseudouracil, 2-thio-dihydrouracil, 2-thio-pseudouracil, 4-methoxy-2-thio-pseudouracil, 4-methoxy-pseudouracil, 4-thio-1-methyl-pseudouracil, 4-thio-pseudouracil, 5-aza-uracil, dihydropseudouracil, 5-methyluracil, 5-methoxyuracil, 2′-O-methyl uracil, 1-methyl-pseudouracil (m1ψ), 1-ethyl-pseudouracil (e1ψ), 5-methoxy-uracil (mo5U), 5-methyl-cytosine (m5C), α-thio-guanine, α-thio-adenine, 5-cyano uracil, 4′-thio uracil, 7-deaza-adenine, 1-methyl-adenine (m1A), 2-methyl-adenine (m2A), N6-methyl-adenine (m6A), and 2,6-Diaminopurine, 1-methyl-inosine (m1I), wyosine (imG), methylwyosine (mimG), 7-deaza-guanine, 7-cyano-7-deaza-guanine (preQ0), 7-aminomethyl-7-deaza-guanine (preQ1), 7-methyl-guanine (m7G), 1-methyl-guanine (m1G), 8-oxo-guanine, 7-methyl-8-oxo-guanine, and two or more combinations thereof.

E42. The composition of E40 or E41, wherein the at least one chemically modified nucleobases is selected from the group consisting of pseudouracil (ψ), 1-methyl-pseudouracil (m1ψ), 1-ethyl-pseudouracil (e1ψ), 5-methylcytosine, 5-methoxyuracil, and any combination thereof.

E43. The composition of any one of E39 to E42, wherein the nucleobases in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

E44. The composition of any one of E40 to E43, wherein the chemically modified nucleobases in the mRNA are selected from the group consisting of uracil, adenine, cytosine, guanine, and any combination thereof.

E45. The composition of any one of E39 to E44, wherein the uricils in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

E46. The composition of any one of E39 to E45, wherein the adenines in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

E47. The composition of any one of E39 to E46, wherein the cytosines in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

E48. The composition of any one of E39 to E47, wherein the guanines in mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

E49. The composition of any one of E39 to E48, wherein the mRNA is sequence-optimized.

E50. The composition of any one of E39 to E49, wherein the mRNA further comprises a 5′ UTR.

E51. The composition of E50, wherein the 5′ UTR is sequence-optimized.

E52. The composition of any one of E39 to E51, wherein the mRNA further comprises a 3′ UTR.

E53. The composition of E52, wherein the 3′ UTR is sequence-optimized.

E54. The composition of any one of E39 to E53, wherein the mRNA further comprises a 5′ terminal cap.

E55. The composition of E54, wherein the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

E56. The composition of any one of E39 to E55, wherein the mRNA further comprises a 3′ polyA tail.

E57. The composition of any one of E39 to E56, wherein the mRNA is in vitro transcribed (IVT).

E58. The composition of any one of E39 to E57, wherein the mRNA is chimeric.

E59. The composition of any one of E39 to E58, wherein the mRNA is circular.

E60. The composition of any one of E1 to E59, wherein the polynucleotide encodes a polypeptide when administered intratumorally to a tumor tissue.

E61. The composition of any one of E1 to E60, wherein the polypeptide comprises a cytokine, a growth factor, a hormone, a cell surface receptor, an antibody or antigen binding portion thereof.

E62. The composition of any one of E1 to E60, wherein the polynucleotide encodes a polypeptide which targets a cancer antigen.

E63. The composition of any one of E1 to E62, wherein the lipid composition is in lipid nanoparticle (LNP) form.

E64. The composition of any one of E1 to E63, wherein the lipid composition encapsulates the polynucleotide.

E65. The composition of any one of E1 to E64, wherein the polynucleotide encodes a polypeptide when administered intratumorally to a tumor tissue, and wherein expression of the polypeptide in the tumor tissue is higher than expression of the polypeptide in a non-tumor tissue.

E66. The composition of E65, wherein a ratio of the protein expression in the tumor tissue to that in the non-tumor tissue is at least about 200:1, at least about 250:1, at least about 300:1, at least about 350:1, at least about 400:1, at least about 450:1, at least about 500:1, at least about 600:1, at least about 700:1, at least about 800:1, at least about 900:1, or at least about 1000:1, when the protein expression is measured 24 hours post administration.

E67. The composition of E65, wherein a ratio of the protein expression in the tumor tissue to that in the non-tumor tissue is at least about 200:1, at least about 250:1, at least about 300:1, at least about 350:1, at least about 400:1, at least about 450:1, at least about 500:1, at least about 600:1, at least about 700:1, at least about 800:1, at least about 900:1, or at least about 1000:1, when the protein expression is measured 48 hours post administration.

E68. The composition of any one of E1 to E25, wherein the polynucleotide encodes a polypeptide when administered intratumorally to a tumor tissue, and wherein the composition increases retention of the polynucleotide in the tumor tissue as compared to a corresponding composition without the quaternary amine compound.

E69. The composition of any one of E1 to E25, wherein the polynucleotide encodes a polypeptide when administered intratumorally to a tumor tissue, and wherein the composition decreases expression of the polypeptide in a non-tumor tissue as compared to a corresponding composition without the quaternary amine compound.

E70. A method of increasing retention of a polynucleotide in a tumor tissue of a subject, comprising administering intratumorally to the tumor tissue the composition of any of E1 to E25, wherein the retention of the polynucleotide in the tumor tissue is increased compared to the retention of the polynucleotide in the tumor tissue by a corresponding composition without the quaternary amine compound.

E71. A method of decreasing expression of a polypeptide in liver of a subject, comprising administering intratumorally to a tumor tissue the composition of any of E1 to E25, wherein the expression of the polypeptide in liver is decreased compared to the expression of the polypeptide in liver by a corresponding composition without the quaternary amine compound.

E72. The method of E71, wherein the polypeptide is expressed at the same level or at a higher level in the tumor tissue compared to the polypeptide expressed by a corresponding composition without the quaternary amine compound.

E73. A method of increasing expression of a polypeptide in a tumor tissue of a subject, comprising administering intratumorally to the tumor tissue the composition of any of E26 to E37, wherein the expression level of the polypeptide in the tumor tissue is increased compared to the expression level of the polypeptide by a corresponding composition with a symmetric phospholipid.

E74. A method of increasing retention of a polynucleotide in a tumor tissue of a subject, comprising administering intratumorally to the tumor tissue the composition of any of E26 to E37, wherein the retention of the polynucleotide in the tumor tissue is increased compared to the retention of the polynucleotide in the tumor tissue by a corresponding composition a symmetric phospholipid.

E75. The method of any one of E70 to E74, wherein the subject is in a mammal.

E76. The method of E75, wherein the mammal is a human.

E77. A lipid composition comprising:

(i) MC3, L608, or

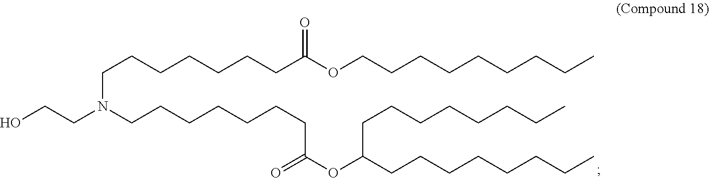 [SMILES: CCCCCCCCCOC(=O)CCCCCCCN(CCO)CCCCCCCC(=O)OC(CCCCCCCC)CCCCCCCC]

(ii) a phospholipid;

(iii) a sterol;

(iv) a PEG-lipid; and

(v) a quaternary amine compound which is DOTAP, DOTMA, DLePC, or DDAB,

wherein the amount of a quaternary amine compound ranges from about 0.01 to about 20 mole % in the lipid composition.

E78. The lipid composition of E77, wherein the phospholipid is MSPC.

E79. The lipid composition of E77 or E78, wherein the quaternary amine compound is DOTAP.

E80. The lipid composition of any one of E77 to E79, wherein the amount of MC3, L608, or

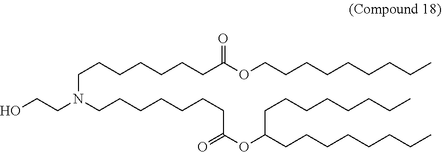 [SMILES: CCCCCCCCCOC(=O)CCCCCCCN(CCO)CCCCCCCC(=O)OC(CCCCCCCC)CCCCCCCC]

ranges from about 30 to about 70 mole % in the lipid composition.

E81. The lipid composition of any one of E77 to E80, wherein the amount of the phospholipid ranges from about 1 to about 20 mole % in the lipid composition.

E82. The lipid composition of any one of E77 to E81, wherein the amount of the sterol ranges from about 20 to about 60 mole % in the lipid composition.

E83. The lipid composition of any one of E77 to E82, wherein the amount of the PEG-lipid ranges from about 0.1 to about 5 mole % in the lipid composition.

E84. The lipid composition of E77 comprising:

(i) about 50 mole % of MC3;

(ii) about 10 mole % of DSPC or MSPC;

(iii) about 33.5 mole % of cholesterol;

(iv) about 1.5 mole % of PEG-DMG; and

(v) about 5 mole % of DOTAP.

E85. The lipid composition of E77 comprising:

(i) about 50 mole % of

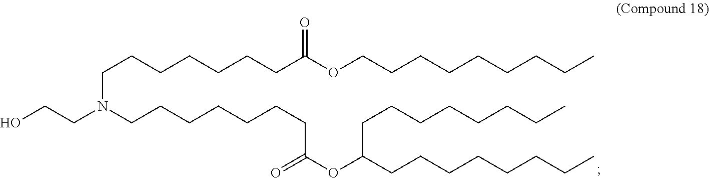 [SMILES: CCCCCCCCCOC(=O)CCCCCCCN(CCO)CCCCCCCC(=O)OC(CCCCCCCC)CCCCCCCC]

(ii) about 10 mole % of DSPC or MSPC;

(iii) about 33.5 mole % of cholesterol;

(iv) about 1.5 mole % of PEG-DMG; and

(v) about 5 mole % of DOTAP.

E86. A lipid composition comprising

(i) MC3 or

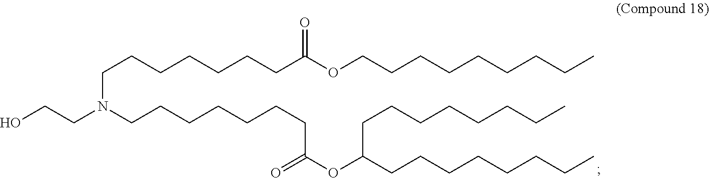 [SMILES: CCCCCCCCCOC(=O)CCCCCCCN(CCO)CCCCCCCC(=O)OC(CCCCCCCC)CCCCCCCC]

(ii) an asymmetric phospholipid;

(iii) a sterol; and

(iv) a PEG-lipid.

E87. The lipid composition of E86 comprising:

(i) about 50 mole % of MC3 or

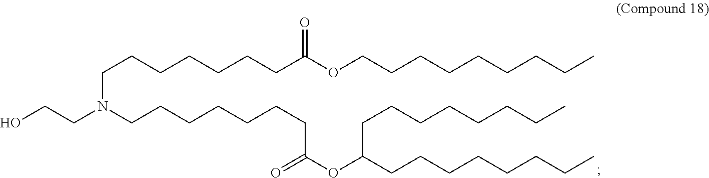 [SMILES: CCCCCCCCCOC(=O)CCCCCCCN(CCO)CCCCCCCC(=O)OC(CCCCCCCC)CCCCCCCC]

(ii) about 10 mole % of MSPC;

(iii) about 38.5 mole % of cholesterol; and

(iv) about 1.5 mole % of PEG-DMG.

E88. A method of producing a composition comprising a polynucleotide comprising formulating the polynucleotide in the lipid composition of any one of E77 to E87.

E89. A method of delivering a polynucleotide comprising formulating the polynucleotide in the lipid composition of any one of E77 to E87.

E90. The composition of any one of E1 to E69, wherein the ionizable amino lipid comprises two different tail groups.

E91. The composition of E90, wherein at least one tail group is branched.

E92. A method of intratumorally delivering a polynucleotide comprising administering to a tumor tissue the composition of any one of E1 to E69 and E77 to E87.

In addition, the present disclosure also includes the following embodiments numbered E101 through E192. This list of embodiments is presented as an exemplary list and the application is not limited to these embodiments.

E101. A pharmaceutical composition for intratumoral delivery comprising:

(a) a lipid composition comprising:
(i) a compound having the formula (I)

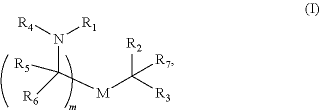

wherein

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H; each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof, wherein alkyl and alkenyl groups may be linear or branched;

provided when R4 is —(CH2)nQ, —(CH2)nCHQR, —CHQR, or —CQ(R)2, then (i) Q is not —N(R)2 when n is 1, 2, 3, 4 or 5, or (ii) Q is not 5, 6, or 7-membered heterocycloalkyl when n is 1 or 2;

and

(b) a therapeutic agent or a polynucleotide encoding a therapeutic agent.

E102. The pharmaceutical composition of E101, wherein the compound of formula (I) is Formula (IA):

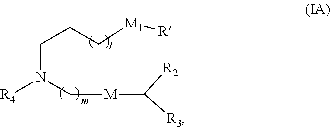

or a salt or stereoisomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which n is 1, 2, 3, 4, or 5 and Q is OH, —NHC(S)N(R)₂, or —NHC(O)N(R)₂; M and M′ are independently selected from —C(O)O—, —OC(O)—, —C(O)N(R′)—, —P(O)(OR′)O—, an aryl group, and a heteroaryl group; and

R₂ and R₃ are independently selected from the group consisting of H, C₁₋₁₄ alkyl, and C₂₋₁₄ alkenyl.

E103. The pharmaceutical composition of E101, wherein the compound of formula (I) is Formula (II):

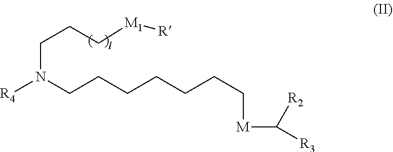

or a salt or stereoisomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, or —NHC(O)N(R)₂; M and M′ are independently selected from —C(O)O—, —OC(O)—, —C(O)N(R′)—, —P(O)(OR′)O—, an aryl group, and a heteroaryl group; and

R₂ and R₃ are independently selected from the group consisting of H, C₁₋₁₄ alkyl, and C₂₋₁₄ alkenyl.

E104. The pharmaceutical composition of E101, wherein the compound of formula (I) is of the formula (IIa),

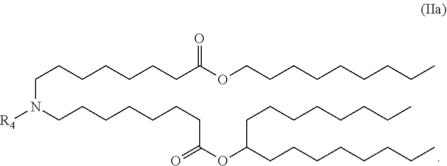

E105. The pharmaceutical composition of E101, wherein the compound of formula (I) is of the formula (IIb),

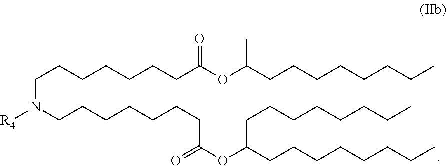

E106. The pharmaceutical composition of E101, wherein the compound of formula (I) is of the formula (IIc),

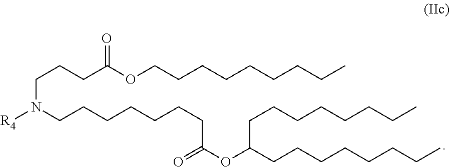

E107. The pharmaceutical composition of E101, wherein the compound of formula (I) is of the formula (IIe),

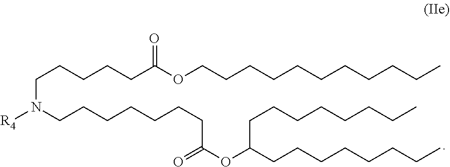

E108. The pharmaceutical compound of any one of E104 to E107, wherein R₄ is selected from —(CH₂)ₙQ and —(CH₂)ₙCHQR, wherein Q, R, and n are as defined above in E101.

E109. The pharmaceutical composition of E105, wherein Q is selected from the group consisting of —OR, —OH, —O(CH₂)ₙN(R)₂, —OC(O)R, —CX₃, —CN,
—N(R)C(O)R, —N(H)C(O)R, —N(R)S(O)₂R, —N(H)S(O)₂R, —N(R)C(O)N(R)₂,
—N(H)C(O)N(R)₂, —N(H)C(O)N(H)(R), —N(R)C(S)N(R)₂, —N(H)C(S)N(R)₂,
—N(H)C(S)N(H)(R), and a heterocycle, wherein R and n are as defined above in E101.

E110. The pharmaceutical composition of E108 or E109, wherein n is 1 or 2.

E111. The pharmaceutical composition of E101, wherein the compound of formula (I) is of the formula (IId),

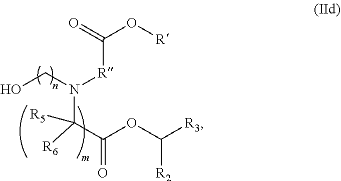

wherein R₂ and R₃ are independently selected from the group consisting of C₅₋₁₄ alkyl and C₅₋₁₄ alkenyl, n is selected from 2, 3, and 4, and R′, R″, R₅, R₆ and m are as defined in E101.

E112. The pharmaceutical composition of E111, wherein R₂ is C₈ alkyl.

E113. The pharmaceutical composition of E111 or E112, wherein R₃ is C₅-C₉ alkyl.

E114. The pharmaceutical composition of any one of E111 to E113, wherein m is 5, 7, or 9.

E115. The pharmaceutical composition of any one of E111 to E114, wherein each R₅ is H.

E116. The pharmaceutical composition of E115, wherein each R₆ is H.

E117. The pharmaceutical composition of E101, wherein the compound is selected from Compound 1 to Compound 147.

E118. The pharmaceutical composition of any one of E101-E117, wherein the lipid composition further comprises a phospholipid.

E119. The pharmaceutical composition of E118, wherein the phospholipid is a glycerophospholipid, a phosphosphingolipid, or any combination thereof.

E120. The pharmaceutical composition of E118, wherein the phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1,2-di-O-octadecenyl-sn-glycero-3-phosphocholine (18:0 Diether PC),
1,2-dilinolenoyl-sn-glycero-3-phosphocholine (DLnPC),
1,2-diarachidonoyl-sn-glycero-3-phosphocholine (DAPC),
1,2-didocosahexaenoyl-sn-glycero-3-phosphocholine (DHAPC),
1,2-dioleoyl-sn-glycero-3-phosphoethanolamine (DOPE),
1,2-diphytanoyl-sn-glycero-3-phosphoethanolamine (ME 16:0 PE),
1,2-distearoyl-sn-glycero-3-phosphoethanolamine (DSPE),
1,2-dilinoleoyl-sn-glycero-3-phosphoethanolamine (DLPE),
1,2-dilinolenoyl-sn-glycero-3-phosphoethanolamine (DLnPE),
1,2-diarachidonoyl-sn-glycero-3-phosphoethanolamine (DAPE),
1,2-didocosahexaenoyl-sn-glycero-3-phosphoethanolamine (DHAPE),
1,2-dioleoyl-sn-glycero-3-phospho-rac-(1-glycerol)sodium salt (DOPG), and any combination thereof.

E121. The pharmaceutical composition of E118, wherein the phospholipid is an asymmetric phospholipid.

E122. The pharmaceutical composition of E118, wherein the phospholipid is an asymmetric phospholipid selected from the group consisting of
1-myristoyl-2-palmitoyl-sn-glycero-3-phosphocholine (14:0-16:0 PC, MPPC),
1-myristoyl-2-stearoyl-sn-glycero-3-phosphocholine (14:0-18:0 PC, MSPC),
1-palmitoyl-2-myristoyl-sn-glycero-3-phosphocholine (16:0-14:0 PC, PMPC),
1-palmitoyl-2-stearoyl-sn-glycero-3-phosphocholine (16:0-18:0 PC, PSPC),
1-stearoyl-2-myristoyl-sn-glycero-3-phosphocholine (18:0-14:0 PC, SMPC),
1-stearoyl-2-palmitoyl-sn-glycero-3-phosphocholine (18:0-16:0 PC, SPPC), and any combination thereof.

E123. The pharmaceutical composition of E122, wherein the asymmetric phospholipid is 1-myristoyl-2-stearoyl-sn-glycero-3-phosphocholine (MSPC).

E124. The pharmaceutical composition of any one of E101 to E123, wherein the lipid composition further comprises a quaternary amine compound.

E125. The pharmaceutical composition of E124, wherein the quaternary amine compound is selected from the group consisting of
1,2-dioleoyl-3-trimethylammonium-propane (DOTAP),
N-[1-(2,3-dioleoyloxy)propyl]-N,N,N-trimethylammonium chloride (DOTMA),
1-[2-(oleoyloxy)ethyl]-2-oleyl-3-(2-hydroxyethyl)imidazolinium chloride (DOTIM),
2,3-dioleyloxy-N-[2(sperminecarboxamido)ethyl]-N,N-dimethyl-1-propanaminium trifluoroacetate (DOSPA),
N,N-distearyl-N,N-dimethylammonium bromide (DDAB),
N-(1,2-dimyristyloxyprop-3-yl)-N,N-dimethyl-N-hydroxyethyl ammonium bromide (DMRIE),
N-(1,2-dioleoyloxyprop-3-yl)-N,N-dimethyl-N-hydroxyethyl ammonium bromide (DORIE),
N,N-dioleyl-N,N-dimethylammonium chloride (DODAC),
1,2-dilauroyl-sn-glycero-3-ethylphosphocholine (DPePC),
1,2-distearoyl-3-trimethylammonium-propane (DSTAP),
1,2-dipalmitoyl-3-trimethylammonium-propane (DPTAP),
1,2-dilinoleoyl-3-trimethylammonium-propane (DLTAP),
1,2-dimyristoyl-3-trimethylammonium-propane (DMTAP),
1,2-distearoyl-sn-glycero-3-ethylphosphocholine (DSePC),
1,2-dipalmitoyl-sn-glycero-3-ethylphosphocholine (DPePC),
1,2-dimyristoyl-sn-glycero-3-ethylphosphocholine (DMePC),
1,2-dioleoyl-sn-glycero-3-ethylphosphocholine (DOePC),
1,2-di-(9Z-tetradecenoyl)-sn-glycero-3-ethylphosphocholine (14:1 EPC), 1-palmitoyl-2-oleoyl-sn-glycero-3-ethylphosphocholine (16:0-18:1 EPC), and any combination thereof.

E126. The pharmaceutical composition of E124, wherein the quaternary amine compound is 1,2-dioleoyl-3-trimethylammonium-propane (DOTAP).

E127. The pharmaceutical composition of any one of E101 to E126, wherein the lipid composition further comprises a structural lipid.

E128. The pharmaceutical composition of E127, wherein the structural lipid is selected from the group consisting of cholesterol, fecosterol, sitosterol, ergosterol, campesterol, stigmasterol, brassicasterol, tomatidine, ursolic acid, alpha-tocopherol, and mixtures thereof.

E129. The pharmaceutical composition of E128, wherein the structural lipid is cholesterol.

E130. The pharmaceutical composition of any one of E101 to E129, wherein the lipid composition further comprises a polyethylene glycol (PEG) lipid.

E131. The pharmaceutical composition of E130, wherein the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

E132. The pharmaceutical composition of any of E101 to E131, wherein the amount of the compound of formula (I) in the lipid composition ranges from about 1 mol % to 99 mol % in the lipid composition.

E133. The pharmaceutical composition of any of E101 to E132, wherein the amount of the compound of formula (I) ranges from about 30 mol % to about 70 mol % in the lipid composition.

E134. The pharmaceutical composition of any of E101 to E133, wherein the amount of the compound of formula (I) is about 50 mol % in the lipid composition.

E135. The pharmaceutical composition of any of E118 to E134, wherein the amount of the phospholipid ranges from about 1 mol % to about 20 mol % in the lipid composition.

E136. The pharmaceutical composition of any of E118 to E135, wherein the amount of the phospholipid is about 10 mol % in the lipid composition.

E137. The pharmaceutical composition of any of E124 to E136, wherein the amount of the quaternary amine compound in the lipid composition ranges from about 5 mol % to about 10.0 mol %.

E138. The pharmaceutical composition of any of E124 to E137, wherein the amount of the quaternary amine compound in the lipid composition is about 5 mol %.

E139. The pharmaceutical composition of any of E127 to E138, wherein the amount of the structural lipid ranges from about 20 mol % to about 60 mol % in the lipid composition.

E140. The pharmaceutical composition of any of c E127 to E139, wherein the amount of the structural lipid in the composition is about 33.5 mol %.

E141. The pharmaceutical composition of any of E130 to E140, wherein the amount of the PEG-lipid ranges from about 0.1 mol % to about 5.0 mol % in the lipid composition.

E142. The pharmaceutical composition of any of E130 to E141, wherein the amount of the PEG-lipid in the composition is about 1.5 mol %.

E143. The pharmaceutical composition of any one of E101 to E142, wherein the wt/wt ratio of the lipid composition to the polypeptide is from about 10:1 to about 60:1.

E144. The pharmaceutical composition of any of E101 to E143, wherein the polynucleotide is a deoxyribonucleic nucleic acid (DNA) or a ribonucleic acid (RNA).

E145. The pharmaceutical composition of E144, wherein the RNA is selected from the group consisting of a small interfering RNA (siRNA), an asymmetrical interfering RNA (aiRNA), a microRNA (miRNA), a Dicer-substrate RNA (dsRNA), a small hairpin RNA (shRNA), a messenger RNA (mRNA), guide strand RNA, and combinations thereof.

E146. The pharmaceutical composition of E145, wherein the RNA is mRNA.

E147. The pharmaceutical composition of E146, wherein the mRNA is synthetic.

E148. The pharmaceutical composition of any one of E101 to E147, wherein the polynucleotide comprises at least one chemically modified nucleobase.

E149. The pharmaceutical composition of E148, wherein the at least one chemically modified nucleobase is selected from the group consisting of pseudouracil (ψ), 2-thiouracil (s2U), 4′-thiouracil, 5-methylcytosine, 2-thio-1-methyl-1-deaza-pseudouracil, 2-thio-1-methyl-pseudouracil, 2-thio-5-aza-uracil, 2-thio-dihydropseudouracil, 2-thio-dihydrouracil, 2-thio-pseudouracil, 4-methoxy-2-thio-pseudouracil, 4-methoxy-pseudouracil, 4-thio-1-methyl-pseudouracil, 4-thio-pseudouracil, 5-aza-uracil, dihydropseudouracil, 5-methyluracil, 5-methoxyuracil, 2′-O-methyl uracil, 1-methyl-pseudouracil (m1ψ), 1-ethyl-pseudouracil (e1ψ), 5-methoxy-uracil (mo5U), 5-methyl-cytosine (m5C), α-thio-guanine, α-thio-adenine, 5-cyano uracil, 4′-thio uracil, 7-deaza-adenine, 1-methyl-adenine (m1A), 2-methyl-adenine (m2A), N6-methyl-adenine (m6A), and 2,6-Diaminopurine, 1-methyl-inosine (m1I), wyosine (imG), methylwyosine (mimG), 7-deaza-guanine, 7-cyano-7-deaza-guanine (preQ0), 7-aminomethyl-7-deaza-guanine (preQ1), 7-methyl-guanine (m7G), 1-methyl-guanine (m1G), 8-oxo-guanine, 7-methyl-8-oxo-guanine, and two or more combinations thereof.

E150. The pharmaceutical composition of E148 or E149, wherein the at least one chemically modified nucleobases is selected from the group consisting of pseudouracil (ψ), 1-methyl-pseudouracil (m1ψ), 1-ethyl-pseudouracil (e1ψ), 5-methylcytosine, 5-methoxyuracil, and any combination thereof.

E151. The pharmaceutical composition of any one of E146 to E150, wherein the nucleobases in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

E152. The pharmaceutical composition of any one of E148 to E151, wherein the chemically modified nucleobases are selected from the group consisting of uracil, adenine, cytosine, guanine, and any combination thereof.

E153. The pharmaceutical composition of any one of E146 to E152, wherein the uracils in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

E154. The pharmaceutical composition of any one of E146 to E152, wherein the adenines in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

E155. The pharmaceutical composition of any one of E146 to E152, wherein the cytosines in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

E156. The pharmaceutical composition of any one of E146 to E152, wherein the guanines in mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

E157. The pharmaceutical composition of any one of E146 to E156, wherein the mRNA is sequence-optimized.

E158. The pharmaceutical composition of any one of E146 to E157, wherein the mRNA further comprises a 5′ UTR.

E159. The pharmaceutical composition of E158, wherein the 5′ UTR is sequence-optimized.

E160. The pharmaceutical composition of any one of E146 to E159, wherein the mRNA further comprises a 3′ UTR.

E161. The pharmaceutical composition of E160, wherein the 3′ UTR is sequence-optimized.

E162. The pharmaceutical composition of any one of E146 to E161, wherein the mRNA further comprises a 5′ terminal cap.

E163. The pharmaceutical composition of E162, wherein the 5′ terminal cap is a Cap0, Cap1, ARCA, inosine, N1-methyl-guanosine, 2′fluoro-guanosine, 7-deaza-guanosine, 8-oxo-guanosine, 2-amino-guanosine, LNA-guanosine, 2-azidoguanosine, Cap2, Cap4, 5′ methylG cap, or an analog thereof.

E164. The pharmaceutical composition of any one of E146 to E163, wherein the mRNA further comprises a 3′ polyA tail.

E165. The pharmaceutical composition of any one of E146 to E164, wherein the mRNA is in vitro transcribed (IVT).

E166. The pharmaceutical composition of any one of E146 to E165, wherein the mRNA is chimeric.

E167. The pharmaceutical composition of any one of c E146 to E166, wherein the mRNA is circular.

E168. The pharmaceutical composition of any one of E101 to E167, wherein the polynucleotide encodes a polypeptide when administered intratumorally to a tumor tissue.

E169. The pharmaceutical composition according to E101 to E168, wherein the polynucleotide encodes a cytokine, a growth factor, a hormone, a cell surface receptor, or an antibody or antigen binding portion thereof.

E170. The pharmaceutical composition according to any one of E101 to E168, wherein the polynucleotide encodes a polypeptide which targets a tumor antigen.

E171. The pharmaceutical composition of any of E101 to E170, wherein the pharmaceutical composition is in lipid nanoparticle (LNP) form.

E172. The pharmaceutical composition of any one of E101 to E171, wherein the lipid composition encapsulates the therapeutic agent or a polynucleotide encoding the therapeutic agent.

E173. The pharmaceutical composition of any one of E101 to E172, further comprising a pharmaceutically acceptable vehicle or excipient.

E174. The pharmaceutical composition of any one of E101 to E173, wherein the polynucleotide encodes a polypeptide when administered intratumorally to a tumor tissue, and wherein the pharmaceutical composition decreases expression levels of the polypeptide in a non-tumor tissue as compared to expression levels after administering a corresponding reference composition.

E175. The pharmaceutical composition of E174, wherein the non-tumor tissue is peritumoral tissue.

E176. The pharmaceutical composition of E174, wherein the non-tumor tissue is liver tissue.

E177. The pharmaceutical composition of any one of E101 to E176, wherein when the pharmaceutical composition is administered intratumorally to a tumor tissue the retention of the polynucleotide in the tumor tissue is increased compared to the retention of the polynucleotide in the tumor tissue after administering a corresponding reference composition.

E178. The pharmaceutical composition of any one of E101 to E177, wherein immune response caused by the intratumoral administration of the pharmaceutical composition to a subject is not elevated compared to the immune response caused by intratumoral administration of a PBS.

E179. The pharmaceutical composition of E178, wherein the immune response is measured by the concentration of IL-6, G-CSF, GROα, or a combination thereof in plasma.

E180. The pharmaceutical composition of E178 or E179, wherein the immune response is measured at 24 hour post administration.

E181. A method of increasing retention of a polynucleotide in a tumor tissue in a subject, comprising administering intratumorally to the tumor tissue the pharmaceutical composition of any of E101 to E180, wherein the retention of the polynucleotide in the tumor tissue is increased compared to the retention of the polynucleotide in the tumor tissue after administering a corresponding reference composition.

E182. A method of decreasing expression leakage of a polynucleotide administered intratumorally to a subject in need thereof, comprising administering said polynucleotide intratumorally to the tumor tissue as a pharmaceutical composition according to any of E101 to E180, wherein the expression level of the polypeptide in non-tumor tissue is decreased compared to the expression level of the polypeptide in non-tumor tissue after administering a corresponding reference composition.

E183. The method of E182, wherein the non-tumoral tissue is peritumoral tissue.

E184. The method of E182, wherein the non-tumoral tissue is liver tissue.

E185. A method of increasing protein expression of a polypeptide in a tumor tissue of a subject, comprising administering intratumorally to the tumor tissue the pharmaceutical composition of any of E101 to E180, wherein the expression level of the polypeptide in the tumor tissue is increased compared to the expression level of the polypeptide after administering a corresponding reference composition.

E186. A method of delivering a polynucleotide to a subject in need thereof, comprising intratumorally administering to the subject a pharmaceutical composition of any of E101 to E180, wherein immune response caused by the administration of the pharmaceutical composition is not elevated compared to the immune response caused by intratumoral administration of a PBS.

E187. The method of E186, wherein the immune response is measured by the concentration of IL-6, G-CSF, GROα, or a combination thereof in plasma.

E188. The method of E186 or E187, wherein the immune response is measured at 24 hour post administration.

E189. The method of any of E181 to E188, wherein the subject is in a mammal.

E190. The method of E189, wherein the mammal is a human.

E191. A method of producing a pharmaceutical composition for intratumoral delivery comprising formulating a polynucleotide encoding a therapeutic agent or a portion thereof in the lipid composition of the pharmaceutical composition of any one of E101 to E180.

E192. A method of producing a lipid nanoparticle for intratumoral delivery comprising encapsulating a polynucleotide encoding a therapeutic agent or a portion thereof in the lipid composition of the pharmaceutical composition of any one of E101 to E180.

E193. A lipid nanoparticle comprising a polynucleotide encoding a therapeutic agent or a portion thereof encapsulated in the lipid composition of the pharmaceutical composition of any one of E101 to E180.

### BRIEF DESCRIPTION OF THE DRAWINGS/FIGURES

FIGS. 1A and 1B show the GFP expression levels in tumor and liver in animals with Hep 3B tumors administered intratumorally with lipid compositions containing a polynucleotide encoding GFP (0.5 mg/kg dose).

FIG. 2 shows the GFP expression levels in tumor and liver in animals with Hep 3B tumors administered intratumorally with lipid compositions containing a polynucleotide encoding GFP (2.5 μg/mouse dose).

FIG. 3 shows Bioluminescenes measurement of tumor and liver in animals with A20 tumors administered intratumorally with lipid compositions containing a polynucleotide encoding luciferase (12.5 μg/mouse dose).

FIG. 4 shows the luciferase expression levels in tumor in animals with A20 tumors administered intratumorally with lipid compositions containing a polynucleotide encoding luciferase (12.5 μg/mouse dose).

FIG. 5 shows the GFP expression levels in tumor in animals with MC38 tumors administered intratumorally with lipid compositions containing a polynucleotide encoding GFP (2.5 μg/mouse dose).

FIG. 6 shows the GFP expression levels in liver in animals with MC38 tumors administered intratumorally with lipid compositions containing a polynucleotide encoding GFP (2.5 μg/mouse dose).

FIG. 7 shows the GFP expression levels in tumor 24 hours post administration in animals with Hep3B tumors administered intratumorally with lipid compositions containing a polynucleotide encoding GFP (2.5 μg/mouse dose).

FIG. 8 shows the GFP expression levels in liver 24 hours post administration in animals with Hep3B tumors administered intratumorally with lipid compositions containing a polynucleotide encoding GFP (2.5 μg/mouse dose).

FIG. 9 shows the GFP expression levels in tumor 24 hours post administration in animals with Hep3B tumors administered intratumorally with lipid compositions containing a polynucleotide encoding GFP (2.5 μg/mouse dose).

FIG. 10 shows the GFP expression levels in liver 24 hours post administration in animals with Hep3B tumors administered intratumorally with lipid compositions containing a polynucleotide encoding GFP (2.5 μg/mouse dose).

FIG. 11 shows GFP expression levels in tumor at 6 hours post administration in animals with MC38 tumors after lipid formulations containing a polynucleotide encoding GFP (0.5 and 2.5 μg/mouse dose) were administered intratumorally.

FIG. 12 shows GFP expression levels in tumor at 24 hours post administration in animals with MC38 tumors after lipid formulations containing a polynucleotide encoding GFP (0.5 μg/mouse dose) were administered intratumorally.

FIG. 13 shows GFP expression levels in tumor at 24 hours post administration in animals with MC38 tumors after lipid formulations containing a polynucleotide encoding GFP (2.5 μg/mouse dose) were administered intratumorally.

FIG. 14 shows a summary of GFP expression results in tumor in animals with MC38 tumors after various lipid formulations containing a polynucleotide encoding GFP (0.5 or 2.5 μg/mouse dose) were administered intratumorally. Expression level measurements were conducted a 6 hours and 24 hours post administration.

FIG. 15 shows a summary of GFP expression results in liver in animals with MC38 tumors after various lipid formulations containing a polynucleotide encoding GFP (0.5 or 2.5 μg/mouse dose) were administered intratumorally. Expression level measurements were conducted a 6 hours and 24 hours post administration.

FIGS. 16A and 16B show IL-6 cytokine induction following intratumoral administration of lipid nanoparticles comprising an mRNA encoding GFP. IL-6 levels were measured in plasma (FIG. 16A) 6 hours and 24 hours after intratumoral administration of lipid formulations containing a polynucleotide encoding GFP (2.5 μg/mouse dose), and in tumor tissue (FIG. 16B) 24 hours after intratumoral administration of lipid formulations containing a polynucleotide encoding GFP (2.5 μg/mouse dose).

FIGS. 17A and 17B show GROα (CXCL1) cytokine induction following intratumoral administration of lipid nanoparticles comprising an mRNA encoding GFP. GROα (CXCL1) levels were measured in plasma (FIG. 17A) 6 hours and 24 hours after administration of lipid formulations containing a polynucleotide encoding GFP (2.5 μg/mouse dose), and in tumor tissue (FIG. 17B) 24 hours after administration of lipid formulations containing a polynucleotide encoding GFP (2.5 μg/mouse dose).

FIGS. 18A and 18B show IFNγ cytokine induction following intratumoral administration of lipid nanoparticles comprising an mRNA encoding GFP. IFNγ levels were measured in plasma (FIG. 18A) 6 hours and 24 hours after administration of lipid formulations containing a polynucleotide encoding GFP (2.5 μg/mouse dose), and in tumor tissue (FIG. 18B) 24 hours after administration of lipid formulations containing a polynucleotide encoding GFP (2.5 μg/mouse dose).

FIGS. 19A and 19B show TNFα cytokine induction following intratumoral administration of lipid nanoparticles comprising an mRNA encoding GFP. TNFα levels were measured in plasma (FIG. 19A) 6 hours and 24 hours after administration of lipid formulations containing a polynucleotide encoding GFP (2.5 μg/mouse dose), and in tumor tissue (FIG. 19B) 24 hours after administration of lipid formulations containing a polynucleotide encoding GFP (2.5 μg/mouse dose).

FIG. 20 shows IP-10 cytokine induction following intratumoral administration of lipid nanoparticles comprising an mRNA encoding GFP. IP-10 levels were measured in plasma 6 hours and 24 hours after administration of lipid formulations containing a polynucleotide encoding GFP (2.5 μg/mouse dose).

FIGS. 21A and 21B show G-CSF cytokine induction following intratumoral administration of lipid nanoparticles comprising an mRNA encoding GFP. G-CSF levels were measured in plasma (FIG. 21A) 6 hours and 24 hours after administration of lipid formulations containing a polynucleotide encoding GFP (2.5 μg/mouse dose), and in tumor tissue (FIG. 21B) 24 hours after administration of lipid formulations containing a polynucleotide encoding GFP (2.5 μg/mouse dose).

FIG. 22 shows IL-6 cytokine induction following intratumoral administration of lipid nanoparticles comprising an mRNA encoding GFP. IL-6 levels were measured in plasma 6 hours and 24 hours after intratumoral administration of lipid formulations containing a polynucleotide encoding GFP (0.5, 2.5, and 12.5 μg/mouse doses).

FIG. 23 shows G-CSF cytokine induction following intratumoral administration of lipid nanoparticles comprising an mRNA encoding GFP. G-CSF levels were measured in plasma 6 hours and 24 hours after intratumoral administration of lipid formulations containing a polynucleotide encoding GFP (0.5, 2.5, and 12.5 μg/mouse doses).

FIG. 24 shows GROα cytokine induction following intratumoral administration of lipid nanoparticles comprising an mRNA encoding GFP. G-CSF levels were measured in plasma 6 hours and 24 hours after intratumoral administration of lipid formulations containing a polynucleotide encoding GFP (0.5, 2.5, and 12.5 μg/mouse doses).

FIG. 25 shows GFP expression levels in tumor at 24 hours post administration in animals with MC38 tumors after lipid formulations containing a polynucleotide encoding GFP (2.5 μg/mouse dose) were administered intratumorally.

FIG. 26 shows GFP expression levels in liver at 24 hours post administration in animals with MC38 tumors after lipid formulations containing a polynucleotide encoding GFP (2.5 μg/mouse dose) were administered intratumorally.

FIG. 27A shows the level of an expressed protein and IL-6 in tumors after mice having tumors were administered intratumorally a formulation containing a lipid and an mRNA encoding the protein. FIG. 27B shows the protein to IL-6 ratios for each formulation.

FIG. 28 shows the change in the concentration of compound 18 in plasma after a single IV infusion of a formulation containing compound 18 and an mRNA encoding a protein.

FIG. 29 shows the change in the concentration of compound 18 in liver tissues after weekly dosing of a formulation containing compound 18 and an mRNA encoding a protein.

FIG. 30 shows the percentage of Ly6G⁺ in live cells 24 hours after mice having A20 tumors were administered intratumorally a formulation containing compound 18 and an mRNA encoding a protein (0.5, 2.5, and 12.5 μg/mouse doses).

FIG. 31 shows the proportion of a transmembrane target protein expressed across cell types 24 hours after mice having A20 tumors were administered intratumorally a formulation containing compound 18 and an mRNA encoding the protein (0.5, 2.5, and 12.5 μg/mouse doses).

FIG. 32 shows the sequence of GFP.

FIG. 33 shows the sequence of luciferase.

### DETAILED DESCRIPTION

The present disclosure is directed to a composition comprising (1) a lipid composition comprising an ionizable amino lipid and a quaternary amine compound and (2) a polynucleotide. In one embodiment, the amount of the quaternary amine compound ranges from about 0.01 to about 20 mole % in the lipid composition. In another embodiment, the mole ratio of the ionizable amino lipid to the quaternary amine compound is about 100:1 to about 2.5:1.

The present disclosure is also directed to a composition comprising (1) a lipid composition comprising an asymmetric phospholipid, an ionizable amino lipid, and optionally a quaternary amine compound and (2) a polynucleotide, wherein the composition is formulated for intratumoral delivery of the polynucleotide.

In another aspect, the present application provides a lipid composition (e.g., a lipid nanoparticle (LNP)) comprising (1) an ionizable amino lipid, (2) a quaternary amine compound, (3) optionally a helper lipid, (4) optionally a sterol, and (5) optionally a lipid conjugate.

In another aspect, the present application provides a lipid composition (e.g., a lipid nanoparticle (LNP)) comprising (1) an asymmetric phospholipid, (2) an ionizable amino lipid, (3) optionally a quaternary amine compound, (4) optionally a sterol, and (5) optionally a lipid conjugate.

In exemplary embodiments, the lipid composition (e.g., LNP) encapsulates a polynucleotide.

In addition, the present disclosure is directed to pharmaceutical compositions for intratumoral delivery comprising (1) a lipid composition comprising an ionizable amino lipid of formula I as disclosed below, e.g., Compound 18; and (2) a therapeutic agent or a polynucleotide encoding a therapeutic agent, e.g., an mRNA. In some aspects of the present disclosure, the lipid composition component of the pharmaceutical composition comprises additional lipids. For example, the lipid composition can include one or more phospholipids, e.g., MSPC or DSPC. The lipid composition can also comprise a quaternary amine compound such as DOTAP.

Also provided are methods to improve the retention of a therapeutic agent in a tumor after it has been administered intratumorally using a pharmaceutical composition disclosed herein. Intratumoral delivery of a polynucleotide encoding a therapeutic agent using the pharmaceutical compositions disclosed herein can result in increased expression levels of the therapeutic agent in tumor tissue with respect to the expression levels observed using reference compositions comprising compounds such as heptatriaconta-6,9,28,31-tetraen-19-yl 4-(dimethylamino)butanoate (MC3).

Additionally, using the disclosed pharmaceutical composition for intratumoral delivery can result in improved retention of the therapeutic agent in the tumor and lowered leakage of the therapeutic agent to peritumoral tissue or to other tissues such as liver tissue.

The headings provided herein are not limitations of the various aspects or aspects of the disclosure, which can be defined by reference to the specification as a whole. Accordingly, the terms defined immediately below are more fully defined by reference to the specification in its entirety. Before describing the present disclosure in detail, it is to be understood that this invention is not limited to specific compositions or process steps, as such can vary.

### I. Definition

In order that the present disclosure can be more readily understood, certain terms are first defined. As used in this application, except as otherwise expressly provided herein, each of the following terms shall have the meaning set forth below. Additional definitions are set forth throughout the application.

The invention includes embodiments in which exactly one member of the group is present in, employed in, or otherwise relevant to a given product or process. The invention includes embodiments in which more than one, or all of the group members are present in, employed in, or otherwise relevant to a given product or process.

In this specification and the appended claims, the singular forms “a”, “an” and “the” include plural referents unless the context clearly dictates otherwise. The terms “a” (or “an”), as well as the terms “one or more,” and “at least one” can be used interchangeably herein. In certain aspects, the term “a” or “an” means “single.” In other aspects, the term “a” or “an” includes “two or more” or “multiple.”

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Wherever aspects are described herein with the language “comprising,” otherwise analogous aspects described in terms of “consisting of” and/or “consisting essentially of” are also provided.

Units, prefixes, and symbols are denoted in their Systeème International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the invention. Where a value is explicitly recited, it is to be understood that values which are about the same quantity or amount as the recited value are also within the scope of the invention. Where a combination is disclosed, each subcombination of the elements of that combination is also specifically disclosed and is within the scope of the invention. Conversely, where different elements or groups of elements are individually disclosed, combinations thereof are also disclosed. Where any element of an invention is disclosed as having a plurality of alternatives, examples of that invention in which each alternative is excluded singly or in any combination with the other alternatives are also hereby disclosed; more than one element of an invention can have such exclusions, and all combinations of elements having such exclusions are hereby disclosed.

Nucleotides are referred to by their commonly accepted single-letter codes. Unless otherwise indicated, nucleic acids are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, U represents uracil.

Amino acids are referred to herein by either their commonly known three letter symbols or by the one-letter symbols recommended by the IUPAC-IUB Biochemical Nomenclature Commission. Unless otherwise indicated, amino acid sequences are written left to right in amino to carboxy orientation.

About: The term “about” as used in connection with a numerical value throughout the specification and the claims denotes an interval of accuracy, familiar and acceptable to a person skilled in the art, such interval of accuracy is ±10%.

Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

Amino acid substitution: The term “amino acid substitution” refers to replacing an amino acid residue present in a parent sequence (e.g., a consensus sequence) with another amino acid residue. In the context of the present disclosure, substitutions (even when they referred to as amino acid substitution) are conducted at the nucleic acid level, i.e., substituting an amino acid residue with an alternative amino acid residue is conducted by substituting the codon encoding the first amino acid with a codon encoding the second amino acid.

Codon substitution: The terms “codon substitution” or “codon replacement” in the context of codon optimization refer to replacing a codon present in a candidate nucleotide sequence (e.g., an mRNA encoding a therapeutic agent) with another codon. Thus, a codon can be substituted in a candidate nucleic acid sequence, for example, a nucleic acid sequence encoding a therapeutic agent via chemical peptide synthesis or through recombinant methods known in the art. Accordingly, references to a “substitution” or “replacement” at a certain location in a nucleic acid sequence (e.g., an mRNA) or within a certain region or subsequence of a nucleic acid sequence (e.g., an mRNA) refer to the substitution of a codon at such location or region with an alternative codon. The goal in codon optimization is to produce a synonymous nucleotide sequence than encodes the same polypeptide sequence encoded by the candidate nucleotide sequence. Thus, there are no amino acid substitutions in the polypeptide encoded by the codon optimized nucleotide sequence with respect to the polypeptide encoded by the candidate nucleotide sequence. A candidate nucleic acid sequence can be codon-optimized by replacing all or part of its codons according to a substitution table map. As used herein, the terms “candidate nucleic acid sequence” and “candidate nucleotide sequence” refer to a nucleotide sequence (e.g., a nucleotide sequence encoding an antibody or a functional fragment thereof) that can be codon-optimized, for example, to improve its translation efficacy. In some aspects, the candidate nucleotide sequence is optimized for improved translation efficacy after in vivo administration, e.g., intratumoral administration.

Compound: As used herein, the term “compound,” is meant to include all stereoisomers and isotopes of the structure depicted. As used herein, the term “stereoisomer” means any geometric isomer, enantiomer, or diastereomer of a compound. The present disclosure encompasses any and all stereoisomers of the compounds described herein, including stereomerically pure forms (e.g., geometrically pure, enantiomerically pure, or diastereomerically pure) and enantiomeric and stereoisomeric mixtures, e.g., racemates. Enantiomeric and stereomeric mixtures of compounds and means of resolving them into their component enantiomers or stereoisomers are well-known. “Isotopes” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods.

Corresponding Composition Without the Quaternary Amine Compound: As used herein, the term “corresponding composition without the quaternary amine compound” refers to a composition that contains all of the same ingredients except for the quaternary amine compound.

Corresponding Lipid Composition Without the Quaternary Amine Compound: As used herein, the term “corresponding lipid composition without the quaternary amine compound” refers to a lipid composition that contains all of the same ingredients except for the quaternary amine compound.

Corresponding Composition With a Symmetric Phospholipid: As used herein, the term “corresponding composition with a symmetric phospholipid” refers to a composition that contains all of the same ingredients except that the asymmetric phospholipid is replaced with a symmetric phospholipid selected from the group consisting of DSPC, DPPC, DOPC, DMPS, and DLPS.

Corresponding reference composition: The term “corresponding reference composition” refers to a pharmaceutical composition comprising the same components as a pharmaceutical composition disclosed herein, but in which the compound of formula (I) (e.g., Compound 18) has been replaced by another ionizable amino lipid. In some aspects, the term “corresponding reference composition” refers to a pharmaceutical composition for intratumoral delivery in which the lipid composition consists, or consists essentially of the ionizable amino lipid heptatriaconta-6,9,28,31-tetraen-19-yl-4-(dimethylamino)butanoate (MC3).

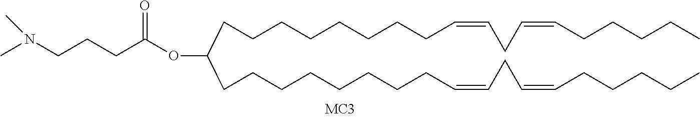 [SMILES: CCCCC/C=C\C/C=C\CCCCCCCCC(CCCCCCCC/C=C\C/C=C\CCCCC)OC(=O)CCCN(C)C]

In some aspects, the term “corresponding reference composition” refers to a pharmaceutical composition in which a compound of formula (I) as disclosed herein (e.g., Compound 18) has been replaced with MC3.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats a tumor, an effective amount of an agent is, for example, an amount sufficient to reduce or decrease a size of a tumor or to inhibit a tumor growth, as compared to the response obtained without administration of the agent. The term “effective amount” can be used interchangeably with “effective dose,” “therapeutically effective amount,” or “therapeutically effective dose.”

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Helper Lipid: As used herein, the term “helper lipid” refers to a compound or molecule that includes a lipidic moiety (for insertion into a lipid layer, e.g., lipid bilayer) and a polar moiety (for interaction with physiologic solution at the surface of the lipid layer). Typically the helper lipid is a phospholipid. A function of the helper lipid is to “complement” the amino lipid and increase the fusogenicity of the, bilayer and/or to help facilitate endosomal escape, e.g., of nucleic acid delivered to cells. Helper lipids are also believed to be a key structural component to the surface of the LNP.

Immune response: The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

Inflammatory cytokines: The term “inflammatory cytokine” refers to cytokines that are elevated in an inflammatory response. Examples of inflammatory cytokines include interleukin-6 (IL-6), CXCL1 (chemokine (C—X—C motif) ligand 1; also known as GROα, interferon-γ (IFNγ), tumor necrosis factor α (TNFα), interferon y-induced protein 10 (IP-10), or granulocyte-colony stimulating factor (G-CSF). The term inflammatory cytokines includes also other cytokines associated with inflammatory responses known in the art, e.g., interleukin-1 (IL-1), interleukin-8 (IL-8), interleukin-12 (IL-12), interleukin-13 (IL-13), interferon α (IFN-α), etc.

Ionizable amino lipid: The term “ionizable amino lipid” includes those lipids having one, two, three, or more fatty acids or fatty alkyl chains and a pH-titratable amino head group (e.g., an alkylamino or dialkylamino head group). An ionizable amino lipid is typically protonated (i.e., positively charged) at a pH below the pKa of the amino head group and is substantially not charged at a pH above the pKa. Such ionizable amino lipids include, but are not limited to MC3 and (13Z,165Z)—N,N-dimethyl-3-nonydocosa-13-16-dien-1-amine (L608).

Isolated: As used herein, the term “isolated” refers to a substance or entity that has been separated from at least some of the components with which it was associated (whether in nature or in an experimental setting). Isolated substances (e.g., nucleotide sequence or protein sequence) can have varying levels of purity in reference to the substances from which they have been associated. Isolated substances and/or entities can be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

Salts: In some aspects, the pharmaceutical composition for intratumoral delivery disclosed herein comprises salts of some of their lipid constituents. The term “salt” includes any anionic and cationic complex. Non-limiting examples of anions include inorganic and organic anions, e.g., fluoride, chloride, bromide, iodide, oxalate (e.g., hemioxalate), phosphate, phosphonate, hydrogen phosphate, dihydrogen phosphate, oxide, carbonate, bicarbonate, nitrate, nitrite, nitride, bisulfite, sulfide, sulfite, bisulfate, sulfate, thiosulfate, hydrogen sulfate, borate, formate, acetate, benzoate, citrate, tartrate, lactate, acrylate, polyacrylate, fumarate, maleate, itaconate, glycolate, gluconate, malate, mandelate, tiglate, ascorbate, salicylate, polymethacrylate, perchlorate, chlorate, chlorite, hypochlorite, bromate, hypobromite, iodate, an alkylsulfonate, an arylsulfonate, arsenate, arsenite, chromate, dichromate, cyanide, cyanate, thiocyanate, hydroxide, peroxide, permanganate, and mixtures thereof.

Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof.

A polynucleotide, vector, polypeptide, cell, or any composition disclosed herein which is “isolated” is a polynucleotide, vector, polypeptide, cell, or composition which is in a form not found in nature. Isolated polynucleotides, vectors, polypeptides, or compositions include those which have been purified to a degree that they are no longer in a form in which they are found in nature. In some aspects, a polynucleotide, vector, polypeptide, or composition which is isolated is substantially pure.

Nucleic acid sequence: The terms “nucleic acid sequence,” “nucleotide sequence,” or “polynucleotide” are used interchangeably and refer to a contiguous nucleic acid sequence. The sequence can be either single stranded or double stranded DNA or RNA, e.g., an mRNA. The phrase “polynucleotide sequence encoding” and variants thereof refers to the nucleic acid (e.g., an mRNA or DNA molecule) coding sequence that comprises a nucleotide sequence which encodes a polypeptide or functional fragment thereof as set forth herein. The coding sequence can further include initiation and termination signals operably linked to regulatory elements including a promoter and polyadenylation signal capable of directing expression in the cells of an individual or mammal to which the nucleic acid is administered. The coding sequence can further include sequences that encode signal peptides.

Open reading frame: As used herein, “open reading frame” or “ORF” refers to a sequence which does not contain a stop codon in a given reading frame.

Operably linked: As used herein, the phrase “operably linked” refers to a functional connection between two or more molecules, constructs, transcripts, entities, moieties or the like.

Polynucleotide: The term “polynucleotide” as used herein refers to polymers of nucleotides of any length, including ribonucleotides, deoxyribonucleotides, analogs thereof, or mixtures thereof. This term refers to the primary structure of the molecule. Thus, the term includes triple-, double- and single-stranded deoxyribonucleic acid (“DNA”), as well as triple-, double- and single-stranded ribonucleic acid (“RNA”). It also includes modified, for example by alkylation, and/or by capping, and unmodified forms of the polynucleotide. More particularly, the term “polynucleotide” includes polydeoxyribonucleotides (containing 2-deoxy-D-ribose), polyribonucleotides (containing D-ribose), including tRNA, rRNA, hRNA, siRNA and mRNA, whether spliced or unspliced, any other type of polynucleotide which is an N- or C-glycoside of a purine or pyrimidine base, and other polymers containing normucleotidic backbones, for example, polyamide (e.g., peptide nucleic acids “PNAs”) and polymorpholino polymers, and other synthetic sequence-specific nucleic acid polymers providing that the polymers contain nucleobases in a configuration which allows for base pairing and base stacking, such as is found in DNA and RNA. In particular aspects, the polynucleotide comprises an mRNA. In other aspect, the mRNA is a synthetic mRNA. In some aspects, the synthetic mRNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine). In some aspects, the polynucleotide (e.g., a synthetic RNA or a synthetic DNA) comprises only natural nucleobases, i.e., A, C, T and U in the case of a synthetic DNA, or A, C, T, and U in the case of a synthetic RNA.

The skilled artisan will appreciate that the T bases in the codon maps disclosed herein are present in DNA, whereas the T bases would be replaced by U bases in corresponding RNAs. For example, a codon-nucleotide sequence disclosed herein in DNA form, e.g., a vector or an in-vitro translation (IVT) template, would have its T bases transcribed as U based in its corresponding transcribed mRNA. In this respect, both codon-optimized DNA sequences (comprising T) and their corresponding RNA sequences (comprising U) are considered codon-optimized nucleotide sequence of the present disclosure. A skilled artisan would also understand that equivalent codon-maps can be generated by replaced one or more bases with non-natural bases. Thus, e.g., a TTC codon (DNA map) would correspond to a UUC codon (RNA map), which in turn would correspond to a ΨΨC codon (RNA map in which U has been replaced with pseudouridine).

Standard A-T and G-C base pairs form under conditions which allow the formation of hydrogen bonds between the N3-H and C4-oxy of thymidine and the N1 and C6-NH2, respectively, of adenosine and between the C2-oxy, N3 and C4-NH2, of cytidine and the C2-NH2, N′—H and C6-oxy, respectively, of guanosine. Thus, for example, guanosine (2-amino-6-oxy-9-β-D-ribofuranosyl-purine) can be modified to form isoguanosine (2-oxy-6-amino-9-β-D-ribofuranosyl-purine). Such modification results in a nucleoside base which will no longer effectively form a standard base pair with cytosine. However, modification of cytosine (1-β-D-ribofuranosyl-2-oxy-4-amino-pyrimidine) to form isocytosine (1-β-D-ribofuranosyl-2-amino-4-oxy-pyrimidine-) results in a modified nucleotide which will not effectively base pair with guanosine but will form a base pair with isoguanosine (U.S. Pat. No. 5,681,702 to Collins et al.). Isocytosine is available from Sigma Chemical Co. (St. Louis, Mo.); isocytidine can be prepared by the method described by Switzer et al. (1993) Biochemistry 32:10489-10496 and references cited therein; 2′-deoxy-5-methyl-isocytidine can be prepared by the method of Tor et al., 1993, J. Am. Chem. Soc. 115:4461-4467 and references cited therein; and isoguanine nucleotides can be prepared using the method described by Switzer et al., 1993, supra, and Mantsch et al., 1993, Biochem. 14:5593-5601, or by the method described in U.S. Pat. No. 5,780,610 to Collins et al. Other nonnatural base pairs can be synthesized by the method described in Piccirilli et al., 1990, Nature 343:33-37, for the synthesis of 2,6-diaminopyrimidine and its complement (1-methylpyrazolo-[4,3]pyrimidine-5,7-(4H,6H)-dione. Other such modified nucleotide units which form unique base pairs are known, such as those described in Leach et al. (1992) J. Am. Chem. Soc. 114:3675-3683 and Switzer et al., supra.

Polypeptide: The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids such as homocysteine, ornithine, p-acetylphenylalanine, D-amino acids, and creatine), as well as other modifications known in the art.

The term, as used herein, refers to proteins, polypeptides, and peptides of any size, structure, or function. Polypeptides include gene products, naturally occurring polypeptides, synthetic polypeptides, homologs, orthologs, paralogs, fragments and other equivalents, variants, and analogs of the foregoing. A polypeptide can be a single OX40L polypeptide or can be a multi-molecular complex such as a dimer, trimer or tetramer. They can also comprise single chain or multichain polypeptides. Most commonly disulfide linkages are found in multichain polypeptides. The term polypeptide can also apply to amino acid polymers in which one or more amino acid residues are an artificial chemical analogue of a corresponding naturally occurring amino acid.

Prophylaxis: As used herein, a “prophylaxis” refers to a measure taken to maintain health and prevent the spread of disease. An “immune prophylaxis” refers to a measure to produce active or passive immunity to prevent the spread of disease.

Pseudouridine: As used herein, pseudouridine refers to the C-glycoside isomer of the nucleoside uridine. A “pseudouridine analog” is any modification, variant, isoform or derivative of pseudouridine. For example, pseudouridine analogs include but are not limited to 1-carboxymethyl-pseudouridine, 1-propynyl-pseudouridine, 1-taurinomethyl-pseudouridine, 1-taurinomethyl-4-thio-pseudouridine, 1-methylpseudouridine (m¹ψ), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydropseudouridine, 2-thio-dihydropseudouridine, 2-methoxyuridine, 2-methoxy-4-thio-uridine, 4-methoxy-pseudouridine, 4-methoxy-2-thio-pseudouridine, N1-methyl-pseudouridine, 1-methyl-3-(3-amino-3-carboxypropyl)pseudouridine (acp³ ψ), and 2′-O-methyl-pseudouridine (ψm).

Subject: By “subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; bears, food animals such as cows, pigs, and sheep; ungulates such as deer and giraffes; rodents such as mice, rats, hamsters and guinea pigs; and so on. In certain embodiments, the mammal is a human subject. In other embodiments, a subject is a human patient. In a particular embodiment, a subject is a human patient in need of a cancer treatment.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Therapeutic agent: As used herein, the term “therapeutic agent” is used in a broad sense to include any molecule (e.g., polypeptide, polynucleotide,or small molecule) that can provide a significant therapeutic benefit to a subject in need thereof, e.g., a subject with a disease or condition associated with the presence of tumors. Thus, the term therapeutic agent includes for example molecules (e.g., polypeptides, polynucleotides, or small molecule) that deplete target cells in a patient, e.g., cells in a tumor. In some embodiments, the therapeutic agent can be, for example, an antibody or a polynucleotide encoding such antibody, i.e., a therapeutic antibody, or a portion thereof. Therapeutic antibodies can be directed, for example, to epitopes of surface proteins which are overexpressed by tumoral cells.

A therapeutic agent according to the present disclosure can be, for example, any molecule (e.g., polypeptides, polynucleotides, or small molecules) that can treat or ameliorate any diseases or conditions characterized by the presence of tumors (both benign and malignant tumors), wherein the agent is administered intratumorally.

The term therapeutic agent can also encompass prophylactic, diagnostic, or imaging agents wherein the agent is administered intratumorally. Intratumorally delivered therapeutic agents of the present disclosure include not only agents that act as antineoplastic agents, but also agents that can ameliorate any symptom associated with the presence of a tumor. Thus, as defined herein, the term therapeutic agent would include, for example, agents that can reduce or suppress inflammation, agents that reduce pain, agents that can promote an immune response against the tumor, agents targeting tumor vascularization, agents capable of binding to molecules present in the tumor such as tumor antigens (e.g., antibodies), agents capable of promoting, suppressing, or modulating of the levels of specific molecules in the tumor and surrounding tissue, etc.

Transfection: As used herein, “transfection” refers to the introduction of a polynucleotide (e.g., an RNA) into a cell in a target tissue wherein a therapeutic agent encoded by the polynucleotide would be expressed (e.g., mRNA) or the therapeutic agent would modulate a cellular function (e.g., siRNA, miRNA). As used herein, “expression” of a nucleic acid sequence refers to translation of an mRNA into a polypeptide or protein and/or post-translational modification of a polypeptide or protein.

Target tissue: As used herein, “target tissue” refers to any one or more tissue types of interest in which the intratumoral delivery of a therapeutic agent or a polynucleotide encoding a therapeutic agent would result in a desired biological and/or pharmacological effect. In particular applications, the target tissue can be tumor tissue. In some applications, the target tissue can be non-tumoral tissue. The term “peritumoral tissue” refers to healthy tissue surrounding the tumor. The term “off-target tissue” refers to any one or more tissue types in which the activity of therapeutic agent or polynucleotide encoding the therapeutic expression) does not result in a desired biological and/or pharmacological effect. In particular applications, off-target tissues can include liver and spleen. In some embodiments, the off-target tissue is peritumoral tissue. The term “expression leakage” refers to the expression of a polynucleotide in a location different from the target tissue. Similarly, the expression “leakage” is applied to refer to the presence of a therapeutic agent (e.g., a polypeptide) in a location different from the target tissue.

The presence of a therapeutic agent in an off-target issue can be the result of:

(i) leakage of a therapeutic agent (e.g., a polypeptide) from the intratumoral administration site of such therapeutic agent to peritumoral tissue or distant off-target tissue (e.g., liver) via diffusion or through the bloodstream;

(ii) leakage of a polynucleotide from the intratumoral administration site to peritumoral tissue or distant off-target tissue (e.g., liver) via diffusion or through the bloodstream (e.g., a polynucleotide intended to express a polypeptide in the tumor would reach the liver and the polypeptide would be expressed in the liver); or

(iii) leakage of a polypeptide after intratumoral administration of a polynucleotide encoding such polypeptide to peritumoral tissue or distant off-target tissue (e.g., liver) via diffusion or through the bloodstream (e.g., a polynucleotide would express a polypeptide in the tumor, and the polypeptide would diffuse to peritumoral tissue).

Treating, treatment, therapy: As used herein, the term “treating” or “treatment” or “therapy” refers to partially or completely alleviating, ameliorating, improving, relieving, delaying onset of, inhibiting progression of, reducing severity of, and/or reducing incidence of one or more symptoms or features of disease or condition associated with the presence of tumors (e.g., a hyper-proliferative disease such as cancer). For example, “treating” tumors can refer to inhibiting growth and/or spread of a tumor. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition, wherein such disease, disorder and/or condition is associated with the presence of tumors.

Unmodified: As used herein, “unmodified” refers to any substance, compound or molecule prior to being changed in any way. Unmodified can, but does not always, refer to the wild type or native form of a biomolecule. Molecules can undergo a series of modifications whereby each modified molecule can serve as the “unmodified” starting molecule for a subsequent modification.

### II. Compositions

### Formulation, Administration, Delivery and Dosing

The present application provides a composition comprising (1) a lipid composition which comprises an ionizable amino lipid and a quaternary amine compound and (2) a polynucleotide. In some embodiments of the present disclosure, the present application provides that a particular concentration (or concentration ranges) of a quaternary amine compound in combination with an ionizable amine lipid can improve one or more properties of the lipid composition.

The present application also provides provides a composition comprising (1) a lipid composition comprising an asymmetric phospholipid, an ionizable amino lipid, and optionally a quaternary amine compound and (2) a polynucleotide, wherein the composition is formulated for intratumoral delivery of the polynucleotide.

Without being bound by the theory, the inclusion of a quaternary amine compound to the lipid composition can increase positive charge on the surface of the lipid nanoparticles. An optimized positive charge of a lipid composition increases retention of the polynucleotide delivered to a tumor tissue, increases expression of the polypeptide in a tumor tissue, and/or decreases expression of the polypeptide in a non-tumor tissue, e.g., liver. In one embodiment, the net positive charge of the lipid composition is increased compared to the net positive charge of a corresponding lipid composition without the quaternary amine compound. The composition formulated as described herein, e.g., comprising an ionizable amino lipid and a quaternary amine compound, or comprising an asymmetric phospholipid and an ionizable amino lipid, can have one or more improved properties: (1) increase stability; (2) increase cell transfection; (3) permit the sustained or delayed release (e.g., from a depot formulation of the polynucleotide); (4) alter the biodistribution (e.g., target the polynucleotide to specific tissues or cell types); (5) increase the translation of encoded protein in vivo; and/or (6) alter the release profile of encoded protein in vivo.

The compositions disclosed herein (e.g., in lipid nanoparticle form) can be used for intratumoral delivery of a polynucleotide (e.g., an mRNA). The compositions can, for example:

(i) increase the retention of the polynucleotide in the tumor;

(ii) increase the levels of expressed polypeptide in the tumor;

(iii) decrease spillage of the polynucleotide or expressed polypeptide to a non-tumor tissue (e.g., liver tissue); or

(iv) any combination thereof,

wherein the increase or decrease observed for a certain property is relative to a corresponding composition without the quaternary amine compound or a corresponding composition without the quaternary amine compound with a symmetric phospholipid.

Formulations of the pharmaceutical compositions described herein can be prepared by any method known or hereafter developed in the art of pharmacology. In general, such preparatory methods include the step of associating the active ingredient with an excipient and/or one or more other accessory ingredients.

A pharmaceutical composition in accordance with the present disclosure can be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, the formulations described herein contain at least one polynucleotide. As a non-limiting example, the formulations contain 1, 2, 3, 4 or 5 polynucleotides, e.g., mRNA. In other embodiments, the polynucleotide is formulated for intratumoral delivery in a tumor of a patient in need thereof.

Pharmaceutical formulations can additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006). The use of a conventional excipient medium can be contemplated within the scope of the present disclosure, except insofar as any conventional excipient medium can be incompatible with a substance or its derivatives, such as by producing any undesirable biological effect or otherwise interacting in a deleterious manner with any other component(s) of the pharmaceutical composition.

In some embodiments, the particle size of the lipid nanoparticle is increased and/or decreased. The change in particle size can be able to help counter biological reaction such as, but not limited to, inflammation or can increase the biological effect of the modified mRNA delivered to mammals.

Pharmaceutically acceptable excipients used in the manufacture of pharmaceutical compositions include, but are not limited to, inert diluents, surface active agents and/or emulsifiers, preservatives, buffering agents, lubricating agents, and/or oils. Such excipients can optionally be included in the pharmaceutical formulations of the present disclosure.

### III. Lipid Composition

In vivo delivery of nucleic acids can be affected by many parameters, including, but not limited to, the formulation composition, nature of particle PEGylation, degree of loading, polynucleotide to lipid ratio, and biophysical parameters such as, but not limited to, particle size (Akinc et al., Mol Ther. 2009 17:872-879). As an example, small changes in the anchor chain length of poly(ethylene glycol) (PEG) lipids can result in significant effects on in vivo efficacy. Formulations with the different lipidoids, including, but not limited to penta[3-(1-laurylaminopropionyl)]-triethylenetetramine hydrochloride (TETA-5LAP; aka 98N12-5, see Murugaiah et al., Analytical Biochemistry, 401:61 (2010)), C12-200 (including derivatives and variants), and MD1, can be tested for in vivo activity.

The lipidoid referred to herein as “98N12-5” is disclosed by Akinc et al., Mol Ther. 2009 17:872-879.

The lipidoid referred to herein as “C12-200” is disclosed by Love et al., Proc Natl Acad Sci USA. 2010 107:1864-1869 and Liu and Huang, Molecular Therapy. 2010 669-670. The lipidoid formulations can include particles comprising either 3 or 4 or more components in addition to polynucleotides.

Lipidoids and polynucleotide formulations comprising lipidoids are described in International Patent Application No. PCT/US2014/097077.

Liposomes are artificially-prepared vesicles which can primarily be composed of a lipid bilayer and can be used as a delivery vehicle for the administration of pharmaceutical formulations. Liposomes can be of different sizes such as, but not limited to, a multilamellar vesicle (MLV) which can be hundreds of nanometers in diameter and can contain a series of concentric bilayers separated by narrow aqueous compartments, a small unicellular vesicle (SUV) which can be smaller than 50 nm in diameter, and a large unilamellar vesicle (LUV) which can be between 50 and 500 nm in diameter. Liposome design can include, but is not limited to, opsonins or ligands in order to improve the attachment of liposomes to unhealthy tissue or to activate events such as, but not limited to, endocytosis. Liposomes can contain a low or a high pH in order to improve the delivery of the pharmaceutical formulations.

The formation of liposomes can depend on the physicochemical characteristics such as, but not limited to, the pharmaceutical formulation entrapped and the liposomal ingredients, the nature of the medium in which the lipid vesicles are dispersed, the effective concentration of the entrapped substance and its potential toxicity, any additional processes involved during the application and/or delivery of the vesicles, the optimization size, polydispersity and the shelf-life of the vesicles for the intended application, and the batch-to-batch reproducibility and possibility of large-scale production of safe and efficient liposomal products.

### Ionizable Amino Lipid

The term “ionizable amino lipid” is used to include those lipids having one, two, three, or more fatty acid or fatty alkyl chains and a pH-titratable amino head group (e.g., an alkylamino or dialkylamino head group). An ionizable amino lipid is typically protonated (i.e., positively charged) at a pH below the pKa of the amino head group and is substantially not charged at a pH above the pKa. In some embodiments, the ionizable amino lipids comprise: (1) a protonatable tertiary amine (e.g., pH-titratable) head group; and (2) at least one hydrophobic tail group comprising (i) C₈₋₄₀ linear or branched hydrocarbon chains, wherein each hydrocarbon chain independently has 0 to 3 (e.g., 0, 1, 2, or 3) double bonds and (ii) optionally ether, ester, carbonyl or ketal linkages between the head group and the hydrocarbon chains. In some embodiments, the ionizable amino lipid comprises two identical tail groups. In some embodiments, the ionizable amino lipid comprises two different tail groups. In some embodiments, the tail groups are linear. In some embodiments, the tail groups are branched. In some embodiments, the ionizable amino lipid comprises at least one branched tail group.

The term “alkylamino” includes a group of formula —N(H)R^{a}, wherein R^{a} is an alkyl as defined herein.

The term “dialkylamino” includes a group of formula —N(R^{a})₂, wherein each R^{a} is independently an alkyl as defined herein.

The term “alkyl” includes a straight chain or branched, noncyclic or cyclic, saturated aliphatic hydrocarbon containing from 1 to 24 carbon atoms. Representative saturated straight chain alkyls include, but are not limited to, methyl, ethyl, n-propyl, n-butyl, n-pentyl, n-hexyl, and the like, while saturated branched alkyls include, without limitation, isopropyl, sec-butyl, isobutyl, tert-butyl, isopentyl, and the like. Representative saturated cyclic alkyls include, but are not limited to, cyclopropyl, cyclobutyl, cyclopentyl, cyclohexyl, and the like, while unsaturated cyclic alkyls include, without limitation, cyclopentenyl, cyclohexenyl, and the like.

Ionizable amino lipids include, but are not limited to, DLin-MC3-DMA (MC3), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, and DLin-2-DMAP. In some embodiments, the ionizable amino lipids include, but not limited to 3-(didodecylamino)-N1,N1,4-tridodecyl-1-piperazineethanamine (KL10), N1-[2-(didodecylamino)ethyl]-N1,N4,N4-tridodecyl-1,4-piperazinediethanamine (KL22), 14,25 ditridecyl-15,18,21,24-tetraaza-octatriacontane (KL25), Octyl-CLinDMA, Octyl-CLinDMA (2R), and Octyl-CLinDMA (2S).

Ionizable amino lipids also include, but are not limited to (13Z,16Z)—N,N-dimethyl-3-nonyldocosa-13,16-dien-1-amine (L608), (20Z,23Z)—N,N-dimethylnonacosa-20,23-dien-10-amine, (17Z,20Z)—N,N-dimemylhexacosa-17,20-dien-9-amine, (16Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)—N,N-dimethyl eptacos-15-en-10-amine, (14Z)—N,N-dimethylnonacos-14-en-10-amine, (17Z)—N,N-dimethylnonacos-17-en-10-amine, (24Z)—N,N-dimethyltritriacont-24-en-10-amine, (20Z)—N,N-dimethylnonacos-20-en-10-amine, (22Z)—N,N-dimethylhentriacont-22-en-10-amine, (16Z)—N,N-dimethylpentacos-16-en-8-amine, (12Z,15Z)—N,N-dimethyl-2-nonylhenicosa-12,15-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)-N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yl oxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3,7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, and (11E,20Z,23Z)—N,N-dimethylnonacosa-11,20,2-trien-10-amine. In one embodiment, the ionizable amino lipid is MC3. In one embodiment, the ionizable amino lipid is L608.

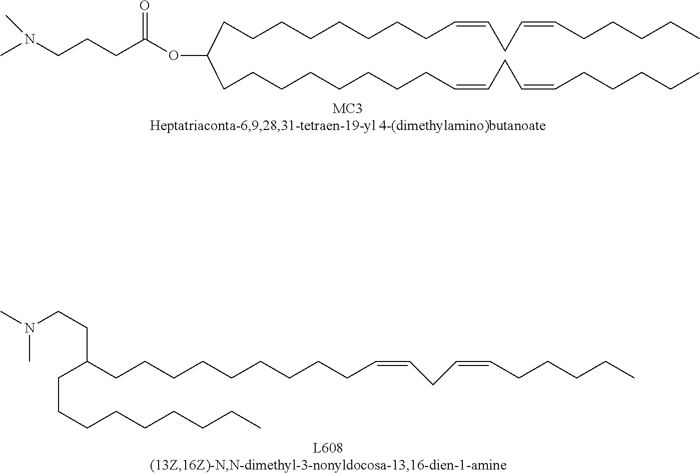

Ionizable amino lipids are known in the art, such as those described in WO 2015/130584, WO 2015/011633 A1, WO 2012/040184, US 2011/0224447, US 2012/0295832, and US 2015/0315112 A1, which are incorporated herein by reference in their entirety.

In some embodiments, the ionizable amino lipid can be a compound having a structure of Formula (I):

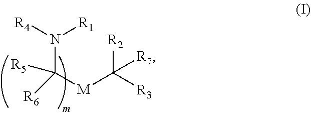

wherein

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and m is selected from 5, 6, 7, 8, 9, 10, 11, 12, and 13,

or salts or stereoisomers thereof, wherein alkyl and alkenyl groups may be linear or branched.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In certain embodiments, a subset of compounds of Formula (I) includes those of Formula (IA):

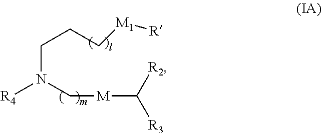

or a salt or stereoisomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, or —NHC(O)N(R)₂; M and M′ are independently selected from —C(O)O—, —OC(O)—, —C(O)N(R′)—, —P(O)(OR′)O—, an aryl group, and a heteroaryl group; and

R₂ and R₃ are independently selected from the group consisting of H, C₁₋₁₄ alkyl, and C₂₋₁₄ alkenyl.

In certain embodiments, a subset of compounds of Formula (I) includes those of Formula (II):

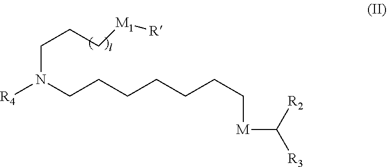

or a salt or stereoisomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, or —NHC(O)N(R)₂; M and M′ are independently selected from —C(O)O—, —OC(O)—, —C(O)N(R′)—, —P(O)(OR′)O—, an aryl group, and a heteroaryl group; and

R₂ and R₃ are independently selected from the group consisting of H, C₁₋₁₄ alkyl, and C₂₋₁₄ alkenyl.

In some embodiments, a subset of compounds of formula (I) is of the formula (IIa),

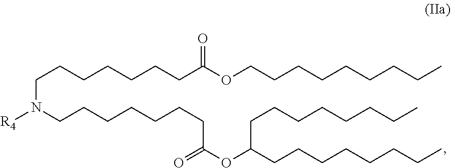

or a salt thereof, wherein R₄ is as described above.

In some embodiments, a subset of compounds of formula (I) is of the formula (IIb),

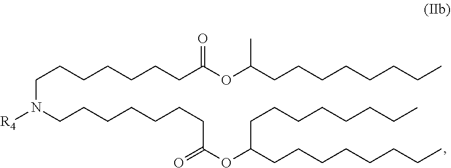

or a salt thereof, wherein R₄ is as described above.

In some embodiments, a subset of compounds of formula (I) is of the formula (IIc),

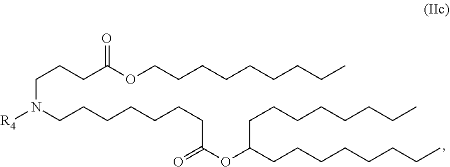

or a salt thereof, wherein R₄ is as described above.

In some embodiments, a subset of compounds of formula (I) is of the formula (IIe):

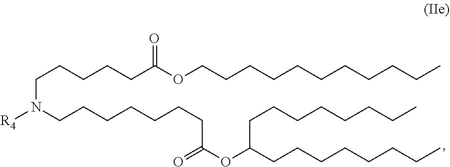

or a salt thereof, wherein R₄ is as described above.

In some embodiments, the compound of formula (IIa), (IIb), (IIc), or (IIe) comprises an R₄ which is selected from —(CH₂)ₙQ and —(CH₂)ₙCHQR, wherein Q, R and n are as defined above.

In some embodiments, Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle, wherein R is as defined above. In some aspects, n is 1 or 2. In some embodiments, Q is OH, —NHC(S)N(R)₂, or —NHC(O)N(R)₂.

In some embodiments, a subset of compounds of formula (I) is of the formula (IId),

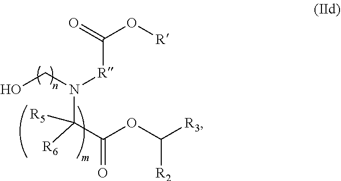

or a salt thereof, wherein R₂ and R₃ are independently selected from the group consisting of C₅₋₁₄ alkyl and C₅₋₁₄ alkenyl, n is selected from 2, 3, and 4, and R′, R″, R₅, R₆ and m are as defined above.

In some aspects of the compound of formula (IId), R₂ is C₈ alkyl. In some aspects of the compound of formula (IId), R₃ is C₅-C₉ alkyl. In some aspects of the compound of formula (IId), m is 5, 7, or 9. In some aspects of the compound of formula (IId), each R₅ is H. In some aspects of the compound of formula (IId), each R₆ is H.

For example, the ionizable amino lipids of formula (I) include, but not limited to:

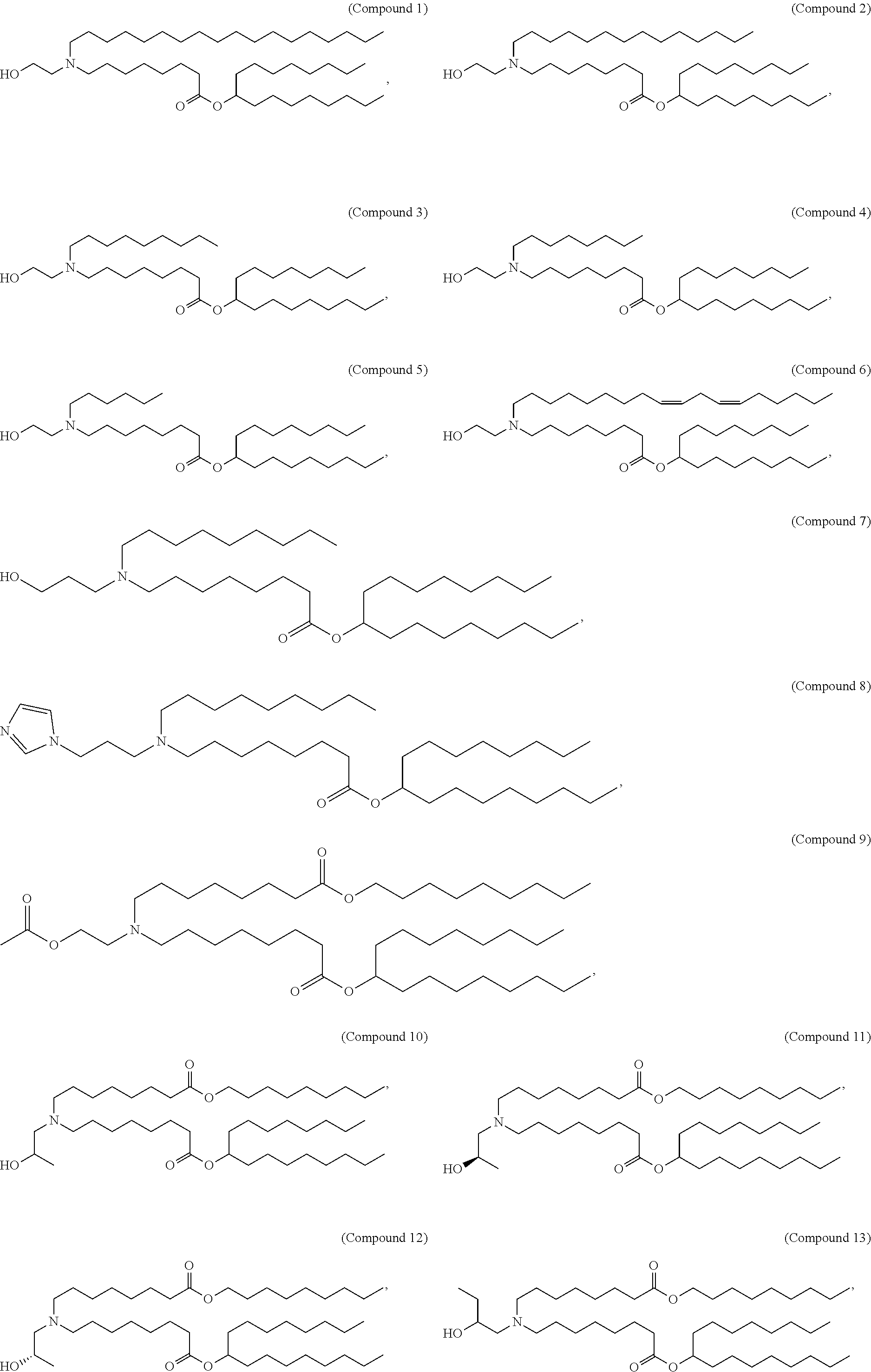

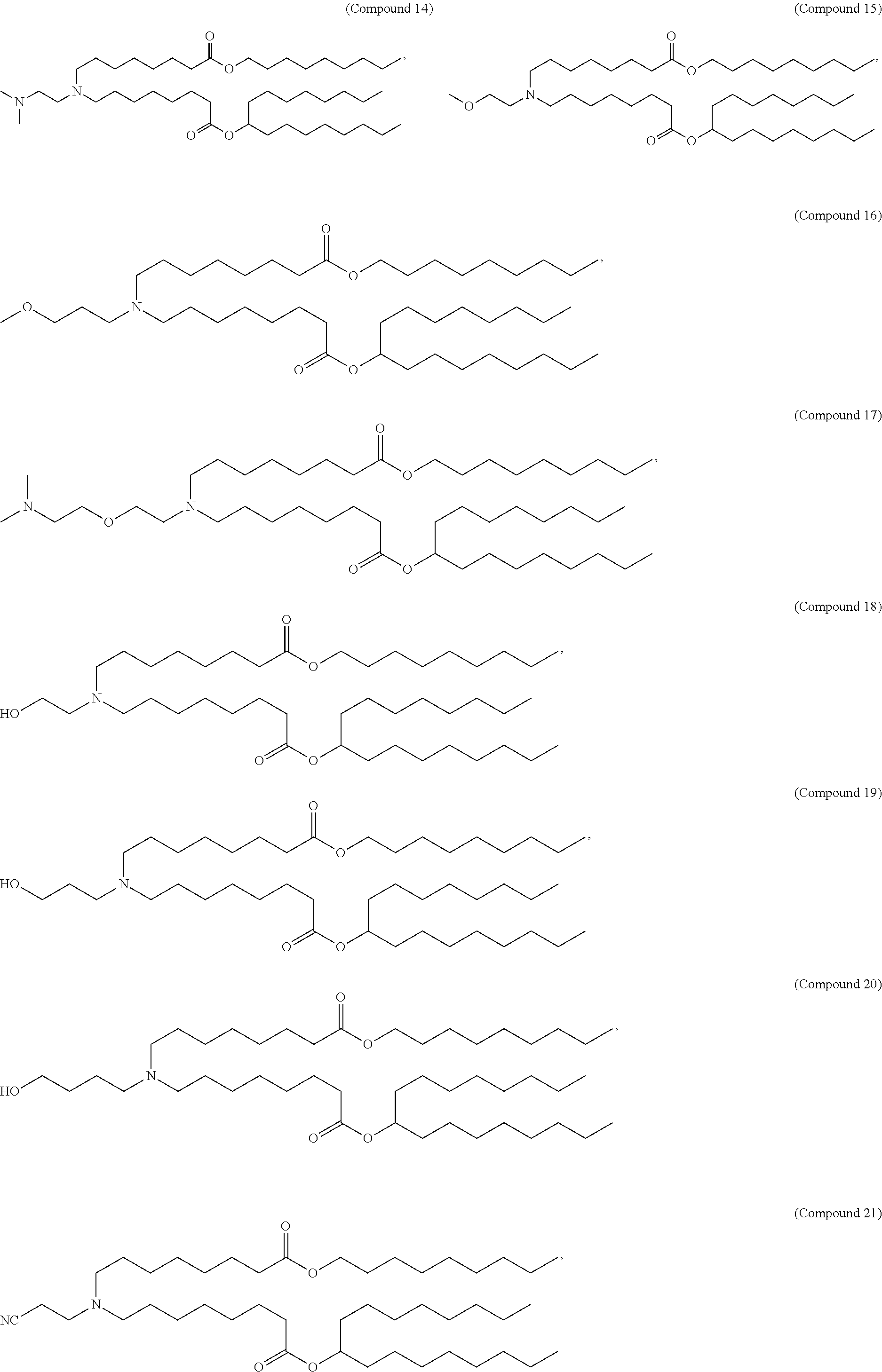

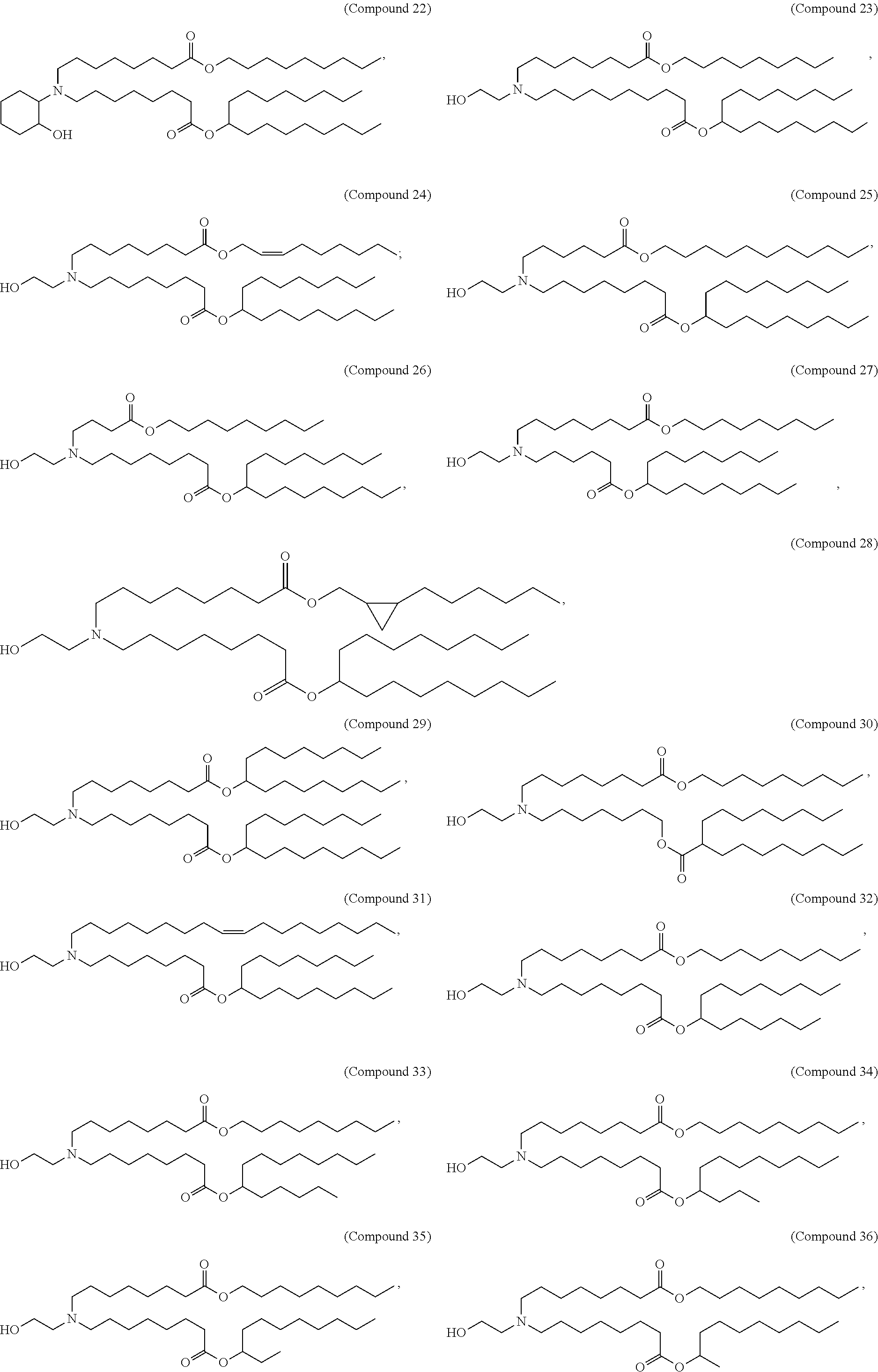

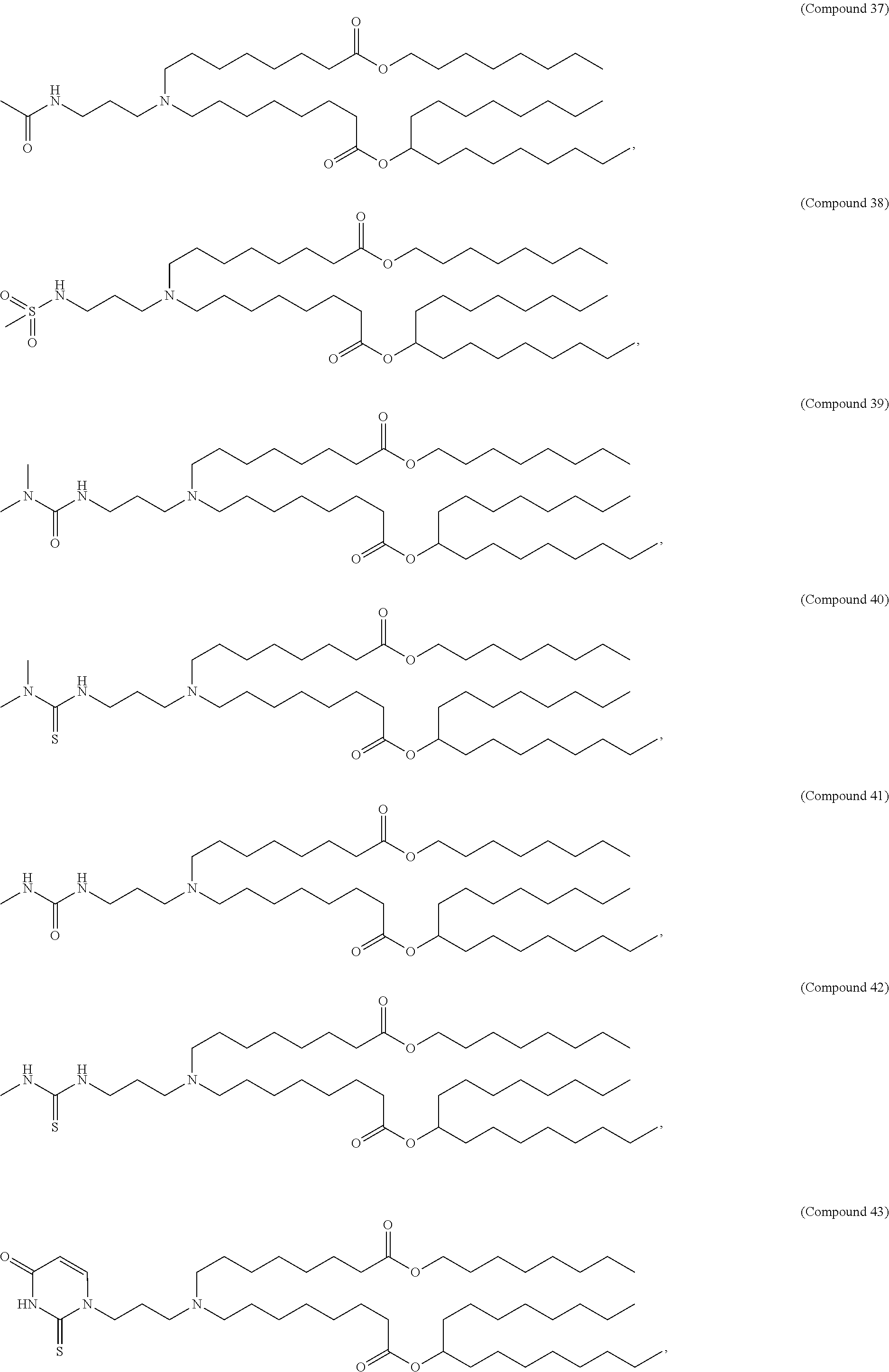

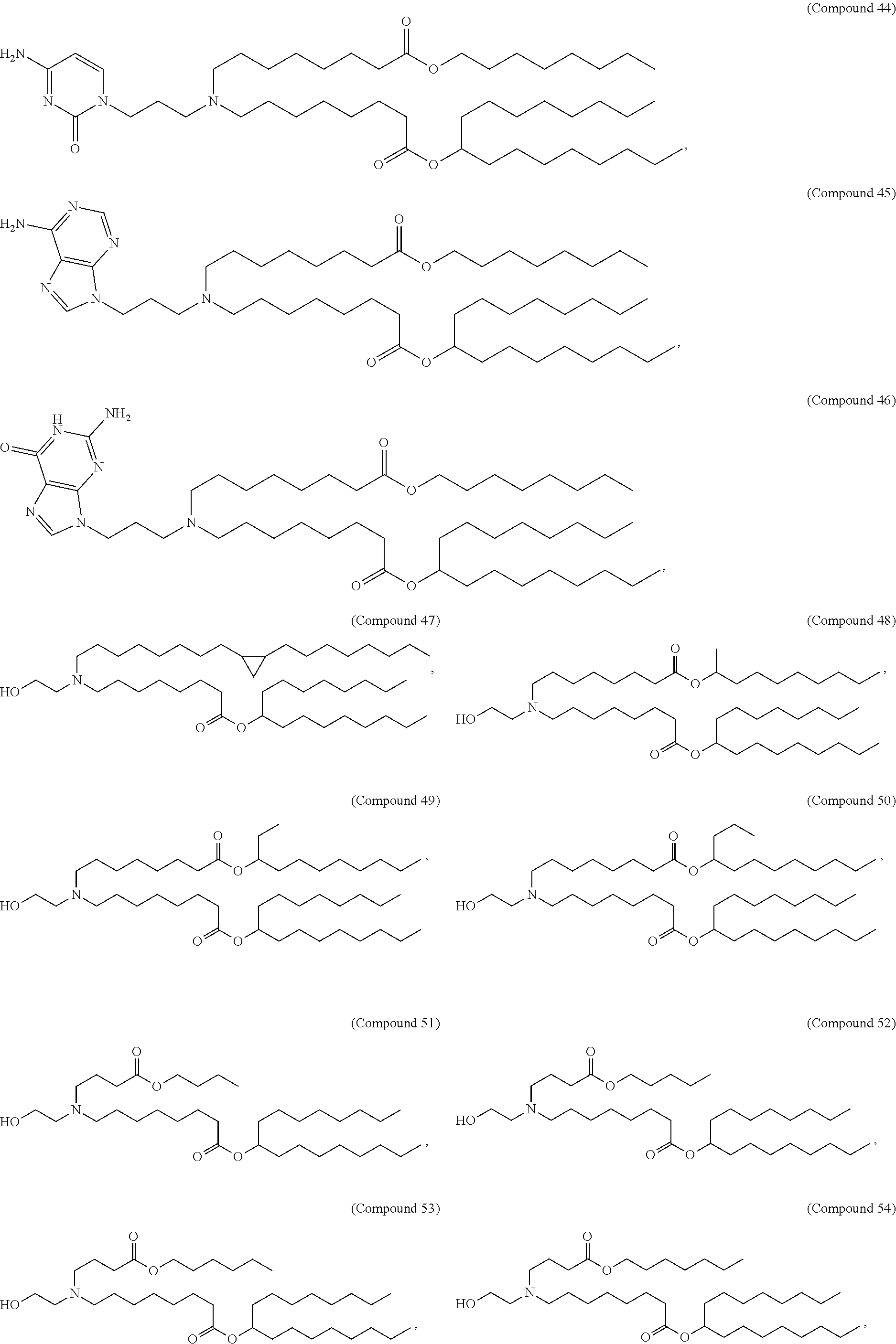

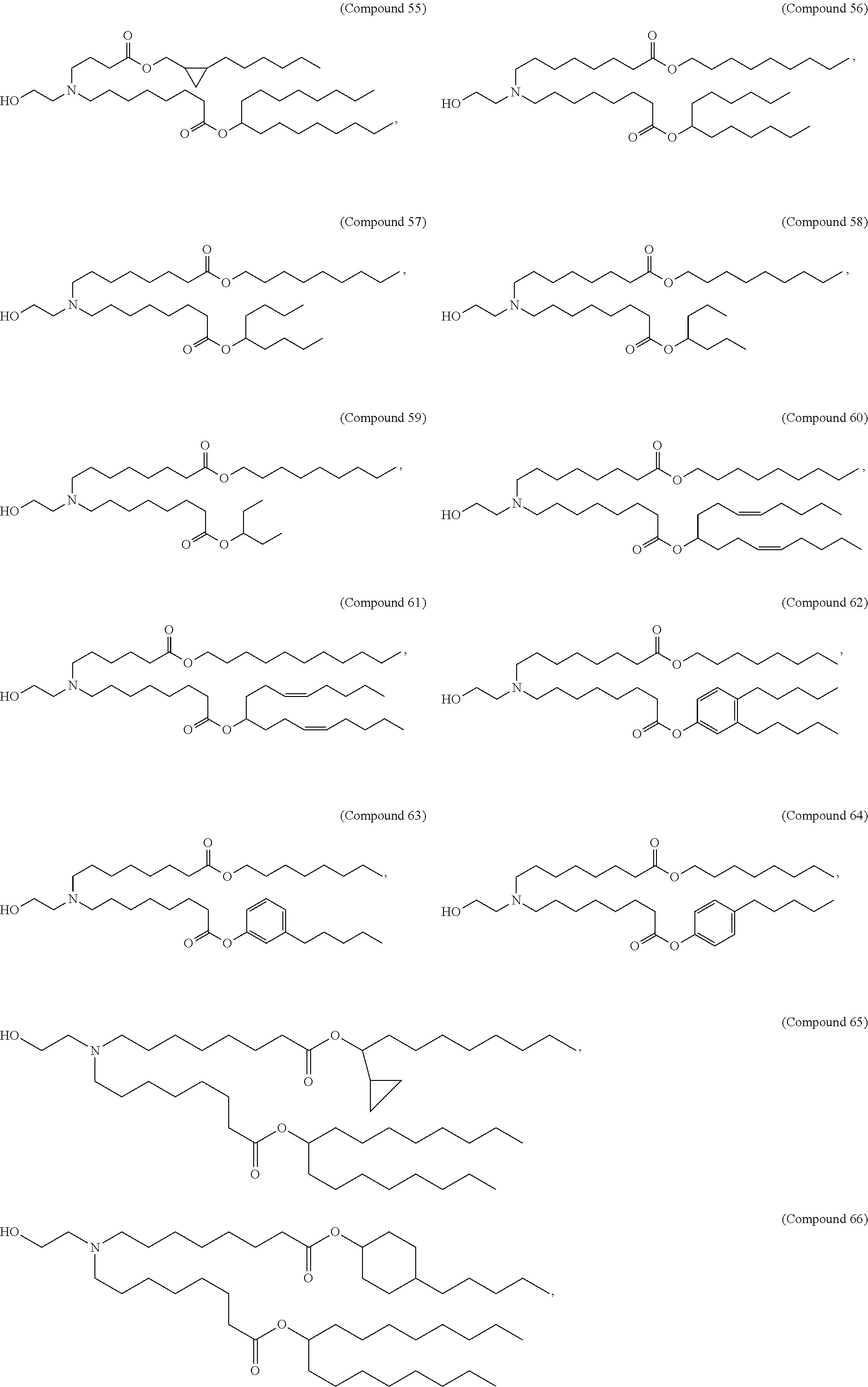

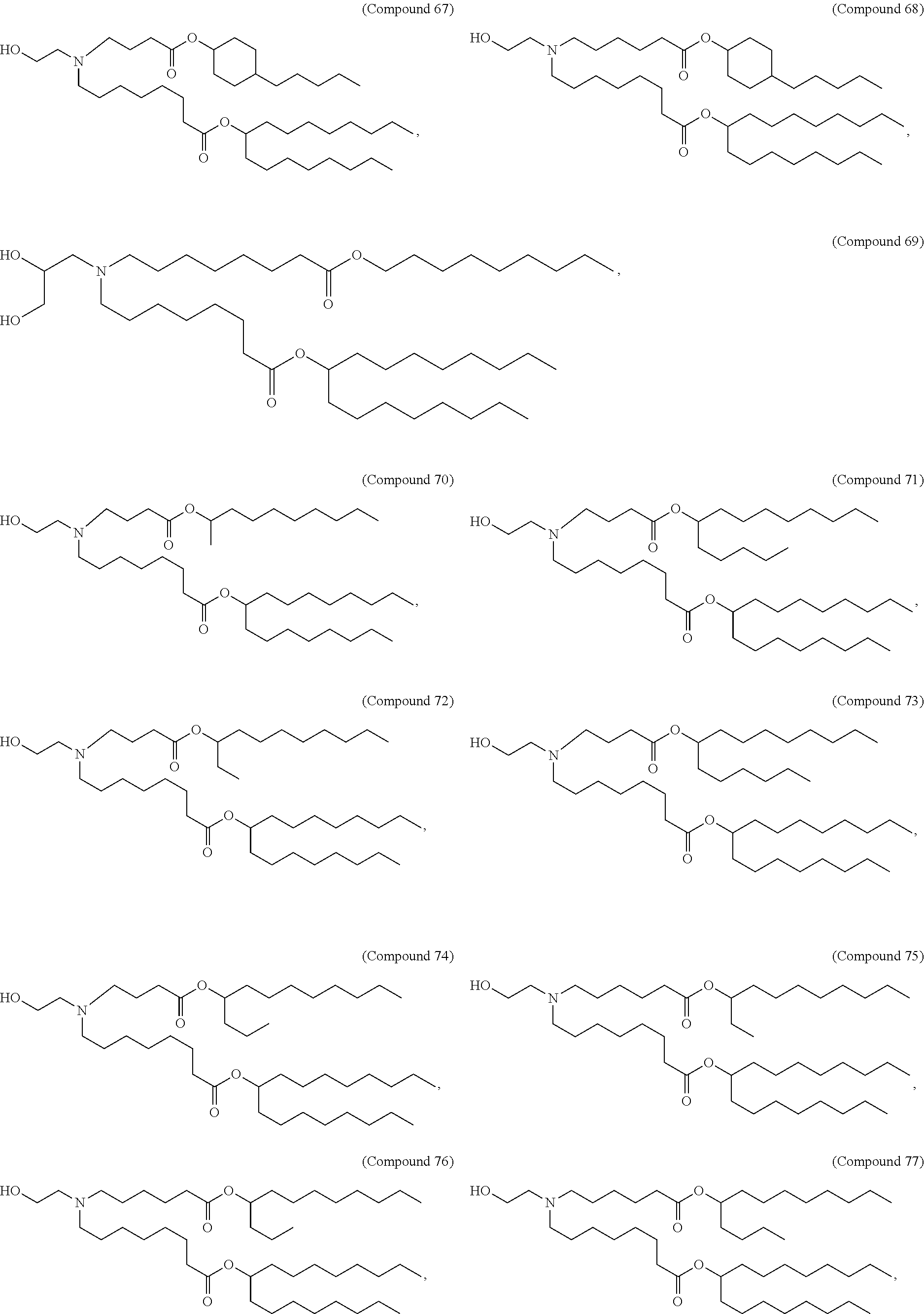

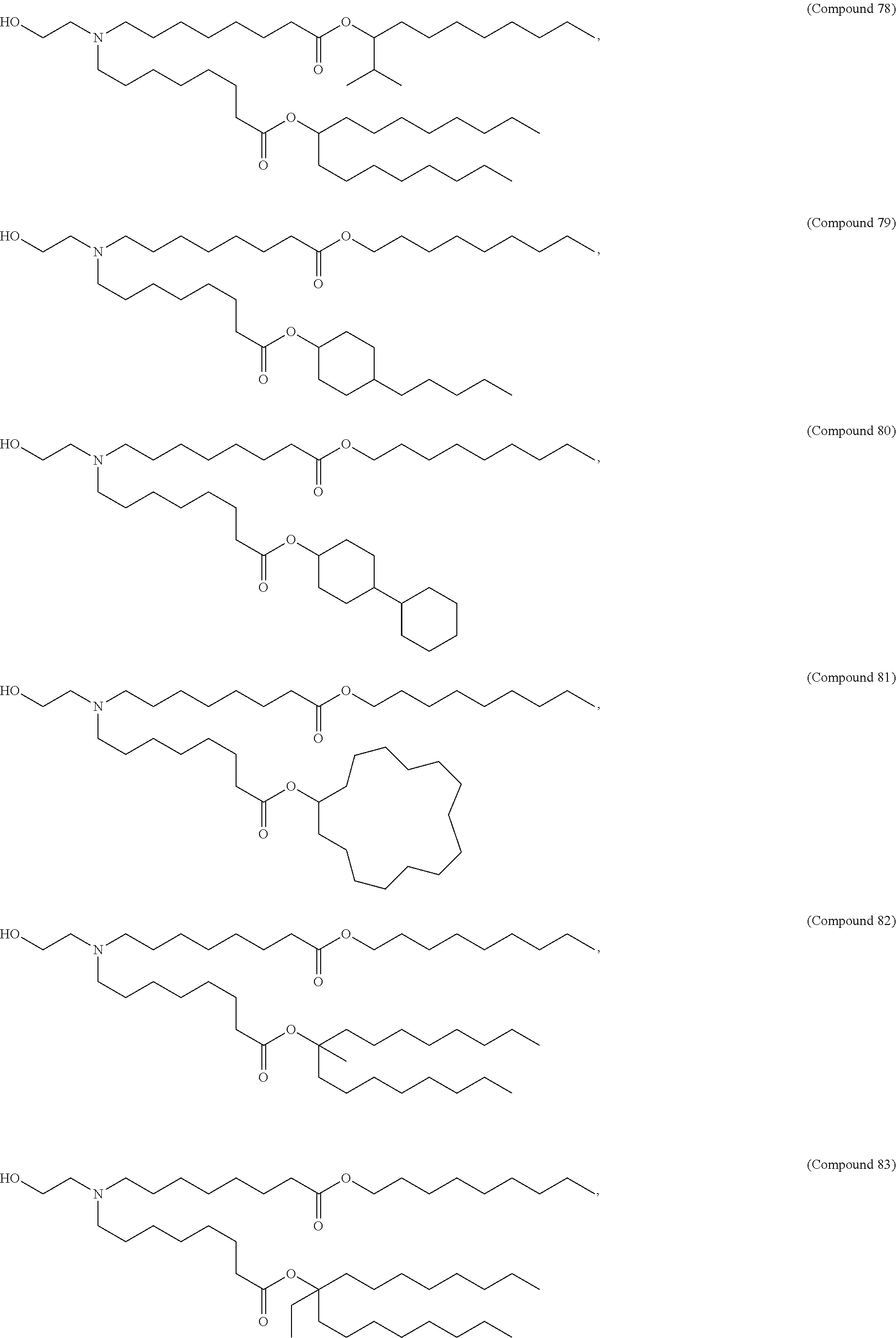 [SMILES: CCCCCCCCC(CCCCCCCC)OC(=O)CCCCCCCN(CCO)CCCCCCCC(=O)OC(CCCCCCCC)C(C)C ; CCCCCCCCCOC(=O)CCCCCCCN(CCO)CCCCCCCC(=O)OC1CCC(CCCCC)CC1 ; CCCCCCCCCOC(=O)CCCCCCCN(CCO)CCCCCCCC(=O)OC1CCC(C2CCCCC2)CC1 ; CCCCCCCCCOC(=O)CCCCCCCN(CCO)CCCCCCCC(=O)OC1CCCCCCCCCCCCCC1 ; CCCCCCCCCOC(=O)CCCCCCCN(CCO)CCCCCCCC(=O)OC(C)(CCCCCCCC)CCCCCCCC ; CCCCCCCCCOC(=O)CCCCCCCN(CCO)CCCCCCCC(=O)OC(CC)(CCCCCCCC)CCCCCCCC]

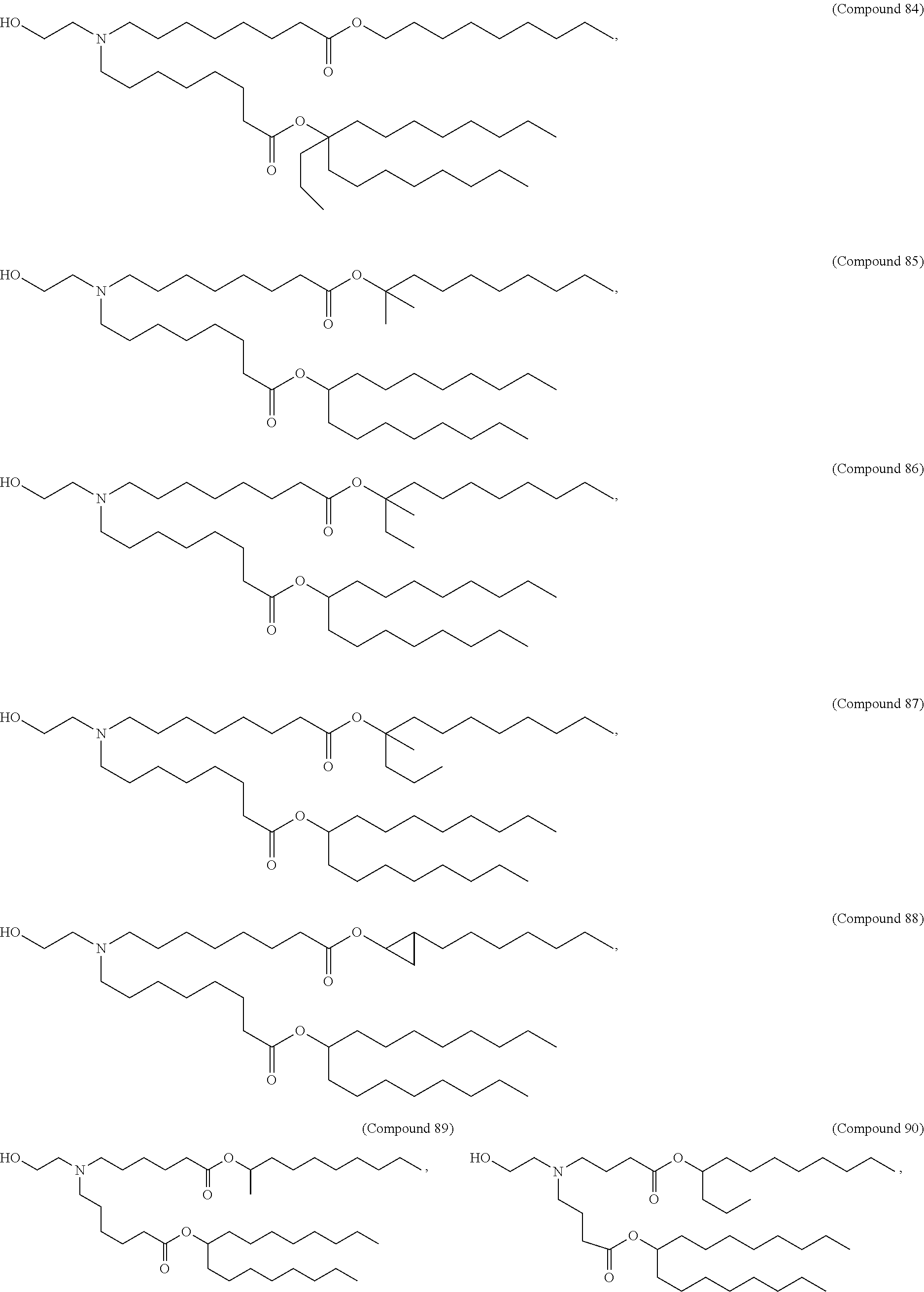

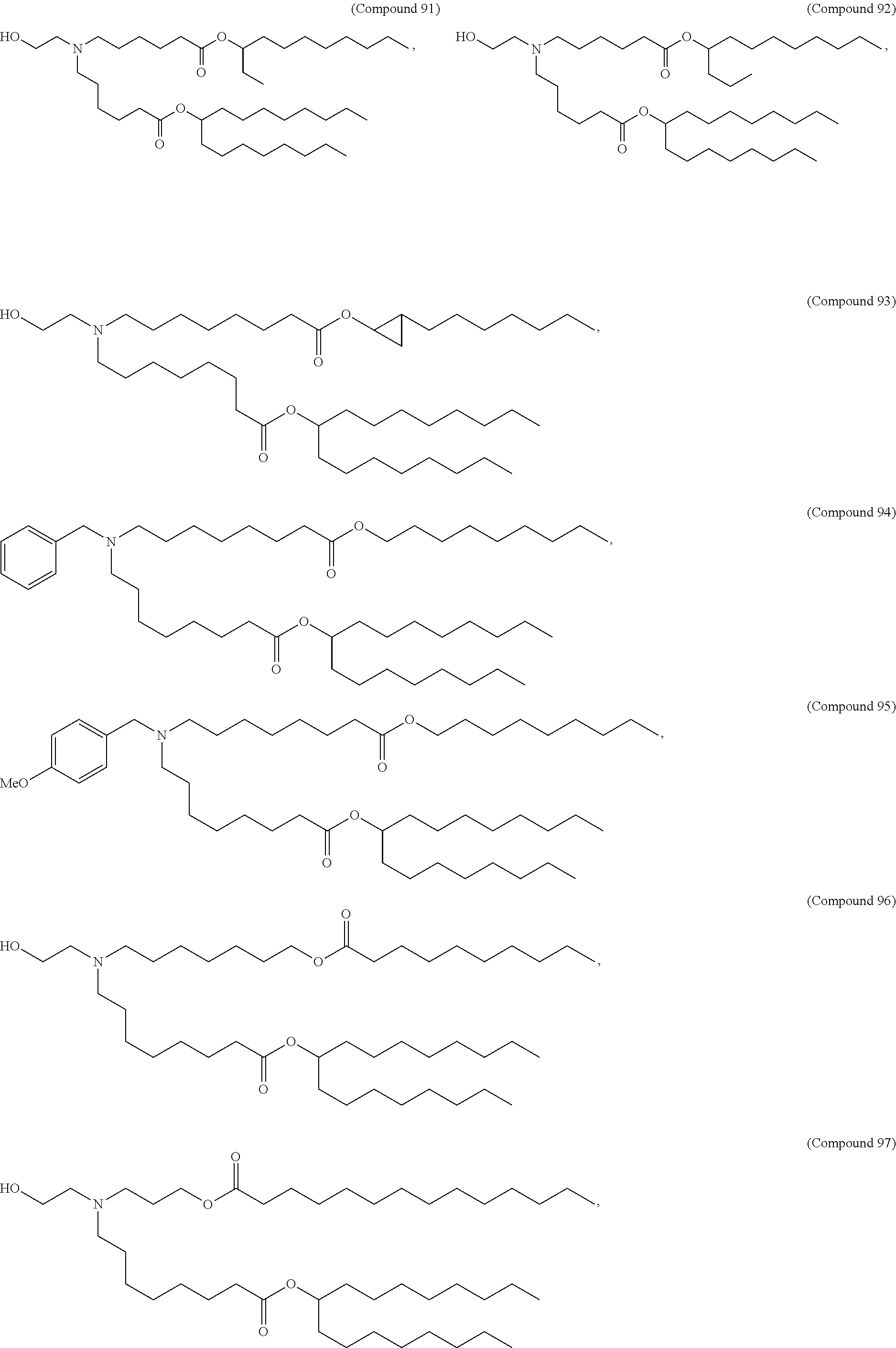

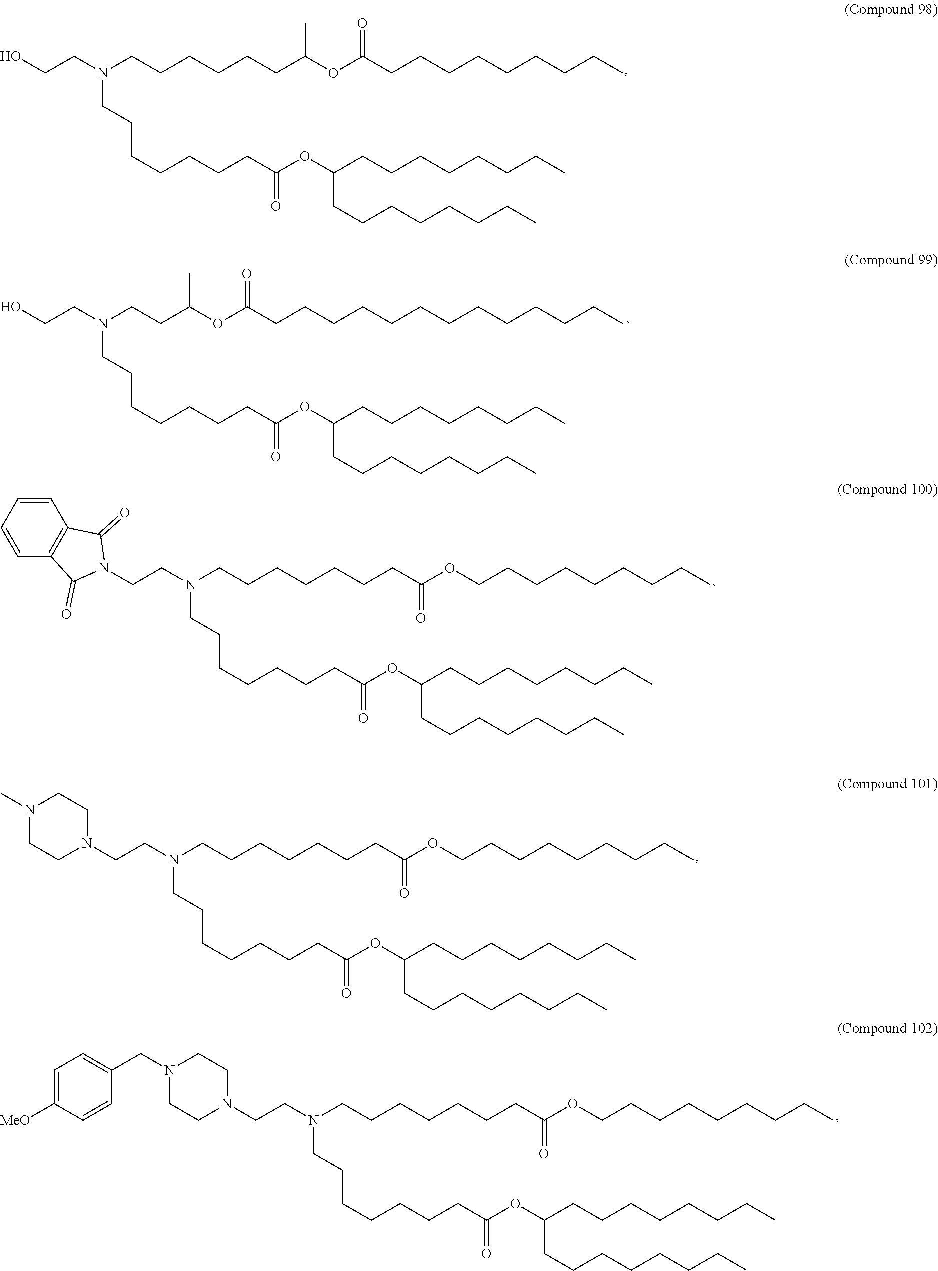

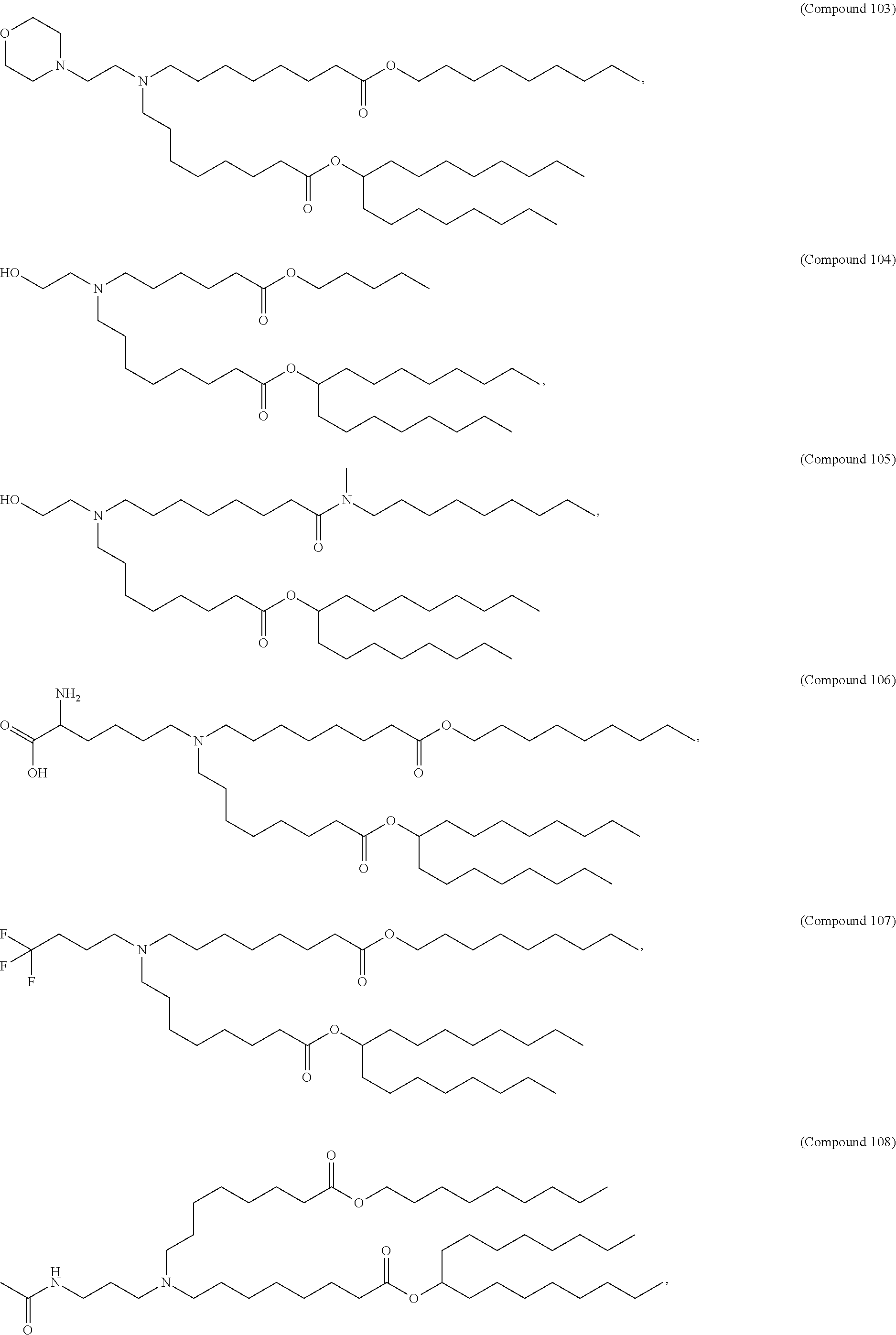

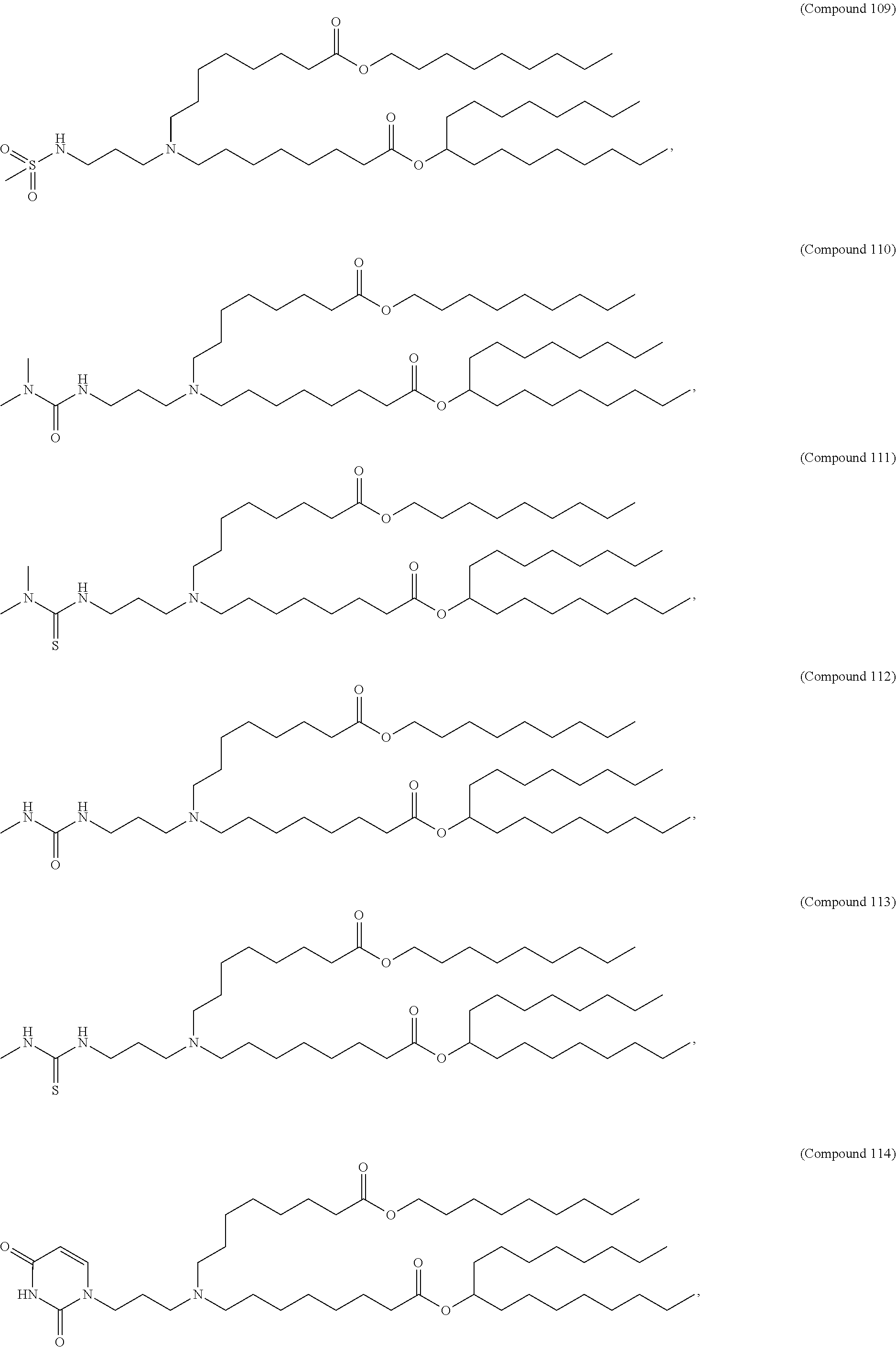

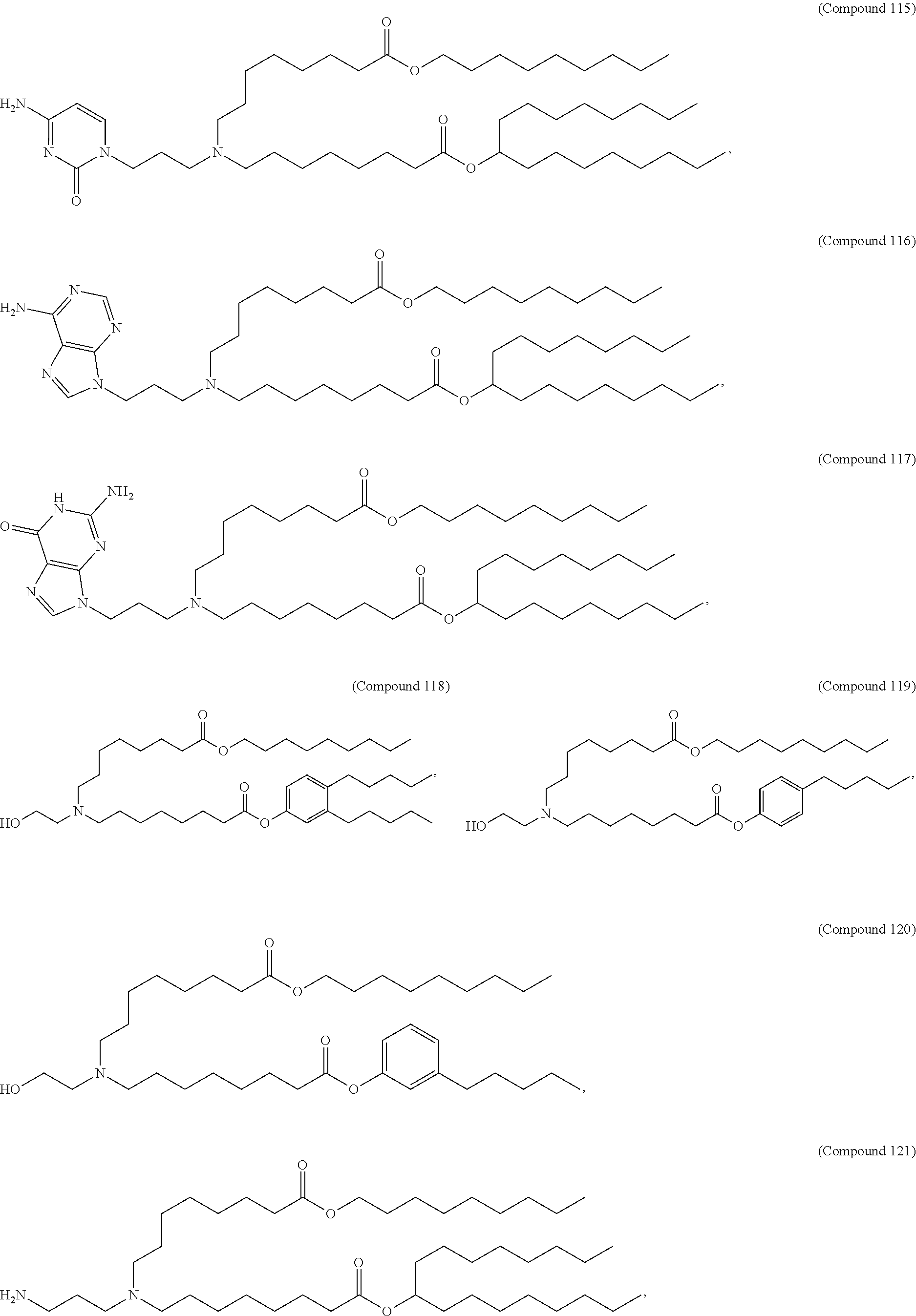

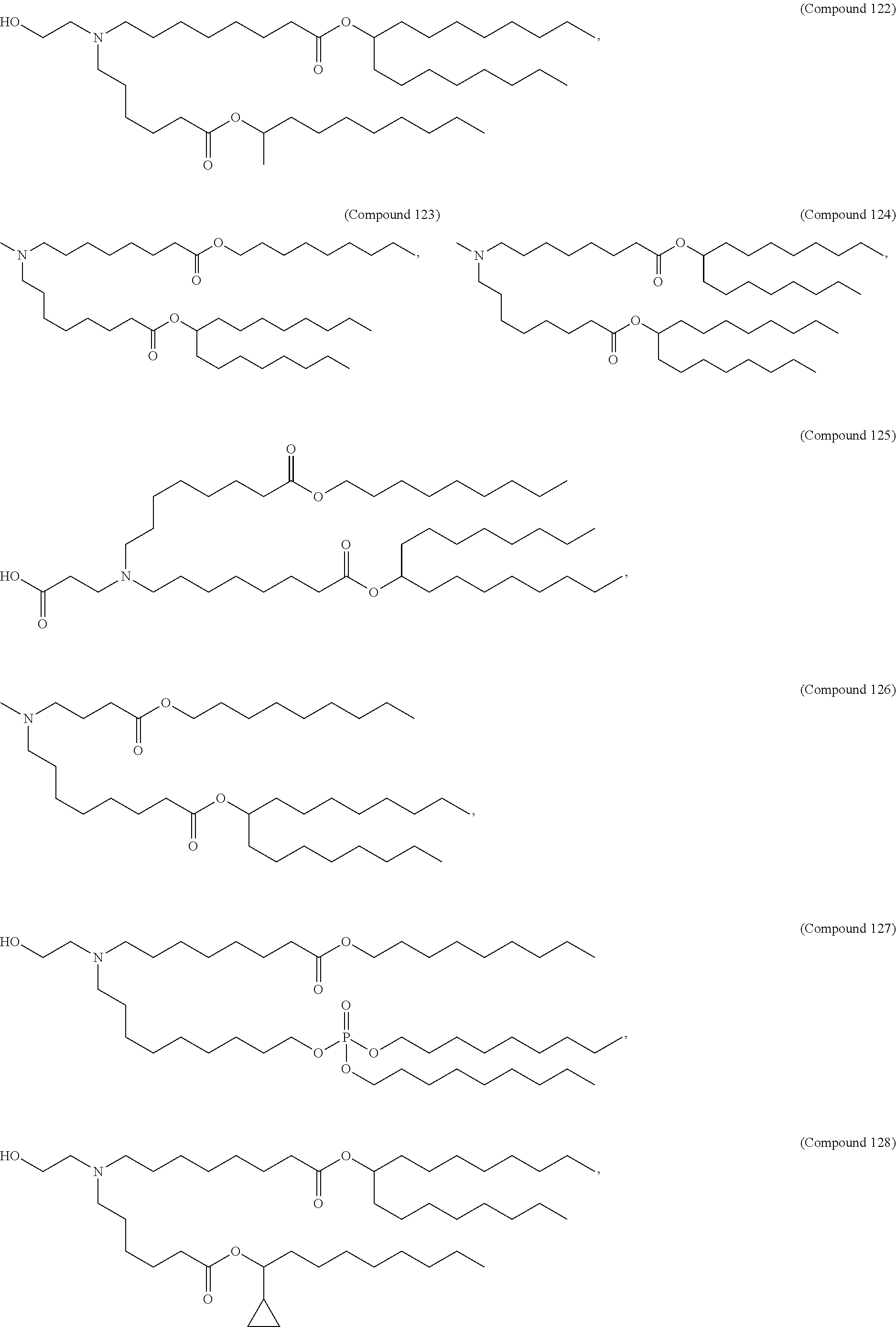

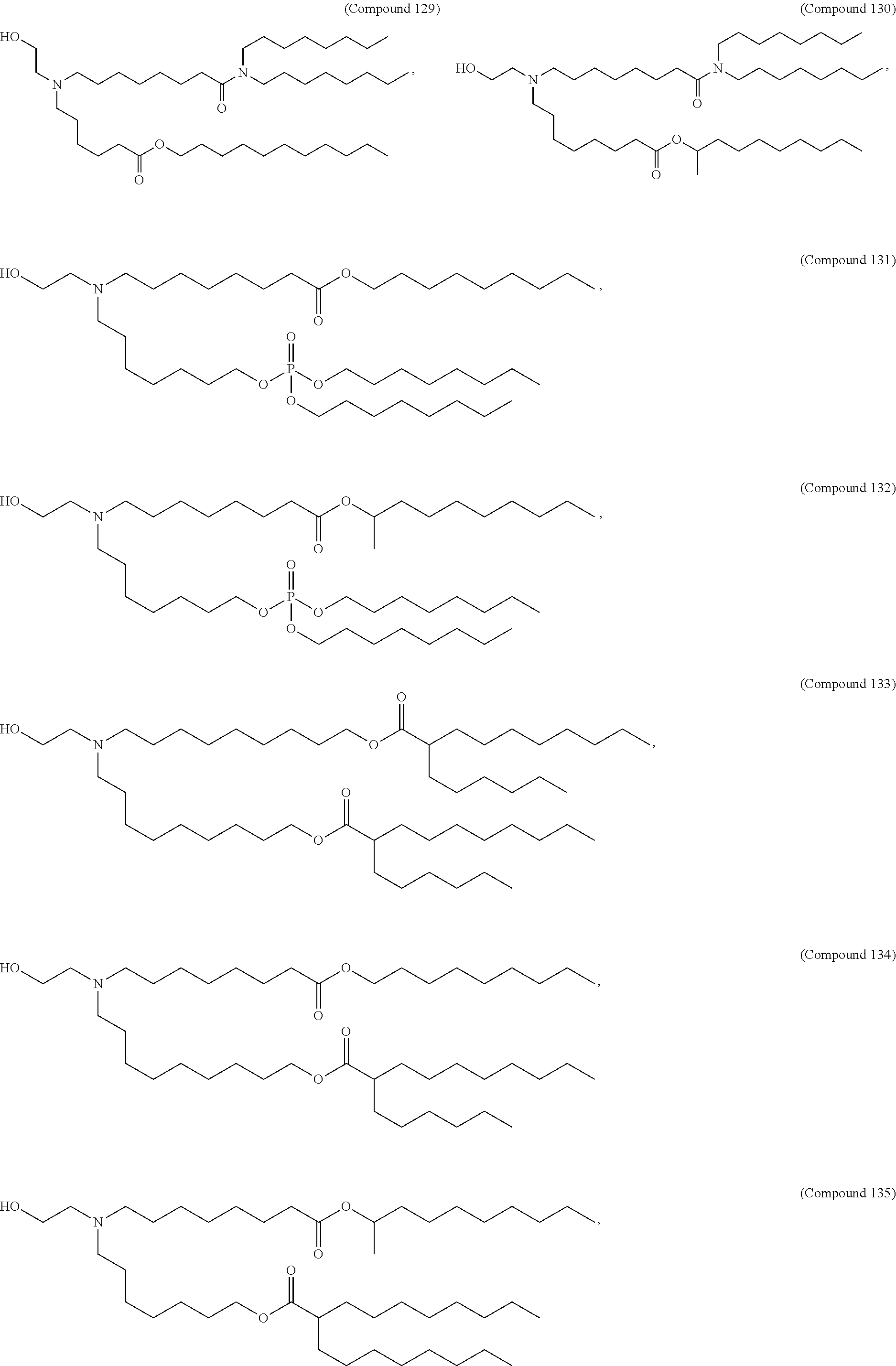

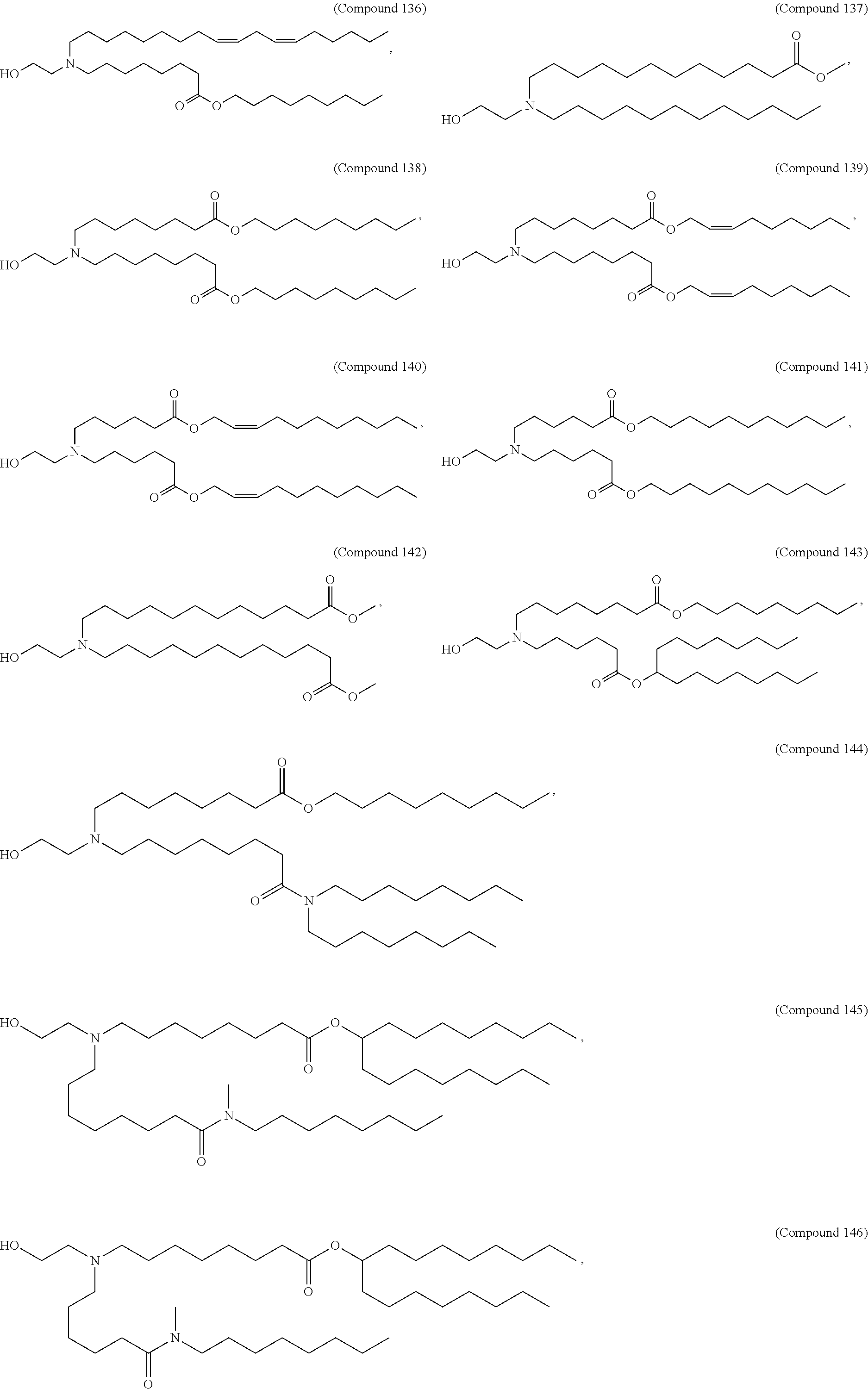

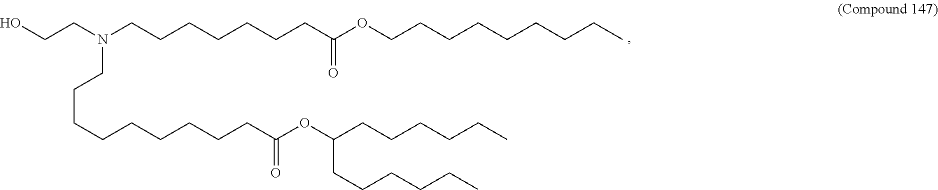 [SMILES: CCCCCCCCCOC(=O)CCCCCCCN(CCO)CCCCCCCCCC(=O)OC(CCCCCC)CCCCCC]

and salts or stereoisomers thereof.

In one embodiment, the ionizable amino lipid is Compound 18.

In some embodiments, the ionizable amino lipid can be the compounds disclosed in International Publication No. WO 2015/199952 A1, hereby incorporated by reference in its entirety. The ionizable amino lipid can be a compound having a structure of Formula (III):

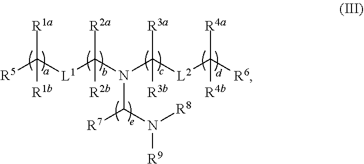

or a pharmaceutically acceptable salt, tautomer, prodrug or stereoisomer thereof, wherein:
L¹ and L² are each independently —O(C═O)—, —(C═O)O— or a carbon-carbon double bond;
R^{1a} and R^{1b} are, at each occurrence, independently either (a) H or C₁-C₁₂ alkyl, or (b) R^{1a} is H or C₁-C₁₂ alkyl, and R^{1b} together with the carbon atom to which it is bound is taken together with an adjacent R^{1b} and the carbon atom to which it is bound to form a carbon-carbon double bond;
R^{2a} and R^{2b} are, at each occurrence, independently either (a) H or C₁-C₁₂ alkyl, or (b) R^{2a} is H or C₁-C₁₂ alkyl, and R^{2b} together with the carbon atom to which it is bound is taken together with an adjacent R^{2b} and the carbon atom to which it is bound to form a carbon-carbon double bond;
R^{3a} and R^{3b} are, at each occurrence, independently either (a) H or C₁-C₁₂ alkyl, or (b) R^{3a} is H or C₁-C₁₂ alkyl, and R^{3b} together with the carbon atom to which it is bound is taken together with an adjacent R^{3b} and the carbon atom to which it is bound to form a carbon-carbon double bond;
R^{4a} and R^{4b} are, at each occurrence, independently either (a) H or C₁-C₁₂ alkyl, or (b) R^{4a} is H or C₁-C₁₂ alkyl, and R^{4b} together with the carbon atom to which it is bound is taken together with an adjacent R^{4b} and the carbon atom to which it is bound to form a carbon-carbon double bond;
R⁵ and R⁶ are each independently methyl or cycloalkyl;
R⁷ is, at each occurrence, independently H or C₁-C₁₂ alkyl;
R⁸ and R⁹ are each independently unsubstituted C₁-C₁₂ alkyl; or R⁸ and R⁹, together with the nitrogen atom to which they are attached, form a 5, 6 or 7-membered heterocyclic ring comprising one nitrogen atom;
a and d are each independently an integer from 0 to 24;
b and c are each independently an integer from 1 to 24; and
e is 1 or 2,

provided that:
at least one of R^{1a}, R^{2a}, R^{3a} or R^{4a} is C₁-C₁₂ alkyl, or at least one of L¹ or L² is —O(C═O)— or —(C═O)O—; and
R^{1a} and R^{1b} are not isopropyl when a is 6 or n-butyl when a is 8.

In some embodiments, the ionizable amino lipid is a compound having a structure of Formula (III) wherein one of L¹ or L² is —O(C═O)—, or —(C═O)O—. In some embodiments, one of L¹ or L² is a carbon-carbon double bond.

For example, the ionizable amino lipids of Formula (III) include, but not limited to:

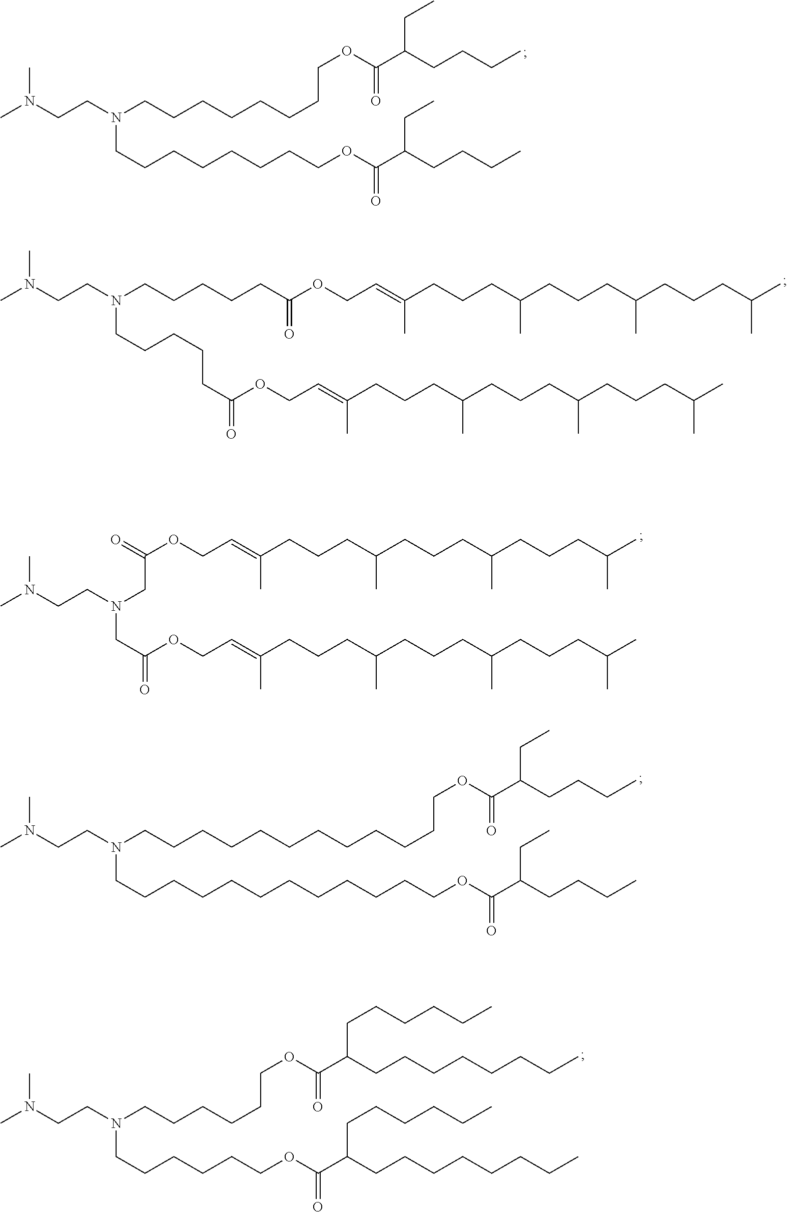

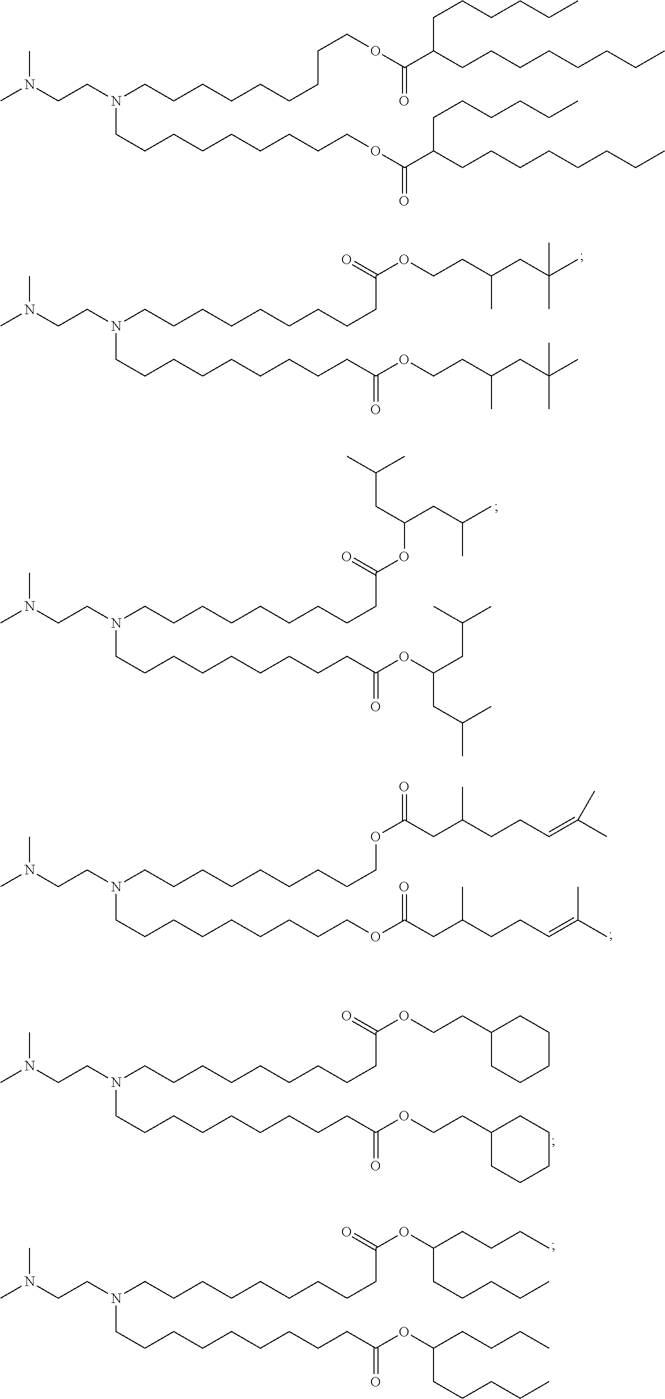

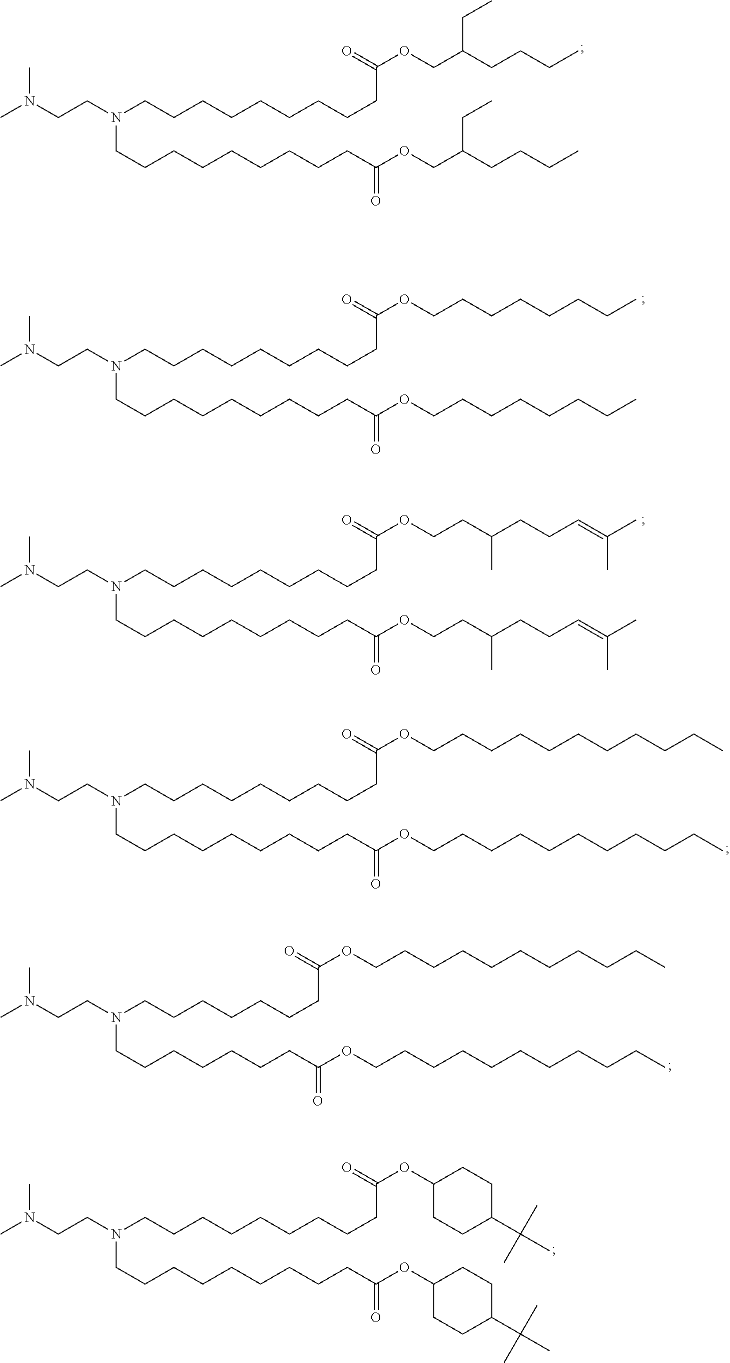

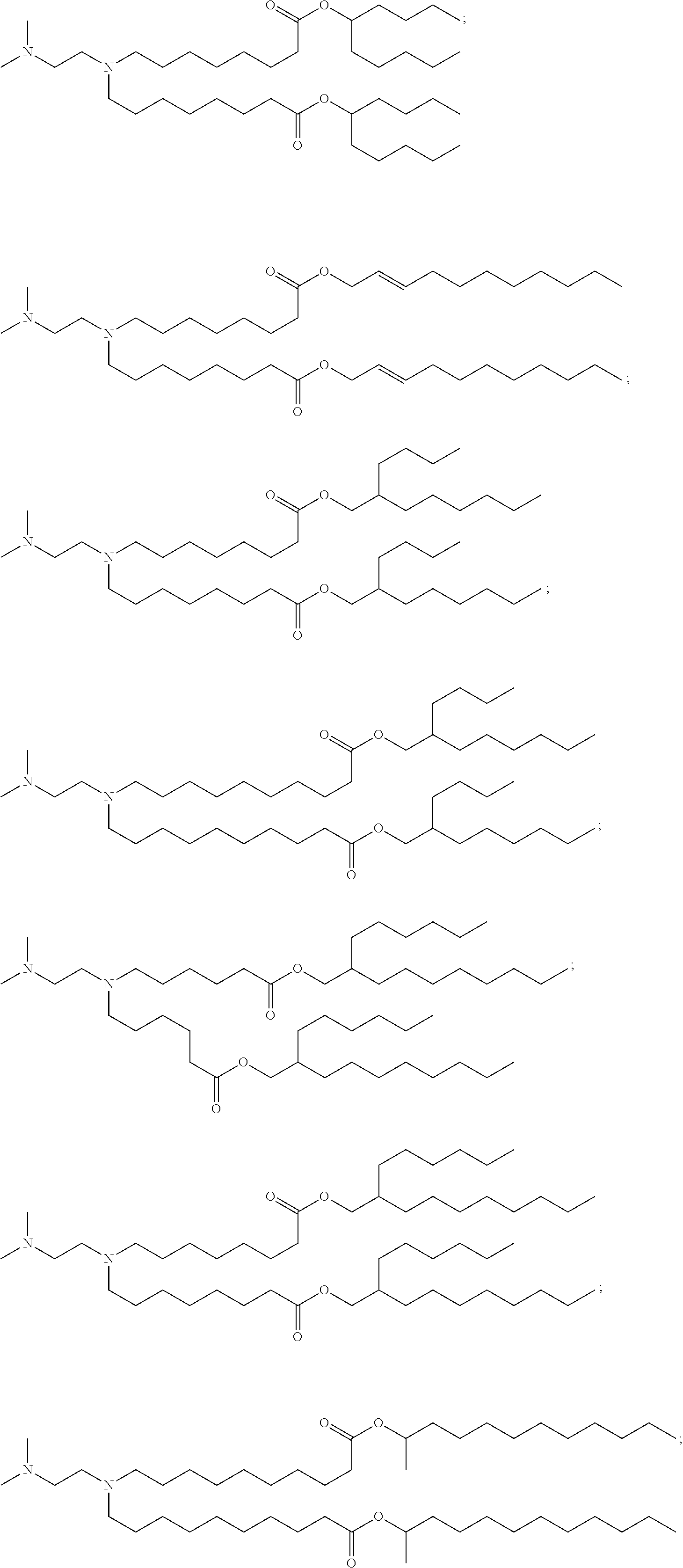

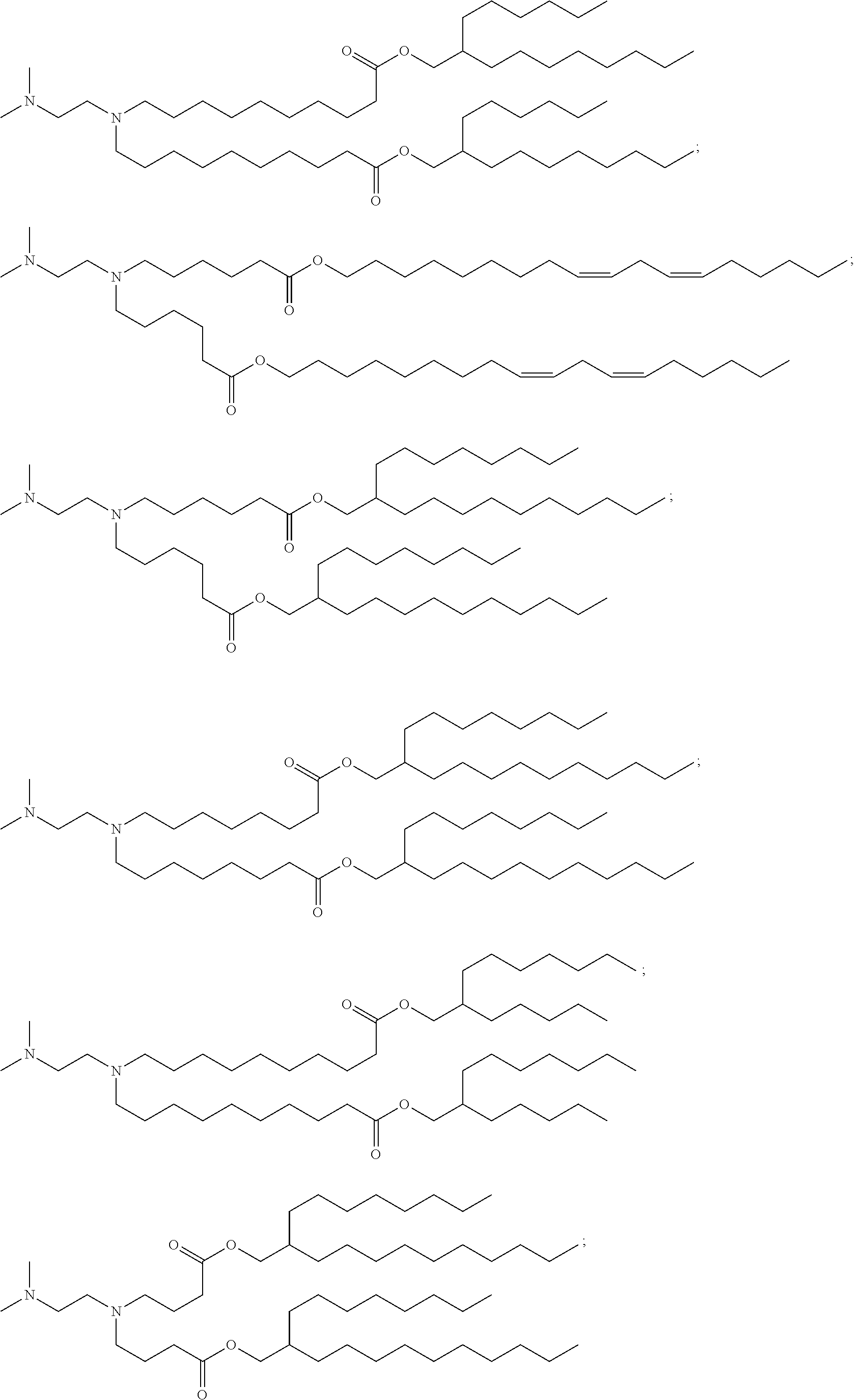

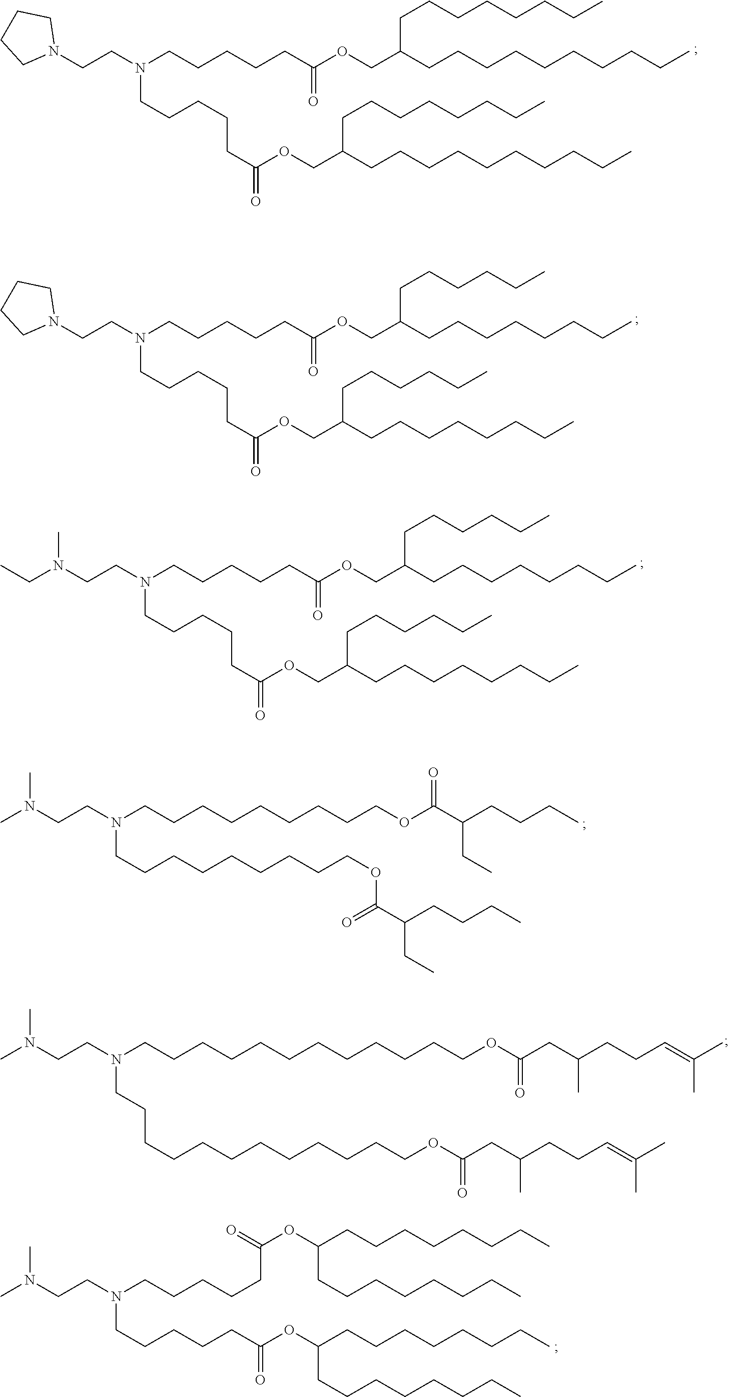

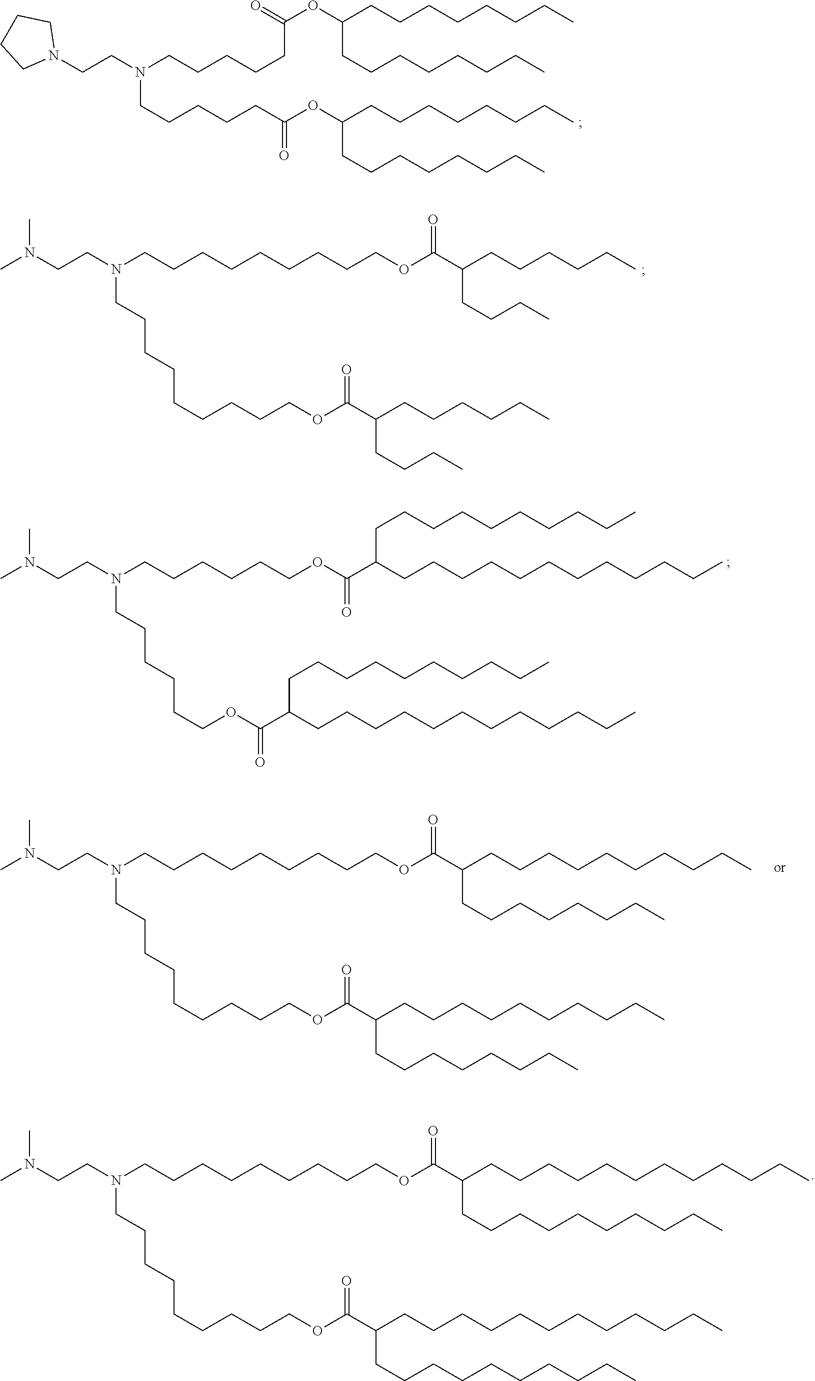

In some embodiments, the ionizable amino lipid of Formula (III) is

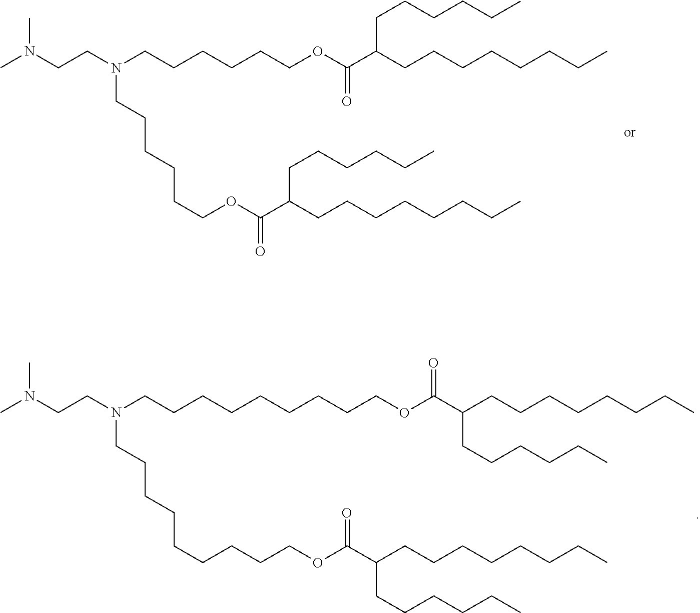

In one embodiment, the amount of the ionizable amino lipid ranges from about 30 to about 70 mole %, from about 35 to about 65 mole %, from about 40 to about 60 mole %, and from about 45 to about 55 mole % in the lipid composition. In one embodiment, the amount of the ionizable amino lipid is about 50 mole % in the lipid composition.

### IV. Pharmaceutical Compositions for Intratumoral Delivery

The present application also provides pharmaceutical compositions for intratumoral delivery with advantageous properties over pharmaceutical compositions known in the art, such as improved retention of therapeutic agents in tumoral tissue. In particular, the present disclosure provides a pharmaceutical composition for intratumoral delivery comprising:

(a) a lipid composition comprising:
(i) a compound having the formula (I)

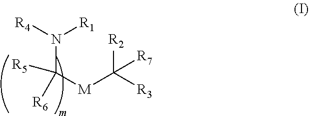

wherein

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and m is selected from 5, 6, 7, 8, 9, 10, 11, 12, and 13,

or salts or stereoisomers thereof, wherein alkyl and alkenyl groups may be linear or branched.

In some embodiments, a subset of compounds of Formula (I) includes those in which when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2.

In another embodiments, another subset of compounds of Formula (I) includes those in which

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, and a 5- to 14-membered heterocycloalkyl having one or more heteroatoms selected from N, O, and S which is substituted with one or more substituents selected from oxo (═O), OH, amino, and C₁₋₃ alkyl, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In yet another embodiments, another subset of compounds of Formula (I) includes those in which

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heterocycle having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, and each n is independently selected from 1, 2, 3, 4, and 5; and when Q is a 5- to 14-membered heterocycle and (i) R₄ is —(CH₂)ₙQ in which n is 1 or 2, or (ii) R₄ is —(CH₂)ₙCHQR in which n is 1, or (iii) R₄ is —CHQR, and —CQ(R)₂, then Q is either a 5- to 14-membered heteroaryl or 8- to 14-membered heterocycloalkyl;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In still another embodiments, another subset of compounds of Formula (I) includes those in which

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a C₃₋₆ carbocycle, a 5- to 14-membered heteroaryl having one or more heteroatoms selected from N, O, and S, —OR, —O(CH₂)ₙN(R)₂, —C(O)OR, —OC(O)R, —CX₃, —CX₂H, —CXH₂, —CN, —C(O)N(R)₂, —N(R)C(O)R, —N(R)S(O)₂R, —N(R)C(O)N(R)₂, —N(R)C(S)N(R)₂, —CRN(R)₂C(O)OR, and each n is independently selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₂₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In yet another embodiments, a subset of compounds of Formula (I) includes those in which

R₁ is selected from the group consisting of C₅₋₂₀ alkyl, C₅₋₂₀ alkenyl, —R*YR″, —YR″, and —R″M′R′;

R₂ and R₃ are independently selected from the group consisting of H, C₂₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is —(CH₂)ₙQ or —(CH₂)ₙCHQR, where Q is —N(R)₂, and n is selected from 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H;

each R is independently selected from the group consisting of C₁₋₃ alkyl, C₂₋₃ alkenyl, and H;

each R′ is independently selected from the group consisting of C₁₋₁₈ alkyl, C₂₋₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In still another embodiments, a subset of compounds of Formula (I) includes those in which

R₁ is selected from the group consisting of C₅₋₂₀ alkyl, C₅₋₂₀ alkenyl, —R*YR″, —YR″, and —R″M′R′;

R₂ and R₃ are independently selected from the group consisting of C₁₋₁₄ alkyl, C₂₋₁₄ alkenyl, —R*YR″, —YR″, and —R*OR″, or R₂ and R₃, together with the atom to which they are attached, form a heterocycle or carbocycle;

R₄ is selected from the group consisting of —(CH₂)ₙQ, —(CH₂)ₙCHQR, —CHQR, and —CQ(R)₂, where Q is —N(R)₂, and n is selected from 1, 2, 3, 4, and 5;

each R₅ is independently selected from the group consisting of C₁₋₃ alkyl, C₂₋₃ alkenyl, and H;

each R₆ is independently selected from the group consisting of C₁₋₃ alkyl, C₂₋₃ alkenyl, and H;

M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;

R₇ is selected from the group consisting of C₁₋₃ alkyl, C₂₋₃ alkenyl, and H; each R is independently selected from the group consisting of C₁₋₃ alkyl, C₂₋₃ alkenyl, and H;

each R′ is independently selected from the group consisting of C₁₋₁₈ alkyl, C₂-₁₈ alkenyl, —R*YR″, —YR″, and H;

each R″ is independently selected from the group consisting of C₃₋₁₄ alkyl and C₃₋₁₄ alkenyl;

each R* is independently selected from the group consisting of C₁₋₁₂ alkyl and C₁₋₁₂ alkenyl;

each Y is independently a C₃₋₆ carbocycle;

each X is independently selected from the group consisting of F, Cl, Br, and I; and

m is selected from 5, 6, 7, 8, 9, 10, 11, 12, and 13,

or salts or stereoisomers thereof.

In certain embodiments, a subset of compounds of Formula (I) includes those of Formula (IA):

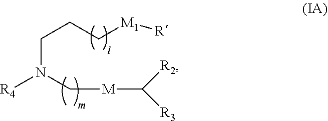

or a salt or stereoisomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which Q is OH, —NHC(S)N(R)₂, or —NHC(O)N(R)₂; M and M′ are independently selected from —C(O)O—, —OC(O)—, —C(O)N(R′)—, —P(O)(OR′)O—, an aryl group, and a heteroaryl group; and

R₂ and R₃ are independently selected from the group consisting of H, C₁₋₁₄ alkyl, and C₂₋₁₄ alkenyl.

In certain embodiments, a subset of compounds of Formula (I) includes those of Formula (II):

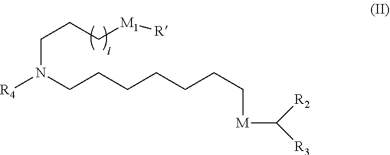

or a salt or stereoisomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, or —NHC(O)N(R)₂; M and M′ are independently selected from —C(O)O—, —OC(O)—, —C(O)N(R′)—, —P(O)(OR′)O—, an aryl group, and a heteroaryl group; and

R₂ and R₃ are independently selected from the group consisting of H, C₁₋₁₄ alkyl, and C₂₋₁₄ alkenyl.

In some embodiments, a subset of compounds of formula (I) is of the formula (IIa),

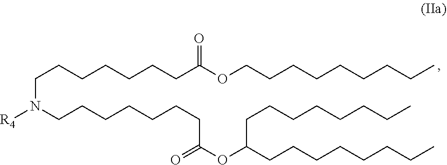

or a salt thereof, wherein R₄ is as described above.

In some embodiments, a subset of compounds of formula (I) is of the formula (IIb),

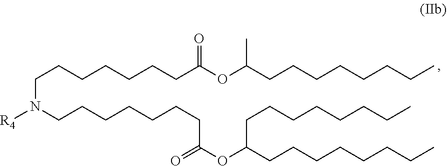

or a salt thereof, wherein R₄ is as described above.

In some embodiments, a subset of compounds of formula (I) is of the formula (IIc),

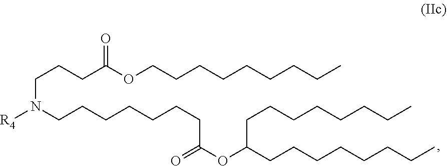

or a salt thereof, wherein R₄ is as described above.

In some embodiments, a subset of compounds of formula (I) is of the formula (IIe):

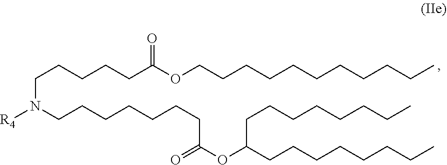

or a salt thereof, wherein R₄ is as described above.

In some embodiments, the compound of formula (IIa), (IIb), (IIc), or (IIe) comprises an R₄ which is selected from —(CH₂)ₙQ and —(CH₂)ₙCHQR, wherein Q, R and n are as defined above.

In some embodiments, Q is selected from the group consisting of —OR, —OH,
—O(CH₂)ₙN(R)₂, —OC(O)R, —CX₃, —CN, —N(R)C(O)R, —N(H)C(O)R, —N(R)S(O)₂R,
—N(H)S(O)₂R, —N(R)C(O)N(R)₂, —N(H)C(O)N(R)₂, —N(H)C(O)N(H)(R),
—N(R)C(S)N(R)₂, —N(H)C(S)N(R)₂, —N(H)C(S)N(H)(R), and a heterocycle, wherein R is as defined above. In some aspects, n is 1 or 2. In some embodiments, Q is OH, —NHC(S)N(R)₂, or —NHC(O)N(R)₂.

In some embodiments, the compound of formula (I) is of the formula (IId),

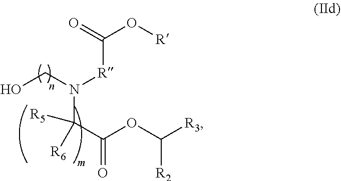

or a salt thereof, wherein R₂ and R₃ are independently selected from the group consisting of C₅₋₁₄ alkyl and C₅₋₁₄ alkenyl, n is selected from 2, 3, and 4, and R′, R″, R₅, R₆ and m are as defined above.

In some aspects of the compound of formula (IId), R₂ is C₈ alkyl. In some aspects of the compound of formula (IId), R₃ is C₅-C₉ alkyl. In some aspects of the compound of formula (IId), m is 5, 7, or 9. In some aspects of the compound of formula (IId), each R₅ is H. In some aspects of the compound of formula (IId), each R₆ is H.

In some aspects of the pharmaceutical compositions of the present disclosure, the compound of formula (I) is selected from the group consisting of compounds 1-147.

The central amine moiety of a lipid according to formula (I) may be protonated at a physiological pH. Thus, a lipid may have a positive or partial positive charge at physiological pH. Such lipids may be referred to ionizable (amino) lipids.

The disclosed pharmaceutical compositions (e.g., in lipid nanoparticle form) comprising the compounds of formula (I) described herein can be used for intratumoral delivery of a therapeutic agent (e.g., a polypeptide, small molecule, siRNA, etc) or a polynucleotide (e.g., a mRNA) encoding a therapeutic agent. These pharmaceutical compositions for intratumoral administration can:

(i) increase the retention of the therapeutic agent in the tumor;

(ii) increase the retention of the polynucleotide encoding a therapeutic agent in the tumor;

(iii) increase the levels of expressed polypeptide in the tumor compared to the levels of expressed polypeptide in peritumoral tissue;

(iv) decrease leakage of the polynucleotide or expressed product to off-target tissue (e.g., peritumoral tissue, or to other tissues or organs, e.g., liver tissue); or,

(v) any combination thereof,

wherein the increase or decrease observed for a certain property is relative to a corresponding reference composition (e.g., composition in which compounds of formula (I) are not present or have been substituted by another ionizable amino lipid, e.g., MC3).

In one embodiment, a decrease in leakage can be quantified as increase in the ratio of polypeptide expression in the tumor to polypeptide expression in non-tumor tissues, such as peritumoral tissue or to another tissue or organ, e.g., liver tissue.

In one specific embodiment, the compound of formula (I) is Compound 18.

In some embodiments, the amount the compound of formula (I) ranges from about 1 mol % to 99 mol % in the lipid composition.

In one embodiment, the amount of compound of formula (I) is at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 mol % in the lipid composition.

In one embodiment, the amount of the compound of formula (I) ranges from about 30 mol % to about 70 mol %, from about 35 mol % to about 65 mol %, from about 40 mol % to about 60 mol %, and from about 45 mol % to about 55 mol % in the lipid composition.

In one specific embodiment, the amount of the compound of formula (I) is about 50 mol % in the lipid composition.

In addition to the compound of formula (I), the lipid composition component of the pharmaceutical compositions for intratumoral delivery disclosed herein can comprise additional components such as phospholipids, structural lipids, quaternary amine compounds, PEG-lipids, and any combination thereof.

### Additional Lipid Composition Components

### A. Phospholipids

The lipid composition component of a pharmaceutical composition for intratumoral administration disclosed herein can comprise one or more phospholipids, for example, one or more saturated or (poly)unsaturated phospholipids or a combination thereof. In general, phospholipids comprise a phospholipid moiety and one or more fatty acid moieties. For example, a phospholipid can be a lipid according to formula (III):

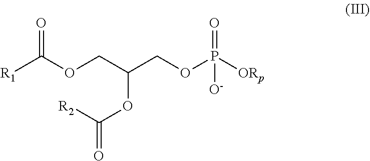

in which Rₚ represents a phospholipid moiety and R₁ and R₂ represent fatty acid moieties with or without unsaturation that may be the same or different.

A phospholipid moiety can be selected, for example, from the non-limiting group consisting of phosphatidyl choline, phosphatidyl ethanolamine, phosphatidyl glycerol, phosphatidyl serine, phosphatidic acid, 2 lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements (e.g., a therapeutic agent) of a lipid-containing composition (e.g., a nanoparticle) to pass through the membrane permitting, e.g., delivery of the one or more elements to a target tissue (e.g., tumoral tissue).

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group may undergo a copper-catalyzed cycloaddition upon exposure to an azide. Such reactions may be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin. In some embodiments, a pharmaceutical composition for intratumoral delivery disclosed herein can comprise more than one phospholipid. When more than one phospholipid is used, such phospholipids can belong to the same phospholipid class (e.g., MSPC and DSPC) or different classes (e.g., MSPC and MSPE).

Phospholipids can be of a symmetric or an asymmetric type. As used herein, the term “symmetric phospholipid” includes glycerophospholipids having matching fatty acid moieties and sphingolipids in which the variable fatty acid moiety and the hydrocarbon chain of the sphingosine backbone include a comparable number of carbon atoms. As used herein, the term “asymmetric phospholipid” includes lysolipids, glycerophospholipids having different fatty acid moieties (e.g., fatty acid moieties with different numbers of carbon atoms and/or unsaturations (e.g., double bonds)), and sphingolipids in which the variable fatty acid moiety and the hydrocarbon chain of the sphingosine backbone include a dissimilar number of carbon atoms (e.g., the variable fatty acid moiety include at least two more carbon atoms than the hydrocarbon chain or at least two fewer carbon atoms than the hydrocarbon chain).

In some embodiments, the lipid composition component of a pharmaceutical composition for intratumoral delivery disclosed herein comprises at least one symmetric phospholipid. Symmetric phospholipids can be selected from the non-limiting group consisting of
1,2-dipropionyl-sn-glycero-3-phosphocholine (03:0 PC),
1,2-dibutyryl-sn-glycero-3-phosphocholine (04:0 PC),
1,2-dipentanoyl-sn-glycero-3-phosphocholine (05:0 PC),
1,2-dihexanoyl-sn-glycero-3-phosphocholine (06:0 PC),
1,2-diheptanoyl-sn-glycero-3-phosphocholine (07:0 PC),
1,2-dioctanoyl-sn-glycero-3-phosphocholine (08:0 PC),
1,2-dinonanoyl-sn-glycero-3-phosphocholine (09:0 PC),
1,2-didecanoyl-sn-glycero-3-phosphocholine (10:0 PC),
1,2-diundecanoyl-sn-glycero-3-phosphocholine (11:0 PC, DUPC),
1,2-dilauroyl-sn-glycero-3-phosphocholine (12:0 PC),
1,2-ditridecanoyl-sn-glycero-3-phosphocholine (13:0 PC),
1,2-dimyristoyl-sn-glycero-3-phosphocholine (14:0 PC, DMPC),
1,2-dipentadecanoyl-sn-glycero-3-phosphocholine (15:0 PC),
1,2-dipalmitoyl-sn-glycero-3-phosphocholine (16:0 PC, DPPC),
1,2-diphytanoyl-sn-glycero-3-phosphocholine (4ME 16:0 PC),
1,2-diheptadecanoyl-sn-glycero-3-phosphocholine (17:0 PC),
1,2-distearoyl-sn-glycero-3-phosphocholine (18:0 PC, DSPC),
1,2-dinonadecanoyl-sn-glycero-3-phosphocholine (19:0 PC),
1,2-diarachidoyl-sn-glycero-3-phosphocholine (20:0 PC),
1,2-dihenarachidoyl-sn-glycero-3-phosphocholine (21:0 PC),
1,2-dibehenoyl-sn-glycero-3-phosphocholine (22:0 PC),
1,2-ditricosanoyl-sn-glycero-3-phosphocholine (23:0 PC),
1,2-dilignoceroyl-sn-glycero-3-phosphocholine (24:0 PC),
1,2-dimyristoleoyl-sn-glycero-3-phosphocholine (14:1 (49-Cis) PC),
1,2-dimyristelaidoyl-sn-glycero-3-phosphocholine (14:1 (49-Trans) PC),
1,2-dipalmitoleoyl-sn-glycero-3-phosphocholine (16:1 (49-Cis) PC),
1,2-dipalmitelaidoyl-sn-glycero-3-phosphocholine (16:1 (49-Trans) PC),
1,2-dipetroselenoyl-sn-glycero-3-phosphocholine (18:1 (46-Cis) PC),
1,2-dioleoyl-sn-glycero-3-phosphocholine (18:1 (49-Cis) PC, DOPC),
1,2-dielaidoyl-sn-glycero-3-phosphocholine (18:1 (49-Trans) PC),
1,2-dilinoleoyl-sn-glycero-3-phosphocholine (18:2 (Cis) PC, DLPC),
1,2-dilinolenoyl-sn-glycero-3-phosphocholine (18:3 (Cis) PC, DLnPC),
1,2-dieicosenoyl-sn-glycero-3-phosphocholine (20:1 (Cis) PC),
1,2-diarachidonoyl-sn-glycero-3-phosphocholine (20:4 (Cis) PC, DAPC),
1,2-dierucoyl-sn-glycero-3-phosphocholine (22:1 (Cis) PC),
1,2-didocosahexaenoyl-sn-glycero-3-phosphocholine (22:6 (Cis) PC, DHAPC),
1,2-dinervonoyl-sn-glycero-3-phosphocholine (24:1 (Cis) PC),
1,2-dihexanoyl-sn-glycero-3-phosphoethanolamine (06:0 PE),
1,2-dioctanoyl-sn-glycero-3-phosphoethanolamine (08:0 PE),
1,2-didecanoyl-sn-glycero-3-phosphoethanolamine (10:0 PE),
1,2-dilauroyl-sn-glycero-3-phosphoethanolamine (12:0 PE),
1,2-dimyristoyl-sn-glycero-3-phosphoethanolamine (14:0 PE),
1,2-dipentadecanoyl-sn-glycero-3-phosphoethanolamine (15:0 PE),
1,2-dipalmitoyl-sn-glycero-3-phosphoethanolamine (16:0 PE),
1,2-diphytanoyl-sn-glycero-3-phosphoethanolamine (4ME 16:0 PE),
1,2-diheptadecanoyl-sn-glycero-3-phosphoethanolamine (17:0 PE),
1,2-distearoyl-sn-glycero-3-phosphoethanolamine (18:0 PE, DSPE),
1,2-dipalmitoleoyl-sn-glycero-3-phosphoethanolamine (16:1 PE),
1,2-dioleoyl-sn-glycero-3-phosphoethanolamine (18:1 (49-Cis) PE, DOPE),
1,2-dielaidoyl-sn-glycero-3-phosphoethanolamine (18:1 (49-Trans) PE),
1,2-dilinoleoyl-sn-glycero-3-phosphoethanolamine (18:2 PE, DLPE),
1,2-dilinolenoyl-sn-glycero-3-phosphoethanolamine (18:3 PE, DLnPE),
1,2-diarachidonoyl-sn-glycero-3-phosphoethanolamine (20:4 PE, DAPE),
1,2-didocosahexaenoyl-sn-glycero-3-phosphoethanolamine (22:6 PE, DHAPE),
1,2-di-O-octadecenyl-sn-glycero-3-phosphocholine (18:0 Diether PC),
1,2-dioleoyl-sn-glycero-3-phospho-rac-(1-glycerol) sodium salt (DOPG), and any combination thereof.

In some embodiments, the lipid composition component of a pharmaceutical composition for intratumoral delivery disclosed herein comprises at least one symmetric phospholipid selected from the non-limiting group consisting of DLPC, DMPC, DOPC, DPPC, DSPC, DUPC, 18:0 Diether PC, DLnPC, DAPC, DHAPC, DOPE, 4ME 16:0 PE, DSPE, DLPE, DLnPE, DAPE, DHAPE, DOPG, and any combination thereof.

In some embodiments, the lipid composition component of a pharmaceutical composition for intratumoral delivery disclosed herein comprises at least one asymmetric phospholipid. Asymmetric phospholipids can be selected from the non-limiting group consisting of
1-myristoyl-2-palmitoyl-sn-glycero-3-phosphocholine (14:0-16:0 PC, MPPC),
1-myristoyl-2-stearoyl-sn-glycero-3-phosphocholine (14:0-18:0 PC, MSPC),
1-palmitoyl-2-acetyl-sn-glycero-3-phosphocholine (16:0-02:0 PC),
1-palmitoyl-2-myristoyl-sn-glycero-3-phosphocholine (16:0-14:0 PC, PMPC),
1-palmitoyl-2-stearoyl-sn-glycero-3-phosphocholine (16:0-18:0 PC, PSPC),
1-palmitoyl-2-oleoyl-sn-glycero-3-phosphocholine (16:0-18:1 PC, POPC),
1-palmitoyl-2-linoleoyl-sn-glycero-3-phosphocholine (16:0-18:2 PC, PLPC),
1-palmitoyl-2-arachidonoyl-sn-glycero-3-phosphocholine (16:0-20:4 PC),
1-palmitoyl-2-docosahexaenoyl-sn-glycero-3-phosphocholine (14:0-22:6 PC),
1-stearoyl-2-myristoyl-sn-glycero-3-phosphocholine (18:0-14:0 PC, SMPC),
1-stearoyl-2-palmitoyl-sn-glycero-3-phosphocholine (18:0-16:0 PC, SPPC),
1-stearoyl-2-oleoyl-sn-glycero-3-phosphocholine (18:0-18:1 PC, SOPC),
1-stearoyl-2-linoleoyl-sn-glycero-3-phosphocholine (18:0-18:2 PC),
1-stearoyl-2-arachidonoyl-sn-glycero-3-phosphocholine (18:0-20:4 PC),
1-stearoyl-2-docosahexaenoyl-sn-glycero-3-phosphocholine (18:0-22:6 PC),
1-oleoyl-2-myristoyl-sn-glycero-3-phosphocholine (18:1-14:0 PC, OMPC),
1-oleoyl-2-palmitoyl-sn-glycero-3-phosphocholine (18:1-16:0 PC, OPPC),
1-oleoyl-2-stearoyl-sn-glycero-3-phosphocholine (18:1-18:0 PC, OSPC),
1-palmitoyl-2-oleoyl-sn-glycero-3-phosphoethanolamine (16:0-18:1 PE, POPE),
1-palmitoyl-2-linoleoyl-sn-glycero-3-phosphoethanolamine (16:0-18:2 PE),
1-palmitoyl-2-arachidonoyl-sn-glycero-3-phosphoethanolamine (16:0-20:4 PE),
1-palmitoyl-2-docosahexaenoyl-sn-glycero-3-phosphoethanolamine (16:0-22:6 PE),
1-stearoyl-2-oleoyl-sn-glycero-3-phosphoethanolamine (18:0-18:1 PE),
1-stearoyl-2-linoleoyl-sn-glycero-3-phosphoethanolamine (18:0-18:2 PE),
1-stearoyl-2-arachidonoyl-sn-glycero-3-phosphoethanolamine (18:0-20:4 PE),
1-stearoyl-2-docosahexaenoyl-sn-glycero-3-phosphoethanolamine (18:0-22:6 PE),
1-oleoyl-2-cholesterylhemisuccinoyl-sn-glycero-3-phosphocholine (OChemsPC), and any combination thereof.

Asymmetric lipids useful in nanoparticle compositions can also be lysolipids. Lysolipids can be selected from the non-limiting group consisting of
1-hexanoyl-2-hydroxy-sn-glycero-3-phosphocholine (06:0 Lyso PC),
1-heptanoyl-2-hydroxy-sn-glycero-3-phosphocholine (07:0 Lyso PC),
1-octanoyl-2-hydroxy-sn-glycero-3-phosphocholine (08:0 Lyso PC),
1-nonanoyl-2-hydroxy-sn-glycero-3-phosphocholine (09:0 Lyso PC),
1-decanoyl-2-hydroxy-sn-glycero-3-phosphocholine (10:0 Lyso PC),
1-undecanoyl-2-hydroxy-sn-glycero-3-phosphocholine (11:0 Lyso PC),
1-lauroyl-2-hydroxy-sn-glycero-3-phosphocholine (12:0 Lyso PC),
1-tridecanoyl-2-hydroxy-sn-glycero-3-phosphocholine (13:0 Lyso PC),
1-myristoyl-2-hydroxy-sn-glycero-3-phosphocholine (14:0 Lyso PC),
1-pentadecanoyl-2-hydroxy-sn-glycero-3-phosphocholine (15:0 Lyso PC),
1-palmitoyl-2-hydroxy-sn-glycero-3-phosphocholine (16:0 Lyso PC),
1-heptadecanoyl-2-hydroxy-sn-glycero-3-phosphocholine (17:0 Lyso PC),
1-stearoyl-2-hydroxy-sn-glycero-3-phosphocholine (18:0 Lyso PC),
1-oleoyl-2-hydroxy-sn-glycero-3-phosphocholine (18:1 Lyso PC),
1-nonadecanoyl-2-hydroxy-sn-glycero-3-phosphocholine (19:0 Lyso PC),
1-arachidoyl-2-hydroxy-sn-glycero-3-phosphocholine (20:0 Lyso PC),
1-behenoyl-2-hydroxy-sn-glycero-3-phosphocholine (22:0 Lyso PC),
1-lignoceroyl-2-hydroxy-sn-glycero-3-phosphocholine (24:0 Lyso PC),
1-hexacosanoyl-2-hydroxy-sn-glycero-3-phosphocholine (26:0 Lyso PC),
1-myristoyl-2-hydroxy-sn-glycero-3-phosphoethanolamine (14:0 Lyso PE),
1-palmitoyl-2-hydroxy-sn-glycero-3-phosphoethanolamine (16:0 Lyso PE),
1-stearoyl-2-hydroxy-sn-glycero-3-phosphoethanolamine (18:0 Lyso PE),
1-oleoyl-2-hydroxy-sn-glycero-3-phosphoethanolamine (18:1 Lyso PE),
1-hexadecyl-sn-glycero-3-phosphocholine (C16 Lyso PC), and any combination thereof.

In some embodiment, the lipid composition component of a pharmaceutical composition for intratumoral delivery disclosed herein comprises at least one asymmetric phospholipid selected from the group consisting of MPPC, MSPC, PMPC, PSPC, SMPC, SPPC, and any combination thereof. In some embodiments, the asymmetric phospholipid is 1-myristoyl-2-stearoyl-sn-glycero-3-phosphocholine (MSPC).

In some embodiments, the lipid compositions disclosed herein can contain one or more symmetric phospholipids, one or more asymmetric phospholipids, or a combination thereof. When multiple phospholipids are present, they can be present in equimolar ratios, or non-equimolar ratios.

In one embodiment, the lipid composition component of the pharmaceutical composition for intratumoral delivery disclosed herein comprises a total amount of phospholipid (e.g., MSPC) which ranges from about 1 mol % to about 20 mol %, from about 5 mol % to about 20 mol %, from about 10 mol % to about 20 mol %, from about 15 mol % to about 20 mol %, from about 1 mol % to about 15 mol %, from about 5 mol % to about 15 mol %, from about 10 mol % to about 15 mol %, from about 5 mol % to about 10 mol % in the lipid composition. In one embodiment, the amount of the phospholipid (e.g., MSPC) is about 10 mol % in the lipid composition.

In some aspects, the amount of a specific phospholipid (e.g., MSPC) is at least about 1, 2, 3, 4, 5, 6, 7, 8, 9, 10, 11, 12, 13, 14, 15, 16, 17, 18, 19 or 20 mol % in the lipid composition.

### B. Quaternary Amine Compounds

The lipid composition component of a pharmaceutical composition for intratumoral delivery disclosed herein can comprise one or more quaternary amine compounds (e.g., DOTAP).

The term “quaternary amine compound” as used herein includes those compounds having one or more quaternary amine groups (e.g., trialkylamino groups) and permanently carrying a positive charge and exists in a form of a salt. For example, the one or more quaternary amine groups can be present in a lipid or a polymer (e.g., PEG). In some embodiments, the quaternary amine compound comprises (1) a quaternary amine group and (2) at least one hydrophobic tail group comprising (i) a hydrocarbon chain, linear or branched, and saturated or unsaturated, and (ii) optionally an ether, ester, or ketal linkage between the quaternary amine group and the hydrocarbon chain. In some embodiments, the quaternary amine group can be a trimethylammonium group. In some embodiments, the quaternary amine compound comprises two identical hydrocarbon chains. In some embodiments, the quaternary amine compound comprises two different hydrocarbon chains.

In some embodiments, the lipid composition component of a pharmaceutical composition for intratumoral delivery disclosed herein comprises at least one quaternary amine compound. Quaternary amine compound can be selected from the non-limiting group consisting of
1,2-dioleoyl-3-trimethylammonium-propane (DOTAP),
N-[1-(2,3-dioleoyloxy)propyl]-N,N,N-trimethylammonium chloride (DOTMA),
1-[2-(oleoyloxy)ethyl]-2-oleyl-3-(2-hydroxyethyl)imidazolinium chloride (DOTIM),
2,3-dioleyloxy-N-[2(sperminecarboxamido)ethyl]-N,N-dimethyl-1-propanaminium trifluoroacetate (DOSPA),
N,N-distearyl-N,N-dimethylammonium bromide (DDAB),
N-(1,2-dimyristyloxyprop-3-yl)-N,N-dimethyl-N-hydroxyethyl ammonium bromide (DMRIE),
N-(1,2-dioleoyloxyprop-3-yl)-N,N-dimethyl-N-hydroxyethyl ammonium bromide (DORIE),
N,N-dioleyl-N,N-dimethylammonium chloride (DODAC),
1,2-dilauroyl-sn-glycero-3-ethylphosphocholine (DLePC),
1,2-distearoyl-3-trimethylammonium-propane (DSTAP),
1,2-dipalmitoyl-3-trimethylammonium-propane (DPTAP),
1,2-dilinoleoyl-3-trimethylammonium-propane (DLTAP),
1,2-dimyristoyl-3-trimethylammonium-propane (DMTAP),
1,2-distearoyl-sn-glycero-3-ethylphosphocholine (DSePC),
1,2-dipalmitoyl-sn-glycero-3-ethylphosphocholine (DPePC),
1,2-dimyristoyl-sn-glycero-3-ethylphosphocholine (DMePC),
1,2-dioleoyl-sn-glycero-3-ethylphosphocholine (DOePC),
1,2-di-(9Z-tetradecenoyl)-sn-glycero-3-ethylphosphocholine (14:1 EPC),
1-palmitoyl-2-oleoyl-sn-glycero-3-ethylphosphocholine (16:0-18:1 EPC), and any combination thereof.

In one embodiment, the quaternary amine compound is 1,2-dioleoyl-3-trimethylammonium-propane (DOTAP).

Quaternary amine compounds include those known in the art, such as those described in US 2013/0245107 A1, US 2014/0363493 A1, U.S. Pat. No. 8,158,601, WO 2015/123264 A1, and WO 2015/148247 A1, which are incorporated herein by reference in their entirety.

In one embodiment, the amount of the quaternary amine compound (e.g., DOTAP) in the lipid composition component of a pharmaceutical composition for intratumoral delivery disclosed herein ranges from about 0.01 mol % to about 20 mol %.

In one embodiment, the amount of the quaternary amine compound (e.g., DOTAP) in the lipid composition component of a pharmaceutical composition for intratumoral delivery disclosed herein ranges from about 0.5 mol % to about 20 mol %, from about 0.5 mol % to about 15 mol %, from about 0.5 mol % to about 10 mol %, from about 1 mol % to about 20 mol %, from about 1 mol % to about 15 mol %, from about 1 mol % to about 10 mol %, from about 2 mol % to about 20 mol %, from about 2 mol % to about 15 mol %, from about 2 mol % to about 10 mol %, from about 3 mol % to about 20 mol %, from about 3 mol % to about 15 mol %, from about 3 mol % to about 10 mol %, from about 4 mol % to about 20 mol %, from about 4 mol % to about 15 mol %, from about 4 mol % to about 10 mol %, from about 5 mol % to about 20 mol %, from about 5 mol % to about 15 mol %, from about 5 mol % to about 10 mol %, from about 6 mol % to about 20 mol %, from about 6 mol % to about 15 mol %, from about 6 mol % to about 10 mol %, from about 7 mol % to about 20 mol %, from about 7 mol % to about 15 mol %, from about 7 mol % to about 10 mol %, from about 8 mol % to about 20 mol %, from about 8 mol % to about 15 mol %, from about 8 mol % to about 10 mol %, from about 9 mol % to about 20 mol %, from about 9 mol % to about 15 mol %, from about 9 mol % to about 10 mol %.

In one embodiment, the amount of the quaternary amine compound (e.g., DOTAP) in the lipid composition component of a pharmaceutical composition for intratumoral delivery disclosed herein ranges from about 5 mol % to about 10 mol %.

In one embodiment, the amount of the quaternary amine compound (e.g., DOTAP) in the lipid composition component of a pharmaceutical composition for intratumoral delivery disclosed herein is about 5 mol %. In one embodiment, the amount of the quaternary amine compound (e.g., DOTAP) in the lipid composition component of a pharmaceutical composition for intratumoral delivery disclosed herein is about 10 mol %.

In some embodiments, the amount of the quaternary amine compound (e.g., DOTAP) is at least about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20 mol % in the lipid composition component of a pharmaceutical composition for intratumoral delivery disclosed herein.

In one embodiment, the mole ratio of the compound of formula (I) (e.g., Compound 18) to the quaternary amine compound (e.g., DOTAP) is about 100:1 to about 2.5:1. In one embodiment, the mole ratio of the compound of formula (I) (e.g., Compound 18) to the quaternary amine compound (e.g., DOTAP) is about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, or about 2.5:1. In one embodiment, the mole ratio of the compound of formula (I) (e.g., Compound 18) to the quaternary amine compound (e.g., DOTAP) in the lipid composition component of a pharmaceutical composition for intratumoral delivery disclosed herein is about 10:1.

### C. Structural Lipids

The lipid composition component of a pharmaceutical composition for intratumoral delivery disclosed herein can comprise one or more structural lipids. As used herein, the term “structural lipid” refers to sterols and also to lipids containing sterol moieties. In some embodiments, the structural lipid is selected from the group consisting of cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol.

In one embodiment, the amount of the structural lipid (e.g., an sterol such as cholesterol) in the lipid composition component of a pharmaceutical composition for intratumoral delivery disclosed herein ranges from about 20 mol % to about 60 mol %, from about 25 mol % to about 55 mol %, from about 30 mol % to about 50 mol %, or from about 35 mol % to about 45 mol %.

In one embodiment, the amount of the structural lipid (e.g., an sterol such as cholesterol) in the lipid composition component of a pharmaceutical composition for intratumoral delivery disclosed herein ranges from about 25 mol % to about 30 mol %, from about 30 mol % to about 35 mol %, or from about 35 mol % to about 40 mol %.

In one embodiment, the amount of the structural lipid (e.g., a sterol such as cholesterol) in the lipid composition component of a pharmaceutical composition for intratumoral delivery disclosed herein is about 28.5 mol %, about 33.5 mol %, or about 38.5 mol %.

In some embodiments, the amount of the structural lipid (e.g., an sterol such as cholesterol) in the lipid composition component of a pharmaceutical composition for intratumoral delivery disclosed herein is at least about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 mol %.

### D. Polyethylene Glycol (PEG)-Lipids

The lipid composition component of a pharmaceutical composition for intratumoral delivery disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxlpropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C₁₄ to about C₂₂, preferably from about C₁₄ to about C₁₆. In some embodiments, a PEG moiety, for example an mPEG-NH₂, has a size of about 1000, 2000, 5000, 10,000, 15,000 or 20,000 daltons. In one embodiment, the PEG-lipid is PEG₂ₖ-DMG.

PEG-lipids include those known in the art, such as those described in U.S. Pat. No. 8,158,601 and, WO 2015/130584 A2, which are incorporated herein by reference in their entirety.

In one embodiment, the amount of PEG-lipid in the lipid composition component of a pharmaceutical composition for intratumoral delivery disclosed herein ranges from about 0.1 mol % to about 5.0 mol %, from about 0.5 mol % to about 5 mol %, from about 1 mol % to about 5.0 mol %, from about 1.5 mol % to about 5 mol %, from about 2 mol % to about 5 mol % mol %, from about 0.1 mol % to about 4 mol %, from about 0.5 mol % to about 4 mol %, from about 1 mol % to about 4 mol %, from about 1.5 mol % to about 4 mol %, from about 2 mol % to about 4 mol %, from about 0.1 mol % to about 3 mol %, from about 0.5 mol % to about 3 mol %, from about 1 mol % to about 3 mol %, from about 1.5 mol % to about 3 mol %, from about 2 mol % to about 3 mol %, from about 0.1 mol % to about 2 mol %, from about 0.5 mol % to about 2 mol %, from about 1 mol % to about 2 mol %, from about 1.5 mol % to about 2 mol %, from about 0.1 mol % to about 1.5 mol %, from about 0.5 mol % to about 1.5 mol %, or from about 1 mol % to about 1.5 mol %.

In one embodiment, the amount of PEG-lipid in the lipid composition component of a pharmaceutical composition for intratumoral delivery disclosed herein is about 1.5 mol %.

In one embodiment, the amount of PEG-lipid in the lipid composition component of a pharmaceutical composition for intratumoral delivery disclosed herein is at least about 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, or 5 mol %.

### E. Other Lipid Composition Components

The lipid composition component of a pharmaceutical composition for intratumoral delivery disclosed herein can include one or more components in addition to those described above. For example, the lipid composition can include one or more permeability enhancer molecules, carbohydrates, polymers, surface altering agents (e.g., surfactants), or other components. For example, a permeability enhancer molecule can be a molecule described in US2005/0222064 (herein incorporated by reference in its entirety). Carbohydrates can include simple sugars (e.g., glucose) and polysaccharides (e.g., glycogen and derivatives and analogs thereof).

A polymer can be included in and/or used to encapsulate or partially encapsulate a pharmaceutical composition disclosed herein (e.g., a pharmaceutical composition in lipid nanoparticle form). A polymer can be biodegradable and/or biocompatible. A polymer can be selected from, but is not limited to, polyamines, polyethers, polyamides, polyesters, polycarbamates, polyureas, polycarbonates, polystyrenes, polyimides, polysulfones, polyurethanes, polyacetylenes, polyethylenes, polyethyleneimines, polyisocyanates, polyacrylates, polymethacrylates, polyacrylonitriles, and polyarylates, or mixtures thereof.

### Theraperaputic Agents and Polynucleotides Encoding Therapeutic Agents

The pharmaceutical compositions for intratumoral delivery disclosed herein comprise a therapeutic agent or a polynucleotide encoding a therapeutic agent (e.g., an mRNA). As discussed in the definition section above, the term therapeutic agent is used broadly and includes polypeptides, polynucleotides, and other compounds such as small molecules.

In some embodiments, a therapeutic agent is a polynucleotide or nucleic acid (e.g., ribonucleic acid or deoxyribonucleic acid). Exemplary polynucleotides for use in accordance with the present disclosure include, but are not limited to, one or more of deoxyribonucleic acid (DNA), ribonucleic acid (RNA) including messenger mRNA (mRNA), hybrids thereof, RNAi-inducing agents, RNAi agents, siRNAs, shRNAs, miRNAs, antisense RNAs, ribozymes, catalytic DNA, RNAs that induce triple helix formation, aptamers, vectors, etc.

In some embodiments, a therapeutic agent is RNA. RNAs useful in the compositions and methods described herein can be selected from the group consisting of, but are not limited to, shortmers, antagomirs, antisense, ribozymes, small interfering RNA (siRNA), asymmetrical interfering RNA (aiRNA), microRNA (miRNA), Dicer substrate RNA (dsRNA), small hairpin RNA (shRNA), transfer RNA (tRNA), messenger RNA (mRNA), guide strand RNA, and mixtures thereof. In certain embodiments, the RNA is an mRNA.

In certain embodiments, a therapeutic agent is an mRNA. An mRNA may encode any polypeptide of interest, including any naturally or non-naturally occurring or otherwise modified polypeptide. A polypeptide encoded by an mRNA can be of any size and can have any secondary structure or activity. In some embodiments, a polypeptide encoded by an mRNA can have a therapeutic effect when expressed in a cell. In some embodiments, the polypeptide is, for example, a cytokine, a growth factor, a hormone, a cell surface receptor, or an antibody or antigen binding portion thereof.

In other embodiments, a therapeutic agent is a siRNA. A siRNA can be capable of selectively knocking down or down regulating expression of a gene of interest. For example, a siRNA could be selected to silence a gene associated with a particular disease, disorder, or condition upon administration to a subject in need thereof of a nanoparticle composition including the siRNA. A siRNA can comprise a sequence that is complementary to an mRNA sequence that encodes a gene or protein of interest. In some embodiments, the siRNA can be an immunomodulatory siRNA.

In some embodiments, a therapeutic agent is a shRNA or a vector or plasmid encoding the same. A shRNA can be produced inside a target cell upon delivery of an appropriate construct to the nucleus. Constructs and mechanisms relating to shRNA are well known in the relevant arts.

In some embodiments, the nucleic acids can include one or more messenger RNAs (mRNAs) having one or more alternative nucleoside or nucleotides (i.e., alternative mRNA molecules).

In some embodiments, the therapeutic agent is a polypeptide, for example, a cytokine, a growth factor, a hormone, a cell surface receptor, or an antibody or antigen binding portion thereof.

The ratio between the lipid composition and the therapeutic agent or polynucleotide encoding a therapeutic agent can range from about 10:1 to about 60:1 (wt/wt).

In some embodiments, the ratio between the lipid composition and the therapeutic agent or polynucleotide encoding a therapeutic agent can be about 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1 or 60:1 (wt/wt). In some embodiments, the wt/wt ratio of the lipid composition to the therapeutic agent or polynucleotide encoding a therapeutic agent is about 20:1.

In some embodiments, the pharmaceutical composition for intratumoral delivery disclosed herein can contain more than one therapeutic agent (e.g., multiple polypeptides). For example, the pharmaceutical composition for intratumoral delivery disclosed herein can contain 2 or more therapeutic agents. In some embodiments, the pharmaceutical composition for intratumoral delivery disclosed herein can contain one or more polynucleotides encoding a therapeutic agent (e.g., an mRNA). For example, a pharmaceutical composition disclosed herein can contain 2 or more polynucleotides (e.g., mRNA or siRNA).

### V. Nanoparticle Compositions

In some embodiments, the compositions disclosed herein are formulated as lipid nanoparticles (LNP). In such a nanoparticle composition, the lipid composition disclosed herein can encapsule a polynucleotide (e.g., mRNA).

In some embodiments, the pharmaceutical compositions for intratumoral delivery disclosed herein are formulated as lipid nanoparticles (LNP). Accordingly, the present disclosure also provides nanoparticle compositions comprising (i) a lipid composition comprising a compound of formula (I) as described herein, and (ii) a therapeutic agent or polynucleotide encoding a therapeutic agent. In such nanoparticle composition, the lipid composition component of the pharmaceutical composition for intratumoral delivery disclosed herein can encapsule the therapeutic agent or polynucleotide encoding a therapeutic agent.

As used herein, a “nanoparticle composition” is a composition comprising one or more lipids. Nanoparticle compositions are typically sized in the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, lipid vesicles, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

Nanoparticle compositions of the present disclosure can comprise at least one compound according to formula (I). For example, the nanoparticle composition can include one or more of Compounds 1-147. Nanoparticle compositions can also include a variety of other components. For example, the lipid component of a nanoparticle composition can include one or more other lipids in addition to a lipid according to formula (I), for example (i) at least one phospholipid, (ii) at least one quaternary amine compound, (iii) at least one structural lipid, (iv) at least one PEG-lipid, or (v) any combination thereof. In some embodiments, one or more components disclosed above are not present in a nanoparticle composition of the present disclosure.

In some embodiments, the nanoparticle composition comprises a compound of formula (I) (e.g., Compound 18). In some embodiments, the nanoparticle composition comprises a compound of formula (I) (e.g., Compound 18) and a phospholipid (e.g., DSPC or MSPC). In some embodiments, the nanoparticle composition comprises a compound of formula (I) (e.g., Compound 18), a phospholipid (e.g., DSPC or MSPC), and a quaternary amine compound (e.g., DOTAP). In some embodiments, the nanoparticle composition comprises a compound of formula (I) (e.g., Compound 18), and a quaternary amine compound (e.g., DOTAP).

In some embodiments, the nanoparticle composition comprises a lipid composition consisting or consisting essentially of compound of formula (I) (e.g., Compound 18). In some embodiments, the nanoparticle composition comprises a lipid composition consisting or consisting essentially of a compound of formula (I) (e.g., Compound 18) and a phospholipid (e.g., DSPC or MSPC). In some embodiments, the nanoparticle composition comprises a lipid composition consisting or consisting essentially of a compound of formula (I) (e.g., Compound 18), a phospholipid (e.g., DSPC or MSPC), and a quaternary amine compound (e.g., DOTAP). In some embodiments, the nanoparticle composition comprises a lipid composition consisting or consisting essentially of a compound of formula (I) (e.g., Compound 18), and a quaternary amine compound (e.g., DOTAP).

Nanoparticle compositions can be characterized by a variety of methods. For example, microscopy (e.g., transmission electron microscopy or scanning electron microscopy) can be used to examine the morphology and size distribution of a nanoparticle composition. Dynamic light scattering or potentiometry (e.g., potentiometric titrations) can be used to measure zeta potentials. Dynamic light scattering can also be utilized to determine particle sizes. Instruments such as the Zetasizer Nano ZS (Malvern Instruments Ltd, Malvern, Worcestershire, UK) can also be used to measure multiple characteristics of a nanoparticle composition, such as particle size, polydispersity index, and zeta potential.

The size of the nanoparticles can help counter biological reactions such as, but not limited to, inflammation, or can increase the biological effect of a therapeutic agent or polynucleotide encoding a therapeutic agent when administered intratumorally to a tumor in a subject. As used herein, “size” or “mean size” in the context of nanoparticle compositions refers to the mean diameter of a nanoparticle composition.

In one embodiment, the therapeutic agent or polynucleotide encoding a therapeutic agent (e.g., a polynucleotide comprising an mRNA encoding a polypeptide) are formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm or about 90 to about 100 nm.

In one embodiment, the nanoparticles in the nanoparticle composition have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 μm or shorter (e.g., 1 μm, 900 nm, 800 nm, 700 nm, 600 nm, 500 nm, 400 nm, 300 nm, 200 nm, 175 nm, 150 nm, 125 nm, 100 nm, 75 nm, 50 nm, or shorter), e.g., when measured by dynamic light scattering (DLS), transmission electron microscopy, scanning electron microscopy, or another method.

A nanoparticle composition may be relatively homogenous. A polydispersity index can be used to indicate the homogeneity of a nanoparticle composition, e.g., the particle size distribution of the nanoparticle compositions. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition can be from about 0.10 to about 0.20.

The zeta potential of a nanoparticle composition can be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

The term “encapsulation efficiency” of a therapeutic agent or polynucleotide encoding a therapeutic agent (e.g., mRNA) describes the amount of therapeutic agent or polynucleotide encoding a therapeutic agent (e.g., mRNA) that is encapsulated or otherwise associated with a nanoparticle composition after preparation, relative to the initial amount provided. As used herein, “encapsulation” can refer to complete, substantial, or partial enclosure, confinement, surrounding, or encasement.

Encapsulation efficiency is desirably high (e.g., close to 100%). The encapsulation efficiency can be measured, for example, by comparing the amount of therapeutic agent or polynucleotide encoding a therapeutic agent (e.g., mRNA) in a solution containing the nanoparticle composition before and after breaking up the nanoparticle composition with one or more organic solvents or detergents.

Fluorescence can be used to measure the amount of free therapeutic agent or polynucleotide encoding a therapeutic agent (e.g., mRNA) in a solution. For the nanoparticle compositions described herein, the encapsulation efficiency of a therapeutic agent or polynucleotide encoding a therapeutic agent (e.g., mRNA) can be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency can be at least 80%. In certain embodiments, the encapsulation efficiency can be at least 90%.

The amount of a therapeutic agent or polynucleotide encoding a therapeutic agent present in a pharmaceutical composition for intratumoral delivery disclosed herein can depend on multiple factors such as the size of the therapeutic agent or polynucleotide encoding a therapeutic agent (e.g., size of polypeptide or polynucleotide), desired target and/or application, or other properties of the nanoparticle composition as well as on the properties of the therapeutic agent or polynucleotide encoding a therapeutic agent present.

For example, the amount of an RNA useful in a nanoparticle composition can depend on the size (expressed as length, or molecular mass), sequence, and other characteristics of the RNA. The relative amounts of a therapeutic agent or polynucleotide encoding a therapeutic agent and other elements (e.g., lipids) in a nanoparticle composition can also vary.

The relative amounts of lipid composition and therapeutic agent or polynucleotide encoding a therapeutic agent present in a lipid nanoparticle composition of the present disclosure can be optimized according to considerations of efficacy and tolerability. For compositions including an RNA as a polynucleotide, the N:P ratio can serve as a useful metric.

As used herein, the “N:P ratio” is the molar ratio of ionizable (in the physiological pH range) nitrogen atoms in a lipid to phosphate groups in a polynucleotide (e.g., RNA), e.g., in a nanoparticle composition including a lipid component and an mRNA.

As the N:P ratio of a nanoparticle composition controls both expression and tolerability, nanoparticle compositions with low N:P ratios and strong expression are desirable. N:P ratios vary according to the ratio of lipids to RNA in a nanoparticle composition. N:P ratios are calculated for each nanoparticle composition assuming a single protonated nitrogen atom.

In some embodiments, a nanoparticle composition includes one or more RNAs, and the one or more RNAs, lipids, and amounts thereof can be selected to provide a specific N:P ratio. The N:P ratio of the composition refers to the molar ratio of nitrogen atoms in one or more lipids to the number of phosphate groups in an RNA. In general, a lower N:P ratio is preferred. The one or more RNA, lipids, and amounts thereof can be selected to provide an N:P ratio from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In certain embodiments, the N:P ratio can be from about 2:1 to about 8:1. In other embodiments, the N:P ratio is from about 5:1 to about 8:1. In certain embodiments, the N:P ratio is between 5:1 and 6:1. In one specific aspect, the N:P ratio is about is about 5.67:1.

In addition to providing nanoparticle compositions, the present disclosure also provides methods of producing lipid nanoparticles for intratumoral delivery comprising encapsulating a therapeutic agent or a polynucleotide encoding a therapeutic agent or a portion thereof. Such method comprises using any of the pharmaceutical compositions for intratumoral delivery disclosed herein and producing lipid nanoparticles in accordance with methods of production of lipid nanoparticles known in the art. See, e.g., Wang et al. (2015) “Delivery of oligonucleotides with lipid nanoparticles” Adv. Drug Deliv. Rev. 87:68-80; Silva et al. (2015) “Delivery Systems for Biopharmaceuticals. Part I: Nanoparticles and Microparticles” Curr. Pharm. Technol. 16: 940-954; Naseri et al. (2015) “Solid Lipid Nanoparticles and Nanostructured Lipid Carriers: Structure, Preparation and Application” Adv. Pharm. Bull. 5:305-13; Silva et al. (2015) “Lipid nanoparticles for the delivery of biopharmaceuticals” Curr. Pharm. Biotechnol. 16:291-302, and references cited therein.

### VI. Targets for Pharmaceutical Compositions

In some embodiments, the polynucleotides (e.g., mRNA) encoding a polypeptide can be used to treat or prevent a disease or condition. In other embodiments, the composition of the present disclosure can reduce or decrease a size of a tumor or inhibit a tumor growth in a subject in need thereof.

In some embodiments, the pharmaceutical compositions disclosed herein are suitable for administration to tumors. The term “tumor” is used herein in a broad sense and refers to any abnormal new growth of tissue that possesses no physiological function and arises from uncontrolled usually rapid cellular proliferation. The term “tumor” as used herein relates to both benign tumors and to malignant tumors.

In some embodiments, the tumor is a benign tumor. A benign tumor is a mass of cells (tumor) that lacks the ability to invade neighboring tissue or metastasize. These characteristics are required for a tumor to be defined as cancerous and therefore benign tumors are non-cancerous. Benign tumors are typically surrounded by an outer surface (fibrous sheath of connective tissue) or remain with the epithelium. Common examples of benign tumors include moles and uterine fibroids. In some embodiments, the benign tumor can include, but is not limited to, cholangioma, colonic polip, glandular adenoma, papilloma, cytadenoma, liver cell adenoma, hydatiform mole, renal tubular adenoma, squamous cell papilloma, gastric polyp, hemangioma, osteoma, chondroma, lipoma, fibroma, lymphangioma, leiomyoma, rhabdomyoma, astrocytoma, nevus, meningioma, or glanglioneuroma.

In some embodiments, the benign tumor can be caused by a genetic mutation. In that respect, in some embodiments the tumors are caused by PTEN hamartoma syndrome (this disease comprises Cowden syndrome, Bannayan-Riley-Ruvalcaba syndrome, Proteus syndrome or Proteus-like syndrome, resulting multiple benign hamartomas such as trichilemmomas and mucocutaneous papillomatous papules, hamartomatous intestinal polyps, lipomas, hemangiomas, nevi, cystadenomas, or adenomas), familial adenomatous polyposis (in this disorder adenomatous polyps are present in the colon that invariably progress into colon cancer), tuberous sclerosis complex (this disorder presents with many benign hamartomatous tumors including angiofibromas, renal angiomyolipomas, pulmonary lymphangiomyomatosis) or Von Hippel-Lindau disease (dominantly inherited cancer syndrome that increases the risk of various tumors including benign hemangioblastomas and malignant pheochromocytomas, renal cell carcinomas, pancreatic endocrine tumors and endolymphatic sac tumors).

In some embodiments, tumors are malignant tumors caused by cancer. The term “cancer” refers to a broad group of various diseases characterized by the uncontrolled growth of abnormal cells in the body with the potential to invade or spread to other parts of the body. Unregulated cell division and growth results in the formation of malignant tumors that invade neighboring tissues and can also metastasize to distant parts of the body through the lymphatic system or bloodstream. A “cancer” or “cancer tissue” can include a tumor at various stages. In certain embodiments, the cancer or tumor is stage 0, such that, e.g., the cancer or tumor is very early in development and has not metastasized. In some embodiments, the cancer or tumor is stage I, such that, e.g., the cancer or tumor is relatively small in size, has not spread into nearby tissue, and has not metastasized. In other embodiments, the cancer or tumor is stage II or stage III, such that, e.g., the cancer or tumor is larger than in stage 0 or stage I, and it has grown into neighboring tissues but it has not metastasized, except potentially to the lymph nodes. In other embodiments, the cancer or tumor is stage IV, such that, e.g., the cancer or tumor has metastasized. Stage IV can also be referred to as advanced or metastatic cancer.

In some embodiments, the malignant tumor is a primary tumor, i.e., a tumor growing at the anatomical site where tumor progression began and proceeded to yield a cancerous mass. In other embodiments, the malignant tumor is secondary tumor (metastasis).

In some embodiments, the cancer can include, but is not limited to, adrenal cortical cancer, advanced cancer, anal cancer, bileduct cancer, bladder cancer, bone cancer, bone metastasis, brain tumors, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, Hodgkin disease, Kaposi sarcoma, renal cell carcinoma, laryngeal and hypopharyngeal cancer, liver cancer, non-small cell lung cancer, small cell lung cancer, lung carcinoid tumor, lymphoma of the skin, malignant mesothelioma, multiple myeloma, myelodysplastic syndrome, nasal cavity and paranasal sinus cancer, nasopharyngeal cancer, neuroblastoma, non-Hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal and squamous cell skin cancer, melanoma, small intestine cancer, stomach cancer, testicular cancer, throat cancer, thymus cancer, thyroid cancer, uterine sarcoma, vaginal cancer, vulvar cancer, Wilms tumor and secondary cancers caused by cancer treatment.

In some embodiments, the tumor is a solid tumor. A “solid tumor” includes, but is not limited to, sarcoma, melanoma, carcinoma, or other solid tumor cancer. “Sarcoma” refers to a tumor which is made up of a substance like the embryonic connective tissue and is generally composed of closely packed cells embedded in a fibrillar or homogeneous substance. Sarcomas include, but are not limited to, chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, or telangiectaltic sarcoma.

The term “melanoma” refers to a tumor arising from the melanocytic system of the skin and other organs. Melanomas include, for example, acra-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, metastatic melanoma, nodular melanoma, subungal melanoma, or superficial spreading melanoma.

The term “carcinoma” refers to a malignant new growth made up of epithelial cells tending to infiltrate the surrounding tissues and give rise to metastases. Exemplary carcinomas include, e.g., acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidernoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, or carcinoma viflosum.

Additional cancers that can be treated include, e.g., neuroblastoma, breast cancer, ovarian cancer, lung cancer, rhabdomyosarcoma, small-cell lung tumors, primary brain tumors, stomach cancer, colon cancer, malignant pancreatic insulanoma, malignant carcinoid, urinary bladder cancer, premalignant skin lesions, testicular cancer, thyroid cancer, papillary thyroid cancer, neuroblastoma, neuroendocrine cancer, esophageal cancer, genitourinary tract cancer, malignant hypercalcemia, cervical cancer, endometrial cancer, adrenal cortical cancer, prostate cancer, Müllerian cancer, ovarian cancer, peritoneal cancer, fallopian tube cancer, or uterine papillary serous carcinoma.

### VII. Methods of Producing a Pharmaceutical Formulation

The pharmaceutical compositions for intratumoral delivery disclosed herein, or specification formulation of those compositions, e.g., the nanoparticles discussed above, can further include one or more pharmaceutically acceptable excipients, vehicles, or accessory ingredients such as those described herein.

The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient,” as used herein, refers to any ingredient other than the pharmaceutical compositions for intratumoral administration disclosed herein and having the properties of being substantially nontoxic and non-inflammatory in a subject. For example, a pharmaceutically acceptable excipient can refer to a vehicle capable of suspending, complexing, or dissolving the therapeutic agent or polynucleotide encoding a therapeutic agent as disclosed herein.

General guidelines for the formulation and manufacture of pharmaceutical compositions and agents are available, for example, in Remington's The Science and Practice of Pharmacy, 21st Edition, A. R. Gennaro; Lippincott, Williams & Wilkins, Baltimore, Md., 2006. Conventional excipients and accessory ingredients can be used in any pharmaceutical composition, except insofar as any conventional excipient or accessory ingredient may be incompatible with one or more components of a nanoparticle composition. An excipient or accessory ingredient may be incompatible with a component of a nanoparticle composition if its combination with the component may result in any undesirable biological effect or otherwise deleterious effect.

In some embodiments, one or more excipients or accessory ingredients can make up greater than 50% of the total mass or volume of a pharmaceutical composition for intratumoral delivery disclosed herein, including a nanoparticle composition. For example, the one or more excipients or accessory ingredients can make up 50%, 60%, 70%, 80%, 90%, or more of a pharmaceutical convention.

In some embodiments, a pharmaceutically acceptable excipient is at least 95%, at least 96%, at least 97%, at least 98%, at least 99%, or 100% pure. In some embodiments, an excipient is approved for use in humans and for veterinary use. In some embodiments, an excipient is approved by United States Food and Drug Administration. In some embodiments, an excipient is pharmaceutical grade. In some embodiments, an excipient meets the standards of the United States Pharmacopoeia (USP), the European Pharmacopoeia (EP), the British Pharmacopoeia, and/or the International Pharmacopoeia.

Relative amounts of a pharmaceutical composition for intratumoral delivery disclosed herein (e.g., in nanoparticle composition form), one or more pharmaceutically acceptable excipients, and/or any additional ingredients will vary, depending upon the identity, size, and/or condition of the subject treated. By way of example, a pharmaceutical composition can comprise between 0.1% and 100% (wt/wt) of one or more nanoparticle compositions.

Pharmaceutical compositions and nanoparticle compositions disclosed herein can be administered to any patient or subject. Although the descriptions provided herein of pharmaceutical compositions and nanoparticle compositions are principally directed to compositions which are suitable for administration to humans, it will be understood by the skilled artisan that such compositions are generally suitable for administration to any other mammal. Modification of compositions suitable for administration to humans in order to render the compositions suitable for administration to various animals is well understood, and the ordinarily skilled veterinary pharmacologist can design and/or perform such modification with merely ordinary, if any, experimentation.

Subjects to which administration of the pharmaceutical composition for intratumoral delivery disclosed herein are contemplated include, but are not limited to, humans, other primates, and other mammals, including commercially relevant mammals such as cattle, pigs, hoses, sheep, cats, dogs, mice, and/or rats.

Pharmaceutical composition for intratumoral delivery disclosed herein can be prepared by any method known or hereafter developed in the art of pharmacology. In general, such preparatory methods include bringing the active ingredient into association with an excipient and/or one or more other accessory ingredients, and then, if desirable or necessary, dividing, shaping, and/or packaging the product into a desired single- or multi-dose unit.

A pharmaceutical composition for intratumoral delivery disclosed herein can be prepared, packaged, and/or sold in bulk, as a single unit dose, and/or as a plurality of single unit doses. As used herein, a “unit dose” is discrete amount of the pharmaceutical composition comprising a predetermined amount of the active ingredient (e.g., therapeutic agent or polynucleotide encoding a therapeutic agent). The amount of the active ingredient is generally equal to the dosage of the active ingredient which would be administered to a subject and/or a convenient fraction of such a dosage such as, for example, one-half or one-third of such a dosage.

Pharmaceutical compositions can be prepared in a variety of forms suitable for a variety of routes and methods of administration. For example, pharmaceutical compositions can be prepared in injectable forms for intratumoral delivery.

Injectable preparations, for example, sterile injectable aqueous or oleaginous suspensions can be formulated according to the known art using suitable dispersing agents, wetting agents, and/or suspending agents. Sterile injectable preparations can be sterile injectable solutions, suspensions, and/or emulsions in nontoxic parenterally acceptable diluents and/or solvents, for example, as a solution in 1,3-butanediol. Among the acceptable vehicles and solvents that can be employed are water, Ringer's solution, U.S.P., and isotonic sodium chloride solution. Sterile, fixed oils are conventionally employed as a solvent or suspending medium. For this purpose any fixed oil can be employed including synthetic mono- or diglycerides. Fatty acids such as oleic acid can be used in the preparation of injectables.

Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, the pharmaceutical compositions for intratumoral administration disclosed herein can be in kit form. For example, the lipid composition component can be packaged separately from the therapeutic agent component of the composition, and both components can be combined prior to administration. The present disclosure also contemplates a pharmaceutical composition for intratumoral delivery comprising only the lipid component, which could be combined subsequently with a suitable therapeutic agent.

The present disclosure also provides a method of producing a pharmaceutical composition for delivery, e.g., for intratumoral delivery, comprising a therapeutic agent or a polynucleotide encoding a therapeutic agent, comprising formulating the therapeutic agent or polynucleotide encoding a therapeutic agent in the lipid composition, i.e., a lipid composition comprising at least one compound of formula (I) (e.g., Compound 18). In some embodiments, the invention provides a method of formulating a therapeutic agent or a polynucleotide encoding a therapeutic agent in a lipid nanoparticle comprising a compound of formula (I) (e.g., Compound 18) and a phospholipid (e.g., MSPC). In some embodiments, the invention provides a method of formulating a therapeutic agent or a polynucleotide encoding a therapeutic agent in a lipid nanoparticle comprising a compound of formula (I) (e.g., Compound 18), a phospholipid (e.g., MSPC), and a quaternary amine compound (e.g., DOTAP). In some embodiments, the resulting pharmaceutical compositions for administration, e.g., for intratumoral administration, can:

(i) increase the retention of the therapeutic agent in the tumor;

(ii) increase the retention of the polynucleotide encoding a therapeutic agent in the tumor;

(iii) increase the levels of expressed polypeptide in the tumor compared to the levels of expressed polypeptide in peritumoral tissue;

(iv) decrease leakage of the polynucleotide or expressed product to off-target tissue (e.g., peritumoral tissue, or to distant locations, e.g., liver tissue); or,

(v) any combination thereof,

wherein the increase or decrease observed for a certain property is relative to a corresponding reference composition (e.g., composition in which compounds of formula (I) are not present or have been substituted by another ionizable amino lipid, e.g., MC3).

In one embodiment, a decrease in leakage can be quantified as increase in the ratio of polypeptide expression in the tumor to polypeptide expression in non-tumor tissues, such as peritumoral tissue or to another tissue or organ, e.g., liver tissue.

### VIII. Methods of Intratumoral Delivery

The present disclosure provides methods of delivering a polynucleotide (e.g., an mRNA) to a cell, tissue, or organ. For example, the present disclosure provides methods of delivering a polynucleotide to a tumor. Delivery of a polynucleotide to a cell, tissue, or organ involves administering a lipid composition including the polynucleotide to a subject, where administration of the composition involves contacting the cell, tissue, or, organ with the composition. In the instance that a polynucleotide is an mRNA, upon contacting a cell with the lipid composition, a translatable mRNA can be translated in the cell to produce a polypeptide of interest.

The present disclosure also provides a method of delivering a therapeutic agent or polynucleotide encoding a therapeutic agent to a tumor, comprising formulating the therapeutic agent or polynucleotide encoding a therapeutic agent in the pharmaceutical composition described herein, e.g., in lipid nanoparticle form, and administering the pharmaceutical composition to a tumor. The administration of the pharmaceutical composition to the tumor can be performed using any method known in the art (e.g., bolus injection, perfusion, surgical implantation, etc.).

The delivery of the therapeutic agent or polynucleotide encoding a therapeutic agent to a tumor using a pharmaceutical compositions for intratumoral administration disclosed herein can:

(i) increase the retention of the polynucleotide encoding a therapeutic agent in the tumor;

(ii) increase the levels of expressed polypeptide in the tumor compared to the levels of expressed polypeptide in peritumoral tissue;

(iii) decrease leakage of the polynucleotide or expressed product to off-target tissue (e.g., peritumoral tissue, or to distant locations, e.g., liver tissue); or,

(v) any combination thereof,

wherein the increase or decrease observed for a certain property is relative to a corresponding reference composition (e.g., composition in which compounds of formula (I) are not present or have been substituted by another ionizable amino lipid, e.g., MC3).

In one embodiment, a decrease in leakage can be quantified as increase in the ratio of polypeptide expression in the tumor to polypeptide expression in non-tumor tissues, such as peritumoral tissue or to another tissue or organ, e.g., liver tissue.

Delivery of a therapeutic agent or polynucleotide encoding a therapeutic agent to a tumor involves administering a pharmaceutical composition disclosed herein, e.g., in nanoparticle form, including the therapeutic agent or polynucleotide encoding a therapeutic agent to a subject, where administration of the pharmaceutical composition involves contacting the tumor with the composition.

For example, a protein, cytotoxic agent, radioactive ion, chemotherapeutic agent, or nucleic acid (such as an RNA, e.g., mRNA) can be delivered to a tumor. In the instance that a therapeutic agent or polynucleotide encoding a therapeutic agent is an mRNA, upon contacting a cell in the tumor with the pharmaceutical composition, a translatable mRNA can be translated in the cell to produce a polypeptide of interest. However, mRNAs that are substantially not translatable can also be delivered to tumors. Substantially non-translatable mRNAs can be useful as vaccines and/or can sequester translational components of a cell to reduce expression of other species in the cell.

The pharmaceutical compositions disclosed herein can increase specific delivery.

As used herein, the term “specific delivery,” means delivery of more (e.g., at least 1.5 fold more, at least 2-fold more, at least 3-fold more, at least 4-fold more, at least 5-fold more, at least 6-fold more, at least 7-fold more, at least 8-fold more, at least 9-fold more, at least 10-fold more) of a therapeutic agent or polynucleotide encoding a therapeutic agent by pharmaceutical composition disclosed herein (e.g., in nanoparticle form) to a target tissue of interest (e.g., a tumor) compared to an off-target tissue (e.g., mammalian liver).

The level of delivery of a nanoparticle to a particular tissue can be measured, for example, by comparing

(i) the amount of protein expressed from a polynucleotide encoding a therapeutic agent in a tissue to the weight of said tissue;

(ii) comparing the amount of therapeutic agent in a tissue to the weight of said tissue; or

(iii) comparing the amount of protein expressed from a polynucleotide encoding a therapeutic agent in a tissue to the amount of total protein in said tissue.

Specific delivery to a tumor or a particular class of cells in the tumor implies that a higher proportion of pharmaceutical composition including a therapeutic agent or polynucleotide encoding a therapeutic agent is delivered to the target tissues relative to other off-target tissues upon administration of a pharmaceutical composition to a subject.

The present disclosure also provides methods to achieve improved intratumoral delivery of a therapeutic agent or polynucleotide encoding a therapeutic agent when a pharmaceutical composition disclosed herein (e.g., in nanoparticle form) are administered to a tumor. The improvement in delivery can be due, for example, to

(i) increased retention of the polynucleotide encoding a therapeutic agent in the tumor;

(ii) increased levels of expressed polypeptide in the tumor compared to the levels of expressed polypeptide in peritumoral tissue;

(iii) decreased leakage of the polynucleotide or expressed product to off-target tissue (e.g., peritumoral tissue, or to distant locations, e.g., liver tissue); or,

(iv) any combination thereof,

wherein the increase or decrease observed for a certain property is relative to a corresponding reference composition (e.g., composition in which compounds of formula (I) are not present or have been substituted by another ionizable amino lipid, e.g., MC3).

In one embodiment, a decrease in leakage can be quantified as increase in the ratio of polypeptide expression in the tumor to polypeptide expression in non-tumor tissues, such as peritumoral tissue or to another tissue or organ, e.g., liver tissue.

Another improvement in delivery caused as a result of using the pharmaceutical compositions disclosed herein is a reduction in immune response with respect to the immune response observed when other lipid components are used to deliver the same therapeutic agent or polynucleotide encoding a therapeutic agent.

Accordingly, the present disclosure provides a method of increasing retention of a therapeutic agent (e.g., a polypeptide administered as part of the pharmaceutical composition) in a tumor tissue in a subject, comprising administering intratumorally to the tumor tissue a pharmaceutical composition disclosed herein, wherein the retention of the therapeutic agent in the tumor tissue is increased compared to the retention of the therapeutic agent in the tumor tissue after administering a corresponding reference composition.

Also provided is a method of increasing retention of a polynucleotide in a tumor tissue in a subject, comprising administering intratumorally to the tumor tissue a pharmaceutical composition disclosed herein, wherein the retention of the polynucleotide in the tumor tissue is increased compared to the retention of the polynucleotide in the tumor tissue after administering a corresponding reference composition.

Also provided is a method of increasing retention of an expressed polypeptide in a tumor tissue in a subject, comprising administering to the tumor tissue a pharmaceutical composition disclosed herein, wherein the pharmaceutical composition comprises a polynucleotide encoding the expressed polypeptide, and wherein the retention of the expressed polypeptide in the tumor tissue is increased compared to the retention of the polypeptide in the tumor tissue after administering a corresponding reference composition.

The present disclosure also provides a method of decreasing expression leakage of a polynucleotide administered intratumorally to a subject in need thereof, comprising administering said polynucleotide intratumorally to the tumor tissue as a pharmaceutical composition disclosed herein, wherein the expression level of the polypeptide in non-tumor tissue is decreased compared to the expression level of the polypeptide in non-tumor tissue after administering a corresponding reference composition.

Also provided is a method of decreasing expression leakage of a therapeutic agent (e.g., a polypeptide administered as part of the pharmaceutical composition) administered intratumorally to a subject in need thereof, comprising administering said therapeutic agent intratumorally to the tumor tissue as a pharmaceutical composition disclosed herein, wherein the amount of therapeutic agent in non-tumor tissue is decreased compared to the amount of therapeutic in non-tumor tissue after administering a corresponding reference composition.

Also provided is a method of decreasing expression leakage of an expressed polypeptide in a tumor in a subject, comprising administering to the tumor tissue a pharmaceutical composition disclosed herein, wherein the pharmaceutical composition comprises a polynucleotide encoding the expressed polypeptide, and wherein the amount of expressed polypeptide in non-tumor tissue is decreased compared to the amount of expressed polypeptide in non-tumor tissue after administering a corresponding reference composition.

In some embodiments, the non-tumoral tissue is peritumoral tissue. In other embodiments, the non-tumoral tissue is liver tissue.

The present disclosure also provides a method to reduce or prevent the immune response caused by the intratumoral administration of a pharmaceutical composition, e.g., a pharmaceutical composition comprising lipids known in the art, by replacing one or all the lipids in such composition with a compound of Formula (I). For example, the immune response caused by the administration of a therapeutic agent or a polynucleotide encoding a therapeutic agent in a pharmaceutical composition comprising MC3 (or other lipids known in the art) can be prevented (avoided) or ameliorated by replacing MC3 with a compound of formula (I), e.g., Compound 18.

In some embodiments, the immune response observed after a therapeutic agent or a polynucleotide encoding a therapeutic agent is administered in a pharmaceutical composition disclosed herein is not elevated compared to the immune response observed when the therapeutic agent or a polynucleotide encoding a therapeutic agent is administered in phosphate buffered saline (PBS) or another physiological buffer solution (e.g., Ringer's solution, Tyrode's solution, Hank's balanced salt solution, etc.).

In some embodiments, the immune response observed after a therapeutic agent or a polynucleotide encoding a therapeutic agent is administered in a pharmaceutical composition disclosed herein is not elevated compared to the immune response observed when PBS or another physiological buffer solution is administered alone.

In some embodiments, no immune response is observed when a pharmaceutical composition disclosed herein is administered intratumorally to a subject.

In other embodiments, the intratumoral delivery of a pharmaceutical composition of the invention exhibits an increased target protein expression and/or a reduced cytokine expression (e.g., IL6 or G-CSF) compared to the delivery of PBS or a pharmaceutical composition comprising another lipid, e.g., MC3. In one embodiment, the protein to cytokine (e.g., IL6) expression ratio after the intratumoral delivery of a pharmaceutical composition of the invention is at least 1, at least 5, at least 10, at least 20, at least 30, at least 40, at least 50, at least 60, at least 70, at least 80, at least 90, at least 100, at least 110, at least 120, at least 130, at least 140, at least 150, or at least 200.

In other embodiments, the delivery of a pharmaceutical composition of the invention exhibits a short tissue half-life compared to a pharmaceutical composition comprising MC3. In some embodiments, the delivery of a pharmaceutical composition of the invention exhibits a short plasma half-life compared to a pharmaceutical composition comprising MC3.

In certain embodiments, the intratumoral delivery of the pharmaceutical composition is well tolerated. For example, the intratumoral delivery of a pharmaceutical composition of the invention shows less injection site reactions, less systemic inflammation, less systemic inflammation induced stress, higher STD₁₀ (the dose that results in 10% mortality over the duration of the study), higher HNSTD (the highest non-severly toxic dose), or any combination thereof than the delivery of a pharmaceutical composition comprising MC3.

In other embodiments, the intratumoral delivery of a pharmaceutical composition of the invention exhibits a higher protein expression in tumor cells or one or more immune cells (e.g., B cells, NK cells, CD11b+ myeloid cells, CD8+ T cells, CD4+ T cells, or any combination thereof) compared to the delivery of a pharmaceutical composition comprising MC3.

In yet other embodiments, the intratumoral delivery of a pharmaceutical composition of the invention exhibits a lesser increase in tumor myeloid-derived suppressor cells (MDSCs, e.g., Ly6G+ cells) than the delivery of a pharmaceutical composition comprising MC3.

Accordingly, the present disclosure also provides a method of delivering a therapeutic agent or a polynucleotide encoding a therapeutic agent to a subject in need thereof, comprising administering intratumorally to the subject a pharmaceutical composition disclosed herein, wherein the immune response caused by the administration of the pharmaceutical composition is not elevated compared to the immune response caused by the intratumoral administration of

(i) PBS alone, or another physiological buffer solution (e.g., Ringer's solution, Tyrode's solution, Hank's balanced salt solution, etc.);

(ii) the therapeutic agent or polynucleotide encoding a therapeutic agent in PBS or another physiological buffer solution; or,

(iii) a corresponding reference composition, i.e., the same pharmaceutical composition in which the compound of formula (I) is substituted by another ionizable amino lipid, e.g., MC3.

In some embodiments, the immune response is an inflammatory response. In some embodiments, the inflammatory response can be measured by quantifying the concentration of one or more inflammatory cytokines in plasma, tumor tissue, or peritumoral tissue. In some embodiments, the quantified antiinflammatory cytokines are, e.g., interleukin-6 (IL-6), CXCL1 (chemokine (C—X—C motif) ligand 1; also known as GROα, interferon-γ (IFNγ), tumor necrosis factor α (TNFα), interferon y-induced protein 10 (IP-10), granulocyte-colony stimulating factor (G-CSF), or a combination thereof. In some embodiments, the quantified inflammatory cytokines are IL-6, G-CSF, GROα, or a combination thereof. In a specific embodiment the presence of an immune response is determine by the presence of elevated levels of IL-6; or the presence of elevated levels of IL-6 and G-CSG; or the presence of elevated levels of IL-6, G-CSF, and GROα. The levels of inflammatory cytokines can be quantified using any methods known in the art, e.g., ELISA.

In some embodiments, the immune response or inflammatory response is measured, e.g., 6, 12, 18, 24, or 48 hours after administration of the pharmaceutical composition.

### IX. Methods of Improved In Situ Expression of a Therapeutic Agent

The present disclosure also provides methods to improve the in situ expression of a polypeptide in a tumor comprising delivering to the tumor a pharmaceutical composition comprising a lipid component comprising a compound of Formula (I). Methods of producing polypeptides in a tumor, e.g., a therapeutic agent such as an antibody or a fragment thereof, involve contacting a tumor cell with a pharmaceutical composition disclosed herein (e.g., in nanoparticle form), wherein the pharmaceutical composition comprises an mRNA encoding the polypeptide of interest. Upon contacting the cell with the pharmaceutical composition, the mRNA can be taken up and translated in the cell to produce the polypeptide of interest.

In some embodiments, the present disclosure provides a method of increasing protein expression of a polypeptide in a tumor tissue of a subject, comprising administering intratumorally to the tumor tissue a pharmaceutical composition disclosed herein, wherein the expression level of the polypeptide in the tumor tissue is increased compared to the expression level of the polypeptide after administering a corresponding reference composition.

The amount of pharmaceutical composition contacted with a tumor cell, and/or the amount of mRNA therein, may depend on the type of cell or tissue being contacted, the means of administration, the physiochemical characteristics of the pharmaceutical composition (e.g., characteristics of a nanoparticle with the pharmaceutical composition is in nanoparticle form) and the mRNA (e.g., size, charge, and chemical composition) therein, and other factors. In general, an effective amount of the pharmaceutical composition will allow for efficient polypeptide production in the cell. Metrics for efficiency can include polypeptide translation (indicated by polypeptide expression), level of mRNA degradation, and immune response indicators.

The step of contacting a pharmaceutical composition including an mRNA with a tumor cell may involve or cause transfection. The presence of a compound of Formula (I), a phospholipid, or other component included in the lipid composition component of the pharmaceutical composition may facilitate transfection and/or increase transfection efficiency, for example, by interacting and/or fusing with a cellular or intracellular membrane. Transfection may allow for the translation of the mRNA within the tumor cell.

In some embodiments, the pharmaceutical composition disclosed herein can be used therapeutically. For example, an mRNA included in a pharmaceutical composition can encode a therapeutic polypeptide (e.g., in a translatable region) and produce the therapeutic polypeptide upon contacting and/or entry (e.g., transfection) into a cell. In other embodiments, an mRNA included in a pharmaceutical composition disclosed herein can encode a polypeptide that can improve or increase the immunity of a subject.

In certain embodiments, an mRNA included in a pharmaceutical composition disclosed herein can encode a recombinant polypeptide that can replace one or more polypeptides that can be substantially absent in a tumor cell contacted with the pharmaceutical composition disclosed herein. The one or more substantially absent polypeptides may be lacking due to a genetic mutation of the encoding gene or a regulatory pathway thereof. Alternatively, a recombinant polypeptide produced by translation of the mRNA may antagonize the activity of an endogenous protein present in, on the surface of, or secreted from the tumor cell. An antagonistic recombinant polypeptide may be desirable to combat deleterious effects caused by activities of the endogenous protein, such as altered activities or localization caused by mutation. In another alternative, a recombinant polypeptide produced by translation of the mRNA may indirectly or directly antagonize the activity of a biological moiety present in, on the surface of, or secreted from the tumor cell. Antagonized biological moieties can include, but are not limited to, lipids (e.g., cholesterol), lipoproteins (e.g., low density lipoprotein), nucleic acids, carbohydrates, and small molecule toxins. Recombinant polypeptides produced by translation of the mRNA may be engineered for localization within the tumor cell, such as within a specific compartment such as the nucleus, or may be engineered for secretion from the tumor cell or for translocation to the plasma membrane of the tumor cell.

Additionally, efficiency of polypeptide production (e.g., translation) in the tumor can be optionally determined, and the tumor can be re-contacted with the pharmaceutical composition disclosed repeatedly until a target protein production efficiency is achieved.

### X. Generation of Polynucleotides for Intratumoral Delivery

The pharmaceutical compositions for intratumoral delivery disclosed herein can contain a polynucleotide, in addition to the lipid composition component comprising a compound of Formula (I). In some aspects, such polynucleotide encodes a therapeutic agent, e.g., a polypeptide. Such polynucleotides include, e.g., plasmid DNA, linear DNA selected from poly and oligo-nucleotides, chromosomal DNA, messenger RNA (mRNA), antisense DNA/RNA, siRNA, microRNA (miRNA), ribosomal RNA, oligonucleotide DNA (ODN) single and double strand, CpG imunostimulating sequence (ISS), locked nucleic acid (LNA), and ribozyme.

In one embodiment, the polynucleotide comprises mRNA. In some embodiments, the mRNA is sequence-optimized for expression in a mammal. In a particular embodiment, the mRNA is sequence-optimized for expression in a human. In some embodiments, the mRNA is modified. In some embodiments, the mRNA comprises at least one chemically modified nucleoside. In some embodiments, the mRNA is modified to enhance its stability or half-life. In some embodiments, the modified mRNA has increased stability or half-life in an in vivo setting.

### IVT Polynucleotide Architecture

In some embodiments, the pharmaceutical compositions for intratumoral delivery disclosed herein comprise a polynucleotide encoding a therapeutic agent (e.g., a polypeptide), wherein the polynucleotide is an mRNA. In some embodiments, the mRNA encoding a polypeptide is an IVT (in vitro translation) polynucleotide. Such IVT polynucleotides can be used of optimize an mRNA encoding a therapeutic agent prior to its use in a formulation for intratumoral delivery.

Traditionally, the basic components of an mRNA molecule include at least a coding region, a 5′UTR, a 3′UTR, a 5′ cap and a poly-A tail. The IVT polynucleotides can function as mRNA but are distinguished from wild-type mRNA in their functional and/or structural design features which serve, e.g., to overcome existing problems of effective polypeptide production using nucleic-acid based therapeutics.

The primary construct of an IVT polynucleotide comprises a first region of linked nucleotides that is flanked by a first flanking region and a second flaking region. This first region can include, but is not limited to, the encoded polypeptide. The first flanking region can include a sequence of linked nucleosides which function as a 5′ untranslated region (UTR) such as the 5′ UTR of any of the nucleic acids encoding the native 5′ UTR of the polypeptide or a non-native 5′UTR such as, but not limited to, a heterologous 5′ UTR or a synthetic 5′ UTR. The IVT encoding a polypeptide can comprise at its 5 terminus a signal sequence region encoding one or more signal sequences. The flanking region can comprise a region of linked nucleotides comprising one or more complete or incomplete 5′ UTRs sequences. The flanking region can also comprise a 5′ terminal cap. The second flanking region can comprise a region of linked nucleotides comprising one or more complete or incomplete 3′ UTRs which can encode the native 3′ UTR of the polypeptide or a non-native 3′ UTR such as, but not limited to, a heterologous 3′ UTR or a synthetic 3′ UTR. The flanking region can also comprise a 3′ tailing sequence. The 3′ tailing sequence can be, but is not limited to, a polyA tail, a polyA-G quartet and/or a stem loop sequence.

Bridging the 5′ terminus of the first region and the first flanking region is a first operational region. Traditionally, this operational region comprises a Start codon. The operational region can alternatively comprise any translation initiation sequence or signal including a Start codon.

Bridging the 3′ terminus of the first region and the second flanking region is a second operational region. Traditionally this operational region comprises a Stop codon. The operational region can alternatively comprise any translation initiation sequence or signal including a Stop codon. Multiple serial stop codons can also be used in the IVT polynucleotide. In some embodiments, the operation region of the present application can comprise two stop codons. The first stop codon can be “TGA” or “UGA” and the second stop codon can be selected from the group consisting of “TAA,” “TGA,” “TAG,” “UAA,” “UGA” or “UAG.”

The IVT polynucleotide primary construct comprises a first region of linked nucleotides that is flanked by a first flanking region and a second flaking region. As used herein, the “first region” can be referred to as a “coding region” or “region encoding” or simply the “first region.” This first region can include, but is not limited to, the encoded polypeptide of interest. In one aspect, the first region can include, but is not limited to, the open reading frame encoding at least one polypeptide of interest. The open reading frame can be codon optimized in whole or in part. The flanking region can comprise a region of linked nucleotides comprising one or more complete or incomplete 5′ UTRs sequences which can be completely codon optimized or partially codon optimized. The flanking region can include at least one nucleic acid sequence including, but not limited to, miR sequences, TERZAK™ sequences and translation control sequences. The flanking region can also comprise a 5′ terminal cap 138. The 5′ terminal capping region can include a naturally occurring cap, a synthetic cap or an optimized cap. The second flanking region can comprise a region of linked nucleotides comprising one or more complete or incomplete 3′ UTRs. The second flanking region can be completely codon optimized or partially codon optimized. The flanking region can include at least one nucleic acid sequence including, but not limited to, miR sequences and translation control sequences. After the second flanking region the polynucleotide primary construct can comprise a 3′ tailing sequence. The 3′ tailing sequence can include a synthetic tailing region and/or a chain terminating nucleoside. Non-liming examples of a synthetic tailing region include a polyA sequence, a polyC sequence, a polyA-G quartet. Non-limiting examples of chain terminating nucleosides include 2′-O methyl, F and locked nucleic acids (LNA).

Bridging the 5′ terminus of the first region and the first flanking region is a first operational region. Traditionally this operational region comprises a Start codon. The operational region can alternatively comprise any translation initiation sequence or signal including a Start codon.

Bridging the 3′ terminus of the first region and the second flanking region is a second operational region. Traditionally this operational region comprises a Stop codon. The operational region can alternatively comprise any translation initiation sequence or signal including a Stop codon. According to the present disclosure, multiple serial stop codons can also be used.

In some embodiments, the first and second flanking regions of the IVT polynucleotide can range independently from 15-1,000 nucleotides in length (e.g., greater than 30, 40, 45, 50, 55, 60, 70, 80, 90, 100, 120, 140, 160, 180, 200, 250, 300, 350, 400, 450, 500, 600, 700, 800, 900, 1,000, 1,500, 2,000, 2,500, 3,000, 3,500, 4,000, 4,500, 5,000, 5,500 nucleotides or at least 30, 40, 45, 50, 55, 60, 70, 80, 90, 100, 120, 140, 160, 180, 200, 250, 300, 350, 400, 450, 500, 600, 700, 800, 900, 1,000, 1,500, 2,000, 2,500, 3,000, 3,500, 4,000, 4,500, 5,000, 5,500 nucleotides).

In some embodiments, the tailing sequence of the IVT polynucleotide can range from absent to 500 nucleotides in length (e.g., at least 60, 70, 80, 90, 120, 140, 160, 180, 200, 250, 300, 350, 400, 450, or 500 nucleotides). Where the tailing region is a polyA tail, the length can be determined in units of or as a function of polyA Binding Protein binding. In this embodiment, the polyA tail is long enough to bind at least 4 monomers of PolyA Binding Protein. PolyA Binding Protein monomers bind to stretches of approximately 38 nucleotides. As such, it has been observed that polyA tails of about 80 nucleotides and 160 nucleotides are functional.

In some embodiments, the capping region of the IVT polynucleotide can comprise a single cap or a series of nucleotides forming the cap. In this embodiment the capping region can be from 1 to 10, e.g. 2-9, 3-8, 4-7, 1-5, 5-10, or at least 2, or 10 or fewer nucleotides in length. In some embodiments, the cap is absent.

In some embodiments, the first and second operational regions of the IVT polynucleotide can range from 3 to 40, e.g., 5-30, 10-20, 15, or at least 4, or 30 or fewer nucleotides in length and can comprise, in addition to a Start and/or Stop codon, one or more signal and/or restriction sequences.

In some embodiments, the IVT polynucleotides can be structurally modified or chemically modified. When the IVT polynucleotides are chemically and/or structurally modified the polynucleotides can be referred to as “modified IVT polynucleotides.”

In some embodiments, if the IVT polynucleotides are chemically modified they can have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., pseudouridine or 5-methoxyuridine. In another embodiment, the IVT polynucleotides can have a uniform chemical modification of two, three, or four of the same nucleoside type throughout the entire polynucleotide (such as all uridines and all cytosines, etc. are modified in the same way).

In some embodiments, the IVT polynucleotides can include a sequence encoding a self-cleaving peptide, described herein, such as but not limited to the 2A peptide. The polynucleotide sequence of the 2A peptide in the IVT polynucleotide can be modified or codon optimized by the methods described herein and/or are known in the art. In some embodiments, this sequence can be used to separate the coding region of two or more polypeptides of interest in the IVT polynucleotide.

### Chimeric Polynucleotide Architecture

In some embodiments, the polynucleotide is a chimeric polynucleotide. The chimeric polynucleotides or RNA constructs disclosed herein maintain a modular organization similar to IVT polynucleotides, but the chimeric polynucleotides comprise one or more structural and/or chemical modifications or alterations which impart useful properties to the polynucleotide. As such, the chimeric polynucleotides which are modified mRNA molecules of the present disclosure are termed “chimeric modified mRNA” or “chimeric mRNA.”

Chimeric polynucleotides have portions or regions which differ in size and/or chemical modification pattern, chemical modification position, chemical modification percent or chemical modification population and combinations of the foregoing.

Examples of parts or regions, where the chimeric polynucleotide functions as an mRNA, but is not limited to, untranslated regions (UTRs, such as the 5′ UTR or 3′ UTR), coding regions, cap regions, polyA tail regions, start regions, stop regions, signal sequence regions, and combinations thereof. Regions or parts that join or lie between other regions can also be designed to have subregions.

### Circular Polynucleotide

The polynucleotide (e.g., mRNA) encoding a polypeptide can be circular or cyclic. As used herein, “circular polynucleotides” or “circP” means a single stranded circular polynucleotide which acts substantially like, and has the properties of, an RNA. The term “circular” is also meant to encompass any secondary or tertiary configuration of the circP. Circular polynucleotides are circular in nature meaning that the termini are joined in some fashion, whether by ligation, covalent bond, common association with the same protein or other molecule or complex or by hybridization.

Circular polynucleotides, formulations and compositions comprising circular polynucleotides, and methods of making, using and administering circular polynucleotides are also disclosed in International Patent Application No. PCT/US2014/53904.

Polynucleotides having Untranslated Regions (UTRs)

The polynucleotide (e.g., mRNA) encoding a polypeptide can further comprise a nucleotide sequence encoding one or more heterologous polypeptides. In one embodiment, the one or more heterologous polypeptides improves a pharmacokinetic property or pharmacodynamics property of the polypeptide or a polynucleotide encoding the polypeptide.

In one embodiment, the mRNA encodes an extracellular portion of a polypeptide and one or more heterologous polypeptides.

The polynucleotide (e.g., mRNA) encoding a polypeptide can further comprise one or more regions or parts which act or function as an untranslated region. By definition, wild type untranslated regions (UTRs) of a gene are transcribed but not translated. In mRNA, the 5′UTR starts at the transcription start site and continues to the start codon but does not include the start codon; whereas, the 3′UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into the polynucleotides of the present disclosure to, among other things, enhance the stability of the molecule. The specific features can also be incorporated to ensure controlled down-regulation of the transcript in case they are misdirected to undesired organs sites.

Untranslated regions (UTRs) are nucleic acid sections of a polynucleotide before a start codon (5′UTR) and after a stop codon (3′UTR) that are not translated. In some embodiments, a polynucleotide (e.g., a ribonucleic acid (RNA), e.g., a messenger RNA (mRNA)) comprising an open reading frame (ORF) encoding a polypeptide further comprises UTR (e.g., a 5′UTR or functional fragment thereof, a 3′UTR or functional fragment thereof, or a combination thereof).

A UTR can be homologous or heterologous to the coding region in a polynucleotide. In some embodiments, the UTR is homologous to the ORF encoding a polypeptide. In some embodiments, the UTR is heterologous to the ORF encoding a polypeptide. In some embodiments, the polynucleotide comprises two or more 5′UTRs or functional fragments thereof, each of which have the same or different nucleotide sequences. In some embodiments, the polynucleotide comprises two or more 3′UTRs or functional fragments thereof, each of which have the same or different nucleotide sequences.

In some embodiments, the 5′UTR or functional fragment thereof, 3′ UTR or functional fragment thereof, or any combination thereof is sequence optimized.

In some embodiments, the 5′UTR or functional fragment thereof, 3′ UTR or functional fragment thereof, or any combination thereof comprises at least one chemically modified nucleobase, e.g., 5-methoxyuracil.

UTRs can have features that provide a regulatory role, e.g., increased or decreased stability, localization and/or translation efficiency. A polynucleotide comprising a UTR can be administered to a cell, tissue, or organism, and one or more regulatory features can be measured using routine methods. In some embodiments, a functional fragment of a 5′UTR or 3′UTR comprises one or more regulatory features of a full length 5′ or 3′ UTR, respectively.

Natural 5′UTRs bear features that play roles in translation initiation. They harbor signatures like Kozak sequences that are commonly known to be involved in the process by which the ribosome initiates translation of many genes. Kozak sequences have the consensus CCR(A/G)CCAUGG, where R is a purine (adenine or guanine) three bases upstream of the start codon (AUG), which is followed by another ‘G’. 5′UTRs also have been known to form secondary structures that are involved in elongation factor binding.

By engineering the features typically found in abundantly expressed genes of specific target organs, one can enhance the stability and protein production of a polynucleotide. For example, introduction of 5′UTR of liver-expressed mRNA, such as albumin, serum amyloid A, Apolipoprotein A/B/E, transferrin, alpha fetoprotein, erythropoietin, or Factor VIII, can enhance expression of polynucleotides in hepatic cell lines or liver. Likewise, use of 5′UTR from other tissue-specific mRNA to improve expression in that tissue is possible for muscle (e.g., MyoD, Myosin, Myoglobin, Myogenin, Herculin), for endothelial cells (e.g., Tie-1, CD36), for myeloid cells (e.g., C/EBP, AML1, G-CSF, GM-CSF, CD11b, MSR, Fr-1, i-NOS), for leukocytes (e.g., CD45, CD18), for adipose tissue (e.g., CD36, GLUT4, ACRP30, adiponectin) and for lung epithelial cells (e.g., SP-A/B/C/D).

In some embodiments, UTRs are selected from a family of transcripts whose proteins share a common function, structure, feature or property. For example, an encoded polypeptide can belong to a family of proteins (i.e., that share at least one function, structure, feature, localization, origin, or expression pattern), which are expressed in a particular cell, tissue or at some time during development. The UTRs from any of the genes or mRNA can be swapped for any other UTR of the same or different family of proteins to create a new polynucleotide.

In some embodiments, the 5′UTR and the 3′UTR can be heterologous. In some embodiments, the 5′UTR can be derived from a different species than the 3′UTR. In some embodiments, the 3′UTR can be derived from a different species than the 5′UTR.

Co-owned International Patent Application No. PCT/US2014/021522 (Publ. No. WO/2014/164253, incorporated herein by reference in its entirety) provides a listing of exemplary UTRs that can be utilized in the polynucleotide of the present invention as flanking regions to an ORF.

Exemplary UTRs of the application include, but are not limited to, one or more 5′UTR and/or 3′UTR derived from the nucleic acid sequence of: a globin, such as an α- or β-globin (e.g., a Xenopus, mouse, rabbit, or human globin); a strong Kozak translational initiation signal; a CYBA (e.g., human cytochrome b-245 α polypeptide); an albumin (e.g., human albumin7); a HSD17B4 (hydroxysteroid (17-β) dehydrogenase); a virus (e.g., a tobacco etch virus (TEV), a Venezuelan equine encephalitis virus (VEEV), a Dengue virus, a cytomegalovirus (CMV) (e.g., CMV immediate early 1 (IE1)), a hepatitis virus (e.g., hepatitis B virus), a sindbis virus, or a PAV barley yellow dwarf virus); a heat shock protein (e.g., hsp70); a translation initiation factor (e.g., elF4G); a glucose transporter (e.g., hGLUT1 (human glucose transporter 1)); an actin (e.g., human α or β actin); a GAPDH; a tubulin; a histone; a citric acid cycle enzyme; a topoisomerase (e.g., a 5′UTR of a TOP gene lacking the 5′ TOP motif (the oligopyrimidine tract)); a ribosomal protein Large 32 (L32); a ribosomal protein (e.g., human or mouse ribosomal protein, such as, for example, rps9); an ATP synthase (e.g., ATP5A1 or the β subunit of mitochondrial H+-ATP synthase); a growth hormone e (e.g., bovine (bGH) or human (hGH)); an elongation factor (e.g., elongation factor 1 α1 (EEF1A1)); a manganese superoxide dismutase (MnSOD); a myocyte enhancer factor 2A (MEF2A); a β-F1-ATPase, a creatine kinase, a myoglobin, a granulocyte-colony stimulating factor (G-CSF); a collagen (e.g., collagen type I, alpha 2 (Col1A2), collagen type I, alpha 1 (Col1A1), collagen type VI, alpha 2 (Col6A2), collagen type VI, alpha 1 (Col6A1)); a ribophorin (e.g., ribophorin I (RPNI)); a low density lipoprotein receptor-related protein (e.g., LRP1); a cardiotrophin-like cytokine factor (e.g., Nnt1); calreticulin (Calr); a procollagen-lysine, 2-oxoglutarate 5-dioxygenase 1 (Plod1); and a nucleobindin (e.g., Nucb1).

Other exemplary 5′ and 3′ UTRs include, but are not limited to, those described in Karikó et al., Mol. Ther. 2008 16(11):1833-1840; Karikó et al., Mol. Ther. 2012 20(5):948-953; Karikó et al., Nucleic Acids Res. 2011 39(21):e142; Strong et al., Gene Therapy 1997 4:624-627; Hansson et al., J. Biol. Chem. 2015 290(9):5661-5672; Yu et al., Vaccine 2007 25(10):1701-1711; Cafri et al., Mol. Ther. 2015 23(8):1391-1400; Andries et al., Mol. Pharm. 2012 9(8):2136-2145; Crowley et al., Gene Ther. 2015 Jun. 30, doi:10.1038/gt.2015.68; Ramunas et al., FASEB J. 2015 29(5):1930-1939; Wang et al., Curr. Gene Ther. 2015 15(4):428-435; Holtkamp et al., Blood 2006 108(13):4009-4017; Kormann et al., Nat. Biotechnol. 2011 29(2):154-157; Poleganov et al., Hum. Gen. Ther. 2015 26(11):751-766; Warren et al., Cell Stem Cell 2010 7(5):618-630; Mandal and Rossi, Nat. Protoc. 2013 8(3):568-582; Holcik and Liebhaber, PNAS 1997 94(6):2410-2414; Ferizi et al., Lab Chip. 2015 15(17):3561-3571; Thess et al., Mol. Ther. 2015 23(9):1456-1464; Boros et al., PLoS One 2015 10(6):e0131141; Boros et al., J. Photochem. Photobiol. B. 2013 129:93-99; Andries et al., J. Control. Release 2015 217:337-344; Zinckgraf et al., Vaccine 2003 21(15):1640-9; Garneau et al., J. Virol. 2008 82(2):880-892; Holden and Harris, Virology 2004 329(1):119-133; Chiu et al., J. Virol. 2005 79(13):8303-8315; Wang et al., EMBO J. 1997 16(13):4107-4116; Al-Zoghaibi et al., Gene 2007 391(1-2):130-9; Vivinus et al., Eur. J. Biochem. 2001 268(7):1908-1917; Gan and Rhoads, J. Biol. Chem. 1996 271(2):623-626; Boado et al., J. Neurochem. 1996 67(4):1335-1343; Knirsch and Clerch, Biochem. Biophys. Res. Commun. 2000 272(1):164-168; Chung et al., Biochemistry 1998 37(46):16298-16306; Izquierdo and Cuevza, Biochem. J. 2000 346 Pt 3:849-855; Dwyer et al., J. Neurochem. 1996 66(2):449-458; Black et al., Mol. Cell. Biol. 1997 17(5):2756-2763; Izquierdo and Cuevza, Mol. Cell. Biol. 1997 17(9):5255-5268; U.S. Pat. Nos. 8,278,036; 8,748,089; 8,835,108; 9,012,219; US2010/0129877; US2011/0065103; US2011/0086904; US2012/0195936; US2014/020675; US2013/0195967; US2014/029490; US2014/0206753; WO2007/036366; WO2011/015347; WO2012/072096; WO2013/143555; WO2014/071963; WO2013/185067; WO2013/182623; WO2014/089486; WO2013/185069; WO2014/144196; WO2014/152659; 2014/152673; WO2014/152940; WO2014/152774; WO2014/153052; WO2014/152966, WO2014/152513; WO2015/101414; WO2015/101415; WO2015/062738; and WO2015/024667; the contents of each of which are incorporated herein by reference in their entirety.

In some embodiments, the 5′UTR is selected from the group consisting of a β-globin 5′UTR; a 5′UTR containing a strong Kozak translational initiation signal; a cytochrome b-245 α polypeptide (CYBA) 5′UTR; a hydroxysteroid (17-β) dehydrogenase (HSD17B4) 5′UTR; a Tobacco etch virus (TEV) 5′UTR; a Venezuelen equine encephalitis virus (TEEV) 5′UTR; a 5′ proximal open reading frame of rubella virus (RV) RNA encoding nonstructural proteins; a Dengue virus (DEN) 5′UTR; a heat shock protein 70 (Hsp70) 5′UTR; a eIF4G 5′UTR; a GLUT1 5′UTR; functional fragments thereof and any combination thereof.

In some embodiments, the 3′UTR is selected from the group consisting of a β-globin 3′UTR; a CYBA 3′UTR; an albumin 3′UTR; a growth hormone (GH) 3′UTR; a VEEV 3′UTR; a hepatitis B virus (HBV) 3′UTR; a-globin 3′UTR; a DEN 3′UTR; a PAV barley yellow dwarf virus (BYDV-PAV) 3′UTR; an elongation factor 1 α1 (EEF1A1) 3′UTR; a manganese superoxide dismutase (MnSOD) 3′UTR; a β subunit of mitochondrial H(+)-ATP synthase ((3-mRNA) 3′UTR; a GLUT1 3′UTR; a MEF2A 3′UTR; a β-F1-ATPase 3′UTR; functional fragments thereof and combinations thereof.

Other exemplary UTRs include, but are not limited to, one or more of the UTRs, including any combination of UTRs, disclosed in WO2014/164253, the contents of which are incorporated herein by reference in their entirety. Shown in Table 21 of U.S. Provisional Application No. 61/775,509 and in Table 22 of U.S. Provisional Application No. 61/829,372, the contents of each are incorporated herein by reference in their entireties, is a listing start and stop sites for 5′UTRs and 3′UTRs. In Table 21, each 5′UTR (5′-UTR-005 to 5′-UTR 68511) is identified by its start and stop site relative to its native or wild-type (homologous) transcript (ENST; the identifier used in the ENSEMBL database).

Wild-type UTRs derived from any gene or mRNA can be incorporated into the polynucleotides. In some embodiments, a UTR can be altered relative to a wild type or native UTR to produce a variant UTR, e.g., by changing the orientation or location of the UTR relative to the ORF; or by inclusion of additional nucleotides, deletion of nucleotides, swapping or transposition of nucleotides. In some embodiments, variants of 5′ or 3′ UTRs can be utilized, for example, mutants of wild type UTRs, or variants wherein one or more nucleotides are added to or removed from a terminus of the UTR.

Additionally, one or more synthetic UTRs can be used in combination with one or more non-synthetic UTRs. See, e.g., Mandal and Rossi, Nat. Protoc. 2013 8(3):568-82, the contents of which are incorporated herein by reference in their entirety, and sequences available at www.addgene.org/Derrick_Rossi/, last accessed Apr. 16, 2016. UTRs or portions thereof can be placed in the same orientation as in the transcript from which they were selected or can be altered in orientation or location. Hence, a 5′ and/or 3′ UTR can be inverted, shortened, lengthened, or combined with one or more other 5′ UTRs or 3′ UTRs.

In some embodiments, the polynucleotide comprises multiple UTRs, e.g., a double, a triple or a quadruple 5′UTR or 3′UTR. For example, a double UTR comprises two copies of the same UTR either in series or substantially in series. For example, a double beta-globin 3′UTR can be used (see US2010/0129877, the contents of which are incorporated herein by reference in its entirety).

In certain embodiments, the polynucleotides of the present disclosure comprise a 5′UTR and/or a 3′UTR selected from any of the UTRs disclosed herein. In some embodiments, the 5′UTR comprises:

5′UTR-001 (Upstream UTR)
(SEQ ID NO. 1)
(GGGAAATAAGAGAGAAAAGAAGAGTAAGAAGAAATATAAGAGCCACC);
5′UTR-002 (Upstream UTR)
(SEQ ID NO. 2)
(GGGAGATCAGAGAGAAAAGAAGAGTAAGAAGAAATATAAGAGCCACC);
5′UTR-003 (Upstream UTR)
(SEQ ID NO. 3)
(GGAATAAAAGTCTCAACACAACATATACAAAACAAACGAATCTCAAGCAAT
CAAGCATTCTACTTCTATTGCAGCAATTTAAATCATTTCTTTTAAAGCAAAAG
CAATTTTCTGAAAATTTTCACCATTTACGAACGATAGCAAC);
5′UTR-004 (Upstream UTR)
(SEQ ID NO. 4)
(GGGAGACAAGCUUGGCAUUCCGGUACUGUUGGUAAAGCCACC);
5′UTR-005 (Upstream UTR)
(SEQ ID NO. 5)
(GGGAGATCAGAGAGAAAAGAAGAGTAAGAAGAAATATAAGAGCCACC);
UTR 5′UTR-006 (Upstream UTR)
(SEQ ID NO. 6)
(GGAATAAAAGTCTCAACACAACATATACAAAACAAACGAATCTCAAGCAAT
CAAGCATTCTACTTCTATTGCAGCAATTTAAATCATTTCTTTTAAAGCAAAAG
CAATTTTCTGAAAATTTTCACCATTTACGAACGATAGCAAC);
5′UTR-007 (Upstream UTR)
(SEQ ID NO. 7)
(GGGAGACAAGCUUGGCAUUCCGGUACUGUUGGUAAAGCCACC);
5′UTR-008 (Upstream UTR)
(SEQ ID NO. 8)
(GGGAATTAACAGAGAAAAGAAGAGTAAGAAGAAATATAAGAGCCACC);
5′UTR-009 (Upstream UTR)
(SEQ ID NO. 9)
(GGGAAATTAGACAGAAAAGAAGAGTAAGAAGAAATATAAGAGCCACC);
UTR 5′UTR-010, Upstream
(SEQ ID NO. 10)
(GGGAAATAAGAGAGTAAAGAACAGTAAGAAGAAATATAAGAGCCACC);
5′UTR-011 (Upstream UTR)
(SEQ ID NO. 11)
(GGGAAAAAAGAGAGAAAAGAAGACTAAGAAGAAATATAAGAGCCACC);
5′UTR-012 (Upstream UTR)
(SEQ ID NO. 12)
(GGGAAATAAGAGAGAAAAGAAGAGTAAGAAGATATATAAGAGCCACC);
5′UTR-013 (Upstream UTR)
(SEQ ID NO. 13)
(GGGAAATAAGAGACAAAACAAGAGTAAGAAGAAATATAAGAGCCACC);
5′UTR-014 (Upstream UTR)
(SEQ ID NO. 14)
(GGGAAATTAGAGAGTAAAGAACAGTAAGTAGAATTAAAAGAGCCACC);
5′UTR-15 (Upstream UTR)
(SEQ ID NO. 15)
(GGGAAATAAGAGAGAATAGAAGAGTAAGAAGAAATATAAGAGCCACC);
5′UTR-016 (Upstream UTR)
(SEQ ID NO. 16)
(GGGAAATAAGAGAGAAAAGAAGAGTAAGAAGAAAATTAAGAGCCACC);
5′UTR-017 (Upstream UTR)
(SEQ ID NO. 17)
(GGGAAATAAGAGAGAAAAGAAGAGTAAGAAGAAATTTAAGAGCCACC);
5′UTR-018 (Upstream UTR)
(SEQ ID NO. 18)
(TCAAGCTTTTGGACCCTCGTACAGAAGCTAATACGACTCACTATAGGGAAA
TAAGAGAGAAAAGAAGAGTAAGAAGAAATATAAGAGCCACC);
142-3p 5′UTR-001 (Upstream UTR including miR142-3p)
(SEQ ID NO. 19)
(TGATAATAGTCCATAAAGTAGGAAACACTACAGCTGGAGCCTCGGTGGCCA
TGCTTCTTGCCCCTTGGGCCTCCCCCCAGCCCCTCCTCCCCTTCCTGCACCCG
TACCCCCGTGGTCTTTGAATAAAGTCTGAGTGGGCGGC);
142-3p 5′UTR-002 (Upstream UTR including miR142-3p)
(SEQ ID NO. 20)
(TGATAATAGGCTGGAGCCTCGGTGGCTCCATAAAGTAGGAAACACTACACA
TGCTTCTTGCCCCTTGGGCCTCCCCCCAGCCCCTCCTCCCCTTCCTGCACCCG
TACCCCCGTGGTCTTTGAATAAAGTCTGAGTGGGCGGC);
142-3p 5′UTR-003 (Upstream UTR including miR142-3p)
(SEQ ID NO. 21)
(TGATAATAGGCTGGAGCCTCGGTGGCCATGCTTCTTGCCCCTTCCATAAAGT
AGGAAACACTACATGGGCCTCCCCCCAGCCCCTCCTCCCCTTCCTGCACCCG
TACCCCCGTGGTCTTTGAATAAAGTCTGAGTGGGCGGC);
142-3p 5′UTR-004 (Upstream UTR including miR142-3p)
(SEQ ID NO. 22)
(TGATAATAGGCTGGAGCCTCGGTGGCCATGCTTCTTGCCCCTTGGGCCTCCC
CCCAGTCCATAAAGTAGGAAACACTACACCCCTCCTCCCCTTCCTGCACCCG
TACCCCCGTGGTCTTTGAATAAAGTCTGAGTGGGCGGC);
142-3p 5′UTR-005 (Upstream UTR including miR142-3p)
(SEQ ID NO. 23)
(TGATAATAGGCTGGAGCCTCGGTGGCCATGCTTCTTGCCCCTTGGGCCTCCC
CCCAGCCCCTCCTCCCCTTCTCCATAAAGTAGGAAACACTACACTGCACCCG
TACCCCCGTGGTCTTTGAATAAAGTCTGAGTGGGCGGC);
142-3p 5′UTR-006 (Upstream UTR including miR142-3p)
(SEQ ID NO. 24)
(TGATAATAGGCTGGAGCCTCGGTGGCCATGCTTCTTGCCCCTTGGGCCTCCC
CCCAGCCCCTCCTCCCCTTCCTGCACCCGTACCCCCTCCATAAAGTAGGAAA
CACTACAGTGGTCTTTGAATAAAGTCTGAGTGGGCGGC);
or
142-3p 5′UTR-007 (Upstream UTR including miR142-3p)
(SEQ ID NO. 25)
(TGATAATAGGCTGGAGCCTCGGTGGCCATGCTTCTTGCCCCTTGGGCCTCCC
CCCAGCCCCTCCTCCCCTTCCTGCACCCGTACCCCCGTGGTCTTTGAATAAAG
TTCCATAAAGTAGGAAACACTACACTGAGTGGGCGGC).
In some embodiments, the 3′UTR comprises:
3′UTR-001 (Creatine Kinase UTR)
(SEQ ID NO. 26)
(GCGCCTGCCCACCTGCCACCGACTGCTGGAACCCAGCCAGTGGGAGGGCCT
GGCCCACCAGAGTCCTGCTCCCTCACTCCTCGCCCCGCCCCCTGTCCCAGAGT
CCCACCTGGGGGCTCTCTCCACCCTTCTCAGAGTTCCAGTTTCAACCAGAGTT
CCAACCAATGGGCTCCATCCTCTGGATTCTGGCCAATGAAATATCTCCCTGG
CAGGGTCCTCTTCTTTTCCCAGAGCTCCACCCCAACCAGGAGCTCTAGTTAAT
GGAGAGCTCCCAGCACACTCGGAGCTTGTGCTTTGTCTCCACGCAAAGCGAT
AAATAAAAGCATTGGTGGCCTTTGGTCTTTGAATAAAGCCTGAGTAGGAAGT
CTAGA);
3′UTR-002 (Myoglobin UTR)
(SEQ ID NO. 27)
(GCCCCTGCCGCTCCCACCCCCACCCATCTGGGCCCCGGGTTCAAGAGAGAG
CGGGGTCTGATCTCGTGTAGCCATATAGAGTTTGCTTCTGAGTGTCTGCTTTG
TTTAGTAGAGGTGGGCAGGAGGAGCTGAGGGGCTGGGGCTGGGGTGTTGAA
GTTGGCTTTGCATGCCCAGCGATGCGCCTCCCTGTGGGATGTCATCACCCTG
GGAACCGGGAGTGGCCCTTGGCTCACTGTGTTCTGCATGGTTTGGATCTGAA
TTAATTGTCCTTTCTTCTAAATCCCAACCGAACTTCTTCCAACCTCCAAACTG
GCTGTAACCCCAAATCCAAGCCATTAACTACACCTGACAGTAGCAATTGTCT
GATTAATCACTGGCCCCTTGAAGACAGCAGAATGTCCCTTTGCAATGAGGAG
GAGATCTGGGCTGGGCGGGCCAGCTGGGGAAGCATTTGACTATCTGGAACTT
GTGTGTGCCTCCTCAGGTATGGCAGTGACTCACCTGGTTTTAATAAAACAAC
CTGCAACATCTCATGGTCTTTGAATAAAGCCTGAGTAGGAAGTCTAGA);
3′UTR-003 (α-actin UTR)
(SEQ ID NO. 28)
(ACACACTCCACCTCCAGCACGCGACTTCTCAGGACGACGAATCTTCTCAATG
GGGGGGCGGCTGAGCTCCAGCCACCCCGCAGTCACTTTCTTTGTAACAACTT
CCGTTGCTGCCATCGTAAACTGACACAGTGTTTATAACGTGTACATACATTA
ACTTATTACCTCATTTTGTTATTTTTCGAAACAAAGCCCTGTGGAAGAAAATG
GAAAACTTGAAGAAGCATTAAAGTCATTCTGTTAAGCTGCGTAAATGGTCTT
TGAATAAAGCCTGAGTAGGAAGTCTAGA);
3′UTR-004 (Albumin UTR)
(SEQ ID NO. 29)
(CATCACATTTAAAAGCATCTCAGCCTACCATGAGAATAAGAGAAAGAAAAT
GAAGATCAAAAGCTTATTCATCTGTTTTTCTTTTTCGTTGGTGTAAAGCCAAC
ACCCTGTCTAAAAAACATAAATTTCTTTAATCATTTTGCCTCTTTTCTCTGTGC
TTCAATTAATAAAAAATGGAAAGAATCTAATAGAGTGGTACAGCACTGTTAT
TTTTCAAAGATGTGTTGCTATCCTGAAAATTCTGTAGGTTCTGTGGAAGTTCC
AGTGTTCTCTCTTATTCCACTTCGGTAGAGGATTTCTAGTTTCTTGTGGGCTA
ATTAAATAAATCATTAATACTCTTCTAATGGTCTTTGAATAAAGCCTGAGTA
GGAAGTCTAGA);
3′UTR-005 (α-globin UTR)
(SEQ ID NO. 30)
(GCTGCCTTCTGCGGGGCTTGCCTTCTGGCCATGCCCTTCTTCTCTCCCTTGCA
CCTGTACCTCTTGGTCTTTGAATAAAGCCTGAGTAGGAAGGCGGCCGCTCGA
GCATGCATCTAGA);
3′UTR-006 (G-CSF UTR)
(SEQ ID NO. 31)
(GCCAAGCCCTCCCCATCCCATGTATTTATCTCTATTTAATATTTATGTCTATT
TAAGCCTCATATTTAAAGACAGGGAAGAGCAGAACGGAGCCCCAGGCCTCT
GTGTCCTTCCCTGCATTTCTGAGTTTCATTCTCCTGCCTGTAGCAGTGAGAAA
AAGCTCCTGTCCTCCCATCCCCTGGACTGGGAGGTAGATAGGTAAATACCAA
GTATTTATTACTATGACTGCTCCCCAGCCCTGGCTCTGCAATGGGCACTGGG
ATGAGCCGCTGTGAGCCCCTGGTCCTGAGGGTCCCCACCTGGGACCCTTGAG
AGTATCAGGTCTCCCACGTGGGAGACAAGAAATCCCTGTTTAATATTTAAAC
AGCAGTGTTCCCCATCTGGGTCCTTGCACCCCTCACTCTGGCCTCAGCCGACT
GCACAGCGGCCCCTGCATCCCCTTGGCTGTGAGGCCCCTGGACAAGCAGAGG
TGGCCAGAGCTGGGAGGCATGGCCCTGGGGTCCCACGAATTTGCTGGGGAA
TCTCGTTTTTCTTCTTAAGACTTTTGGGACATGGTTTGACTCCCGAACATCAC
CGACGCGTCTCCTGTTTTTCTGGGTGGCCTCGGGACACCTGCCCTGCCCCCAC
GAGGGTCAGGACTGTGACTCTTTTTAGGGCCAGGCAGGTGCCTGGACATTTG
CCTTGCTGGACGGGGACTGGGGATGTGGGAGGGAGCAGACAGGAGGAATCA
TGTCAGGCCTGTGTGTGAAAGGAAGCTCCACTGTCACCCTCCACCTCTTCAC
CCCCCACTCACCAGTGTCCCCTCCACTGTCACATTGTAACTGAACTTCAGGAT
AATAAAGTGTTTGCCTCCATGGTCTTTGAATAAAGCCTGAGTAGGAAGGCGG
CCGCTCGAGCATGCATCTAGA);
3′UTR-007 (Col1a2; collagen, type I, alpha 2 UTR)
(SEQ ID NO. 32)
(ACTCAATCTAAATTAAAAAAGAAAGAAATTTGAAAAAACTTTCTCTTTGCC
ATTTCTTCTTCTTCTTTTTTAACTGAAAGCTGAATCCTTCCATTTCTTCTGCAC
ATCTACTTGCTTAAATTGTGGGCAAAAGAGAAAAAGAAGGATTGATCAGAG
CATTGTGCAATACAGTTTCATTAACTCCTTCCCCCGCTCCCCCAAAAATTTGA
ATTTTTTTTTCAACACTCTTACACCTGTTATGGAAAATGTCAACCTTTGTAAG
AAAACCAAAATAAAAATTGAAAAATAAAAACCATAAACATTTGCACCACTT
GTGGCTTTTGAATATCTTCCACAGAGGGAAGTTTAAAACCCAAACTTCCAAA
GGTTTAAACTACCTCAAAACACTTTCCCATGAGTGTGATCCACATTGTTAGGT
GCTGACCTAGACAGAGATGAACTGAGGTCCTTGTTTTGTTTTGTTCATAATAC
AAAGGTGCTAATTAATAGTATTTCAGATACTTGAAGAATGTTGATGGTGCTA
GAAGAATTTGAGAAGAAATACTCCTGTATTGAGTTGTATCGTGTGGTGTATT
TTTTAAAAAATTTGATTTAGCATTCATATTTTCCATCTTATTCCCAATTAAAA
GTATGCAGATTATTTGCCCAAATCTTCTTCAGATTCAGCATTTGTTCTTTGCC
AGTCTCATTTTCATCTTCTTCCATGGTTCCACAGAAGCTTTGTTTCTTGGGCA
AGCAGAAAAATTAAATTGTACCTATTTTGTATATGTGAGATGTTTAAATAAA
TTGTGAAAAAAATGAAATAAAGCATGTTTGGTTTTCCAAAAGAACATAT);
3′UTR-008 (Col6a2; collagen, type VI, alpha 2 UTR)
(SEQ ID NO. 33)
(CGCCGCCGCCCGGGCCCCGCAGTCGAGGGTCGTGAGCCCACCCCGTCCATG
GTGCTAAGCGGGCCCGGGTCCCACACGGCCAGCACCGCTGCTCACTCGGACG
ACGCCCTGGGCCTGCACCTCTCCAGCTCCTCCCACGGGGTCCCCGTAGCCCC
GGCCCCCGCCCAGCCCCAGGTCTCCCCAGGCCCTCCGCAGGCTGCCCGGCCT
CCCTCCCCCTGCAGCCATCCCAAGGCTCCTGACCTACCTGGCCCCTGAGCTCT
GGAGCAAGCCCTGACCCAATAAAGGCTTTGAACCCAT);
3′UTR-009 (RPN1; ribophorin I UTR)
(SEQ ID NO. 34)
(GGGGCTAGAGCCCTCTCCGCACAGCGTGGAGACGGGGCAAGGAGGGGGGT
TATTAGGATTGGTGGTTTTGTTTTGCTTTGTTTAAAGCCGTGGGAAAATGGCA
CAACTTTACCTCTGTGGGAGATGCAACACTGAGAGCCAAGGGGTGGGAGTT
GGGATAATTTTTATATAAAAGAAGTTTTTCCACTTTGAATTGCTAAAAGTGG
CATTTTTCCTATGTGCAGTCACTCCTCTCATTTCTAAAATAGGGACGTGGCCA
GGCACGGTGGCTCATGCCTGTAATCCCAGCACTTTGGGAGGCCGAGGCAGGC
GGCTCACGAGGTCAGGAGATCGAGACTATCCTGGCTAACACGGTAAAACCC
TGTCTCTACTAAAAGTACAAAAAATTAGCTGGGCGTGGTGGTGGGCACCTGT
AGTCCCAGCTACTCGGGAGGCTGAGGCAGGAGAAAGGCATGAATCCAAGAG
GCAGAGCTTGCAGTGAGCTGAGATCACGCCATTGCACTCCAGCCTGGGCAAC
AGTGTTAAGACTCTGTCTCAAATATAAATAAATAAATAAATAAATAAATAAA
TAAATAAAAATAAAGCGAGATGTTGCCCTCAAA);
3′UTR-010 (LRP1; low density lipoprotein receptor-related protein 1 UTR)
(SEQ ID NO. 35)
(GGCCCTGCCCCGTCGGACTGCCCCCAGAAAGCCTCCTGCCCCCTGCCAGTGA
AGTCCTTCAGTGAGCCCCTCCCCAGCCAGCCCTTCCCTGGCCCCGCCGGATG
TATAAATGTAAAAATGAAGGAATTACATTTTATATGTGAGCGAGCAAGCCGG
CAAGCGAGCACAGTATTATTTCTCCATCCCCTCCCTGCCTGCTCCTTGGCACC
CCCATGCTGCCTTCAGGGAGACAGGCAGGGAGGGCTTGGGGCTGCACCTCCT
ACCCTCCCACCAGAACGCACCCCACTGGGAGAGCTGGTGGTGCAGCCTTCCC
CTCCCTGTATAAGACACTTTGCCAAGGCTCTCCCCTCTCGCCCCATCCCTGCT
TGCCCGCTCCCACAGCTTCCTGAGGGCTAATTCTGGGAAGGGAGAGTTCTTT
GCTGCCCCTGTCTGGAAGACGTGGCTCTGGGTGAGGTAGGCGGGAAAGGAT
GGAGTGTTTTAGTTCTTGGGGGAGGCCACCCCAAACCCCAGCCCCAACTCCA
GGGGCACCTATGAGATGGCCATGCTCAACCCCCCTCCCAGACAGGCCCTCCC
TGTCTCCAGGGCCCCCACCGAGGTTCCCAGGGCTGGAGACTTCCTCTGGTAA
ACATTCCTCCAGCCTCCCCTCCCCTGGGGACGCCAAGGAGGTGGGCCACACC
CAGGAAGGGAAAGCGGGCAGCCCCGTTTTGGGGACGTGAACGTTTTAATAA
TTTTTGCTGAATTCCTTTACAACTAAATAACACAGATATTGTTATAAATAAAA
TTGT);
3′UTR-011 (Nnt1; cardiotrophin-like cytokine factor 1 UTR)
(SEQ ID NO. 36)
(ATATTAAGGATCAAGCTGTTAGCTAATAATGCCACCTCTGCAGTTTTGGGAA
CAGGCAAATAAAGTATCAGTATACATGGTGATGTACATCTGTAGCAAAGCTC
TTGGAGAAAATGAAGACTGAAGAAAGCAAAGCAAAAACTGTATAGAGAGAT
TTTTCAAAAGCAGTAATCCCTCAATTTTAAAAAAGGATTGAAAATTCTAAAT
GTCTTTCTGTGCATATTTTTTGTGTTAGGAATCAAAAGTATTTTATAAAAGGA
GAAAGAACAGCCTCATTTTAGATGTAGTCCTGTTGGATTTTTTATGCCTCCTC
AGTAACCAGAAATGTTTTAAAAAACTAAGTGTTTAGGATTTCAAGACAACAT
TATACATGGCTCTGAAATATCTGACACAATGTAAACATTGCAGGCACCTGCA
TTTTATGTTTTTTTTTTCAACAAATGTGACTAATTTGAAACTTTTATGAACTTC
TGAGCTGTCCCCTTGCAATTCAACCGCAGTTTGAATTAATCATATCAAATCA
GTTTTAATTTTTTAAATTGTACTTCAGAGTCTATATTTCAAGGGCACATTTTCT
CACTACTATTTTAATACATTAAAGGACTAAATAATCTTTCAGAGATGCTGGA
AACAAATCATTTGCTTTATATGTTTCATTAGAATACCAATGAAACATACAAC
TTGAAAATTAGTAATAGTATTTTTGAAGATCCCATTTCTAATTGGAGATCTCT
TTAATTTCGATCAACTTATAATGTGTAGTACTATATTAAGTGCACTTGAGTGG
AATTCAACATTTGACTAATAAAATGAGTTCATCATGTTGGCAAGTGATGTGG
CAATTATCTCTGGTGACAAAAGAGTAAAATCAAATATTTCTGCCTGTTACAA
ATATCAAGGAAGACCTGCTACTATGAAATAGATGACATTAATCTGTCTTCAC
TGTTTATAATACGGATGGATTTTTTTTCAAATCAGTGTGTGTTTTGAGGTCTT
ATGTAATTGATGACATTTGAGAGAAATGGTGGCTTTTTTTAGCTACCTCTTTG
TTCATTTAAGCACCAGTAAAGATCATGTCTTTTTATAGAAGTGTAGATTTTCT
TTGTGACTTTGCTATCGTGCCTAAAGCTCTAAATATAGGTGAATGTGTGATG
AATACTCAGATTATTTGTCTCTCTATATAATTAGTTTGGTACTAAGTTTCTCA
AAAAATTATTAACACATGAAAGACAATCTCTAAACCAGAAAAAGAAGTAGT
ACAAATTTTGTTACTGTAATGCTCGCGTTTAGTGAGTTTAAAACACACAGTAT
CTTTTGGTTTTATAATCAGTTTCTATTTTGCTGTGCCTGAGATTAAGATCTGTG
TATGTGTGTGTGTGTGTGTGTGCGTTTGTGTGTTAAAGCAGAAAAGACTTTTT
TAAAAGTTTTAAGTGATAAATGCAATTTGTTAATTGATCTTAGATCACTAGTA
AACTCAGGGCTGAATTATACCATGTATATTCTATTAGAAGAAAGTAAACACC
ATCTTTATTCCTGCCCTTTTTCTTCTCTCAAAGTAGTTGTAGTTATATCTAGAA
AGAAGCAATTTTGATTTCTTGAAAAGGTAGTTCCTGCACTCAGTTTAAACTA
AAAATAATCATACTTGGATTTTATTTATTTTTGTCATAGTAAAAATTTTAATT
TATATATATTTTTATTTAGTATTATCTTATTCTTTGCTATTTGCCAATCCTTTG
TCATCAATTGTGTTAAATGAATTGAAAATTCATGCCCTGTTCATTTTATTTTA
CTTTATTGGTTAGGATATTTAAAGGATTTTTGTATATATAATTTCTTAAATTA
ATATTCCAAAAGGTTAGTGGACTTAGATTATAAATTATGGCAAAAATCTAAA
AACAACAAAAATGATTTTTATACATTCTATTTCATTATTCCTCTTTTTCCAAT
AAGTCATACAATTGGTAGATATGACTTATTTTATTTTTGTATTATTCACTATA
TCTTTATGATATTTAAGTATAAATAATTAAAAAAATTTATTGTACCTTATAGT
CTGTCACCAAAAAAAAAAAATTATCTGTAGGTAGTGAAATGCTAATGTTGAT
TTGTCTTTAAGGGCTTGTTAACTATCCTTTATTTTCTCATTTGTCTTAAATTAG
GAGTTTGTGTTTAAATTACTCATCTAAGCAAAAAATGTATATAAATCCCATT
ACTGGGTATATACCCAAAGGATTATAAATCATGCTGCTATAAAGACACATGC
ACACGTATGTTTATTGCAGCACTATTCACAATAGCAAAGACTTGGAACCAAC
CCAAATGTCCATCAATGATAGACTTGATTAAGAAAATGTGCACATATACACC
ATGGAATACTATGCAGCCATAAAAAAGGATGAGTTCATGTCCTTTGTAGGGA
CATGGATAAAGCTGGAAACCATCATTCTGAGCAAACTATTGCAAGGACAGA
AAACCAAACACTGCATGTTCTCACTCATAGGTGGGAATTGAACAATGAGAAC
ACTTGGACACAAGGTGGGGAACACCACACACCAGGGCCTGTCATGGGGTGG
GGGGAGTGGGGAGGGATAGCATTAGGAGATATACCTAATGTAAATGATGAG
TTAATGGGTGCAGCACACCAACATGGCACATGTATACATATGTAGCAAACCT
GCACGTTGTGCACATGTACCCTAGAACTTAAAGTATAATTAAAAAAAAAAA
GAAAACAGAAGCTATTTATAAAGAAGTTATTTGCTGAAATAAATGTGATCTT
TCCCATTAAAAAAATAAAGAAATTTTGGGGTAAAAAAACACAATATATTGTA
TTCTTGAAAAATTCTAAGAGAGTGGATGTGAAGTGTTCTCACCACAAAAGTG
ATAACTAATTGAGGTAATGCACATATTAATTAGAAAGATTTTGTCATTCCAC
AATGTATATATACTTAAAAATATGTTATACACAATAAATACATACATTAAAA
AATAAGTAAATGTA);
3′UTR-012 (Col6a1; collagen, type VI, alpha 1 UTR)
(SEQ ID NO. 37)
(CCCACCCTGCACGCCGGCACCAAACCCTGTCCTCCCACCCCTCCCCACTCAT
CACTAAACAGAGTAAAATGTGATGCGAATTTTCCCGACCAACCTGATTCGCT
AGATTTTTTTTAAGGAAAAGCTTGGAAAGCCAGGACACAACGCTGCTGCCTG
CTTTGTGCAGGGTCCTCCGGGGCTCAGCCCTGAGTTGGCATCACCTGCGCAG
GGCCCTCTGGGGCTCAGCCCTGAGCTAGTGTCACCTGCACAGGGCCCTCTGA
GGCTCAGCCCTGAGCTGGCGTCACCTGTGCAGGGCCCTCTGGGGCTCAGCCC
TGAGCTGGCCTCACCTGGGTTCCCCACCCCGGGCTCTCCTGCCCTGCCCTCCT
GCCCGCCCTCCCTCCTGCCTGCGCAGCTCCTTCCCTAGGCACCTCTGTGCTGC
ATCCCACCAGCCTGAGCAAGACGCCCTCTCGGGGCCTGTGCCGCACTAGCCT
CCCTCTCCTCTGTCCCCATAGCTGGTTTTTCCCACCAATCCTCACCTAACAGT
TACTTTACAATTAAACTCAAAGCAAGCTCTTCTCCTCAGCTTGGGGCAGCCA
TTGGCCTCTGTCTCGTTTTGGGAAACCAAGGTCAGGAGGCCGTTGCAGACAT
AAATCTCGGCGACTCGGCCCCGTCTCCTGAGGGTCCTGCTGGTGACCGGCCT
GGACCTTGGCCCTACAGCCCTGGAGGCCGCTGCTGACCAGCACTGACCCCGA
CCTCAGAGAGTACTCGCAGGGGCGCTGGCTGCACTCAAGACCCTCGAGATTA
ACGGTGCTAACCCCGTCTGCTCCTCCCTCCCGCAGAGACTGGGGCCTGGACT
GGACATGAGAGCCCCTTGGTGCCACAGAGGGCTGTGTCTTACTAGAAACAAC
GCAAACCTCTCCTTCCTCAGAATAGTGATGTGTTCGACGTTTTATCAAAGGCC
CCCTTTCTATGTTCATGTTAGTTTTGCTCCTTCTGTGTTTTTTTCTGAACCATA
TCCATGTTGCTGACTTTTCCAAATAAAGGTTTTCACTCCTCTC);
3′UTR-013 (Calr; calreticulin UTR)
(SEQ ID NO. 38)
(AGAGGCCTGCCTCCAGGGCTGGACTGAGGCCTGAGCGCTCCTGCCGCAGAG
CTGGCCGCGCCAAATAATGTCTCTGTGAGACTCGAGAACTTTCATTTTTTTCC
AGGCTGGTTCGGATTTGGGGTGGATTTTGGTTTTGTTCCCCTCCTCCACTCTC
CCCCACCCCCTCCCCGCCCTTTTTTTTTTTTTTTTTTAAACTGGTATTTTATCTT
TGATTCTCCTTCAGCCCTCACCCCTGGTTCTCATCTTTCTTGATCAACATCTTT
TCTTGCCTCTGTCCCCTTCTCTCATCTCTTAGCTCCCCTCCAACCTGGGGGGC
AGTGGTGTGGAGAAGCCACAGGCCTGAGATTTCATCTGCTCTCCTTCCTGGA
GCCCAGAGGAGGGCAGCAGAAGGGGGTGGTGTCTCCAACCCCCCAGCACTG
AGGAAGAACGGGGCTCTTCTCATTTCACCCCTCCCTTTCTCCCCTGCCCCCAG
GACTGGGCCACTTCTGGGTGGGGCAGTGGGTCCCAGATTGGCTCACACTGAG
AATGTAAGAACTACAAACAAAATTTCTATTAAATTAAATTTTGTGTCTCC);
3′UTR-014 (Col1a1; collagen, type I, alpha 1 UTR)
(SEQ ID NO. 39)
(CTCCCTCCATCCCAACCTGGCTCCCTCCCACCCAACCAACTTTCCCCCCAAC
CCGGAAACAGACAAGCAACCCAAACTGAACCCCCTCAAAAGCCAAAAAATG
GGAGACAATTTCACATGGACTTTGGAAAATATTTTTTTCCTTTGCATTCATCT
CTCAAACTTAGTTTTTATCTTTGACCAACCGAACATGACCAAAAACCAAAAG
TGCATTCAACCTTACCAAAAAAAAAAAAAAAAAAAGAATAAATAAATAACT
TTTTAAAAAAGGAAGCTTGGTCCACTTGCTTGAAGACCCATGCGGGGGTAAG
TCCCTTTCTGCCCGTTGGGCTTATGAAACCCCAATGCTGCCCTTTCTGCTCCT
TTCTCCACACCCCCCTTGGGGCCTCCCCTCCACTCCTTCCCAAATCTGTCTCC
CCAGAAGACACAGGAAACAATGTATTGTCTGCCCAGCAATCAAAGGCAATG
CTCAAACACCCAAGTGGCCCCCACCCTCAGCCCGCTCCTGCCCGCCCAGCAC
CCCCAGGCCCTGGGGGACCTGGGGTTCTCAGACTGCCAAAGAAGCCTTGCCA
TCTGGCGCTCCCATGGCTCTTGCAACATCTCCCCTTCGTTTTTGAGGGGGTCA
TGCCGGGGGAGCCACCAGCCCCTCACTGGGTTCGGAGGAGAGTCAGGAAGG
GCCACGACAAAGCAGAAACATCGGATTTGGGGAACGCGTGTCAATCCCTTGT
GCCGCAGGGCTGGGCGGGAGAGACTGTTCTGTTCCTTGTGTAACTGTGTTGC
TGAAAGACTACCTCGTTCTTGTCTTGATGTGTCACCGGGGCAACTGCCTGGG
GGCGGGGATGGGGGCAGGGTGGAAGCGGCTCCCCATTTTATACCAAAGGTG
CTACATCTATGTGATGGGTGGGGTGGGGAGGGAATCACTGGTGCTATAGAA
ATTGAGATGCCCCCCCAGGCCAGCAAATGTTCCTTTTTGTTCAAAGTCTATTT
TTATTCCTTGATATTTTTCTTTTTTTTTTTTTTTTTTTGTGGATGGGGACTTGTG
AATTTTTCTAAAGGTGCTATTTAACATGGGAGGAGAGCGTGTGCGGCTCCAG
CCCAGCCCGCTGCTCACTTTCCACCCTCTCTCCACCTGCCTCTGGCTTCTCAG
GCCTCTGCTCTCCGACCTCTCTCCTCTGAAACCCTCCTCCACAGCTGCAGCCC
ATCCTCCCGGCTCCCTCCTAGTCTGTCCTGCGTCCTCTGTCCCCGGGTTTCAG
AGACAACTTCCCAAAGCACAAAGCAGTTTTTCCCCCTAGGGGTGGGAGGAA
GCAAAAGACTCTGTACCTATTTTGTATGTGTATAATAATTTGAGATGTTTTTA
ATTATTTTGATTGCTGGAATAAAGCATGTGGAAATGACCCAAACATAATCCG
CAGTGGCCTCCTAATTTCCTTCTTTGGAGTTGGGGGAGGGGTAGACATGGGG
AAGGGGCTTTGGGGTGATGGGCTTGCCTTCCATTCCTGCCCTTTCCCTCCCCA
CTATTCTCTTCTAGATCCCTCCATAACCCCACTCCCCTTTCTCTCACCCTTCTT
ATACCGCAAACCTTTCTACTTCCTCTTTCATTTTCTATTCTTGCAATTTCCTTG
CACCTTTTCCAAATCCTCTTCTCCCCTGCAATACCATACAGGCAATCCACGTG
CACAACACACACACACACTCTTCACATCTGGGGTTGTCCAAACCTCATACCC
ACTCCCCTTCAAGCCCATCCACTCTCCACCCCCTGGATGCCCTGCACTTGGTG
GCGGTGGGATGCTCATGGATACTGGGAGGGTGAGGGGAGTGGAACCCGTGA
GGAGGACCTGGGGGCCTCTCCTTGAACTGACATGAAGGGTCATCTGGCCTCT
GCTCCCTTCTCACCCACGCTGACCTCCTGCCGAAGGAGCAACGCAACAGGAG
AGGGGTCTGCTGAGCCTGGCGAGGGTCTGGGAGGGACCAGGAGGAAGGCGT
GCTCCCTGCTCGCTGTCCTGGCCCTGGGGGAGTGAGGGAGACAGACACCTGG
GAGAGCTGTGGGGAAGGCACTCGCACCGTGCTCTTGGGAAGGAAGGAGACC
TGGCCCTGCTCACCACGGACTGGGTGCCTCGACCTCCTGAATCCCCAGAACA
CAACCCCCCTGGGCTGGGGTGGTCTGGGGAACCATCGTGCCCCCGCCTCCCG
CCTACTCCTTTTTAAGCTT);
3′UTR-015 (Plod1; procollagen-lysine, 2-oxoglutarate 5-dioxygenase 1 UTR)
(SEQ ID NO. 40)
(TTGGCCAGGCCTGACCCTCTTGGACCTTTCTTCTTTGCCGACAACCACTGCC
CAGCAGCCTCTGGGACCTCGGGGTCCCAGGGAACCCAGTCCAGCCTCCTGGC
TGTTGACTTCCCATTGCTCTTGGAGCCACCAATCAAAGAGATTCAAAGAGAT
TCCTGCAGGCCAGAGGCGGAACACACCTTTATGGCTGGGGCTCTCCGTGGTG
TTCTGGACCCAGCCCCTGGAGACACCATTCACTTTTACTGCTTTGTAGTGACT
CGTGCTCTCCAACCTGTCTTCCTGAAAAACCAAGGCCCCCTTCCCCCACCTCT
TCCATGGGGTGAGACTTGAGCAGAACAGGGGCTTCCCCAAGTTGCCCAGAA
AGACTGTCTGGGTGAGAAGCCATGGCCAGAGCTTCTCCCAGGCACAGGTGTT
GCACCAGGGACTTCTGCTTCAAGTTTTGGGGTAAAGACACCTGGATCAGACT
CCAAGGGCTGCCCTGAGTCTGGGACTTCTGCCTCCATGGCTGGTCATGAGAG
CAAACCGTAGTCCCCTGGAGACAGCGACTCCAGAGAACCTCTTGGGAGACA
GAAGAGGCATCTGTGCACAGCTCGATCTTCTACTTGCCTGTGGGGAGGGGAG
TGACAGGTCCACACACCACACTGGGTCACCCTGTCCTGGATGCCTCTGAAGA
GAGGGACAGACCGTCAGAAACTGGAGAGTTTCTATTAAAGGTCATTTAAACC
A);
3′UTR-016 (Nucb1; nucleobindin 1 UTR)
(SEQ ID NO. 41)
(TCCTCCGGGACCCCAGCCCTCAGGATTCCTGATGCTCCAAGGCGACTGATGG
GCGCTGGATGAAGTGGCACAGTCAGCTTCCCTGGGGGCTGGTGTCATGTTGG
GCTCCTGGGGCGGGGGCACGGCCTGGCATTTCACGCATTGCTGCCACCCCAG
GTCCACCTGTCTCCACTTTCACAGCCTCCAAGTCTGTGGCTCTTCCCTTCTGT
CCTCCGAGGGGCTTGCCTTCTCTCGTGTCCAGTGAGGTGCTCAGTGATCGGCT
TAACTTAGAGAAGCCCGCCCCCTCCCCTTCTCCGTCTGTCCCAAGAGGGTCT
GCTCTGAGCCTGCGTTCCTAGGTGGCTCGGCCTCAGCTGCCTGGGTTGTGGC
CGCCCTAGCATCCTGTATGCCCACAGCTACTGGAATCCCCGCTGCTGCTCCG
GGCCAAGCTTCTGGTTGATTAATGAGGGCATGGGGTGGTCCCTCAAGACCTT
CCCCTACCTTTTGTGGAACCAGTGATGCCTCAAAGACAGTGTCCCCTCCACA
GCTGGGTGCCAGGGGCAGGGGATCCTCAGTATAGCCGGTGAACCCTGATAC
CAGGAGCCTGGGCCTCCCTGAACCCCTGGCTTCCAGCCATCTCATCGCCAGC
CTCCTCCTGGACCTCTTGGCCCCCAGCCCCTTCCCCACACAGCCCCAGAAGG
GTCCCAGAGCTGACCCCACTCCAGGACCTAGGCCCAGCCCCTCAGCCTCATC
TGGAGCCCCTGAAGACCAGTCCCACCCACCTTTCTGGCCTCATCTGACACTG
CTCCGCATCCTGCTGTGTGTCCTGTTCCATGTTCCGGTTCCATCCAAATACAC
TTTCTGGAACAAA);
3′UTR-017 (α-globin)
(SEQ ID NO. 42)
(GCTGGAGCCTCGGTGGCCATGCTTCTTGCCCCTTGGGCCTCCCCCCAGCCCC
TCCTCCCCTTCCTGCACCCGTACCCCCGTGGTCTTTGAATAAAGTCTGAGTGG
GCGGC);
or
3′UTR-018
(SEQ ID NO. 43)
(TGATAATAGGCTGGAGCCTCGGTGGCCATGCTTCTTGCCCCTTGGGCCTCCC
CCCAGCCCCTCCTCCCCTTCCTGCACCCGTACCCCCGTGGTCTTTGAATAAAG
TCTGAGTGGGCGGC).

In certain embodiments, the 5′UTR and/or 3′UTR sequence of the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 5′UTR sequences comprising any of SEQ ID NOs: 1-25 and/or 3′UTR sequences comprises any of SEQ ID NOs: 26-43, and any combination thereof.

The polynucleotides can comprise combinations of features. For example, the ORF can be flanked by a 5′UTR that comprises a strong Kozak translational initiation signal and/or a 3′UTR comprising an oligo(dT) sequence for templated addition of a poly-A tail. A 5′UTR can comprise a first polynucleotide fragment and a second polynucleotide fragment from the same and/or different UTRs (see, e.g., US2010/0293625, herein incorporated by reference in its entirety).

It is also within the scope of the present invention to have patterned UTRs. As used herein “patterned UTRs” include a repeating or alternating pattern, such as ABABAB or AABBAABBAABB or ABCABCABC or variants thereof repeated once, twice, or more than 3 times. In these patterns, each letter, A, B, or C represent a different UTR nucleic acid sequence.

Other non-UTR sequences can be used as regions or subregions within the polynucleotides. For example, introns or portions of intron sequences can be incorporated into the polynucleotides. Incorporation of intronic sequences can increase protein production as well as polynucleotide expression levels. In some embodiments, the polynucleotide comprises an internal ribosome entry site (IRES) instead of or in addition to a UTR (see, e.g., Yakubov et al., Biochem. Biophys. Res. Commun. 2010 394(1):189-193, the contents of which are incorporated herein by reference in their entirety). In some embodiments, the polynucleotide comprises 5′ and/or 3′ sequence associated with the 5′ and/or 3′ ends of rubella virus (RV) genomic RNA, respectively, or deletion derivatives thereof, including the 5′ proximal open reading frame of RV RNA encoding nonstructural proteins (e.g., see Pogue et al., J. Virol. 67(12):7106-7117, the contents of which are incorporated herein by reference in their entirety). Viral capsid sequences can also be used as a translational enhancer, e.g., the 5′ portion of a capsid sequence, (e.g., semliki forest virus and sindbis virus capsid RNAs as described in Sjöberg et al., Biotechnology (NY) 1994 12(11):1127-1131, and Frolov and Schlesinger J. Virol. 1996 70(2):1182-1190, the contents of each of which are incorporated herein by reference in their entirety). In some embodiments, the polynucleotide comprises an IRES instead of a 5′UTR sequence. In some embodiments, the polynucleotide comprises an ORF and a viral capsid sequence. In some embodiments, the polynucleotide comprises a synthetic 5′UTR in combination with a non-synthetic 3′UTR.

In some embodiments, the UTR can also include at least one translation enhancer polynucleotide, translation enhancer element, or translational enhancer elements (collectively, “TEE,” which refers to nucleic acid sequences that increase the amount of polypeptide or protein produced from a polynucleotide. As a non-limiting example, the TEE can include those described in US2009/0226470, incorporated herein by reference in its entirety, and others known in the art. As a non-limiting example, the TEE can be located between the transcription promoter and the start codon. In some embodiments, the 5′UTR comprises a TEE.

In one aspect, a TEE is a conserved element in a UTR that can promote translational activity of a nucleic acid such as, but not limited to, cap-dependent or cap-independent translation. The conservation of these sequences has been shown across 14 species including humans. See, e.g., Panek et al., “An evolutionary conserved pattern of 18S rRNA sequence complementarity to mRNA 5′UTRs and its implications for eukaryotic gene translation regulation,” Nucleic Acids Research 2013, doi:10.1093/nar/gkt548, incorporated herein by reference in its entirety.

In one non-limiting example, the TEE comprises the TEE sequence in the 5′-leader of the Gtx homeodomain protein. See Chappell et al., PNAS 2004 101:9590-9594, incorporated herein by reference in its entirety.

In another non-limiting example, the TEE comprises a TEE having one or more of the sequences of SEQ ID NOs: 1-35 in US2009/0226470, US2013/0177581, and WO2009/075886; SEQ ID NOs: 1-5 and 7-645 in WO2012/009644; and SEQ ID NO: 1 WO1999/024595, U.S. Pat. Nos. 6,310,197, and 6,849,405; the contents of each of which are incorporated herein by reference in their entireties.

In some embodiments, the TEE is an internal ribosome entry site (IRES), HCV-IRES, or an IRES element such as, but not limited to, those described in: U.S. Pat. No. 7,468,275, US2007/0048776, US2011/0124100, WO2007/025008, and WO2001/055369; the contents of each of which re incorporated herein by reference in their entirety. The IRES elements can include, but are not limited to, the Gtx sequences (e.g., Gtx9-nt, Gtx8-nt, Gtx7-nt) as described by Chappell et al., PNAS 2004 101:9590-9594, Zhou et al., PNAS 2005 102:6273-6278, US2007/0048776, US2011/0124100, and WO2007/025008; the contents of each of which are incorporated herein by reference in their entireties.

“Translational enhancer polynucleotide” or “translation enhancer polynucleotide sequence” refer to a polynucleotide that includes one or more of the TEE provided herein and/or known in the art (see. e.g., U.S. Pat. Nos. 6,310,197, 6,849,405, 7,456,273, 7,183,395, US2009/0226470, US2007/0048776, US2011/0124100, US2009/0093049, US2013/0177581, WO2009/075886, WO2007/025008, WO2012/009644, WO2001/055371, WO1999/024595, EP2610341A1, and EP2610340A1; the contents of each of which are incorporated herein by reference in their entirety), or their variants, homologs, or functional derivatives. In some embodiments, the polynucleotide comprises one or multiple copies of a TEE. The TEE in a translational enhancer polynucleotide can be organized in one or more sequence segments. A sequence segment can harbor one or more of the TEEs provided herein, with each TEE being present in one or more copies. When multiple sequence segments are present in a translational enhancer polynucleotide, they can be homogenous or heterogeneous. Thus, the multiple sequence segments in a translational enhancer polynucleotide can harbor identical or different types of the TEE provided herein, identical or different number of copies of each of the TEE, and/or identical or different organization of the TEE within each sequence segment. In one embodiment, the polynucleotide comprises a translational enhancer polynucleotide sequence.

In some embodiments, a 5′UTR and/or 3′UTR of a polynucleotide comprises at least one TEE or portion thereof that is disclosed in: WO1999/024595, WO2012/009644, WO2009/075886, WO2007/025008, WO1999/024595, WO2001/055371, EP2610341A1, EP2610340A1, U.S. Pat. Nos. 6,310,197, 6,849,405, 7,456,273, 7,183,395, US2009/0226470, US2011/0124100, US2007/0048776, US2009/0093049, or US2013/0177581, the contents of each are incorporated herein by reference in their entirety.

In some embodiments, a 5′UTR and/or 3′UTR of a polynucleotide comprises a TEE that is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 96%, at least 97%, at least 98%, at least 99%, or 100% identical to a TEE disclosed in: US2009/0226470, US2007/0048776, US2013/0177581, US2011/0124100, WO1999/024595, WO2012/009644, WO2009/075886, WO2007/025008, EP2610341A1, EP2610340A1, U.S. Pat. Nos. 6,310,197, 6,849,405, 7,456,273, 7,183,395, Chappell et al., PNAS 2004 101:9590-9594, Zhou et al., PNAS 2005 102:6273-6278, and Supplemental Table 1 and in Supplemental Table 2 of Wellensiek et al., “Genome-wide profiling of human cap-independent translation-enhancing elements,” Nature Methods 2013, DOI:10.1038/NMETH.2522; the contents of each of which are incorporated herein by reference in their entirety.

In some embodiments, a 5′UTR and/or 3′UTR of a polynucleotide comprises a TEE which is selected from a 5-30 nucleotide fragment, a 5-25 nucleotide fragment, a 5-20 nucleotide fragment, a 5-15 nucleotide fragment, or a 5-10 nucleotide fragment (including a fragment of 5, 6, 7, 8, 9, 10, 11, 12, 13, 14, 15, 16, 17, 18, 19, 20, 21, 22, 23, 24, 25, 26, 27, 28, 29, or 30 nucleotides) of a TEE sequence disclosed in: US2009/0226470, US2007/0048776, US2013/0177581, US2011/0124100, WO1999/024595, WO2012/009644, WO2009/075886, WO2007/025008, EP2610341A1, EP2610340A1, U.S. Pat. Nos. 6,310,197, 6,849,405, 7,456,273, 7,183,395, Chappell et al., PNAS 2004 101:9590-9594, Zhou et al., PNAS 2005 102:6273-6278, and Supplemental Table 1 and in Supplemental Table 2 of Wellensiek et al., “Genome-wide profiling of human cap-independent translation-enhancing elements,” Nature Methods 2013, DOI:10.1038/NMETH.2522.

In some embodiments, a 5′UTR and/or 3′UTR of a polynucleotide comprises a TEE which is a transcription regulatory element described in any of U.S. Pat. Nos. 7,456,273, 7,183,395, US2009/0093049, and WO2001/055371, the contents of each of which are incorporated herein by reference in their entirety. The transcription regulatory elements can be identified by methods known in the art, such as, but not limited to, the methods described in U.S. Pat. Nos. 7,456,273, 7,183,395, US2009/0093049, and WO2001/055371.

In some embodiments, a 5′UTR and/or 3′UTR comprising at least one TEE described herein can be incorporated in a monocistronic sequence such as, but not limited to, a vector system or a nucleic acid vector. As non-limiting examples, the vector systems and nucleic acid vectors can include those described in U.S. Pat. Nos. 7,456,273, 7,183,395, US2007/0048776, US2009/0093049, US2011/0124100, WO2007/025008, and WO2001/055371.

In some embodiments, a 5′UTR and/or 3′UTR of a polynucleotide comprises a TEE or portion thereof described herein. In some embodiments, the TEEs in the 3′UTR can be the same and/or different from the TEE located in the 5′UTR.

In some embodiments, a 5′UTR and/or 3′UTR of a polynucleotide can include at least 1, at least 2, at least 3, at least 4, at least 5, at least 6, at least 7, at least 8, at least 9, at least 10, at least 11, at least 12, at least 13, at least 14, at least 15, at least 16, at least 17, at least 18 at least 19, at least 20, at least 21, at least 22, at least 23, at least 24, at least 25, at least 30, at least 35, at least 40, at least 45, at least 50, at least 55 or more than 60 TEE sequences. In one embodiment, the 5′UTR of a polynucleotide can include 1-60, 1-55, 1-50, 1-45, 1-40, 1-35, 1-30, 1-25, 1-20, 1-15, 1-10, 9, 8, 7, 6, 5, 4, 3, 2, or 1 TEE sequences. The TEE sequences in the 5′UTR of the polynucleotide can be the same or different TEE sequences. A combination of different TEE sequences in the 5′UTR of the polynucleotide can include combinations in which more than one copy of any of the different TEE sequences are incorporated. The TEE sequences can be in a pattern such as ABABAB or AABBAABBAABB or ABCABCABC or variants thereof repeated one, two, three, or more than three times. In these patterns, each letter, A, B, or C represent a different TEE nucleotide sequence.

In some embodiments, the TEE can be identified by the methods described in US2007/0048776, US2011/0124100, WO2007/025008, WO2012/009644, the contents of each of which are incorporated herein by reference in their entirety.

In some embodiments, the 5′UTR and/or 3′UTR comprises a spacer to separate two TEE sequences. As a non-limiting example, the spacer can be a 15 nucleotide spacer and/or other spacers known in the art. As another non-limiting example, the 5′UTR and/or 3′UTR comprises a TEE sequence-spacer module repeated at least once, at least twice, at least 3 times, at least 4 times, at least 5 times, at least 6 times, at least 7 times, at least 8 times, at least 9 times, at least 10 times, or more than 10 times in the 5′UTR and/or 3′UTR, respectively. In some embodiments, the 5′UTR and/or 3′UTR comprises a TEE sequence-spacer module repeated 1, 2, 3, 4, 5, 6, 7, 8, 9, or 10 times.

In some embodiments, the spacer separating two TEE sequences can include other sequences known in the art that can regulate the translation of the polynucleotide, e.g., miR sequences described herein (e.g., miR binding sites and miR seeds). As a non-limiting example, each spacer used to separate two TEE sequences can include a different miR sequence or component of a miR sequence (e.g., miR seed sequence).

In some embodiments, a polynucleotide comprises a miR and/or TEE sequence. In some embodiments, the incorporation of a miR sequence and/or a TEE sequence into a polynucleotide can change the shape of the stem loop region, which can increase and/or decrease translation. See e.g., Kedde et al., Nature Cell Biology 2010 12(10):1014-20, herein incorporated by reference in its entirety).

In certain embodiments, a polynucleotide of the present invention (e.g., a polynucleotide comprising a nucleotide sequence encoding a polypeptide) further comprises a 3′ UTR.

3′UTR is the section of mRNA that immediately follows the translation termination codon and often contains regulatory regions that post-transcriptionally influence gene expression. Regulatory regions within the 3′UTR can influence polyadenylation, translation efficiency, localization, and stability of the mRNA. In one embodiment, the 3′UTR useful for the invention comprises a binding site for regulatory proteins or microRNAs. In some embodiments, the 3′UTR has a silencer region, which binds to repressor proteins and inhibits the expression of the mRNA. In other embodiments, the 3′UTR comprises an AU-rich element. Proteins bind AREs to affect the stability or decay rate of transcripts in a localized manner or affect translation initiation. In other embodiments, the 3′UTR comprises the sequence AAUAAA that directs addition of several hundred adenine residues called the poly(A) tail to the end of the mRNA transcript.

Natural or wild type 3′UTRs are known to have stretches of Adenosines and Uridines embedded in them. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of polynucleotides. When engineering specific polynucleotides, one or more copies of an ARE can be introduced to make polynucleotides less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using polynucleotides and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

In certain embodiments, the 3′ UTR useful for the polynucleotides comprises a 3′UTR selected from the group consisting of SEQ ID NO: 26-43, or any combination thereof.

In certain embodiments, the 3′ UTR sequence useful for the invention comprises a nucleotide sequence at least about 60%, at least about 70%, at least about 80%, at least about 90%, at least about 95%, at least about 96%, at least about 97%, at least about 98%, at least about 99%, or about 100% identical to a sequence selected from the group consisting of 3′UTR sequences selected from the group consisting of SEQ ID NO: 26-43, or any combination thereof.

Regions having a 5′ Cap

The polynucleotide comprising an mRNA encoding a polypeptide can further comprise a 5′ cap. The 5′ cap useful for the encoding mRNA can bind the mRNA Cap Binding Protein (CBP), thereby increasing mRNA stability. The cap can further assist the removal of 5′ proximal introns removal during mRNA splicing.

In some embodiments, the polynucleotide comprising an mRNA encoding a polypeptide comprises a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with α-thio-guanosine nucleotides according to the manufacturer's instructions to create a phosphorothioate linkage in the 5′-ppp-5′ cap. Additional modified guanosine nucleotides can be used such as a-methyl-phosphonate and seleno-phosphate nucleotides.

In certain embodiments, the 5′ cap comprises 2′-O-methylation of the ribose sugars of 5′-terminal and/or 5′-anteterminal nucleotides on the 2′-hydroxyl group of the sugar ring. In other embodiments, the caps for the encoding mRNA include cap analogs, which herein are also referred to as synthetic cap analogs, chemical caps, chemical cap analogs, or structural or functional cap analogs, differ from natural (i.e. endogenous, wild-type or physiological) 5′-caps in their chemical structure, while retaining cap function. Cap analogs can be chemically (i.e. non-enzymatically) or enzymatically synthesized and/or linked to the polynucleotides.

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m⁷G-3′mppp-G; which can equivalently be designated 3′ O-Me-m7G(5′)ppp(5′)G). The 3′-O atom of the other, unmodified, guanine becomes linked to the 5′-terminal nucleotide of the capped polynucleotide. The N7- and 3′-O-methylated guanine provides the terminal moiety of the capped polynucleotide.

Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G).

In some embodiments, the cap is a dinucleotide cap analog. As a non-limiting example, the dinucleotide cap analog can be modified at different phosphate positions with a boranophosphate group or a phophoroselenoate group such as the dinucleotide cap analogs described in U.S. Pat. No. 8,519,110.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m^{3′-O}G(5)ppp(5′)G cap analog (See, e.g., the various cap analogs and the methods of synthesizing cap analogs described in Kore et al. Bioorganic & Medicinal Chemistry 2013 21:4570-4574). In another embodiment, a cap analog of the present disclosure is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

The encoding mRNA can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects. Non-limiting examples of more authentic 5′cap structures of the present disclosure are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-O-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)N1mpNp (cap 1), and 7mG(5′)-ppp(5′)N1mpN2mp (cap 2).

In one embodiment, 5′ terminal caps can include endogenous caps or cap analogs. In one embodiment, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Poly-A Tails

In some embodiments, the polynucleotide comprising an mRNA encoding a polypeptide further comprises a poly A tail. In further embodiments, terminal groups on the poly-A tail can be incorporated for stabilization. In other embodiments, a poly-A tail comprises des-3′ hydroxyl tails. The useful poly-A tails can also include structural moieties or 2′-Omethyl modifications as taught by Junjie Li, et al. (Current Biology, Vol. 15, 1501-1507, Aug. 23, 2005).

In one embodiment, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides). In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides.

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hr, 24 hr, 48 hr, 72 hr and day 7 post-transfection.

In some embodiments, the polynucleotides are designed to include a polyA-G

Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone.

### Start Codon Region

In some embodiments, the polynucleotide comprising an mRNA encoding a polypeptide further comprises regions that are analogous to or function like a start codon region.

In some embodiments, the translation of a polynucleotide initiates on a codon which is not the start codon AUG. Translation of the polynucleotide can initiate on an alternative start codon such as, but not limited to, ACG, AGG, AAG, CTG/CUG, GTG/GUG, ATA/AUA, ATT/AUU, TTG/UUG (see Touriol et al. Biology of the Cell 95 (2003) 169-178 and Matsuda and Mauro PLoS ONE, 2010 5:11). As a non-limiting example, the translation of a polynucleotide begins on the alternative start codon ACG. As another non-limiting example, polynucleotide translation begins on the alternative start codon CTG or CUG. As yet another non-limiting example, the translation of a polynucleotide begins on the alternative start codon GTG or GUG.

Nucleotides flanking a codon that initiates translation such as, but not limited to, a start codon or an alternative start codon, are known to affect the translation efficiency, the length and/or the structure of the polynucleotide. (See, e.g., Matsuda and Mauro PLoS ONE, 2010 5:11). Masking any of the nucleotides flanking a codon that initiates translation can be used to alter the position of translation initiation, translation efficiency, length and/or structure of a polynucleotide.

In some embodiments, a masking agent is used near the start codon or alternative start codon in order to mask or hide the codon to reduce the probability of translation initiation at the masked start codon or alternative start codon. Non-limiting examples of masking agents include antisense locked nucleic acids (LNA) polynucleotides and exon-junction complexes (EJCs) (See, e.g., Matsuda and Mauro describing masking agents LNA polynucleotides and EJCs (PLoS ONE, 2010 5:11)).

In another embodiment, a masking agent is used to mask a start codon of a polynucleotide in order to increase the likelihood that translation will initiate on an alternative start codon. In some embodiments, a masking agent is used to mask a first start codon or alternative start codon in order to increase the chance that translation will initiate on a start codon or alternative start codon downstream to the masked start codon or alternative start codon.

In some embodiments, a start codon or alternative start codon is located within a perfect complement for a miR binding site. The perfect complement of a miR binding site can help control the translation, length and/or structure of the polynucleotide similar to a masking agent. As a non-limiting example, the start codon or alternative start codon is located in the middle of a perfect complement for a miR-122 binding site. The start codon or alternative start codon can be located after the first nucleotide, second nucleotide, third nucleotide, fourth nucleotide, fifth nucleotide, sixth nucleotide, seventh nucleotide, eighth nucleotide, ninth nucleotide, tenth nucleotide, eleventh nucleotide, twelfth nucleotide, thirteenth nucleotide, fourteenth nucleotide, fifteenth nucleotide, sixteenth nucleotide, seventeenth nucleotide, eighteenth nucleotide, nineteenth nucleotide, twentieth nucleotide or twenty-first nucleotide.

In another embodiment, the start codon of a polynucleotide is removed from the polynucleotide sequence in order to have the translation of the polynucleotide begin on a codon which is not the start codon. Translation of the polynucleotide can begin on the codon following the removed start codon or on a downstream start codon or an alternative start codon. In a non-limiting example, the start codon ATG or AUG is removed as the first 3 nucleotides of the polynucleotide sequence in order to have translation initiate on a downstream start codon or alternative start codon. The polynucleotide sequence where the start codon was removed can further comprise at least one masking agent for the downstream start codon and/or alternative start codons in order to control or attempt to control the initiation of translation, the length of the polynucleotide and/or the structure of the polynucleotide.

### Stop Codon Region

In some embodiments, the polynucleotide comprising an mRNA encoding a polypeptide can further comprise at least one stop codon or at least two stop codons before the 3′ untranslated region (UTR). The stop codon can be selected from UGA, UAA, and UAG. In some embodiments, the polynucleotides of the present disclosure include the stop codon UGA and one additional stop codon. In a further embodiment the addition stop codon can be UAA. In another embodiment, the polynucleotides of the present disclosure include three stop codons, four stop codons, or more.

Modified polynucleotide

As used herein in a polynucleotide comprising an mRNA encoding a polypeptide, the terms “chemical modification” or, as appropriate, “chemically modified” refer to modification with respect to adenosine (A), guanosine (G), uridine (U), thymidine (T) or cytidine (C) ribo- or deoxyribnucleosides in one or more of their position, pattern, percent or population. Generally, herein, these terms are not intended to refer to the ribonucleotide modifications in naturally occurring 5′-terminal mRNA cap moieties.

In a polypeptide, the term “modification” refers to a modification as compared to the canonical set of 20 amino acids.

The modifications can be various distinct modifications. In some embodiments, the regions can contain one, two, or more (optionally different) nucleoside or nucleotide (nucleobase) modifications. In some embodiments, a modified polynucleotide, introduced to a cell can exhibit reduced degradation in the cell, as compared to an unmodified polynucleotide. In other embodiments, the modification is in the nucleobase and/or the sugar structure. In yet other embodiments, the modification is in the backbone structure.

### Chemical Modifications

In some embodiments, the polynucleotides of the present invention are chemically modified. As used herein in reference to a polynucleotide, the terms “chemical modification” or, as appropriate, “chemically modified” refer to modification with respect to adenosine (A), guanosine (G), uridine (U), thymidine (T) or cytidine (C) ribo- or deoxyribonucleosides in one or more of their position, pattern, percent or population. Generally, herein, these terms are not intended to refer to the ribonucleotide modifications in naturally occurring 5′-terminal mRNA cap moieties.

In some embodiments, the polynucleotides of the present invention can have a uniform chemical modification of all or any of the same nucleoside type or a population of modifications produced by mere downward titration of the same starting modification in all or any of the same nucleoside type, or a measured percent of a chemical modification of all any of the same nucleoside type but with random incorporation, such as where all uridines are replaced by a uridine analog, e.g., pseudouridine or 5-methoxyuridine. In another embodiment, the polynucleotides can have a uniform chemical modification of two, three, or four of the same nucleoside type throughout the entire polynucleotide (such as all uridines and all cytosines, etc. are modified in the same way).

Modified nucleotide base pairing encompasses not only the standard adenosine-thymine, adenosine-uracil, or guanosine-cytosine base pairs, but also base pairs formed between nucleotides and/or modified nucleotides comprising non-standard or modified bases, wherein the arrangement of hydrogen bond donors and hydrogen bond acceptors permits hydrogen bonding between a non-standard base and a standard base or between two complementary non-standard base structures. One example of such non-standard base pairing is the base pairing between the modified nucleotide inosine and adenine, cytosine or uracil. Any combination of base/sugar or linker can be incorporated into polynucleotides of the present disclosure.

The skilled artisan will appreciate that, except where otherwise noted, polynucleotide sequences set forth in the instant application will recite “T”s in a representative DNA sequence but where the sequence represents RNA, the “T”s would be substituted for “U”s.

Modifications of polynucleotides (e.g., RNA polynucleotides, such as mRNA polynucleotides) that are useful in the compositions, methods and synthetic processes of the present disclosure include, but are not limited to the following nucleotides, nucleosides, and nucleobases: 2-methylthio-N6-(cis-hydroxyisopentenyl)adenosine; 2-methylthio-N6-methyladenosine; 2-methylthio-N6-threonyl carbamoyladenosine; N6-glycinylcarbamoyladenosine; N6-isopentenyladenosine; N6-methyladenosine; N6-threonylcarbamoyladenosine; 1,2′-O-dimethyladenosine; 1-methyladenosine; 2′-O-methyladenosine; 2′-O-ribosyladenosine (phosphate); 2-methyladenosine; 2-methylthio-N6 isopentenyladenosine; 2-methylthio-N6-hydroxynorvalyl carbamoyladenosine; 2′-O-methyladenosine; 2′-O-ribosyladenosine (phosphate); Isopentenyladenosine; N6-(cis-hydroxyisopentenyl)adenosine; N6,2′-O-dimethyladenosine; N6,2′-O-dimethyladenosine; N6,N6,2′-O-trimethyladenosine; N6,N6-dimethyladenosine; N6-acetyladenosine; N6-hydroxynorvalylcarbamoyladenosine; N6-methyl-N6-threonylcarbamoyladenosine; 2-methyladenosine; 2-methylthio-N6-isopentenyladenosine; 7-deaza-adenosine; N1-methyl-adenosine; N6, N6 (dimethyl)adenine; N6-cis-hydroxy-isopentenyl-adenosine; α-thio-adenosine; 2 (amino)adenine; 2 (aminopropyl)adenine; 2 (methylthio) N6 (isopentenyl)adenine; 2-(alkyl)adenine; 2-(aminoalkyl)adenine; 2-(aminopropyl)adenine; 2-(halo)adenine; 2-(halo)adenine; 2-(propyl)adenine; 2′-Amino-2′-deoxy-ATP; 2′-Azido-2′-deoxy-ATP; 2′-Deoxy-2′-a-aminoadenosine TP; 2′-Deoxy-2′-a-azidoadenosine TP; 6 (alkyl)adenine; 6 (methyl)adenine; 6-(alkyl)adenine; 6-(methyl)adenine; 7 (deaza)adenine; 8 (alkenyl)adenine; 8 (alkynyl)adenine; 8 (amino)adenine; 8 (thioalkyl)adenine; 8-(alkenyl)adenine; 8-(alkyl)adenine; 8-(alkynyl)adenine; 8-(amino)adenine; 8-(halo)adenine; 8-(hydroxyl)adenine; 8-(thioalkyl)adenine; 8-(thiol)adenine; 8-azido-adenosine; aza adenine; deaza adenine; N6 (methyl)adenine; N6-(isopentyl)adenine; 7-deaza-8-aza-adenosine; 7-methyladenine; 1-Deazaadenosine TP; 2′Fluoro-N6-Bz-deoxyadenosine TP; 2′-OMe-2-Amino-ATP; 2′O-methyl-N6-Bz-deoxyadenosine TP; 2′-a-Ethynyladenosine TP; 2-aminoadenine; 2-Aminoadenosine TP; 2-Amino-ATP; 2′-a-Trifluoromethyladenosine TP; 2-Azidoadenosine TP; 2′-b-Ethynyladenosine TP; 2-Bromoadenosine TP; 2′-b-Trifluoromethyladenosine TP; 2-Chloroadenosine TP; 2′-Deoxy-2′,2′-difluoroadenosine TP; 2′-Deoxy-2′-a-mercaptoadenosine TP; 2′-Deoxy-2′-a-thiomethoxyadenosine TP; 2′-Deoxy-2′-b-aminoadenosine TP; 2′-Deoxy-2′-b-azidoadenosine TP; 2′-Deoxy-2′-b-bromoadenosine TP; 2′-Deoxy-2′-b-chloroadenosine TP; 2′-Deoxy-2′-b-fluoroadenosine TP; 2′-Deoxy-2′-b-iodoadenosine TP; 2′-Deoxy-2′-b-mercaptoadenosine TP; 2′-Deoxy-2′-b-thiomethoxyadenosine TP; 2-Fluoroadenosine TP; 2-Iodoadenosine TP; 2-Mercaptoadenosine TP; 2-methoxy-adenine; 2-methylthio-adenine; 2-Trifluoromethyladenosine TP; 3-Deaza-3-bromoadenosine TP; 3-Deaza-3-chloroadenosine TP; 3-Deaza-3-fluoroadenosine TP; 3-Deaza-3-iodoadenosine TP; 3-Deazaadenosine TP; 4′-Azidoadenosine TP; 4′-Carbocyclic adenosine TP; 4′-Ethynyladenosine TP; 5′-Homo-adenosine TP; 8-Aza-ATP; 8-bromo-adenosine TP; 8-Trifluoromethyladenosine TP; 9-Deazaadenosine TP; 2-aminopurine; 7-deaza-2,6-diaminopurine; 7-deaza-8-aza-2,6-diaminopurine; 7-deaza-8-aza-2-aminopurine; 2,6-diaminopurine; 7-deaza-8-aza-adenine, 7-deaza-2-aminopurine; 2-thiocytidine; 3-methylcytidine; 5-formylcytidine; 5-hydroxymethylcytidine; 5-methylcytidine; N4-acetylcytidine; 2′-O-methylcytidine; 2′-O-methylcytidine; 5,2′-O-dimethylcytidine; 5-formyl-2′-O-methylcytidine; Lysidine; N4,2′-O-dimethylcytidine; N4-acetyl-2′-O-methylcytidine; N4-methylcytidine; N4,N4-Dimethyl-2′-OMe-Cytidine TP; 4-methylcytidine; 5-aza-cytidine; Pseudo-iso-cytidine; pyrrolo-cytidine; α-thio-cytidine; 2-(thio)cytosine; 2′-Amino-2′-deoxy-CTP; 2′-Azido-2′-deoxy-CTP; 2′-Deoxy-2′-a-aminocytidine TP; 2′-Deoxy-2′-a-azidocytidine TP; 3 (deaza) 5 (aza)cytosine; 3 (methyl)cytosine; 3-(alkyl)cytosine; 3-(deaza) 5 (aza)cytosine; 3-(methyl)cytidine; 4,2′-O-dimethylcytidine; 5 (halo)cytosine; 5 (methyl)cytosine; 5 (propynyl)cytosine; 5 (trifluoromethyl)cytosine; 5-(alkyl)cytosine; 5-(alkynyl)cytosine; 5-(halo)cytosine; 5-(propynyl)cytosine; 5-(trifluoromethyl)cytosine; 5-bromo-cytidine; 5-iodo-cytidine; 5-propynyl cytosine; 6-(azo)cytosine; 6-aza-cytidine; aza cytosine; deaza cytosine; N4 (acetyl)cytosine; 1-methyl-1-deaza-pseudoisocytidine; 1-methyl-pseudoisocytidine; 2-methoxy-5-methyl-cytidine; 2-methoxy-cytidine; 2-thio-5-methyl-cytidine; 4-methoxy-1-methyl-pseudoisocytidine; 4-methoxy-pseudoisocytidine; 4-thio-1-methyl-1-deaza-pseudoisocytidine; 4-thio-1-methyl-pseudoisocytidine; 4-thio-pseudoisocytidine; 5-aza-zebularine; 5-methyl-zebularine; pyrrolo-pseudoisocytidine; Zebularine; (E)-5-(2-Bromo-vinyl)cytidine TP; 2,2′-anhydro-cytidine TP hydrochloride; 2′Fluor-N4-Bz-cytidine TP; 2′Fluoro-N4-Acetyl-cytidine TP; 2′-O-Methyl-N4-Acetyl-cytidine TP; 2′O-methyl-N4-Bz-cytidine TP; 2′-a-Ethynylcytidine TP; 2′-a-Trifluoromethylcytidine TP; 2′-b-Ethynylcytidine TP; 2′-b-Trifluoromethylcytidine TP; 2′-Deoxy-2′,2′-difluorocytidine TP; 2′-Deoxy-2′-a-mercaptocytidine TP; 2′-Deoxy-2′-a-thiomethoxycytidine TP; 2′-Deoxy-2′-b-aminocytidine TP; 2′-Deoxy-2′-b-azidocytidine TP; 2′-Deoxy-2′-b-bromocytidine TP; 2′-Deoxy-2′-b-chlorocytidine TP; 2′-Deoxy-2′-b-fluorocytidine TP; 2′-Deoxy-2′-b-iodocytidine TP; 2′-Deoxy-2′-b-mercaptocytidine TP; 2′-Deoxy-2′-b-thiomethoxycytidine TP; 2′-O-Methyl-5-(1-propynyl)cytidine TP; 3′-Ethynylcytidine TP; 4′-Azidocytidine TP; 4′-Carbocyclic cytidine TP; 4′-Ethynylcytidine TP; 5-(1-Propynyl)ara-cytidine TP; 5-(2-Chloro-phenyl)-2-thiocytidine TP; 5-(4-Amino-phenyl)-2-thiocytidine TP; 5-Aminoallyl-CTP; 5-Cyanocytidine TP; 5-Ethynylara-cytidine TP; 5-Ethynylcytidine TP; 5′-Homo-cytidine TP; 5-Methoxycytidine TP; 5-Trifluoromethyl-Cytidine TP; N4-Amino-cytidine TP; N4-Benzoyl-cytidine TP; Pseudoisocytidine; 7-methylguanosine; N2,2′-O-dimethylguanosine; N2-methylguanosine; Wyosine; 1,2′-O-dimethylguanosine; 1-methylguanosine; 2′-O-methylguanosine; 2′-O-ribosylguanosine (phosphate); 2′-O-methylguanosine; 2′-O-ribosylguanosine (phosphate); 7-aminomethyl-7-deazaguanosine; 7-cyano-7-deazaguanosine; Archaeosine; Methylwyosine; N2,7-dimethylguanosine; N2,N2,2′-O-trimethylguanosine; N2,N2,7-trimethylguanosine; N2,N2-dimethylguanosine; N2,7,2′-O-trimethylguanosine; 6-thio-guanosine; 7-deaza-guanosine; 8-oxo-guanosine; N1-methyl-guanosine; α-thio-guanosine; 2 (propyl)guanine; 2-(alkyl)guanine; 2′-Amino-2′-deoxy-GTP; 2′-Azido-2′-deoxy-GTP; 2′-Deoxy-2′-a-aminoguanosine TP; 2′-Deoxy-2′-a-azidoguanosine TP; 6 (methyl)guanine; 6-(alkyl)guanine; 6-(methyl)guanine; 6-methyl-guanosine; 7 (alkyl)guanine; 7 (deaza)guanine; 7 (methyl)guanine; 7-(alkyl)guanine; 7-(deaza)guanine; 7-(methyl)guanine; 8 (alkyl)guanine; 8 (alkynyl)guanine; 8 (halo)guanine; 8 (thioalkyl)guanine; 8-(alkenyl)guanine; 8-(alkyl)guanine; 8-(alkynyl)guanine; 8-(amino)guanine; 8-(halo)guanine; 8-(hydroxyl)guanine; 8-(thioalkyl)guanine; 8-(thiol)guanine; aza guanine; deaza guanine; N (methyl)guanine; N-(methyl)guanine; 1-methyl-6-thio-guanosine; 6-methoxy-guanosine; 6-thio-7-deaza-8-aza-guanosine; 6-thio-7-deaza-guanosine; 6-thio-7-methyl-guanosine; 7-deaza-8-aza-guanosine; 7-methyl-8-oxo-guanosine; N2,N2-dimethyl-6-thio-guanosine; N2-methyl-6-thio-guanosine; 1-Me-GTP; 2′Fluoro-N2-isobutyl-guanosine TP; 2′O-methyl-N2-isobutyl-guanosine TP; 2′-a-Ethynylguanosine TP; 2′-a-Trifluoromethylguanosine TP; 2′-b-Ethynylguanosine TP; 2′-b-Trifluoromethylguanosine TP; 2′-Deoxy-2′,2′-difluoroguanosine TP; 2′-Deoxy-2′-a-mercaptoguanosine TP; 2′-Deoxy-2′-a-thiomethoxyguanosine TP; 2′-Deoxy-2′-b-aminoguanosine TP; 2′-Deoxy-2′-b-azidoguanosine TP; 2′-Deoxy-2′-b-bromoguanosine TP; 2′-Deoxy-2′-b-chloroguanosine TP; 2′-Deoxy-2′-b-fluoroguanosine TP; 2′-Deoxy-2′-b-iodoguanosine TP; 2′-Deoxy-2′-b-mercaptoguanosine TP; 2′-Deoxy-2′-b-thiomethoxyguanosine TP; 4′-Azidoguanosine TP; 4′-Carbocyclic guanosine TP; 4′-Ethynylguanosine TP; 5′-Homo-guanosine TP; 8-bromo-guanosine TP; 9-Deazaguanosine TP; N2-isobutyl-guanosine TP; 1-methylinosine; Inosine; 1,2′-O-dimethylinosine; 2′-O-methylinosine; 7-methylinosine; 2′-O-methylinosine; Epoxyqueuosine; galactosyl-queuosine; Mannosylqueuosine; Queuosine; allyamino-thymidine; aza thymidine; deaza thymidine; deoxy-thymidine; 2′-O-methyluridine; 2-thiouridine; 3-methyluridine; 5-carboxymethyluridine; 5-hydroxyuridine; 5-methyluridine; 5-taurinomethyl-2-thiouridine; 5-taurinomethyluridine; Dihydrouridine; Pseudouridine; (3-(3-amino-3-carboxypropyl)uridine; 1-methyl-3-(3-amino-5-carboxypropyl)pseudouridine; 1-methylpseduouridine; 1-ethyl-pseudouridine; 2′-O-methyluridine; 2′-O-methylpseudouridine; 2′-O-methyluridine; 2-thio-2′-O-methyluridine; 3-(3-amino-3-carboxypropyl)uridine; 3,2′-O-dimethyluridine; 3-Methyl-pseudo-Uridine TP; 4-thiouridine; 5-(carboxyhydroxymethyl)uridine; 5-(carboxyhydroxymethyl)uridine methyl ester; 5,2′-O-dimethyluridine; 5,6-dihydro-uridine; 5-aminomethyl-2-thiouridine; 5-carbamoylmethyl-2′-O-methyluridine; 5-carbamoylmethyluridine; 5-carboxyhydroxymethyluridine; 5-carboxyhydroxymethyluridine methyl ester; 5-carboxymethylaminomethyl-2′-O-methyluridine; 5-carboxymethylaminomethyl-2-thiouridine; 5-carboxymethylaminomethyl-2-thiouridine; 5-carboxymethylaminomethyluridine; 5-carboxymethylaminomethyluridine; 5-Carbamoylmethyluridine TP; 5-methoxycarbonylmethyl-2′-O-methyluridine; 5-methoxycarbonylmethyl-2-thiouridine; 5-methoxycarbonylmethyluridine; 5-methyluridine,), 5-methoxyuridine; 5-methyl-2-thiouridine; 5-methylaminomethyl-2-selenouridine; 5-methylaminomethyl-2-thiouridine; 5-methylaminomethyluridine; 5-Methyldihydrouridine; 5-Oxyacetic acid-Uridine TP; 5-Oxyacetic acid-methyl ester-Uridine TP; N1-methyl-pseudo-uracil; N1-ethyl-pseudo-uracil; uridine 5-oxyacetic acid; uridine 5-oxyacetic acid methyl ester; 3-(3-Amino-3-carboxypropyl)-Uridine TP; 5-(iso-Pentenylaminomethyl)-2-thiouridine TP; 5-(i so-Pentenylaminomethyl)-2′-O-methyluridine TP; 5-(iso-Pentenylaminomethyl)uridine TP; 5-propynyl uracil; α-thio-uridine; 1 (aminoalkylamino-carbonylethylenyl)-2(thio)-pseudouracil; 1 (aminoalkylaminocarbonylethylenyl)-2,4-(dithio)pseudouracil; 1 (aminoalkylaminocarbonylethylenyl)-4 (thio)pseudouracil; 1 (aminoalkylaminocarbonylethylenyl)-pseudouracil; 1 (aminocarbonylethylenyl)-2(thio)-pseudouracil; 1 (aminocarbonylethylenyl)-2,4-(dithio)pseudouracil; 1 (aminocarbonylethylenyl)-4 (thio)pseudouracil; 1 (aminocarbonylethylenyl)-pseudouracil; 1 substituted 2(thio)-pseudouracil; 1 substituted 2,4-(dithio)pseudouracil; 1 substituted 4 (thio)pseudouracil; 1 substituted pseudouracil; 1-(aminoalkylamino-carbonylethylenyl)-2-(thio)-pseudouracil; 1-Methyl-3-(3-amino-3-carboxypropyl) pseudouridine TP; 1-Methyl-3-(3-amino-3-carboxypropyl)pseudo-UTP; 1-Methyl-pseudo-UTP; 1-Ethyl-pseudo-UTP; 2 (thio)pseudouracil; 2′ deoxy uridine; 2′ fluorouridine; 2-(thio)uracil; 2,4-(dithio)psuedouracil; 2′ methyl, 2′amino, 2′azido, 2′fluoro-guanosine; 2′-Amino-2′-deoxy-UTP; 2′-Azido-2′-deoxy-UTP; 2′-Azido-deoxyuridine TP; 2′-O-methylpseudouridine; 2′ deoxy uridine; 2′ fluorouridine; 2′-Deoxy-2′-a-aminouridine TP; 2′-Deoxy-2′-a-azidouridine TP; 2-methylpseudouridine; 3 (3 amino-3 carboxypropyl)uracil; 4 (thio)pseudouracil; 4-(thio)pseudouracil; 4-(thio)uracil; 4-thiouracil; 5 (1,3-diazole-1-alkyl)uracil; 5 (2-aminopropyl)uracil; 5 (aminoalkyl)uracil; 5 (dimethylaminoalkyl)uracil; 5 (guanidiniumalkyl)uracil; 5 (methoxycarbonylmethyl)-2-(thio)uracil; 5 (methoxycarbonyl-methyl)uracil; 5 (methyl) 2 (thio)uracil; 5 (methyl) 2,4 (dithio)uracil; 5 (methyl) 4 (thio)uracil; 5 (methylaminomethyl)-2 (thio)uracil; 5 (methylaminomethyl)-2,4 (dithio)uracil; 5 (methylaminomethyl)-4 (thio)uracil; 5 (propynyl)uracil; 5 (trifluoromethyl)uracil; 5-(2-aminopropyl)uracil; 5-(alkyl)-2-(thio)pseudouracil; 5-(alkyl)-2,4 (dithio)pseudouracil; 5-(alkyl)-4 (thio)pseudouracil; 5-(alkyl)pseudouracil; 5-(alkyl)uracil; 5-(alkynyl)uracil; 5-(allylamino)uracil; 5-(cyanoalkyl)uracil; 5-(dialkylaminoalkyl)uracil; 5-(dimethylaminoalkyl)uracil; 5-(guanidiniumalkyl)uracil; 5-(halo)uracil; 5-(1,3-diazole-1-alkyl)uracil; 5-(methoxy)uracil; 5-(methoxycarbonylmethyl)-2-(thio)uracil; 5-(methoxycarbonyl-methyl)uracil; 5-(methyl) 2(thio)uracil; 5-(methyl) 2,4 (dithio)uracil; 5-(methyl) 4 (thio)uracil; 5-(methyl)-2-(thio)pseudouracil; 5-(methyl)-2,4 (dithio)pseudouracil; 5-(methyl)-4 (thio)pseudouracil; 5-(methyl)pseudouracil; 5-(methylaminomethyl)-2 (thio)uracil; 5-(methylaminomethyl)-2,4(dithio)uracil; 5-(methylaminomethyl)-4-(thio)uracil; 5-(propynyl)uracil; 5-(trifluoromethyl)uracil; 5-aminoallyl-uridine; 5-bromo-uridine; 5-iodo-uridine; 5-uracil; 6 (azo)uracil; 6-(azo)uracil; 6-aza-uridine; allyamino-uracil; aza uracil; deaza uracil; N3 (methyl)uracil; Pseudo-UTP-1-2-ethanoic acid; Pseudouracil; 4-Thio-pseudo-UTP; 1-carboxymethyl-pseudouridine; 1-methyl-1-deaza-pseudouridine; 1-propynyl-uridine; 1-taurinomethyl-1-methyl-uridine; 1-taurinomethyl-4-thio-uridine; 1-taurinomethyl-pseudouridine; 2-methoxy-4-thio-pseudouridine; 2-thio-1-methyl-1-deaza-pseudouridine; 2-thio-1-methyl-pseudouridine; 2-thio-5-aza-uridine; 2-thio-dihydropseudouridine; 2-thio-dihydrouridine; 2-thio-pseudouridine; 4-methoxy-2-thio-pseudouridine; 4-methoxy-pseudouridine; 4-thio-1-methyl-pseudouridine; 4-thio-pseudouridine; 5-aza-uridine; Dihydropseudouridine; (±)1-(2-Hydroxypropyl)pseudouridine TP; (2R)-1-(2-Hydroxypropyl)pseudouridine TP; (2S)-1-(2-Hydroxypropyl)pseudouridine TP; (E)-5-(2-Bromo-vinyl)ara-uridine TP; (E)-5-(2-Bromo-vinyl)uridine TP; (Z)-5-(2-Bromo-vinyl)ara-uridine TP; (Z)-5-(2-Bromo-vinyl)uridine TP; 1-(2,2,2-Trifluoroethyl)-pseudo-UTP; 1-(2,2,3,3,3-Pentafluoropropyl)pseudouridine TP; 1-(2,2-Diethoxyethyl)pseudouridine TP; 1-(2,4,6-Trimethylbenzyl)pseudouridine TP; 1-(2,4,6-Trimethyl-benzyl)pseudo-UTP; 1-(2,4,6-Trimethyl-phenyl)pseudo-UTP; 1-(2-Amino-2-carboxyethyl)pseudo-UTP; 1-(2-Amino-ethyl)pseudo-UTP; 1-(2-Hydroxyethyl)pseudouridine TP; 1-(2-Methoxyethyl)pseudouridine TP; 1-(3,4-Bis-trifluoromethoxybenzyl)pseudouridine TP; 1-(3,4-Dimethoxybenzyl)pseudouridine TP; 1-(3-Amino-3-carboxypropyl)pseudo-UTP; 1-(3-Amino-propyl)pseudo-UTP; 1-(3-Cyclopropyl-prop-2-ynyl)pseudouridine TP; 1-(4-Amino-4-carboxybutyl)pseudo-UTP; 1-(4-Amino-benzyl)pseudo-UTP; 1-(4-Amino-butyl)pseudo-UTP; 1-(4-Amino-phenyl)pseudo-UTP; 1-(4-Azidobenzyl)pseudouridine TP; 1-(4-Bromobenzyl)pseudouridine TP; 1-(4-Chlorobenzyl)pseudouridine TP; 1-(4-Fluorobenzyl)pseudouridine TP; 1-(4-Iodobenzyl)pseudouridine TP; 1-(4-Methanesulfonylbenzyl)pseudouridine TP; 1-(4-Methoxybenzyl)pseudouridine TP; 1-(4-Methoxy-benzyl)pseudo-UTP; 1-(4-Methoxy-phenyl)pseudo-UTP; 1-(4-Methylbenzyl)pseudouridine TP; 1-(4-Methyl-benzyl)pseudo-UTP; 1-(4-Nitrobenzyl)pseudouridine TP; 1-(4-Nitro-benzyl)pseudo-UTP; 1(4-Nitro-phenyl)pseudo-UTP; 1-(4-Thiomethoxybenzyl)pseudouridine TP; 1-(4-Trifluoromethoxybenzyl)pseudouridine TP; 1-(4-Trifluoromethylbenzyl)pseudouridine TP; 1-(5-Amino-pentyl)pseudo-UTP; 1-(6-Amino-hexyl)pseudo-UTP; 1,6-Dimethyl-pseudo-UTP; 1-[3-(2-{2-[2-(2-Aminoethoxy)-ethoxy]-ethoxy}-ethoxy)-propionyl]pseudouridine TP; 1-{3-[2-(2-Aminoethoxy)-ethoxy]-propionyl} pseudouridine TP; 1-Acetylpseudouridine TP; 1-Alkyl-6-(1-propynyl)-pseudo-UTP; 1-Alkyl-6-(2-propynyl)-pseudo-UTP; 1-Alkyl-6-allyl-pseudo-UTP; 1-Alkyl-6-ethynyl-pseudo-UTP; 1-Alkyl-6-homoallyl-pseudo-UTP; 1-Alkyl-6-vinyl-pseudo-UTP; 1-Allylpseudouridine TP; 1-Aminomethyl-pseudo-UTP; 1-Benzoylpseudouridine TP; 1-Benzyloxymethylpseudouridine TP; 1-Benzyl-pseudo-UTP; 1-Biotinyl-PEG2-pseudouridine TP; 1-Biotinylpseudouridine TP; 1-Butyl-pseudo-UTP; 1-Cyanomethylpseudouridine TP; 1-Cyclobutylmethyl-pseudo-UTP; 1-Cyclobutyl-pseudo-UTP; 1-Cycloheptylmethyl-pseudo-UTP; 1-Cycloheptyl-pseudo-UTP; 1-Cyclohexylmethyl-pseudo-UTP; 1-Cyclohexyl-pseudo-UTP; 1-Cyclooctylmethyl-pseudo-UTP; 1-Cyclooctyl-pseudo-UTP; 1-Cyclopentylmethyl-pseudo-UTP; 1-Cyclopentyl-pseudo-UTP; 1-Cyclopropylmethyl-pseudo-UTP; 1-Cyclopropyl-pseudo-UTP; 1-Ethyl-pseudo-UTP; 1-Hexyl-pseudo-UTP; 1-Homoallylpseudouridine TP; 1-Hydroxymethylpseudouridine TP; 1-iso-propyl-pseudo-UTP; 1-Me-2-thio-pseudo-UTP; 1-Me-4-thio-pseudo-UTP; 1-Me-alpha-thio-pseudo-UTP; 1-Methanesulfonylmethylpseudouridine TP; 1-Methoxymethylpseudouridine TP; 1-Methyl-6-(2,2,2-Trifluoroethyl)pseudo-UTP; 1-Methyl-6-(4-morpholino)-pseudo-UTP; 1-Methyl-6-(4-thiomorpholino)-pseudo-UTP; 1-Methyl-6-(substituted phenyl)pseudo-UTP; 1-Methyl-6-amino-pseudo-UTP; 1-Methyl-6-azido-pseudo-UTP; 1-Methyl-6-bromo-pseudo-UTP; 1-Methyl-6-butyl-pseudo-UTP; 1-Methyl-6-chloro-pseudo-UTP; 1-Methyl-6-cyano-pseudo-UTP; 1-Methyl-6-dimethylamino-pseudo-UTP; 1-Methyl-6-ethoxy-pseudo-UTP; 1-Methyl-6-ethylcarboxylate-pseudo-UTP; 1-Methyl-6-ethyl-pseudo-UTP; 1-Methyl-6-fluoro-pseudo-UTP; 1-Methyl-6-formyl-pseudo-UTP; 1-Methyl-6-hydroxyamino-pseudo-UTP; 1-Methyl-6-hydroxy-pseudo-UTP; 1-Methyl-6-iodo-pseudo-UTP; 1-Methyl-6-iso-propyl-pseudo-UTP; 1-Methyl-6-methoxy-pseudo-UTP; 1-Methyl-6-methylamino-pseudo-UTP; 1-Methyl-6-phenyl-pseudo-UTP; 1-Methyl-6-propyl-pseudo-UTP; 1-Methyl-6-tert-butyl-pseudo-UTP; 1-Methyl-6-trifluoromethoxy-pseudo-UTP; 1-Methyl-6-trifluoromethyl-pseudo-UTP; 1-Morpholinomethylpseudouridine TP; 1-Pentyl-pseudo-UTP; 1-Phenyl-pseudo-UTP; 1-Pivaloylpseudouridine TP; 1-Propargylpseudouridine TP; 1-Propyl-pseudo-UTP; 1-propynyl-pseudouridine; 1-p-tolyl-pseudo-UTP; 1-tert-Butyl-pseudo-UTP; 1-Thiomethoxymethylpseudouridine TP; 1-Thiomorpholinomethylpseudouridine TP; 1-Trifluoroacetylpseudouridine TP; 1-Trifluoromethyl-pseudo-UTP; 1-Vinylpseudouridine TP; 2,2′-anhydro-uridine TP; 2′-bromo-deoxyuridine TP; 2′-F-5-Methyl-2′-deoxy-UTP; 2′-OMe-5-Me-UTP; 2′-OMe-pseudo-UTP; 2′-a-Ethynyluridine TP; 2′-a-Trifluoromethyluridine TP; 2′-b-Ethynyluridine TP; 2′-b-Trifluoromethyluridine TP; 2′-Deoxy-2′,2′-difluorouridine TP; 2′-Deoxy-2′-a-mercaptouridine TP; 2′-Deoxy-2′-a-thiomethoxyuridine TP; 2′-Deoxy-2′-b-aminouridine TP; 2′-Deoxy-2′-b-azidouridine TP; 2′-Deoxy-2′-b-bromouridine TP; 2′-Deoxy-2′-b-chlorouridine TP; 2′-Deoxy-2′-b-fluorouridine TP; 2′-Deoxy-2′-b-iodouridine TP; 2′-Deoxy-2′-b-mercaptouridine TP; 2′-Deoxy-2′-b-thiomethoxyuridine TP; 2-methoxy-4-thio-uridine; 2-methoxyuridine; 2′-O-Methyl-5-(1-propynyl)uridine TP; 3-Alkyl-pseudo-UTP; 4′-Azidouridine TP; 4′-Carbocyclic uridine TP; 4′-Ethynyluridine TP; 5-(1-Propynyl)ara-uridine TP; 5-(2-Furanyl)uridine TP; 5-Cyanouridine TP; 5-Dimethylaminouridine TP; 5′-Homo-uridine TP; 5-iodo-2′-fluoro-deoxyuridine TP; 5-Phenylethynyluridine TP; 5-Trideuteromethyl-6-deuterouridine TP; 5-Trifluoromethyl-Uridine TP; 5-Vinylarauridine TP; 6-(2,2,2-Trifluoroethyl)-pseudo-UTP; 6-(4-Morpholino)-pseudo-UTP; 6-(4-Thiomorpholino)-pseudo-UTP; 6-(Substituted-Phenyl)-pseudo-UTP; 6-Amino-pseudo-UTP; 6-Azido-pseudo-UTP; 6-Bromo-pseudo-UTP; 6-Butyl-pseudo-UTP; 6-Chloro-pseudo-UTP; 6-Cyano-pseudo-UTP; 6-Dimethylamino-pseudo-UTP; 6-Ethoxy-pseudo-UTP; 6-Ethylcarboxylate-pseudo-UTP; 6-Ethyl-pseudo-UTP; 6-Fluoro-pseudo-UTP; 6-Formyl-pseudo-UTP; 6-Hydroxyamino-pseudo-UTP; 6-Hydroxy-pseudo-UTP; 6-Iodo-pseudo-UTP; 6-iso-Propyl-pseudo-UTP; 6-Methoxy-pseudo-UTP; 6-Methylamino-pseudo-UTP; 6-Methyl-pseudo-UTP; 6-Phenyl-pseudo-UTP; 6-Phenyl-pseudo-UTP; 6-Propyl-pseudo-UTP; 6-tert-Butyl-pseudo-UTP; 6-Trifluoromethoxy-pseudo-UTP; 6-Trifluoromethyl-pseudo-UTP; Alpha-thio-pseudo-UTP; Pseudouridine 1-(4-methylbenzenesulfonic acid) TP; Pseudouridine 1-(4-methylbenzoic acid) TP; Pseudouridine TP 1-[3-(2-ethoxy)]propionic acid; Pseudouridine TP 1-[3-{2-(2-[2-(2-ethoxy)-ethoxy]-ethoxy)-ethoxy}]propionic acid; Pseudouridine TP 1-[3-{2-(2-[2-{2(2-ethoxy)-ethoxy}-ethoxy]-ethoxy)-ethoxy}]propionic acid; Pseudouridine TP 1-[3-{2-(2-[2-ethoxy]-ethoxy)-ethoxy}]propionic acid; Pseudouridine TP 143-{2-(2-ethoxy)-ethoxy}]propionic acid; Pseudouridine TP 1-methylphosphonic acid; Pseudouridine TP 1-methylphosphonic acid diethyl ester; Pseudo-UTP-N1-3-propionic acid; Pseudo-UTP-N1-4-butanoic acid; Pseudo-UTP-N1-5-pentanoic acid; Pseudo-UTP-N1-6-hexanoic acid; Pseudo-UTP-N1-7-heptanoic acid; Pseudo-UTP-N1-methyl-p-benzoic acid; Pseudo-UTP-N1-p-benzoic acid; Wybutosine; Hydroxywybutosine; Isowyosine; Peroxywybutosine; undermodified hydroxywybutosine; 4-demethylwyosine; 2,6-(diamino)purine;1-(aza)-2-(thio)-3-(aza)-phenoxazin-1-yl: 1,3-(diaza)-2-(oxo)-phenthiazin-1-yl;1,3-(diaza)-2-(oxo)-phenoxazin-1-yl;1,3,5-(triaza)-2,6-(dioxa)-naphthalene; 2 (amino)purine; 2,4,5-(trimethyl)phenyl; 2′ methyl, 2′amino, 2′azido, 2′fluoro-cytidine;2′ methyl, 2′amino, 2′azido, 2′fluoro-adenine;2′methyl, 2′amino, 2′azido, 2′fluoro-uridine;2′-amino-2′-deoxyribose; 2-amino-6-Chloro-purine; 2-aza-inosinyl; 2′-azido-2′-deoxyribose; 2′fluoro-2′-deoxyribose; 2′-fluoro-modified bases; 2′-O-methyl-ribose; 2-oxo-7-aminopyridopyrimidin-3-yl; 2-oxo-pyridopyrimidine-3-yl; 2-pyridinone; 3 nitropyrrole; 3-(methyl)-7-(propynyl)isocarbostyrilyl; 3-(methyl)isocarbostyrilyl; 4-(fluoro)-6-(methyl)benzimidazole; 4-(methyl)benzimidazole; 4-(methyl)indolyl; 4,6-(dimethyl)indolyl; 5 nitroindole; 5 substituted pyrimidines; 5-(methyl)isocarbostyrilyl; 5-nitroindole; 6-(aza)pyrimidine; 6-(azo)thymine; 6-(methyl)-7-(aza)indolyl; 6-chloro-purine; 6-phenyl-pyrrolo-pyrimidin-2-on-3-yl; 7-(aminoalkylhydroxy)-1-(aza)-2-(thio)-3-(aza)-phenthiazin-1-yl; 7-(aminoalkylhydroxy)-1-(aza)-2-(thio)-3-(aza)-phenoxazin-1-yl; 7-(aminoalkylhydroxy)-1,3-(diaza)-2-(oxo)-phenoxazin-1-yl; 7-(aminoalkylhydroxy)-1,3-(diaza)-2-(oxo)-phenthiazin-1-yl; 7-(aminoalkylhydroxy)-1,3-(diaza)-2-(oxo)-phenoxazin-1-yl; 7-(aza)indolyl; 7-(guanidiniumalkylhydroxy)-1-(aza)-2-(thio)-3-(aza)-phenoxazinl-yl; 7-(guanidiniumalkylhydroxy)-1-(aza)-2-(thio)-3-(aza)-phenthiazin-1-yl; 7-(guanidiniumalkylhydroxy)-1-(aza)-2-(thio)-3-(aza)-phenoxazin-1-yl; 7-(guanidiniumalkylhydroxy)-1,3-(diaza)-2-(oxo)-phenoxazin-1-yl; 7-(guanidiniumalkyl-hydroxy)-1,3-(diaza)-2-(oxo)-phenthiazin-1-yl; 7-(guanidiniumalkylhydroxy)-1,3-(diaza)-2-(oxo)-phenoxazin-1-yl; 7-(propynyl)isocarbostyrilyl; 7-(propynyl)isocarbostyrilyl, propynyl-7-(aza)indolyl; 7-deaza-inosinyl; 7-substituted 1-(aza)-2-(thio)-3-(aza)-phenoxazin-1-yl; 7-substituted 1,3-(diaza)-2-(oxo)-phenoxazin-1-yl; 9-(methyl)-imidizopyridinyl; Aminoindolyl; Anthracenyl; bis-ortho-(aminoalkylhydroxy)-6-phenyl-pyrrolo-pyrimidin-2-on-3-yl; bis-ortho-substituted-6-phenyl-pyrrolo-pyrimidin-2-on-3-yl; Difluorotolyl; Hypoxanthine; Imidizopyridinyl; Inosinyl; Isocarbostyrilyl; Isoguanisine; N2-substituted purines; N6-methyl-2-amino-purine; N6-substituted purines; N-alkylated derivative; Napthalenyl; Nitrobenzimidazolyl; Nitroimidazolyl; Nitroindazolyl; Nitropyrazolyl; Nubularine; O6-substituted purines; O-alkylated derivative; ortho-(aminoalkylhydroxy)-6-phenyl-pyrrolo-pyrimidin-2-on-3-yl; ortho-substituted-6-phenyl-pyrrolo-pyrimidin-2-on-3-yl; Oxoformycin TP; para-(aminoalkylhydroxy)-6-phenyl-pyrrolo-pyrimidin-2-on-3-yl; para-substituted-6-phenyl-pyrrolo-pyrimidin-2-on-3-yl; Pentacenyl; Phenanthracenyl; Phenyl; propynyl-7-(aza)indolyl; Pyrenyl; pyridopyrimidin-3-yl; pyridopyrimidin-3-yl, 2-oxo-7-amino-pyridopyrimidin-3-yl; pyrrolo-pyrimidin-2-on-3-yl; Pyrrolopyrimidinyl; Pyrrolopyrizinyl; Stilbenzyl; substituted 1,2,4-triazoles; Tetracenyl; Tubercidine; Xanthine; Xanthosine-5′-TP; 2-thio-zebularine; 5-aza-2-thio-zebularine; 7-deaza-2-amino-purine; pyridin-4-one ribonucleoside; 2-Amino-riboside-TP; Formycin A TP; Formycin B TP; Pyrrolosine TP; 2′-OH-ara-adenosine TP; 2′-OH-ara-cytidine TP; 2′-OH-ara-uridine TP; 2′-OH-ara-guanosine TP; 5-(2-carbomethoxyvinyl)uridine TP; and N6-(19-Amino-pentaoxanonadecyl)adenosine TP.

In some embodiments, the polynucleotide (e.g., RNA polynucleotide, such as mRNA polynucleotide) includes a combination of at least two (e.g., 2, 3, 4 or more) of the aforementioned modified nucleobases.

In some embodiments, the mRNA comprises at least one chemically modified nucleoside. In some embodiments, the at least one chemically modified nucleoside is selected from the group consisting of pseudouridine (ψ), 2-thiouridine (s2U), 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methoxyuridine, 2′-O-methyl uridine, 1-methyl-pseudouridine (m1ψ), 1-ethyl-pseudouridine (e1ψ), 5-methoxy-uridine (mo5U), 5-methyl-cytidine (m5C), α-thio-guanosine, α-thio-adenosine, 5-cyano uridine, 4′-thio uridine 7-deaza-adenine, 1-methyl-adenosine (m1A), 2-methyl-adenine (m2A), N6-methyl-adenosine (m6A), and 2,6-Diaminopurine, (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (m1 G), 8-oxo-guanosine, 7-methyl-8-oxo-guanosine, 2,8-dimethyladenosine, 2-geranylthiouridine, 2-lysidine, 2-selenouridine, 3-(3-amino-3-carboxypropyl)-5,6-dihydrouridine, 3-(3-amino-3-carboxypropyl)pseudouridine, 3-methylpseudouridine, 5-(carboxyhydroxymethyl)-2′-O-methyluridine methyl ester, 5-aminomethyl-2-geranylthiouridine, 5-aminomethyl-2-selenouridine, 5-aminomethyluridine, 5-carbamoylhydroxymethyluridine, 5-carbamoylmethyl-2-thiouridine, 5-carboxymethyl-2-thiouridine, 5-carboxymethylaminomethyl-2-geranylthiouridine, 5-carboxymethylaminomethyl-2-selenouridine, 5-cyanomethyluridine, 5-hydroxycytidine, 5-methylaminomethyl-2-geranylthiouridine, 7-aminocarboxypropyl-demethylwyosine, 7-aminocarboxypropylwyosine, 7-aminocarboxypropylwyosine methyl ester, 8-methyladenosine, N4,N4-dimethylcytidine, N6-formyladenosine, N6-hydroxymethyladenosine, agmatidine, cyclic N6-threonylcarbamoyladenosine, glutamyl-queuosine, methylated undermodified hydroxywybutosine, N4,N4,2′-O-trimethylcytidine, geranylated 5-methylaminomethyl-2-thiouridine, geranylated 5-carboxymethylaminomethyl-2-thiouridine, Qbase , preQ0base, preQ1base, and two or more combinations thereof. In some embodiments, the at least one chemically modified nucleoside is selected from the group consisting of pseudouridine, 1-methyl-pseudouridine, 1-ethyl-pseudouridine, 5-methylcytosine, 5-methoxyuridine, and a combination thereof. In some embodiments, the polynucleotide (e.g., RNA polynucleotide, such as mRNA polynucleotide) includes a combination of at least two (e.g., 2, 3, 4 or more) of the aforementioned modified nucleobases.

### (i) Base Modifications

In certain embodiments, the chemical modification is at nucleobases in the polynucleotides (e.g., RNA polynucleotide, such as mRNA polynucleotide). In some embodiments, modified nucleobases in the polynucleotide (e.g., RNA polynucleotide, such as mRNA polynucleotide) are selected from the group consisting of 1-methyl-pseudouridine (m1ψ), 1-ethyl-pseudouridine (e1ψ), 5-methoxy-uridine (mo5U), 5-methyl-cytidine (m5C), pseudouridine (ψ), α-thio-guanosine and α-thio-adenosine. In some embodiments, the polynucleotide includes a combination of at least two (e.g., 2, 3, 4 or more) of the aforementioned modified nucleobases.

In some embodiments, the polynucleotide (e.g., RNA polynucleotide, such as mRNA polynucleotide) comprises pseudouridine (ψ) and 5-methyl-cytidine (m5C). In some embodiments, the polynucleotide (e.g., RNA polynucleotide, such as mRNA polynucleotide) comprises 1-methyl-pseudouridine (m1ψ). In some embodiments, the polynucleotide (e.g., RNA polynucleotide, such as mRNA polynucleotide) comprises 1-ethyl-pseudouridine (e1ψ). In some embodiments, the polynucleotide (e.g., RNA polynucleotide, such as mRNA polynucleotide) comprises 1-methyl-pseudouridine (m1ψ) and 5-methyl-cytidine (m5C). In some embodiments, the polynucleotide (e.g., RNA polynucleotide, such as mRNA polynucleotide) comprises 1-ethyl-pseudouridine (e1ψ) and 5-methyl-cytidine (m5C). In some embodiments, the polynucleotide (e.g., RNA polynucleotide, such as mRNA polynucleotide) comprises 2-thiouridine (s2U). In some embodiments, the polynucleotide (e.g., RNA polynucleotide, such as mRNA polynucleotide) comprises 2-thiouridine and 5-methyl-cytidine (m5C). In some embodiments, the polynucleotide (e.g., RNA polynucleotide, such as mRNA polynucleotide) comprises methoxy-uridine (mo5U). In some embodiments, the polynucleotide (e.g., RNA polynucleotide, such as mRNA polynucleotide) comprises 5-methoxy-uridine (mo5U) and 5-methyl-cytidine (m5C). In some embodiments, the polynucleotide (e.g., RNA polynucleotide, such as mRNA polynucleotide) comprises 2′-O-methyl uridine. In some embodiments, the polynucleotide (e.g., RNA polynucleotide, such as mRNA polynucleotide) comprises 2′-O-methyl uridine and 5-methyl-cytidine (m5C). In some embodiments, the polynucleotide (e.g., RNA polynucleotide, such as mRNA polynucleotide) comprises N6-methyl-adenosine (m6A). In some embodiments, the polynucleotide (e.g., RNA polynucleotide, such as mRNA polynucleotide) comprises N6-methyl-adenosine (m6A) and 5-methyl-cytidine (m5C).

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 5-methyl-cytidine (m5C), meaning that all cytosine residues in the mRNA sequence are replaced with 5-methyl-cytidine (m5C). Similarly, a polynucleotide can be uniformly modified for any type of nucleoside residue present in the sequence by replacement with a modified residue such as any of those set forth above.

In some embodiments, the chemically modified nucleosides in the open reading frame are selected from the group consisting of uridine, adenine, cytosine, guanine, and any combination thereof.

In some embodiments, the modified nucleobase is a modified cytosine. Examples of nucleobases and nucleosides having a modified cytosine include N4-acetyl-cytidine (ac4C), 5-methyl-cytidine (m5C), 5-halo-cytidine (e.g., 5-iodo-cytidine), 5-hydroxymethyl-cytidine (hm5C), 1-methyl-pseudoisocytidine, 2-thio-cytidine (s2C), 2-thio-5-methyl-cytidine.

In some embodiments, a modified nucleobase is a modified uridine. Example nucleobases and nucleosides having a modified uridine include 5-cyano uridine or 4′-thio uridine.

In some embodiments, a modified nucleobase is a modified adenine. Example nucleobases and nucleosides having a modified adenine include 7-deaza-adenine, 1-methyl-adenosine (m1A), 2-methyl-adenine (m2A), N6-methyl-adenine (m6A), and 2,6-Diaminopurine.

In some embodiments, a modified nucleobase is a modified guanine. Example nucleobases and nucleosides having a modified guanine include inosine (I), 1-methyl-inosine (m1I), wyosine (imG), methylwyosine (mimG), 7-deaza-guanosine, 7-cyano-7-deaza-guanosine (preQ0), 7-aminomethyl-7-deaza-guanosine (preQ1), 7-methyl-guanosine (m7G), 1-methyl-guanosine (ml G), 8-oxo-guanosine, 7-methyl-8-oxo-guanosine.

In some embodiments, the nucleobase modified nucleotides in the polynucleotide (e.g., RNA polynucleotide, such as mRNA polynucleotide) are 5-methoxyuridine.

In some embodiments, the polynucleotide (e.g., RNA polynucleotide, such as mRNA polynucleotide) includes a combination of at least two (e.g., 2, 3, 4 or more) of modified nucleobases.

In some embodiments, at least 95% of a type of nucleobases (e.g., uracil) in a polynucleotide are modified nucleobases. In some embodiments, at least 95% of uracil in a polynucleotide is 5-methoxyuracil.

In some embodiments, the polynucleotide (e.g., RNA polynucleotide, such as mRNA polynucleotide) comprises 5-methoxyuridine (5mo5U) and 5-methyl-cytidine (m5C).

In some embodiments, the polynucleotide (e.g., RNA polynucleotide, such as mRNA polynucleotide) is uniformly modified (e.g., fully modified, modified throughout the entire sequence) for a particular modification. For example, a polynucleotide can be uniformly modified with 5-methoxyuridine, meaning that substantially all uridine residues in the mRNA sequence are replaced with 5-methoxyuridine. Similarly, a polynucleotide can be uniformly modified for any type of nucleoside residue present in the sequence by replacement with a modified residue such as any of those set forth above.

In some embodiments, the modified nucleobase is a modified cytosine.

In some embodiments, a modified nucleobase is a modified uracil. Example nucleobases and nucleosides having a modified uracil include 5-methoxyuracil.

In some embodiments, a modified nucleobase is a modified adenine.

In some embodiments, a modified nucleobase is a modified guanine.

In some embodiments, the nucleobases, sugar, backbone, or any combination thereof in the open reading frame are chemically modified by at least 10%, at least 20%, at least 30%, at least 40%, at least 50%, at least 60%, at least 70%, at least 80%, at least 90%, at least 95%, at least 99%, or 100%.

In some embodiments, the uridine nucleosides in the open reading frame are chemically modified by at least 10%, at least 20%, at least 30%, at least 40%, at least 50%, at least 60%, at least 70%, at least 80%, at least 90%, at least 95%, at least 99%, or 100%.

In some embodiments, the adenosine nucleosides in the open reading frame are chemically modified by at least 10%, at least 20%, at least 30%, at least 40%, at least 50%, at least 60%, at least 70%, at least 80%, at least 90%, at least 95%, at least 99%, or 100%.

In some embodiments, the cytidine nucleosides in the open reading frame are chemically modified by at least 10%, at least 20%, at least 30%, at least 40%, at least 50%, at least 60%, at least 70%, at least 80%, at least 90%, at least 95%, at least 99%, or 100%.

In some embodiments, the guanosine nucleosides in the open reading frame are chemically modified by at least 10%, at least 20%, at least 30%, at least 40%, at least 50%, at least 60%, at least 70%, at least 80%, at least 90%, at least 95%, at least 99%, or 100%.

In some embodiments, the polynucleotides can include any useful linker between the nucleosides. Such linkers, including backbone modifications, that are useful in the composition of the present disclosure include, but are not limited to the following: 3′-alkylene phosphonates, 3′-amino phosphoramidate, alkene containing backbones, aminoalkylphosphoramidates, aminoalkylphosphotriesters, boranophosphates, N(CH₃)—CH₂—, —CH₂—N(CH₃)—N(CH₃)—CH₂—, —CH₂—NH-CH₂—, chiral phosphonates, chiral phosphorothioates, formacetyl and thioformacetyl backbones, methylene (methylimino), methylene formacetyl and thioformacetyl backbones, methyleneimino and methylenehydrazino backbones, morpholino linkages, —N(CH₃)—CH₂—CH₂—, oligonucleosides with heteroatom internucleoside linkage, phosphinates, phosphoramidates, phosphorodithioates, phosphorothioate internucleoside linkages, phosphorothioates, phosphotriesters, PNA, siloxane backbones, sulfamate backbones, sulfide sulfoxide and sulfone backbones, sulfonate and sulfonamide backbones, thionoalkylphosphonates, thionoalkylphosphotriesters, and thionophosphoramidates.

The polynucleotide comprising an mRNA encoding a polypeptide can include any useful modification, such as to the sugar, the nucleobase, or the internucleoside linkage (e.g. to a linking phosphate/to a phosphodiester linkage/to the phosphodiester backbone). One or more atoms of a pyrimidine nucleobase can be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present disclosure can be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs), hexitol nucleic acids (HNAs), or hybrids thereof. Additional modifications are described herein. Modified nucleic acids and their synthesis are disclosed in co-pending International Patent Application Pub. No. WO 2013052523.

In some embodiments, the polynucleotide comprising an mRNA encoding a polypeptide does not substantially induce an innate immune response of a cell into which the mRNA is introduced. Features of an induced innate immune response include 1) increased expression of pro-inflammatory cytokines, 2) activation of intracellular PRRs (RIG-I, MDA5, etc, and/or 3) termination or reduction in protein translation.

Any of the regions of the polynucleotide comprising an mRNA encoding a polypeptide can be chemically modified as taught herein or as taught in International Application Pub. No. WO 2013/052523 A1.

### Modifications on the Sugar

The modified nucleosides and nucleotides (e.g., building block molecules), which can be incorporated into a polynucleotide (e.g., RNA or mRNA, as described herein) comprising an mRNA encoding a polypeptide, can be modified on the sugar of the ribonucleic acid. For example, the 2′ hydroxyl group (OH) can be modified or replaced with a number of different substituents. Exemplary substitutions at the 2′-position include, but are not limited to, H, halo, optionally substituted C₁₋₆ alkyl; optionally substituted C₁₋₆ alkoxy; optionally substituted C₆₋₁₀ aryloxy; optionally substituted C₃₋₈ cycloalkyl; optionally substituted C₃₋₈ cycloalkoxy; optionally substituted C₆₋₁₀ aryloxy; optionally substituted C₆₋₁₀ aryl-C₁₋₆ alkoxy, optionally substituted C₁₋₁₂ (heterocyclyl)oxy; a sugar (e.g., ribose, pentose, or any described herein); a polyethyleneglycol (PEG), —O(CH₂CH₂O)ₙCH₂CH₂OR, where R is H or optionally substituted alkyl, and n is an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20); “locked” nucleic acids (LNA) in which the 2′-hydroxyl is connected by a C₁₋₆ alkylene or C₁₋₆ heteroalkylene bridge to the 4′-carbon of the same ribose sugar, where exemplary bridges included methylene, propylene, ether, or amino bridges; aminoalkyl, as defined herein; aminoalkoxy, as defined herein; amino as defined herein; and amino acid, as defined herein.

Generally, RNA includes the sugar group ribose, which is a 5-membered ring having an oxygen. Exemplary, non-limiting modified nucleotides include replacement of the oxygen in ribose (e.g., with S, Se, or alkylene, such as methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for anhydrohexitol, altritol, mannitol, cyclohexanyl, cyclohexenyl, and morpholino that also has a phosphoramidate backbone); multicyclic forms (e.g., tricyclo; and “unlocked” forms, such as glycol nucleic acid (GNA) (e.g., R-GNA or S-GNA, where ribose is replaced by glycol units attached to phosphodiester bonds), threose nucleic acid (TNA, where ribose is replace with α-L-threofuranosyl-(3′→2′)), and peptide nucleic acid (PNA, where 2-amino-ethyl-glycine linkages replace the ribose and phosphodiester backbone). The sugar group can also contain one or more carbons that possess the opposite stereochemical configuration than that of the corresponding carbon in ribose. Thus, a polynucleotide molecule can include nucleotides containing, e.g., arabinose, as the sugar. Such sugar modifications are taught International Patent Application Pub. No. WO 2013052523 and International Patent Application Pub. No. WO 2014/093924.

### Combinations of Modified Sugars, Nucleobases, and Internucleoside Linkages

The polynucleotides comprising an mRNA encoding a polypeptide can include a combination of modifications to the sugar, the nucleobase, and/or the internucleoside linkage. These combinations can include any one or more modifications described herein.

Examples of modified nucleotides and modified nucleotide combinations are provided below in Table 5. These combinations of modified nucleotides can be used to form the polynucleotides. Unless otherwise noted, the modified nucleotides can be completely substituted for the natural nucleotides of the polynucleotides. As a non-limiting example, the natural nucleotide uridine can be substituted with a modified nucleoside described herein. In another non-limiting example, the natural nucleotide uridine can be partially substituted (e.g., about 0.1%, 1%, 5%, 10%, 15%, 20%, 25%, 30%, 35%, 40%, 45%, 50%, 55%, 60%, 65%, 70%, 75%, 80%, 85%, 90%, 95% or 99.9%) with at least one of the modified nucleoside disclosed herein. Any combination of base/sugar or linker can be incorporated into the polynucleotides and such modifications are taught in International Application Patent Publication Nos. WO 2013/052523 and WO 2014/093924 A1.

TABLE 5
Combinations

| Modified Nucleotide | Modified Nucleotide Combination |
|---|---|
| α-thio-cytidine | α-thio-cytidine/5-iodo-uridine |
| | α-thio-cytidine/N1-methyl-pseudouridine |
| | α-thio-cytidine/α-thio-uridine |
| | α-thio-cytidine/5-methyl-uridine |
| | α-thio-cytidine/pseudo-uridine |
| | about 50% of the cytosines are α-thio-cytidine |
| pseudoisocytidine | pseudoisocytidine/5-iodo-uridine |
| | pseudoisocytidine/N1-methyl-pseudouridine |
| | pseudoisocytidine/α-thio-uridine |
| | pseudoisocytidine/5-methyl-uridine |
| | pseudoisocytidine/pseudouridine |
| | about 25% of cytosines are pseudoisocytidine |
| | pseudoisocytidine/about 50% of uridines are N1- |
| | methyl-pseudouridine and about 50% of uridines |
| | are pseudouridine |
| | pseudoisocytidine/about 25% of uridines are N1- |
| | methyl-pseudouridine and about 25% of uridines |
| | are pseudouridine |
| pyrrolo-cytidine | pyrrolo-cytidine/5-iodo-uridine |
| | pyrrolo-cytidine/N1-methyl-pseudouridine |
| | pyrrolo-cytidine/α-thio-uridine |
| | pyrrolo-cytidine/5-methyl-uridine |
| | pyrrolo-cytidine/pseudouridine |
| | about 50% of the cytosines are pyrrolo-cytidine |
| 5-methyl-cytidine | 5-methyl-cytidine/5-iodo-uridine |
| | 5-methyl-cytidine/N1-methyl-pseudouridine |
| | 5-methyl-cytidine/α-thio-uridine |
| | 5-methyl-cytidine/5-methyl-uridine |
| | 5-methyl-cytidine/pseudouridine |
| | about 25% of cytosines are 5-methyl-cytidine |
| | about 50% of cytosines are 5-methyl-cytidine |
| | 5-methyl-cytidine/5-methoxy-uridine |
| | 5-methyl-cytidine/5-bromo-uridine |
| | 5-methyl-cytidine/2-thio-uridine |
| | 5-methyl-cytidine/about 50% of uridines are |
| | 2-thio-uridine |
| | about 50% of uridines are |
| | 5-methyl-cytidine/about |
| | 50% of uridines are 2-thio-uridine |
| N4-acetyl-cytidine | N4-acetyl-cytidine/5-iodo-uridine |
| | N4-acetyl-cytidine/N1-methyl-pseudouridine |
| | N4-acetyl-cytidine/α-thio-uridine |
| | N4-acetyl-cytidine/5-methyl-uridine |
| | N4-acetyl-cytidine/pseudouridine |
| | about 50% of cytosines are N4-acetyl-cytidine |
| | about 25% of cytosines are N4-acetyl-cytidine |
| | N4-acetyl-cytidine/5-methoxy-uridine |
| | N4-acetyl-cytidine/5-bromo-uridine |
| | N4-acetyl-cytidine/2-thio-uridine |
| | about 50% of cytosines are |
| | N4-acetyl-cytidine/about |
| | 50% of uridines are 2-thio-uridine |

Additional examples of modified nucleotides and modified nucleotide combinations are provided below in Table 6.

TABLE 6
Additional combinations

| Uracil | Cytosine | Adenine | Guanine |
|---|---|---|---|
| 5-methoxy-UTP | CTP | ATP | GTP |
| 5-Methoxy-UTP | N4Ac-CTP | ATP | GTP |
| 5-Methoxy-UTP | 5-Methyl-CTP | ATP | GTP |
| 5-Methoxy-UTP | 5-Trifluoromethyl-CTP | ATP | GTP |
| 5-Methoxy-UTP | 5-Hydroxymethyl-CTP | ATP | GTP |
| 5-Methoxy-UTP | 5-Bromo-CTP | ATP | GTP |
| 5-Methoxy-UTP | N4Ac-CTP | ATP | GTP |
| 5-Methoxy-UTP | CTP | ATP | GTP |
| 5-Methoxy-UTP | 5-Methyl-CTP | ATP | GTP |
| 5-Methoxy-UTP | 5-Trifluoromethyl-CTP | ATP | GTP |
| 5-Methoxy-UTP | 5-Hydroxymethyl-CTP | ATP | GTP |
| 5-Methoxy-UTP | 5-Bromo-CTP | ATP | GTP |
| 5-Methoxy-UTP | N4-Ac-CTP | ATP | GTP |
| 5-Methoxy-UTP | 5-Iodo-CTP | ATP | GTP |
| 5-Methoxy-UTP | 5-Bromo-CTP | ATP | GTP |
| 5-Methoxy-UTP | CTP | ATP | GTP |
| 5-Methoxy-UTP | 5-Methyl-CTP | ATP | GTP |
| 75% 5-Methoxy-UTP + | 5-Methyl-CTP | ATP | GTP |
| 25% UTP | | | |
| 50% 5-Methoxy-UTP + | 5-Methyl-CTP | ATP | GTP |
| 50% UTP | | | |
| 25% 5-Methoxy-UTP + | 5-Methyl-CTP | ATP | GTP |
| 75% UTP | | | |
| 5-Methoxy-UTP | 75% 5-Methyl-CTP + 25% | ATP | GTP |
| | CTP | | |
| 5-Methoxy-UTP | 50% 5-Methyl-CTP + 50% | ATP | GTP |
| | CTP | | |
| 5-Methoxy-UTP | 25% 5-Methyl-CTP + 75% | ATP | GTP |
| | CTP | | |
| 75% 5-Methoxy-UTP + | 75% 5-Methyl-CTP + 25% | ATP | GTP |
| 25% UTP | CTP | | |
| 75% 5-Methoxy-UTP + | 50% 5-Methyl-CTP + 50% | ATP | GTP |
| 25% UTP | | CTP | |
| 75% 5-Methoxy-UTP + | 25% 5-Methyl-CTP + 75% | ATP | GTP |
| 25% UTP | CTP | | |
| 50% 5-Methoxy-UTP + | 75% 5-Methyl-CTP + 25% | ATP | GTP |
| 50% UTP | CTP | | |
| 50% 5-Methoxy-UTP + | 50% 5-Methyl-CTP + 50% | ATP | GTP |
| 50% UTP | CTP | | |
| 50% 5-Methoxy-UTP + | 25% 5-Methyl-CTP + 75% | ATP | GTP |
| 50% UTP | CTP | | |
| 25% 5-Methoxy-UTP + | 75% 5-Methyl-CTP + 25% | ATP | GTP |
| 75% UTP | CTP | | |
| 25% 5-Methoxy-UTP + | 50% 5-Methyl-CTP + 50% | ATP | GTP |
| 75% UTP | CTP | | |
| 25% 5-Methoxy-UTP + | 25% 5-Methyl-CTP + 75% | ATP | GTP |
| 75% UTP | CTP | | |
| 75% 5-Methoxy-UTP + | CTP | ATP | GTP |
| 25% UTP | | | |
| 50% 5-Methoxy-UTP + | CTP | ATP | GTP |
| 50% UTP | | | |
| 25% 5-Methoxy-UTP + | CTP | ATP | GTP |
| 75% UTP | | | |
| 5-Methoxy-UTP | CTP | ATP | GTP |
| 5-Methoxy-UTP | CTP | ATP | GTP |
| 5-Methoxy-UTP | CTP | ATP | GTP |
| 5-Methoxy-UTP | 5-Methyl-CTP | ATP | GTP |
| 5-Methoxy-UTP | 5-Methyl-CTP | ATP | GTP |
| 5-Methoxy-UTP | 5-Methyl-CTP | ATP | GTP |
| 5-Methoxy-UTP | CTP | Alpha- | GTP |
| | | thio- | |
| | | ATP | |
| 5-Methoxy-UTP | 5-Methyl-CTP | Alpha- | GTP |
| | | thio- | |
| | | ATP | |
| 5-Methoxy-UTP | CTP | ATP | Alpha- |
| | | | thio- |
| | | | GTP |
| 5-Methoxy-UTP | 5-Methyl- | CTP | ATP |
| | | | Alpha- |
| | | | thio- |
| | | | GTP |
| 5-Methoxy-UTP | CTP | N6-Me- | GTP |
| | | ATP | |
| 5-Methoxy-UTP | 5-Methyl-CTP | N6-Me- | GTP |
| | | ATP | |
| 5-Methoxy-UTP | CTP | ATP | GTP |
| 5-Methoxy-UTP | 5-Methyl-CTP | ATP | GTP |
| 75% 5-Methoxy-UTP + | 5-Methyl-CTP | ATP | GTP |
| 25% UTP | | | |
| 50% 5-Methoxy-UTP + | 5-Methyl-CTP | ATP | GTP |
| 50% UTP | | | |
| 25% 5-Methoxy-UTP + | 5-Methyl-CTP | ATP | GTP |
| 75% UTP | | | |
| 5-Methoxy-UTP | 75% 5-Methyl-CTP + 25% | ATP | GTP |
| | CTP | | |
| 5-Methoxy-UTP | 50% 5-Methyl-CTP + 50% | ATP | GTP |
| | CTP | | |
| 5-Methoxy-UTP | 25% 5-Methyl-CTP + 75% | ATP | GTP |
| | CTP | | |
| 75% 5-Methoxy-UTP + | 75% 5-Methyl-CTP + 25% | ATP | GTP |
| 25% UTP | CTP | | |
| 75% 5-Methoxy-UTP + | 50% 5-Methyl-CTP + 50% | ATP | GTP |
| 25% UTP | CTP | | |
| 75% 5-Methoxy-UTP + | 25% 5-Methyl-CTP + 75% | ATP | GTP |
| 25% UTP | CTP | | |
| 50% 5-Methoxy-UTP + | 75% 5-Methyl-CTP + 25% | ATP | GTP |
| 50% UTP | CTP | | |
| 50% 5-Methoxy-UTP + | 50% 5-Methyl-CTP + 50% | ATP | GTP |
| 50% UTP | CTP | | |
| 50% 5-Methoxy-UTP + | 25% 5-Methyl-CTP + 75% | ATP | GTP |
| 50% UTP | CTP | | |
| 25% 5-Methoxy-UTP + | 75% 5-Methyl-CTP + 25% | ATP | GTP |
| 75% UTP | CTP | | |
| 25% 5-Methoxy-UTP + | 50% 5-Methyl-CTP + 50% | ATP | GTP |
| 75% UTP | CTP | | |
| 25% 5-Methoxy-UTP + | 25% 5-Methyl-CTP + 75% | ATP | GTP |
| 75% UTP | CTP | | |
| 75% 5-Methoxy-UTP + | CTP | ATP | GTP |
| 25% UTP | | | |
| 50% 5-Methoxy-UTP + | CTP | ATP | GTP |
| 50% UTP | | | |
| 25% 5-Methoxy-UTP + | CTP | ATP | GTP |
| 75% UTP | | | |
| 5-Methoxy-UTP | 5-Ethyl-CTP | ATP | GTP |
| 5-Methoxy-UTP | 5-Methoxy-CTP | ATP | GTP |
| 5-Methoxy-UTP | 5-Ethynyl-CTP | ATP | GTP |
| 5-Methoxy-UTP | CTP | ATP | GTP |
| 5-Methoxy-UTP | 5-Methyl-CTP | ATP | GTP |
| 5-Methoxy-UTP | CTP | ATP | GTP |
| 5-Methoxy-UTP | 5-Methyl-CTP | ATP | GTP |
| 75% 5-Methoxy-UTP + | 5-Methyl-CTP | ATP | GTP |
| 25% 1-Methyl-pseudo- | | | |
| UTP | | | |
| 50% 5-Methoxy-UTP + | 5-Methyl-CTP | ATP | GTP |
| 50% 1-Methyl-pseudo- | | | |
| UTP | | | |
| 25% 5-Methoxy-UTP + | 5-Methyl-CTP | ATP | GTP |
| 75% 1-Methyl-pseudo- | | | |
| UTP | | | |
| 5-Methoxy-UTP | 75% 5-Methyl-CTP + 25% | ATP | GTP |
| | CTP | | |
| 5-Methoxy-UTP | 50% 5-Methyl-CTP + 50% | ATP | GTP |
| | CTP | | |
| 5-Methoxy-UTP | 25% 5-Methyl-CTP + 75% | ATP | GTP |
| | CTP | | |
| 75% 5-Methoxy-UTP + | 75% 5-Methyl-CTP + 25% | ATP | GTP |
| 25% 1-Methyl-pseudo- | CTP | | |
| UTP | | | |
| 75% 5-Methoxy-UTP + | 50% 5-Methyl-CTP + 50% | ATP | GTP |
| 25% 1-Methyl-pseudo- | CTP | | |
| UTP | | | |
| 75% 5-Methoxy-UTP + | 25% 5-Methyl-CTP + 75% | ATP | GTP |
| 25% 1-Methyl-pseudo- | CTP | | |
| UTP | | | |
| 50% 5-Methoxy-UTP + | 75% 5-Methyl-CTP + 25% | ATP | GTP |
| 50% 1-Methyl-pseudo- | CTP | | |
| UTP | | | |
| 50% 5-Methoxy-UTP + | 50% 5-Methyl-CTP + 50% | ATP | GTP |
| 50% 1-Methyl-pseudo- | CTP | | |
| UTP | | | |
| 50% 5-Methoxy-UTP + | 25% 5-Methyl-CTP + 75% | ATP | GTP |
| 50% 1-Methyl-pseudo- | CTP | | |
| UTP | | | |
| 25% 5-Methoxy-UTP + | 75% 5-Methyl-CTP + 25% | ATP | GTP |
| 75% 1-Methyl-pseudo- | CTP | | |
| UTP | | | |
| 25% 5-Methoxy-UTP + | 50% 5-Methyl-CTP + 50% | ATP | GTP |
| 75% 1-Methyl-pseudo- | CTP | | |
| UTP | | | |
| 25% 5-Methoxy-UTP + | 25% 5-Methyl-CTP + 75% | ATP | GTP |
| 75% 1-Methyl-pseudo- | CTP | | |
| UTP | | | |
| 75% 5-Methoxy-UTP + | CTP | ATP | GTP |
| 25% 1-Methyl-pseudo- | | | |
| UTP | | | |
| 50% 5-Methoxy-UTP + | CTP | ATP | GTP |
| 50% 1-Methyl-pseudo- | | | |
| UTP | | | |
| 25% 5-Methoxy-UTP + | CTP | ATP | GTP |
| 75% 1-Methyl-pseudo- | | | |
| UTP | | | |
| 5-methoxy-UTP (In | CTP | ATP | GTP |
| House) | | | |
| 5-methoxy-UTP | CTP | ATP | GTP |
| (Hongene) | | | |
| 5-methoxy-UTP | 5-Methyl-CTP | ATP | GTP |
| (Hongene) | | | |
| 5-Methoxy-UTP | CTP | ATP | GTP |
| 5-Methoxy-UTP | 5-Methyl-CTP | ATP | GTP |
| 75% 5-Methoxy-UTP + | 5-Methyl-CTP | ATP | GTP |
| 25% UTP | | | |
| 50% 5-Methoxy-UTP + | 5-Methyl-CTP | ATP | GTP |
| 50% UTP | | | |
| 25% 5-Methoxy-UTP + | 5-Methyl-CTP | ATP | GTP |
| 75% UTP | | | |
| 5-Methoxy-UTP | 75% 5-Methyl-CTP + 25% | ATP | GTP |
| | CTP | | |
| 5-Methoxy-UTP | 50% 5-Methyl-CTP + 50% | ATP | GTP |
| | CTP | | |
| 5-Methoxy-UTP | 25% 5-Methyl-CTP + 75% | ATP | GTP |
| | CTP | | |
| 75% 5-Methoxy-UTP + | 75% 5-Methyl-CTP + 25% | ATP | GTP |
| 25% UTP | CTP | | |
| 75% 5-Methoxy-UTP + | 50% 5-Methyl-CTP + 50% | ATP | GTP |
| 25% UTP | CTP | | |
| 75% 5-Methoxy-UTP + | 25% 5-Methyl-CTP + 75% | ATP | GTP |
| 25% UTP | CTP | | |
| 50% 5-Methoxy-UTP + | 75% 5-Methyl-CTP + 25% | ATP | GTP |
| 50% UTP | CTP | | |
| 50% 5-Methoxy-UTP + | 50% 5-Methyl-CTP + 50% | ATP | GTP |
| 50% UTP | CTP | | |
| 50% 5-Methoxy-UTP + | 25% 5-Methyl-CTP + 75% | ATP | GTP |
| 50% UTP | CTP | | |
| 25% 5-Methoxy-UTP + | 75% 5-Methyl-CTP + 25% | ATP | GTP |
| 75% UTP | CTP | | |
| 25% 5-Methoxy-UTP + | 50% 5-Methyl-CTP + 50% | ATP | GTP |
| 75% UTP | CTP | | |
| 25% 5-Methoxy-UTP + | 25% 5-Methyl-CTP + 75% | ATP | GTP |
| 75% UTP | CTP | | |
| 75% 5-Methoxy-UTP + | CTP | ATP | GTP |
| 25% UTP | | | |
| 50% 5-Methoxy-UTP + | CTP | ATP | GTP |
| 50% UTP | | | |
| 25% 5-Methoxy-UTP + | CTP | ATP | GTP |
| 75% UTP | | | |
| 5-Methoxy-UTP | CTP | ATP | GTP |
| 5-Methoxy-UTP | 5-Methyl-CTP | ATP | GTP |
| 75% 5-Methoxy-UTP + | 5-Methyl-CTP | ATP | GTP |
| 25% UTP | | | |
| 50% 5-Methoxy-UTP + | 5-Methyl-CTP | ATP | GTP |
| 50% UTP | | | |
| 25% 5-Methoxy-UTP + | 5-Methyl-CTP | ATP | GTP |
| 75% UTP | | | |
| 5-Methoxy-UTP | 75% 5-Methyl-CTP + 25% | ATP | GTP |
| | CTP | | |
| 5-Methoxy-UTP | 50% 5-Methyl-CTP + 50% | ATP | GTP |
| | CTP | | |
| 5-Methoxy-UTP | 25% 5-Methyl-CTP + 75% | ATP | GTP |
| | CTP | | |
| 75% 5-Methoxy-UTP + | 75% 5-Methyl-CTP + 25% | ATP | GTP |
| 25% UTP | CTP | | |
| 75% 5-Methoxy-UTP + | 50% 5-Methyl-CTP + 50% | ATP | GTP |
| 25% UTP | CTP | | |
| 75% 5-Methoxy-UTP + | 25% 5-Methyl-CTP + 75% | ATP | GTP |
| 25% UTP | CTP | | |
| 50% 5-Methoxy-UTP + | 75% 5-Methyl-CTP + 25% | ATP | GTP |
| 50% UTP | CTP | | |
| 50% 5-Methoxy-UTP + | 50% 5-Methyl-CTP + 50% | ATP | GTP |
| 50% UTP | CTP | | |
| 50% 5-Methoxy-UTP + | 25% 5-Methyl-CTP + 75% | ATP | GTP |
| 50% UTP | CTP | | |
| 25% 5-Methoxy-UTP + | 75% 5-Methyl-CTP + 25% | ATP | GTP |
| 75% UTP | CTP | | |
| 25% 5-Methoxy-UTP + | 50% 5-Methyl-CTP + 50% | ATP | GTP |
| 75% UTP | CTP | | |
| 25% 5-Methoxy-UTP + | 25% 5-Methyl-CTP + 75% | ATP | GTP |
| 75% UTP | CTP | | |
| 75% 5-Methoxy-UTP + | CTP | ATP | GTP |
| 25% UTP | | | |
| 50% 5-Methoxy-UTP + | CTP | ATP | GTP |
| 50% UTP | | | |
| 25% 5-Methoxy-UTP + | CTP | ATP | GTP |
| 75% UTP | | | |
| 5-Methoxy-UTP | CTP | ATP | GTP |
| 25% 5-Methoxy-UTP + | 75% 5-Methyl-CTP + 25% | ATP | GTP |
| 75% UTP | CTP | | |
| 25% 5-Methoxy-UTP + | 50% 5-Methyl-CTP + 50% | ATP | GTP |
| 75% UTP | CTP | | |
| 25% 5-Methoxy-UTP + | 25% 5-Methyl-CTP + 75% | ATP | GTP |
| 75% UTP | CTP | | |
| 75% 5-Methoxy-UTP + | 75% 5-Methyl-CTP + 25% | ATP | GTP |
| 25% UTP | CTP | | |
| 5-Methoxy-UTP | CTP | ATP | GTP |
| 25% 5-Methoxy-UTP + | 75% 5-Methyl-CTP + 25% | ATP | GTP |
| 75% UTP | CTP | | |
| 25% 5-Methoxy-UTP + | 50% 5-Methyl-CTP + 50% | ATP | GTP |
| 75% UTP | CTP | | |
| 25% 5-Methoxy-UTP + | 25% 5-Methyl-CTP + 75% | ATP | GTP |
| 75% UTP | CTP | | |
| 75% 5-Methoxy-UTP + | 75% 5-Methyl-CTP + 25% | ATP | GTP |
| 25% UTP | CTP | | |
| 5-Methoxy-UTP | CTP | ATP | GTP |
| 25% 5-Methoxy-UTP + | 75% 5-Methyl-CTP + 25% | ATP | GTP |
| 75% UTP | CTP | | |
| 25% 5-Methoxy-UTP + | 50% 5-Methyl-CTP + 50% | ATP | GTP |
| 75% UTP | CTP | | |
| 25% 5-Methoxy-UTP + | 25% 5-Methyl-CTP + 75% | ATP | GTP |
| 75% UTP | CTP | | |
| 75% 5-Methoxy-UTP + | 75% 5-Methyl-CTP + 25% | ATP | GTP |
| 25% UTP | CTP | | |
| 5-Methoxy-UTP | CTP | ATP | GTP |
| 25% 5-Methoxy-UTP + | 75% 5-Methyl-CTP + 25% | ATP | GTP |
| 75% UTP | CTP | | |
| 25% 5-Methoxy-UTP + | 50% 5-Methyl-CTP + 50% | ATP | GTP |
| 75% UTP | CTP | | |
| 25% 5-Methoxy-UTP + | 25% 5-Methyl-CTP + 75% | ATP | GTP |
| 75% UTP | CTP | | |
| 75% 5-Methoxy-UTP + | 75% 5-Methyl-CTP + 25% | ATP | GTP |
| 25% UTP | CTP | | |
| 5-Methoxy-UTP | CTP | ATP | GTP |
| 25% 5-Methoxy-UTP + | 75% 5-Methyl-CTP + 25% | ATP | GTP |
| 75% UTP | CTP | | |
| 25% 5-Methoxy-UTP + | 50% 5-Methyl-CTP + 50% | ATP | GTP |
| 75% UTP | CTP | | |
| 25% 5-Methoxy-UTP + | 25% 5-Methyl-CTP + 75% | ATP | GTP |
| 75% UTP | CTP | | |
| 75% 5-Methoxy-UTP + | 75% 5-Methyl-CTP + 25% | ATP | GTP |
| 25% UTP | CTP | | |
| 5-Methoxy-UTP | CTP | ATP | GTP |
| 25% 5-Methoxy-UTP + | 75% 5-Methyl-CTP + 25% | ATP | GTP |
| 75% UTP | CTP | | |
| 25% 5-Methoxy-UTP + | 50% 5-Methyl-CTP + 50% | ATP | GTP |
| 75% UTP | CTP | | |
| 25% 5-Methoxy-UTP + | 25% 5-Methyl-CTP + 75% | ATP | GTP |
| 75% UTP | CTP | | |
| 75% 5-Methoxy-UTP + | 75% 5-Methyl-CTP + 25% | ATP | GTP |
| 25% UTP | CTP | | |
| 5-Methoxy-UTP | CTP | ATP | GTP |
| 25% 5-Methoxy-UTP + | 75% 5-Methyl-CTP + 25% | ATP | GTP |
| 75% UTP | CTP | | |
| 25% 5-Methoxy-UTP + | 50% 5-Methyl-CTP + 50% | ATP | GTP |
| 75% UTP | CTP | | |
| 25% 5-Methoxy-UTP + | 25% 5-Methyl-CTP + 75% | ATP | GTP |
| 75% UTP | CTP | | |
| 75% 5-Methoxy-UTP + | 75% 5-Methyl-CTP + 25% | ATP | GTP |
| 25% UTP | CTP | | |
| 5-Methoxy-UTP | 5-Fluoro-CTP | ATP | GTP |
| 5-Methoxy-UTP | 5-Phenyl-CTP | ATP | GTP |
| 5-Methoxy-UTP | N4-Bz-CTP | ATP | GTP |
| 5-Methoxy-UTP | CTP | N6- | GTP |
| | | Iso- | |
| | | pentenyl- | |
| | | ATP | |
| 5-Methoxy-UTP | N4-Ac-CTP | ATP | GTP |
| 25% 5-Methoxy-UTP + | 25% N4-Ac-CTP + 75% | ATP | GTP |
| 75% UTP | CTP | | |
| 25% 5-Methoxy-UTP + | 75% N4-Ac-CTP + 25% | ATP | GTP |
| 75% UTP | CTP | | |
| 75% 5-Methoxy-UTP + | 25% N4-Ac-CTP + 75% | ATP | GTP |
| 25% UTP | CTP | | |
| 75% 5-Methoxy-UTP + | 75% N4-Ac-CTP + 25% | ATP | GTP |
| 25% UTP | CTP | | |
| 5-Methoxy-UTP | 5-Hydroxymethyl-CTP | ATP | GTP |
| 25% 5-Methoxy-UTP + | 25% 5-Hydroxymethyl- | ATP | GTP |
| 75% UTP | CTP + 75% CTP | | |
| 25% 5-Methoxy-UTP + | 75% 5-Hydroxymethyl- | ATP | GTP |
| 75% UTP | CTP + 25% CTP | | |
| 75% 5-Methoxy-UTP + | 25% 5-Hydroxymethyl- | ATP | GTP |
| 25% UTP | CTP + 75% CTP | | |
| 75% 5-Methoxy-UTP + | 75% 5-Hydroxymethyl- | ATP | GTP |
| 25% UTP | CTP + 25% CTP | | |
| 5-Methoxy-UTP | N4-Methyl CTP | ATP | GTP |
| 25% 5-Methoxy-UTP + | 25% N4-Methyl | ATP | GTP |
| 75% UTP | CTP + 75% CTP | | |
| 25% 5-Methoxy-UTP + | 75% N4-Methyl | ATP | GTP |
| 75% UTP | CTP + 25% CTP | | |
| 75% 5-Methoxy-UTP + | 25% N4-Methyl | ATP | GTP |
| 25% UTP | CTP + 75% CTP | | |
| 75% 5-Methoxy-UTP + | 75% N4-Methyl | ATP | GTP |
| 25% UTP | CTP + 25% CTP | | |
| 5-Methoxy-UTP | 5-Trifluoromethyl-CTP | ATP | GTP |
| 25% 5-Methoxy-UTP + | 25% 5-Trifluoromethyl- | ATP | GTP |
| 75% UTP | CTP + 75% CTP | | |
| 25% 5-Methoxy-UTP + | 75% 5-Trifluoromethyl- | ATP | GTP |
| 75% UTP | CTP + 25% CTP | | |
| 75% 5-Methoxy-UTP + | 25% 5-Trifluoromethyl- | ATP | GTP |
| 25% UTP | CTP + 75% CTP | | |
| 75% 5-Methoxy-UTP + | 75% 5-Trifluoromethyl- | ATP | GTP |
| 25% UTP | CTP + 25% CTP | | |
| 5-Methoxy-UTP | 5-Bromo-CTP | ATP | GTP |
| 25% 5-Methoxy-UTP + | 25% 5-Bromo-CTP + 75% | ATP | GTP |
| 75% UTP | CTP | | |
| 25% 5-Methoxy-UTP + | 75% 5-Bromo-CTP + 25% | ATP | GTP |
| 75% UTP | CTP | | |
| 75% 5-Methoxy-UTP + | 25% 5-Bromo-CTP + 75% | ATP | GTP |
| 25% UTP | CTP | | |
| 75% 5-Methoxy-UTP + | 75% 5-Bromo-CTP + 25% | ATP | GTP |
| 25% UTP | CTP | | |
| 5-Methoxy-UTP | 5-Iodo-CTP | ATP | GTP |
| 25% 5-Methoxy-UTP + | 25% 5-Iodo-CTP +7 5% | ATP | GTP |
| 75% UTP | CTP | | |
| 25% 5-Methoxy-UTP + | 75% 5-Iodo-CTP + 25% | ATP | GTP |
| 75% UTP | CTP | | |
| 75% 5-Methoxy-UTP + | 25% 5-Iodo-CTP + 75% | ATP | GTP |
| 25% UTP | CTP | | |
| 75% 5-Methoxy-UTP + | 75% 5-Iodo-CTP + 25% | ATP | GTP |
| 25% UTP | CTP | | |
| 5-Methoxy-UTP | 5-Ethyl-CTP | ATP | GTP |
| 25% 5-Methoxy-UTP + | 25% 5-Ethyl-CTP + 75% | ATP | GTP |
| 75% UTP | CTP | | |
| 25% 5-Methoxy-UTP + | 75% 5-Ethyl-CTP + 25% | ATP | GTP |
| 75% UTP | CTP | | |
| 75% 5-Methoxy-UTP + | 25% 5-Ethyl-CTP + 75% | ATP | GTP |
| 25% UTP | CTP | | |
| 75% 5-Methoxy-UTP + | 75% 5-Ethyl-CTP + 25% | ATP | GTP |
| 25% UTP | CTP | | |
| 5-Methoxy-UTP | 5-Methoxy-CTP | ATP | GTP |
| 25% 5-Methoxy-UTP + | 25% 5-Methoxy- | ATP | GTP |
| 75% UTP | CTP + 75% CTP | | |
| 25% 5-Methoxy-UTP + | 75% 5-Methoxy- | ATP | GTP |
| 75% UTP | CTP + 25% CTP | | |
| 75% 5-Methoxy-UTP + | 25% 5-Methoxy- | ATP | GTP |
| 25% UTP | CTP +75% CTP | | |
| 75% 5-Methoxy-UTP + | 75% 5-Methoxy- | ATP | GTP |
| 25% UTP | CTP + 25% CTP | | |
| 5-Methoxy-UTP | 5-Ethynyl-CTP | ATP | GTP |
| 25% 5-Methoxy-UTP + | 25% 5-Ethynyl- | ATP | GTP |
| 75% UTP | CTP + 75% CTP | | |
| 25% 5-Methoxy-UTP + | 75% 5-Ethynyl- | ATP | GTP |
| 75% UTP | CTP + 25% CTP | | |
| 75% 5-Methoxy-UTP + | 25% 5-Ethynyl- | ATP | GTP |
| 25% UTP | CTP + 75% CTP | | |
| 75% 5-Methoxy-UTP + | 75% 5-Ethynyl- | ATP | GTP |
| 25% UTP | CTP + 25% CTP | | |
| 5-Methoxy-UTP | 5-Pseudo-iso-CTP | ATP | GTP |
| 25% 5-Methoxy-UTP + | 25% 5-Pseudo-iso- | ATP | GTP |
| 75% UTP | CTP + 75% CTP | | |
| 25% 5-Methoxy-UTP + | 75% 5-Pseudo-iso- | ATP | GTP |
| 75% UTP | CTP + 25% CTP | | |
| 75% 5-Methoxy-UTP + | 25% 5-Pseudo-iso- | ATP | GTP |
| 25% UTP | CTP + 75% CTP | | |
| 75% 5-Methoxy-UTP + | 75% 5-Pseudo-iso- | ATP | GTP |
| 25% UTP | CTP + 25% CTP | | |
| 5-Methoxy-UTP | 5-Formyl-CTP | ATP | GTP |
| 25% 5-Methoxy-UTP + | 25% 5-Formyl- | ATP | GTP |
| 75% UTP | CTP + 75% CTP | | |
| 25% 5-Methoxy-UTP + | 75% 5-Formyl- | ATP | GTP |
| 75% UTP | CTP + 25% CTP | | |
| 75% 5-Methoxy-UTP + | 25% 5-Formyl- | ATP | GTP |
| 25% UTP | CTP + 75% CTP | | |
| 75% 5-Methoxy-UTP + | 75% 5-Formyl- | ATP | GTP |
| 25% UTP | CTP + 25% CTP | | |
| 5-Methoxy-UTP | 5-Aminoallyl-CTP | ATP | GTP |
| 25% 5-Methoxy-UTP + | 25% 5-Aminoallyl- | ATP | GTP |
| 75% UTP | CTP + 75% CTP | | |
| 25% 5-Methoxy-UTP + | 75% 5-Aminoallyl- | ATP | GTP |
| 75% UTP | CTP + 25% CTP | | |
| 75% 5-Methoxy-UTP + | 25% 5-Aminoallyl- | ATP | GTP |
| 25% UTP | CTP + 75% CTP | | |
| 75% 5-Methoxy-UTP + | 75% 5-Aminoallyl- | ATP | GTP |
| 25% UTP | CTP + 25% CTP | | |

### EXAMPLES

### Example 1

### Synthesis of Compounds According to Formula (I)

### A. General Considerations

All solvents and reagents used were obtained commercially and used as such unless noted otherwise. ¹H NMR spectra were recorded in CDCl₃, at 300 K using a Bruker Ultrashield 300 MHz instrument. Chemical shifts are reported as parts per million (ppm) relative to TMS (0.00) for ¹H. Silica gel chromatographies were performed on ISCO CombiFlash Rf+ Lumen Instruments using ISCO RediSep Rf Gold Flash Cartridges (particle size: 20-40 microns). Reverse phase chromatographies were performed on ISCO CombiFlash Rf+ Lumen Instruments using RediSep Rf Gold C18 High Performance columns. All final compounds were determined to be greater than 85% pure via analysis by reverse phase UPLC-MS (retention times, RT, in minutes) using Waters Acquity UPLC instrument with DAD and ELSD and a ZORBAX Rapid Resolution High Definition (RRHD) SB-C18 LC column, 2.1 mm, 50 mm, 1.8 μm, and a gradient of 65 to 100% acetonitrile in water with 0.1% TFA over 5 minutes at 1.2 mL/min. Injection volume was 5 μL and the column temperature was 80° C. Detection was based on electrospray ionization (ESI) in positive mode using Waters SQD mass spectrometer (Milford, Mass., USA) and evaporative light scattering detector.

The representative procedures described below are useful in the synthesis of Compounds 1-147.

The following abbreviations are employed herein:

THF: Tetrahydrofuran

DMAP: 4-Dimethylaminopyridine

LDA: Lithium Diisopropylamide

rt: Room Temperature

DME: 1,2-Dimethoxyethane

n-BuLi: n-Butyllithium

### B. Compound 2: Heptadecan-9-yl 8-((2-hydroxyethyl)(tetradecyl)amino) octanoate

### Representative Procedure 1:

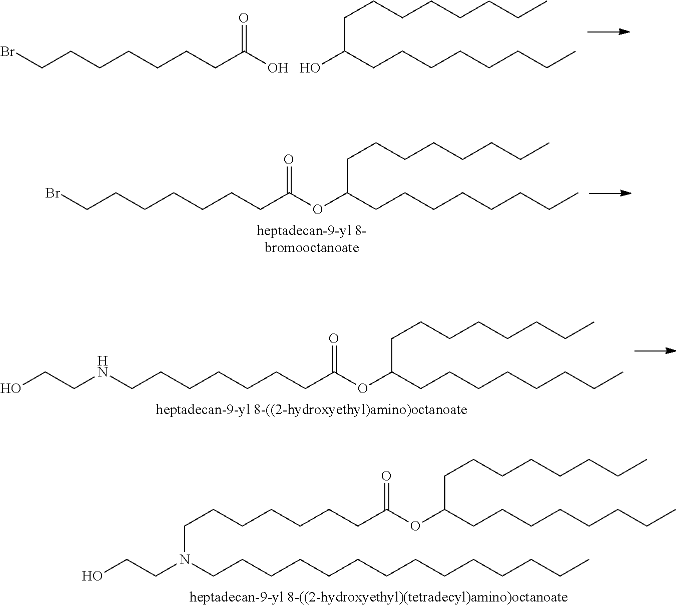

### Heptadecan-9-yl 8-bromooctanoate (Method A)

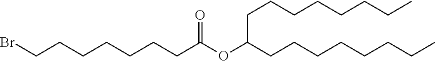 [SMILES: CCCCCCCCC(CCCCCCCC)OC(=O)CCCCCCCBr]

To a solution of 8-bromooctanoic acid (1.04 g, 4.6 mmol) and heptadecan-9-ol (1.5 g, 5.8 mmol) in dichloromethane (20 mL) was added N-(3-dimethylaminopropyl)-N′-ethylcarbodiimide hydrochloride (1.1 g, 5.8 mmol), N,N-diisopropylethylamine (3.3 mL, 18.7 mmol) and DMAP (114 mg, 0.9 mmol). The reaction was allowed to stir at rt for 18 h. The reaction was diluted with dichloromethane and washed with saturated sodium bicarbonate. The organic layer was separated and washed with brine, and dried over MgSO₄. The organic layer was filtered and evaporated in vacuo. The residue was purified by silica gel chromatography (0-10% ethyl acetate in hexanes) to obtain heptadecan-9-yl 8-bromooctanoate (875 mg, 1.9 mmol, 41%).

¹H NMR (300 MHz, CDCl₃) δ: ppm 4.89 (m, 1H); 3.42 (m, 2H); 2.31 (m, 2H); 1.89 (m, 2H); 1.73-1.18 (br. m, 36H); 0.88 (m, 6H).

### Heptadecan-9-yl 8-((2-hydroxyethyl)amino)octanoate (Method B)

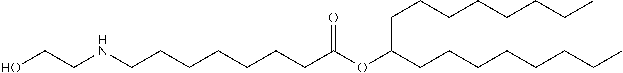 [SMILES: CCCCCCCCC(CCCCCCCC)OC(=O)CCCCCCCNCCO]

A solution of heptadecan-9-yl 8-bromooctanoate (3.8 g, 8.2 mmol) and 2-aminoethan-1-ol (15 mL, 248 mmol) in ethanol (3 mL) was allowed to stir at 62° C. for 18 h. The reaction mixture was concentrated in vacuo and the residue was taken-up in ethyl acetate and water. The organic layer was separated and washed with water, brine and dried over Na₂SO₄. The mixture was filtered and evaporated in vacuo. The residue was purified by silica gel chromatography (0-100% (mixture of 1% NH₄OH, 20% MeOH in dichloromethane) in dichloromethane) to obtain heptadecan-9-yl 8-((2-hydroxyethyl)amino)octanoate (3.1 g, 7 mmol, 85%). UPLC/ELSD: RT=2.67 min. MS (ES): m/z (MH⁺) 442.68 for C₂₇H₅₅NO₃

¹H NMR (300 MHz, CDCl₃) δ: ppm 4.89 (p, 1H); 3.67 (t, 2H); 2.81 (t, 2H); 2.65 (t, 2H); 2.30 (t, 2H); 2.05 (br. m, 2H); 1.72-1.41 (br. m, 8H); 1.40-1.20 (br. m, 30H); 0.88 (m, 6H).

### Heptadecan-9-yl 8-((2-hydroxyethyl)(tetradecyl)amino)octanoate (Method C)

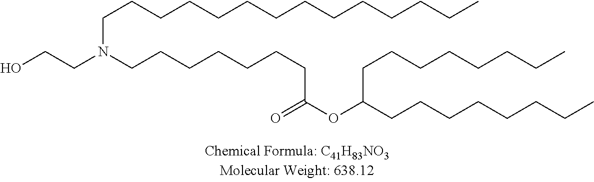 [SMILES: CCCCCCCCCCCCCCN(CCO)CCCCCCCC(=O)OC(CCCCCCCC)CCCCCCCC]

A solution of heptadecan-9-yl 8-((2-hydroxyethyl)amino)octanoate (125 mg, 0.28 mmol), 1-bromotetradecane (94 mg, 0.34 mmol) and N,N-diisopropylethylamine (44 mg, 0.34 mmol) in ethanol was allowed to stir at 65° C. for 18 h. The reaction was cooled to rt and solvents were evaporated in vacuo. The residue was taken-up in ethyl acetate and saturated sodium bicarbonate. The organic layer was separated, dried over Na₂SO₄ and evaporated in vacuo. The residue was purified by silica gel chromatography (0-100% (mixture of 1% NH₄OH, 20% MeOH in dichloromethane) in dichloromethane) to obtain heptadecan-9-yl 8-((2-hydroxyethyl)(tetradecyl)amino)octanoate (89 mg, 0.14 mmol, 50%). UPLC/ELSD: RT=3.61 min. MS (ES): m/z (MH⁻) 638.91 for C₄₁H₈₃NO₃. ¹H NMR (300 MHz, CDCl₃) δ: ppm 4.86 (p, 1H); 3.72-3.47 (br. m, 2H); 2.78-2.40 (br. m, 5H); 2.28 (t, 2H); 1.70-1.40 (m, 10H); 1.38-1.17 (br. m, 54H); 0.88 (m, 9H).

### Synthesis of Intermediates:

### Intermediate A: 2-Octyldecanoic Acid

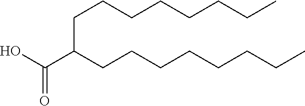 [SMILES: CCCCCCCCC(CCCCCCCC)C(=O)O]

A solution of diisopropylamine (2.92 mL, 20.8 mmol) in THF (10 mL) was cooled to −78° C. and a solution of n-BuLi (7.5 mL, 18.9 mmol, 2.5 M in hexanes) was added. The reaction was allowed to warm to 0° C. To a solution of decanoic acid (2.96 g, 17.2 mmol) and NaH (754 mg, 18.9 mmol, 60%w/w) in THF (20 mL) at 0° C. was added the solution of LDA and the mixture was allowed to stir at rt for 30 min. After this time 1-iodooctane (5 g, 20.8 mmol) was added and the reaction mixture was heated at 45° C. for 6 h. The reaction was quenched with 1N HCl (10 mL). The organic layer was dried over MgSO₄, filtered and evaporated in vacuo. The residue was purified by silica gel chromatography (0-20% ethyl acetate in hexanes) to yield 2-octyldecanoic acid (1.9 g, 6.6 mmol, 38%). ¹H NMR (300 MHz, CDCl₃) δ: ppm 2.38 (br. m, 1H); 1.74-1.03 (br. m, 28H); 0.91 (m, 6H).

### Intermediate B: 7-Bromoheptyl 2-octyldecanoate

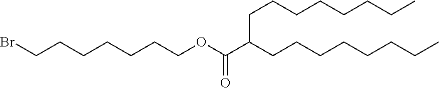 [SMILES: CCCCCCCCC(CCCCCCCC)C(=O)OCCCCCCCBr]

7-bromoheptyl 2-octyldecanoate was synthesized using Method A from 2-octyldecanoic acid and 7-bromoheptan-1-ol. ¹H NMR (300 MHz, CDCl₃) δ: ppm 4.09 (br. m, 2H); 3.43 (br. m, 2H); 2.48-2.25 (br. m, 1H); 1.89 (br. m, 2H); 1.74-1.16 (br. m, 36H); 0.90 (m, 6H).

### Intermediate C: (2-Hexylcyclopropyl)methanol

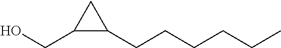

A solution of diethyl zinc (20 mL, 20 mmol, 1 M in hexanes), in dichloromethane (20 mL) was allowed to cool to −40° C. for 5 min. Then a solution of diiodomethane (3.22 mL, 40 mmol) in dichloromethane (10 mL) was added dropwise. After the reaction was allowed to stir for 1 h at −40° C., a solution of trichloro-acetic acid (327 mg, 2 mmol) and DME (1 mL, 9.6 mmol) in dichloromethane (10 mL) was added. The reaction was allowed to warm to −15° C. and stir at this temperature for 1 h. A solution of (Z)-non-2-en-1-ol (1.42 g, 10 mmol) in dichloromethane (10 mL) was then added to the −15° C. solution. The reaction was then slowly allowed to warm to rt and stir for 18 h. After this time saturated NH₄Cl (200 mL) was added and the reaction was extracted with dichloromethane (3×), washed with brine, and dried over Na₂SO₄. The organic layer was filtered, evaporated in vacuo and the residue was purified by silica gel chromatography (0-50% ethyl acetate in hexanes) to yield (2-hexylcyclopropyl)methanol (1.43 g, 9.2 mmol, 92%). ¹H NMR (300 MHz, CDCl₃) δ: ppm 3.64 (m, 2H); 1.57-1.02 (m, 12H); 0.99-0.80 (m, 4H); 0.72 (m, 1H), 0.00 (m, 1H).

### C. Compound 18: Heptadecan-9-yl 8-((2-hydroxyethyl)(8-(nonyloxy)-8-oxooctyl)amino) octanoate

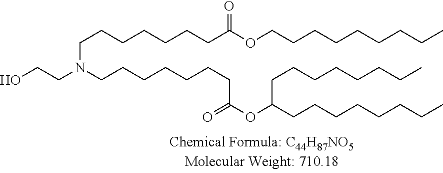 [SMILES: CCCCCCCCCOC(=O)CCCCCCCN(CCO)CCCCCCCC(=O)OC(CCCCCCCC)CCCCCCCC]

Compound 18 was synthesized according to the general procedure and Representative Procedure 1 described above.

UPLC/ELSD: RT=3.59 min. MS (ES): m/z (MH⁺) 710.89 for C₄₄H₈₇NO₅. ¹H NMR (300 MHz, CDCl₃) δ: ppm 4.86 (m, 1H); 4.05 (t, 2H); 3.53 (br. m, 2H); 2.83-2.36 (br. m, 5H); 2.29 (m, 4H); 0.96-1.71 (m, 64H); 0.88 (m, 9H).

### D. Compound 136: Nonyl 8-((2-hydroxyethyl)((9Z,12Z)-octadeca-9,12-dien-1-yl)amino)octanoate

### Representative Procedure 2:

### Nonyl 8-bromooctanoate (Method A)

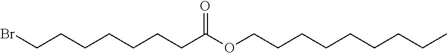 [SMILES: CCCCCCCCCOC(=O)CCCCCCCBr]

To a solution of 8-bromooctanoic acid (5 g, 22 mmol) and nonan-1-ol (6.46 g, 45 mmol) in dichloromethane (100 mL) were added N-(3-Dimethylaminopropyl)-N′-ethylcarbodiimide hydrochloride (4.3 g, 22 mmol) and DMAP (547 mg, 4.5 mmol). The reaction was allowed to stir at rt for 18 h. The reaction was diluted with dichloromethane and washed with saturated sodium bicarbonate. The organic layer was separated and washed with brine, dried over MgSO₄. The organic layer was filtered and evaporated under vacuum. The residue was purified by silica gel chromatography (0-10% ethyl acetate in hexanes) to obtain nonyl 8-bromooctanoate (6.1 g, 17 mmol, 77%).

¹H NMR (300 MHz, CDCl₃) δ: ppm 4.06 (t, 2H); 3.40 (t, 2H); 2.29 (t, 2H); 1.85 (m, 2H); 1.72-0.97 (m, 22H); 0.88 (m, 3H).

### Nonyl 8-((2-hydroxyethyl)amino)octanoate

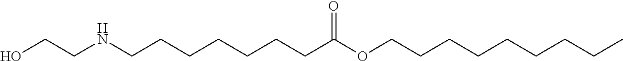 [SMILES: CCCCCCCCCOC(=O)CCCCCCCNCCO]

A solution of nonyl 8-bromooctanoate (1.2 g, 3.4 mmol) and 2-aminoethan-1-ol (5 mL, 83 mmol) in ethanol (2 mL) was allowed to stir at 62° C. for 18 h. The reaction mixture was concentrated in vacuum and the residue was extracted with ethyl acetate and water. The organic layer was separated and washed with water, brine and dried over Na₂SO₄. The organic layer was filtered and evaporated in vacuo. The residue was purified by silica gel chromatography (0-100% (mixture of 1% NH₄OH, 20% MeOH in dichloromethane) in dichloromethane) to obtain nonyl 8-((2-hydroxyethyl)amino)octanoate (295 mg, 0.9 mmol, 26%).

UPLC/ELSD: RT=1.29 min. MS (ES): m/z (MH⁺) 330.42 for C₁₉H₃₉NO₃

¹H NMR (300 MHz, CDCl₃) δ: ppm 4.07 (t, 2H); 3.65 (t, 2H); 2.78 (t, 2H); 2.63 (t, 2H); 2.32-2.19 (m, 4H); 1.73-1.20 (m, 24H); 0.89 (m, 3H)

### Nonyl 8-((2-hydroxyethyl)((9Z,12Z)-octadeca-9,12-dien-1-yl)amino)octanoate

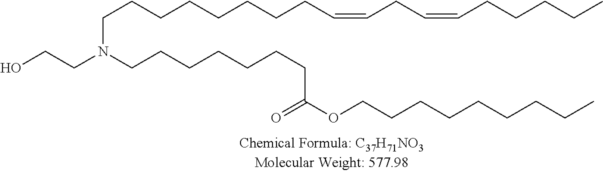 [SMILES: CCCCC/C=C\C/C=C\CCCCCCCCN(CCO)CCCCCCCC(=O)OCCCCCCCCC]

A solution of nonyl 8-((2-hydroxyethyl)amino)octanoate (150 mg, 0.46 mmol), (6Z,9Z)-18-bromooctadeca-6,9-diene (165 mg, 0.5 mmol) and N,N-diisopropylethylamine (65 mg, 0.5 mmol) in ethanol (2 mL) was allowed to stir at reflux for 48 h. The reaction was allowed to cool to rt and solvents were evaporated under vacuum. The residue was purified by silica gel chromatography (0-10% MeOH in dichloromethane) to obtain nonyl 8-((2-hydroxyethyl)((9Z,12Z)-octadeca-9,12-dien-1-yl)amino)octanoate (81 mg, 0.14 mmol, 30%) as a HBr salt.

UPLC/ELSD: RT=3.24 min. MS (ES): m/z (MH⁺) 578.64 for C₃₇H₇₁NO₃

¹H NMR (300 MHz, CDCl₃) δ: ppm 10.71 (br., 1H); 5.36 (br. m, 4H); 4.04 (m, 4H); 3.22-2.96 (br. m, 5H); 2.77 (m, 2H); 2.29 (m, 2H); 2.04 (br. m, 4H); 1.86 (br. m, 4H); 1.66-1.17 (br. m, 40H); 0.89 (m, 6H)

### E. Compound 138: Dinonyl 8,8′-((2-hydroxyethyl)azanediyl)dioctanoate

### Representative Procedure 3:

### Dinonyl 8,8′-((2-hydroxyethyl)azanediyl)dioctanoate

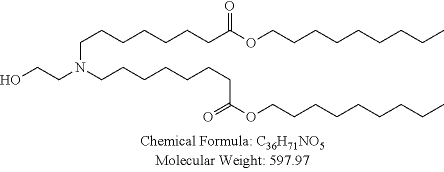 [SMILES: CCCCCCCCCOC(=O)CCCCCCCN(CCO)CCCCCCCC(=O)OCCCCCCCCC]

A solution of nonyl 8-bromooctanoate (200 mg, 0.6 mmol) and 2-aminoethan-1-ol (16 mg, 0.3 mmol) and N,N-diisopropylethylamine (74 mg, 0.6 mmol) in THF/CH₃CN (1:1) (3 mL) was allowed to stir at 63° C. for 72 h. The reaction was cooled to rt and solvents were evaporated under vacuum. The residue was extracted with ethyl acetate and saturated sodium bicarbonate. The organic layer was separated, dried over Na₂SO₄ and evaporated under vacuum. The residue was purified by silica gel chromatography (0-10% MeOH in dichloromethane) to obtain dinonyl 8,8′-((2-hydroxyethyl)azanediyl)dioctanoate (80 mg, 0.13 mmol, 43%).

UPLC/ELSD: RT=3.09 min. MS (ES): m/z (MH⁺) 598.85 for C₃₆H₇₁NO₅

¹H NMR (300 MHz, CDCl₃) δ: ppm 4.05 (m, 4H); 3.57 (br. m, 2H); 2.71-2.38 (br.

m, 6H); 2.29 (m, 4H), 1.71-1.01 (br. m, 49H), 0.88 (m, 6H).

All other compounds of formula (I) of this disclosure can be obtained by a method analogous to Representative Procedures 1-3 as described above.

### Example 2

### Characterization of Nanoparticle Compositions

A Zetasizer Nano ZS (Malvern Instruments Ltd, Malvern, Worcestershire, UK) can be used to determine the particle size, the polydispersity index (PDI) and the zeta potential of the nanoparticle compositions in 1× PBS in determining particle size and 15 mM PBS in determining zeta potential.

Ultraviolet-visible spectroscopy can be used to determine the concentration of a polynucleotide (e.g., RNA) in nanoparticle compositions. 100 μL of the diluted formulation in 1× PBS is added to 900 μL of a 4:1 (v/v) mixture of methanol and chloroform. After mixing, the absorbance spectrum of the solution is recorded, for example, between 230 nm and 330 nm on a DU 800 spectrophotometer (Beckman Coulter, Beckman Coulter, Inc., Brea, Calif.). The concentration of polynucleotide in the nanoparticle composition can be calculated based on the extinction coefficient of the polynucleotide used in the composition and on the difference between the absorbance at a wavelength of, for example, 260 nm and the baseline value at a wavelength of, for example, 330 nm.

For nanoparticle compositions including an RNA, a QUANT-IT™ RIBOGREEN® RNA assay (Invitrogen Corporation Carlsbad, Calif.) can be used to evaluate the encapsulation of an RNA by the nanoparticle composition. The samples are diluted to a concentration of approximately 5 μg/mL in a TE buffer solution (10 mM Tris-HCl, 1 mM EDTA, pH 7.5). 50 μL of the diluted samples are transferred to a polystyrene 96 well plate and either 50 μL of TE buffer or 50 μL of a 2% Triton X-100 solution is added to the wells. The plate is incubated at a temperature of 37° C. for 15 minutes. The RIBOGREEN® reagent is diluted 1:100 in TE buffer, and 100 μL of this solution is added to each well. The fluorescence intensity can be measured using a fluorescence plate reader (Wallac Victor 1420 Multilablel Counter; Perkin Elmer, Waltham, Mass.) at an excitation wavelength of, for example, about 480 nm and an emission wavelength of, for example, about 520 nm. The fluorescence values of the reagent blank are subtracted from that of each of the samples and the percentage of free RNA is determined by dividing the fluorescence intensity of the intact sample (without addition of Triton X-100) by the fluorescence value of the disrupted sample (caused by the addition of Triton X-100).

The compositions described herein are now further detailed with reference to the following examples. These examples are provided for the purpose of illustration only and the embodiments described herein should in no way be construed as being limited to these examples. Rather, the embodiments should be construed to encompass any and all variations which become evident as a result of the teaching provided herein.

### Example 3

### Lipid Nanoparticle Formulations Containing Green Fluorescent Protein (GFP) Gene

The following lipid nanoparticle formulations containing GFP gene were prepared according to the following table. The method of preparing the formulations is analogous to those disclosed in U.S. Patent Application Publication No. 2013/0245107, Example 9.

| For- | Lipids (Mole %) | | | | |
|---|---|---|---|---|---|
| mu- | | | Ionizable | Quaternary | |
| lation | Phos- | | amino | amine | |
| ID # | pholipid | Sterol | lipid | compound | PEG-Lipid |
| 1001 | 10% DSPC | 38.5% CHOL | 50% MC3 | N/A | 1.5% PEG₂ₖ- |
| | | | | | DMG |
| 1002 | 10% DSPC | 33.5% CHOL | 50% MC3 | DOTAP | 1.5% PEG₂ₖ- |
| | | | | (5%) | DMG |
| 1003 | 10% DSPC | 28.5% CHOL | 50% MC3 | DOTAP | 1.5% PEG₂ₖ- |
| | | | | (10%) | DMG |
| 1004 | 10% DSPC | 18.5% CHOL | 50% MC3 | DOTAP | 1.5% PEG₂ₖ- |
| | | | | (20%) | DMG |
| 1005 | 10% DSPC | 38.5% CHOL | 50% L608 | N/A | 1.5% PEG₂ₖ- |
| | | | | | DMG |
| 1006 | N/A | 48.5% CHOL | 50% MC3 | N/A | 1.5% PEG₂ₖ- |
| | | | | | DMG |
| 1007 | 10% SMPC | 38.5% CHOL | 50% MC3 | N/A | 1.5% PEG₂ₖ- |
| | | | | | DMG |
| 1008 | 10% DPPC | 38.5% CHOL | 50% MC3 | N/A | 1.5% PEG₂ₖ- |
| | | | | | DMG |
| 1009 | 10% PLPC | 38.5% CHOL | 50% MC3 | N/A | 1.5% PEG₂ₖ- |
| | | | | | DMG |
| 1010 | 10% POPC | 38.5% CHOL | 50% MC3 | N/A | 1.5% PEG₂ₖ- |
| | | | | | DMG |
| 1011 | 10% MSPC | 38.5% CHOL | 50% MC3 | N/A | 1.5% PEG₂ₖ- |
| | | | | | DMG |
| 1012 | 10% PMPC | 38.5% CHOL | 50% MC3 | N/A | 1.5% PEG₂ₖ- |
| | | | | | DMG |
| 1013 | 10% OMPC | 38.5% CHOL | 50% MC3 | N/A | 1.5% PEG₂ₖ- |
| | | | | | DMG |
| 1014 | 10% | 38.5% CHOL | 50% MC3 | N/A | 1.5% PEG₂ₖ- |
| | C8:PEG | | | | DMG |
| 1015 | 10% MSPC | 33.5% CHOL | 50% MC3 | DOTAP | 1.5% PEG₂ₖ- |
| | | | | (5%) | DMG |
| 1016 | 10% DSPC | 38.5% CHOL | 50% | N/A | 1.5% PEG₂ₖ- |
| | | | Compound | | DMG |
| | | | 18 | | |
| 1017 | 10% MSPC | 38.5% CHOL | 50% | N/A | 1.5% PEG₂ₖ- |
| | | | Compound | | DMG |
| | | | 18 | | |
| 1018 | 10% DSPC | 33.5% CHOL | 50% | DOTAP | 1.5% PEG₂ₖ- |
| | | | Compound | (5%) | DMG |
| | | | 18 | | |
| 1019 | 10% MSPC | 33.5% CHOL | 50% | DOTAP | 1.5% PEG₂ₖ- |
| | | | Compound | (5%) | DMG |
| | | | 18 | | |
| 1024 | 10% DSPC | 38.5% CHOL | 50% | N/A | 1.5% PEG₂ₖ- |
| | | | Compound | | DMG |
| | | | 2 | | |
| 1025 | 10% DSPC | 38.5% CHOL | 50% | N/A | 1.5% PEG₂ₖ- |
| | | | Compound | | DMG |
| | | | 23 | | |
| 1026 | 10% DSPC | 38.5% CHOL | 50% | N/A | 1.5% PEG₂ₖ- |
| | | | Compound | | DMG |
| | | | 27 | | |
| 1027 | 10% DSPC | 38.5% CHOL | 50% | N/A | 1.5% PEG₂ₖ- |
| | | | Compound | | DMG |
| | | | 10 | | |
| 1028 | 10% DSPC | 38.5% CHOL | 50% | N/A | 1.5% PEG₂ₖ- |
| | | | Compound | | DMG |
| | | | 20 | | |

The specified amount of the lipid components were combined in an ethanol solution to a final concentration of 25 mM. A solution of the mRNA encoding GFP at a concentration of 1-2 mg/mL in water was diluted in a 50 mL sodium citrate buffer at a pH of 3 to form a stock mRNA solution. Formulations of the lipid and mRNA were prepared by combining the lipid solution with the mRNA solution at total lipid to mRNA weight ratio of 20:1 unless otherwise specified. The lipid ethanolic solution was rapidly injected into aqueous mRNA solution to afford a suspension containing 33% ethanol. The solutions were injected either manually (MI) or by the aid of a syringe pump (SP) (Harvard Pump 33 Dual Syringe Pump Harvard Apparatus Holliston, Mass.). Then the suspension was further diluted by 3 times volume of PBS or citrate buffer or a mixture of both to further dilute the ethanol concentration to 8.25%.

To remove the ethanol and to achieve the buffer exchange, the formulations were diafiltrated against at least 6 times volume of phosphate buffered saline (PBS), pH 7.4 Pellicon XL50, (Millipore) with a molecular weight cutoff (MWCO) of 100 kD and then concentrated to appropriate volume. The resulting nanoparticle suspension was filtered through 0.2 μm sterile filter (Sarstedt, Nümbrecht, Germany) into glass vials and sealed with a crimp closure.

### Example 4

### In Vivo Expression of GFP in Hep 3B Tumors

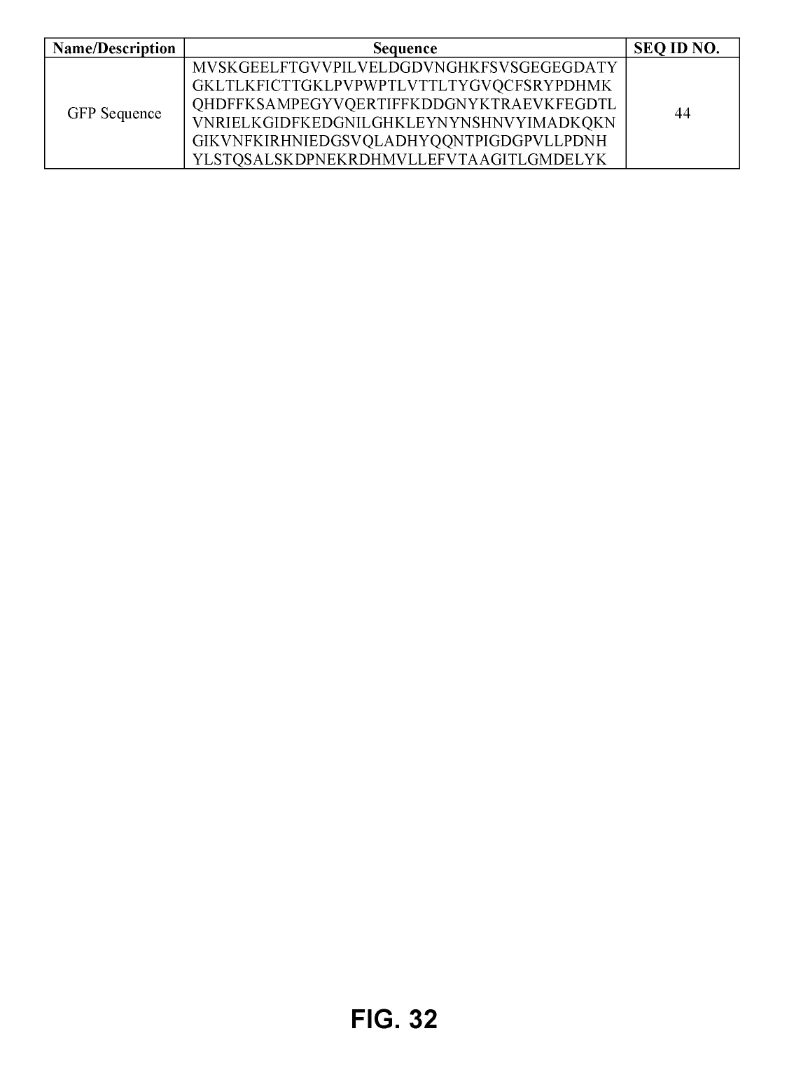

Expression of GFP was measured in cancer cells following treatment with a polynucleotide comprising an mRNA encoding GFP (GFP SEQ ID: 44, FIG. 32).

Human hepatocellular carcinoma tumors were established subcutaneously in mice. Mouse tumor cells (Hep 3B, ATCC No. HB-8064™; ATCC, Manassas, Va.) were cultured according to the vendor's instructions. Cells were inoculated subcutaneously in mice to generate subcutaneous tumors. Tumors were monitored for size and palpability.

Once the tumors reached a mean size of approximately 150˜300 mm³, animals were treated with intratumoral dose of lipid formulations (Formulations 1001, 1002, 1003, and 1005) containing the mRNA encoding GFP. Formulations 1001, 1002, 1003, and 1005 were administered intratumorally at 0.5 mg/kg (about 10 μg/mouse) in 25 μL into sc Hep3B tumors. Control animals were treated with intratumoral dose of PBS. Animals were sacrificed 24 hours after dosing. Tumor and liver tissues were harvested and analyzed for expression of GFP.

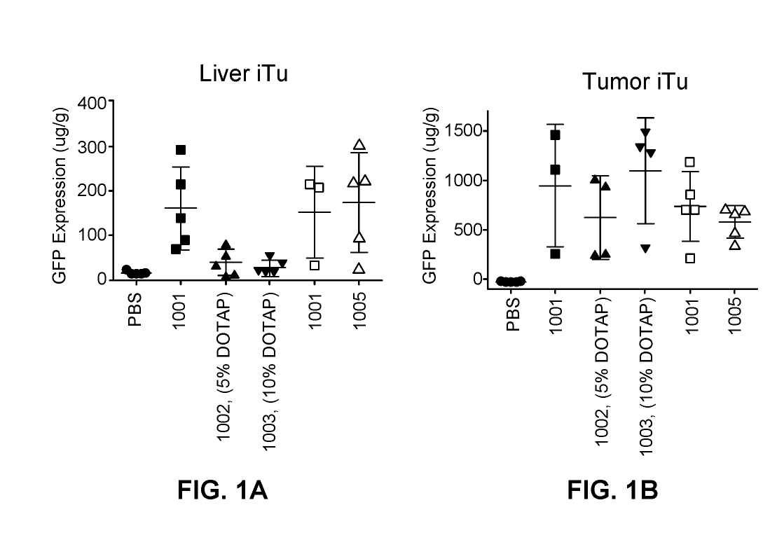

FIGS. 1A and 1B show the GFP expression levels in tumor and liver. The GFP expression levels in the tumor cells were about 600-1100 μg/g for all four formulations. The GFP expression levels in liver for Formulation 1001 (reference) (containing MC3, but not DOTAP) and Formulation 1005 (containing L608, but not DOTAP) were about 150-200 μg/g, while those for Formulations 1002 and 1003 (containing MC3, and 5 or 10 mole % of DOTAP, respectively) were much lower, below 50 μg/g.

These results show that when administered intratumorally, the inclusion of DOTAP in the lipid composition containing mRNA reduces GFP expression in liver while maintaining the GFP expression in the tumor.

### Example 5

### In Vivo Expression of GFP in Hep 3B Tumors at Lower Doses

Similarly to Example 4, Formulations 1001, 1002, and 1003 were administered intratumorally at 2.5 μg/mouse into sc Hep3B tumors. GFP expression levels in tumor and liver were measured 24 hours after administration.

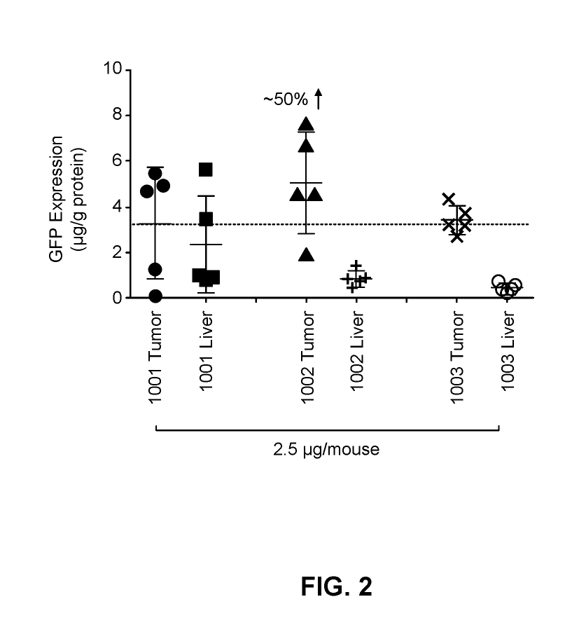

FIG. 2 shows the GFP expression levels in tumor and liver. Formulation 1002 results in a higher GFP expression level in tumor than Formulation 1001. Formulation 1003 resulted in the same level of GFP expression in tumor as Formulation 1001. Regarding GFP expression in liver, both Formulations 1002 and 1003 resulted in a lower level than Formulation 1001.

These results show that the formulation with 5 mole % of DOTAP provided higher GFP expression in tumor and lower GFP expression in liver, while the formulation with 10 mole % of DOTAP lowered protein expression in liver and maintaining protein expression in tumor.

### Example 6

### In Vivo Expression of Luciferase in A20 Tumors

Mouse models of B-cell lymphoma using the A20 cell line are useful for analyzing a tumor microenvironment. (Kim et al., Journal of Immunology 122(2):549-554 (1979); Donnou et al., Advances in Hematology 2012:701704 (2012)). Therefore, in vivo expression of luciferase and the tumor microenvironment were assessed in an A20 B-cell lymphoma tumor model.

B-cell lymphoma tumors were established subcutaneously in BALB/c mice. Mouse B-cell lymphoma cells (A20, ATCC No. TIB-208; ATCC, Manassas, Va.) were cultured according to the vendor's instructions. Cells were inoculated subcutaneously in BALB/c mice to generate subcutaneous tumors. Tumors were monitored for size and palpability.

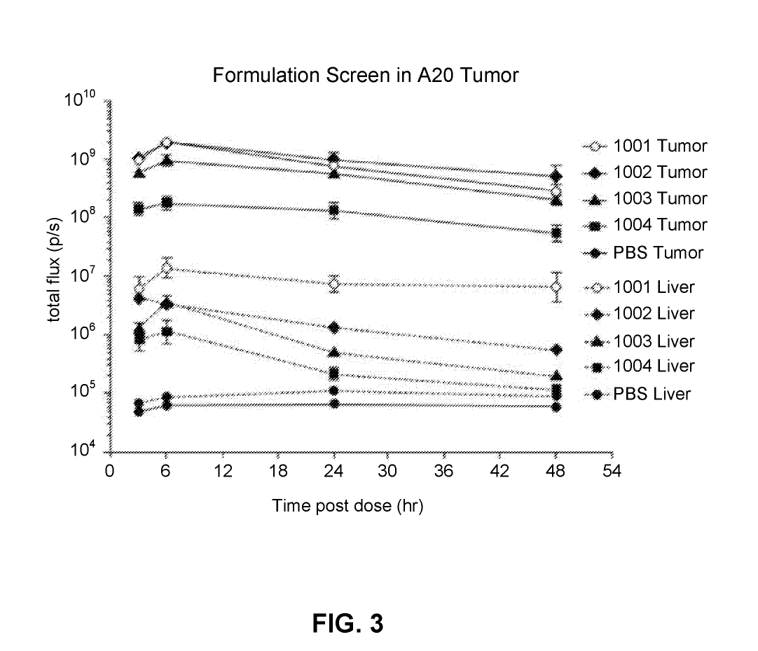

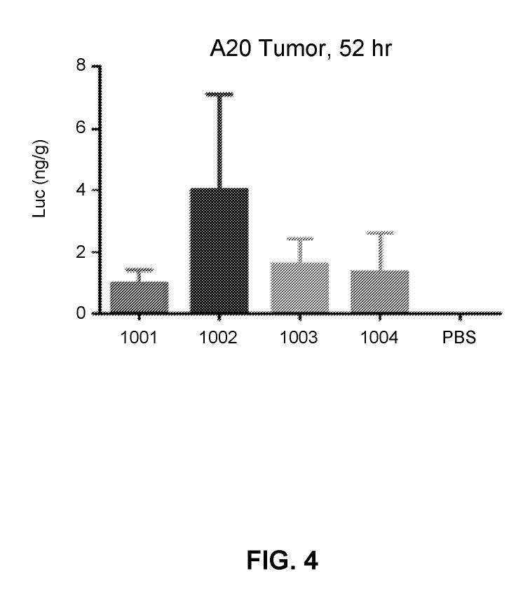

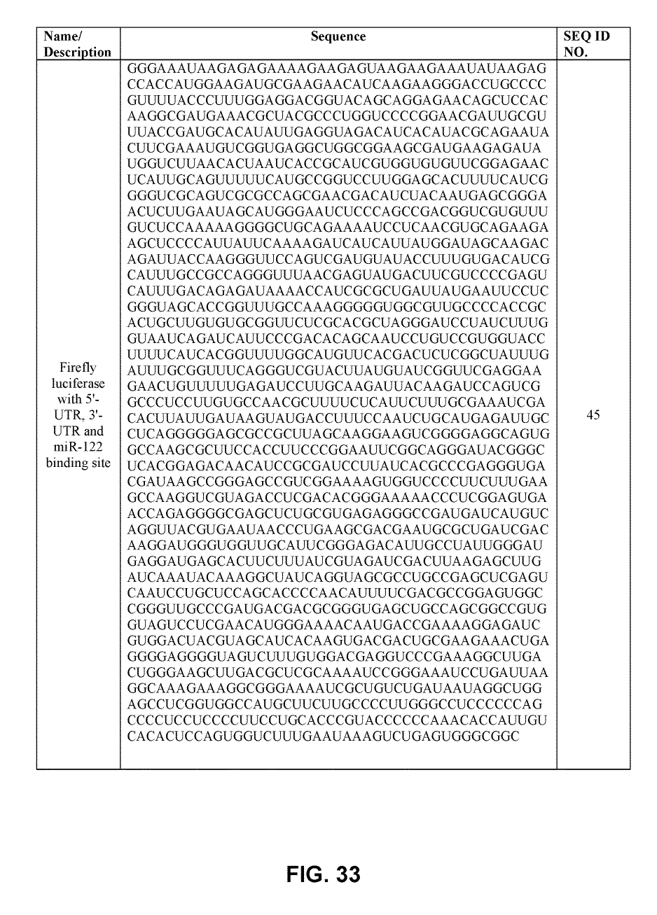

Once the tumors reached a mean size of approximately 300 mm³, animals were treated with intratumoral dose of lipid formulations (Formulations 1001, 1002, 1003, and 1004) containing an mRNA encoding luciferase (RNA SEQ ID NO: 45, FIG. 33) at 12.5 μg/mouse. Control animals were treated with intratumoral dose of PBS. After the treatment, animals were anesthetized, injected with the luciferase substrate D-luciferin and the bioluminescence imaging (BLI) from living animals was evaluated in an IVIS imager. Signals from tumor tissue were obtained and compared with signals from liver tissue in the same animal. Bioluminescenes are measured as total flux (photons/second). Results are shown in FIG. 3. Animals were sacrificed 52 hours after dosing. Tumor tissue was harvested and analyzed for expression of luciferase. Results are shown in FIG. 4.

All formulations demonstrated a great level of protein expression in tumors. Formulations 1002 and 1003 showed similar amount of BLI compared to reference Formulation 1001. Decreased protein expression was observed in Formulation 1004 which contained 20 mole % of DOTAP. FIG. 3 also shows significant decrease in protein expression in liver for Formulations 1002, 1003, and 1004, all containing DOTAP.

FIG. 4 shows that luciferase expression levels in tumor 52 hours post dosing. Formulation 1002 (5 mole % of DOTAP) showed a significantly higher protein expression compared to reference Formulation 1001. Both Formulations 1003 and 1004 showed slightly higher protein expression compared to reference Formulation 1001.

The ratios of the protein expression levels in tumor and liver for each of the four formulations are shown in the table below.

| | | Tumor/Liver ratio | | | |
|---|---|---|---|---|---|
| Group | Formulation | 3 hr | 6 hr | 24 hr | 48 hr |
| 1 | 1001 | 161.09 | 142.11 | 106.22 | 45.36 |
| 2 | 1002 | 255.46 | 577.78 | 742.93 | 951.47 |
| 3 | 1003 | 468.04 | 257.21 | 1197.18 | 1037.68 |
| 4 | 1004 | 169.77 | 157.26 | 607.00 | 463.23 |
| 5 | PBS | 0.71 | 0.74 | 0.60 | 0.68 |

The formulations containing from about 5 to about 20 mole % of DOTAP provide higher luciferase expression in tumor and lower luciferase expression in liver. The ratio of the protein expression levels in tumor and liver (tumor/liver ratio) is significantly higher for the formulations containing DOTAP than that for Formulation 1001. These results indicate a preferential expression of the polypeptide in tumors compared to a corresponding formulation without DOTAP.

### Example 7

### In Vivo Expression of GFP in MC38 Tumors

Expression of GFP was measured in cancer cells following treatment with a polynucleotide comprising an mRNA encoding GFP.

MC-38 colon adenocarcinoma tumors were established subcutaneously in C57BL/6 mice. (Rosenberg et al., Science 233(4770):1318-21 (1986)).

Once the tumors reached a mean size of approximately 100 mm³, animals were treated with intratumoral dose of lipid formulations containing the mRNA encoding GFP. The lipid formulations included Formulations 1001, 1001 (total lipid to mRNA ratio 15:1), 1001 (total lipid to mRNA ratio 10:1), 1015, 1002, 1003, 1007, and 1011. The lipid formulations were administered intratumorally at 2.5 μg/mouse into MC38 sc tumors. Control animals were treated with intratumoral dose of PBS. Animals were sacrificed 24 hours after dosing. Tumor and liver tissues were harvested and analyzed for expression of GFP.

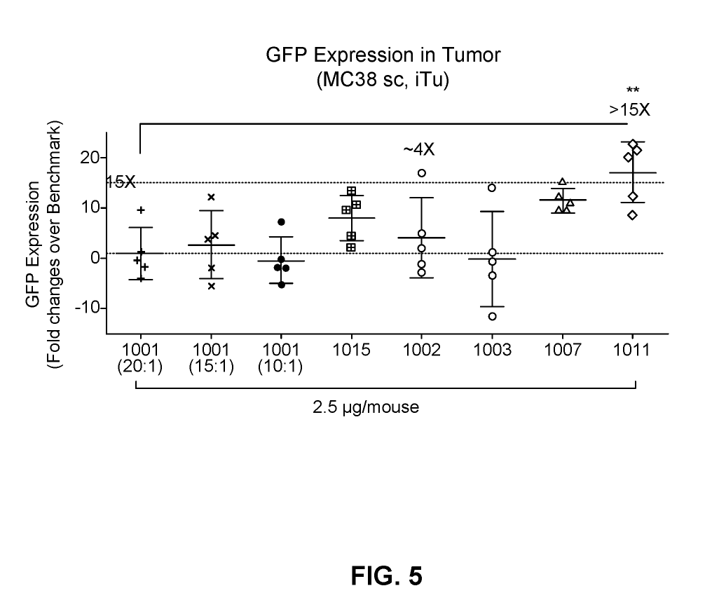

FIG. 5 shows the GFP expression levels in tumor. No obvious decrease of protein expression in tumor was observed when the total lipid to mRNA ratio was reduced from 20:1 to 15:1 and 10:1. Formulation 1011 containing MC3/MSPC showed significant improvement (more than 15 times) compared to reference Formulation 1001 containing MC3/DSPC. Formulation 1007 containing MC3/SMPC showed a smaller increase of protein expression. Formulation 1002 containing 5% DOTAP showed a four-fold increase in protein expression compared to reference Formulation 1001.

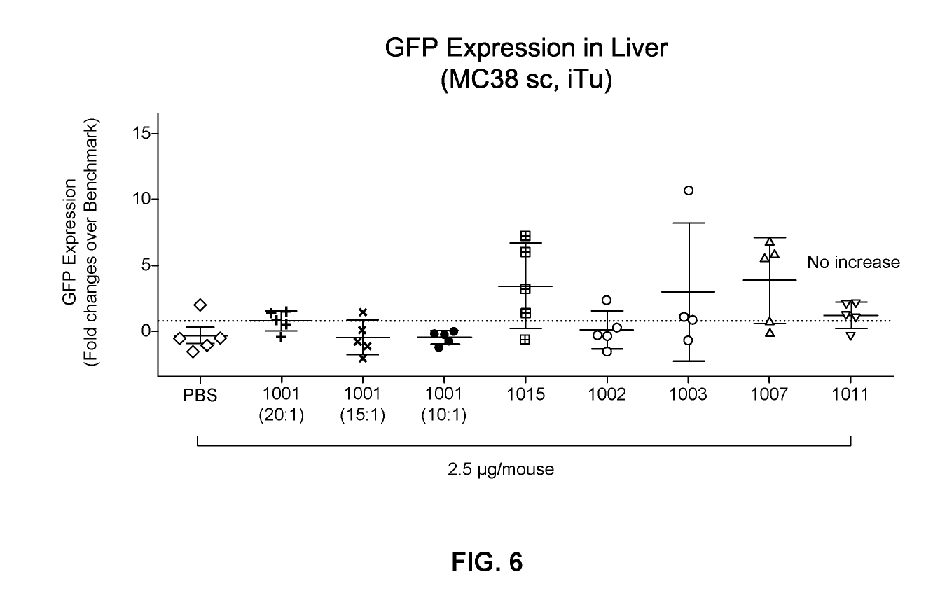

FIG. 6 shows the GFP expression levels in liver. When the total lipid to mRNA ratio was reduced, the protein expression in liver was not changed or slightly decreased. The protein expression in liver did not increase for Formulations 1002 and 1011.

### Example 8

### In Vivo Expression of GFP in Hep 3B Tumors

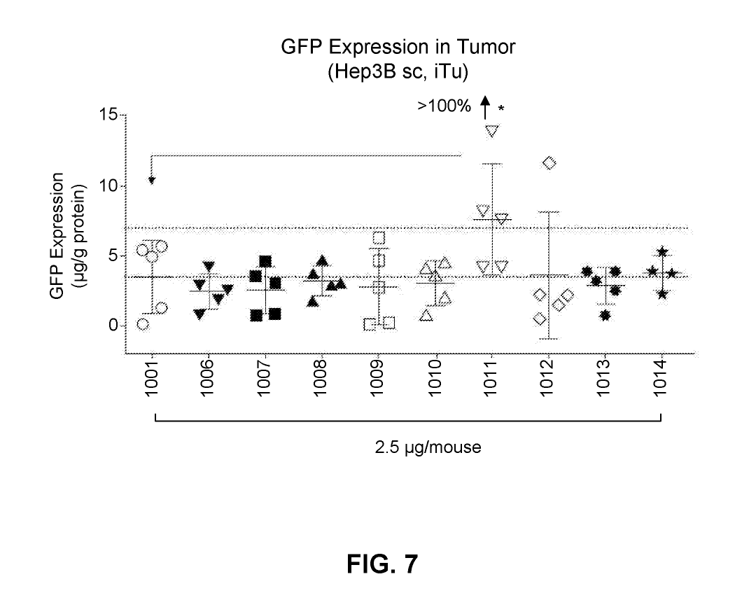

Similarly to Example 4, Formulations 1001 and 1006-1014 were administered intratumorally at 2.5 μg/mouse into sc Hep3B tumors. GFP expression levels in tumor and liver were measured 24 hours after administration. FIG. 7-FIG. 10 show test results.

FIG. 7 shows the GFP expression levels in tumor at 24 hours posting dosing for doses of 2.5 μg/mouse. Significant increase in tumor expression was observed with Formulation 1011 containing MSPC as compared to Formulation 1001.

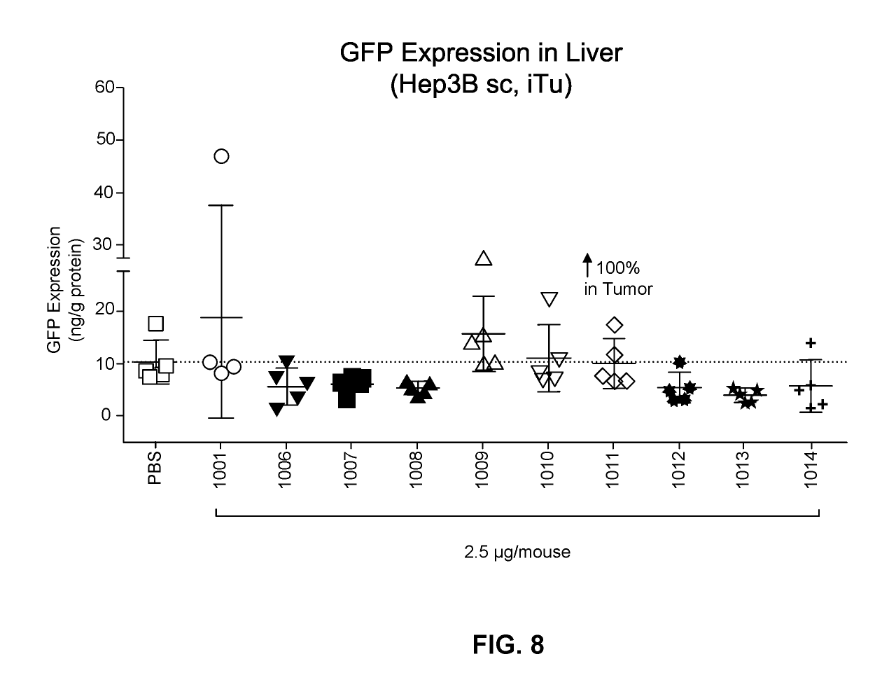

FIG. 8 shows the GFP expression levels in liver at 24 hours posting dosing for doses of 2.5 μg/mouse. No increase in liver expression was observed with Formulation 1011 as compared to Formulation 1001.

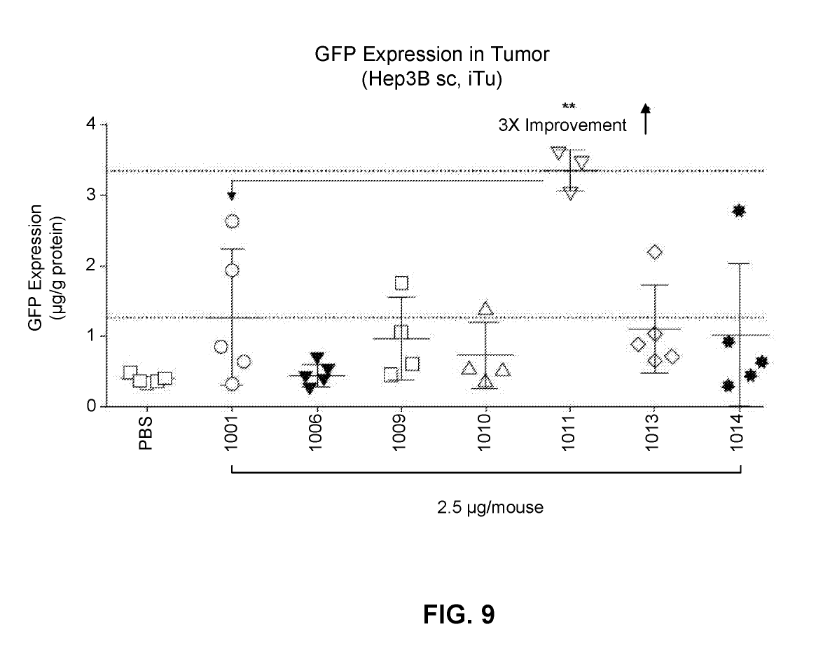

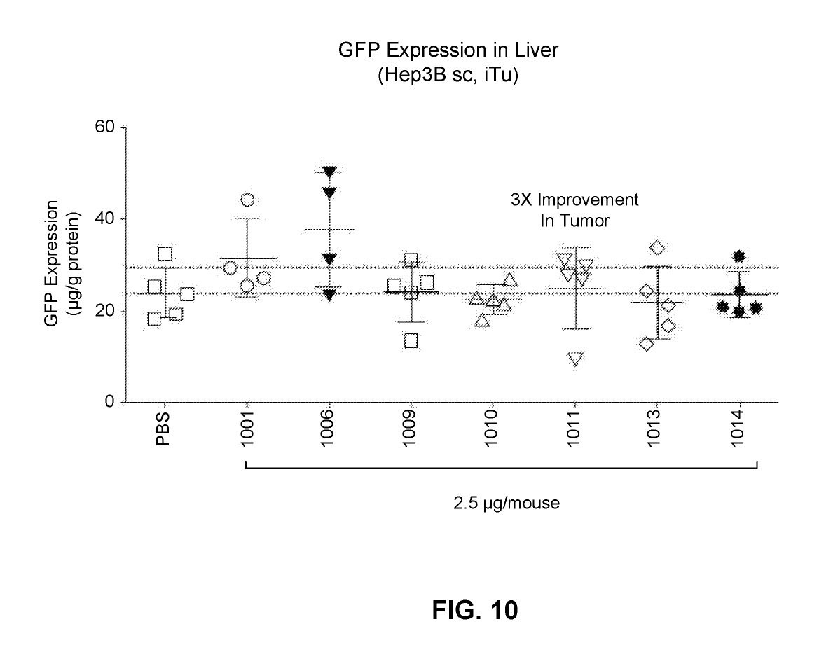

FIG. 9 and FIG. 10 show similar results for GFP expression in tumor and liver.

### Example 9

### Intratumoral Delivery Using Lipid Nanoparticles Comprising Compound 18

Similarly to Example 7, Formulations 1001, 1015, 1016, 1017, 1018, and 1019 were administered intratumorally at 0.5 μg/mouse or 2.5 μg/mouse into MC38 sc tumors. GFP expression levels in tumor and liver were measured 6 hours or 24 hours after administration.

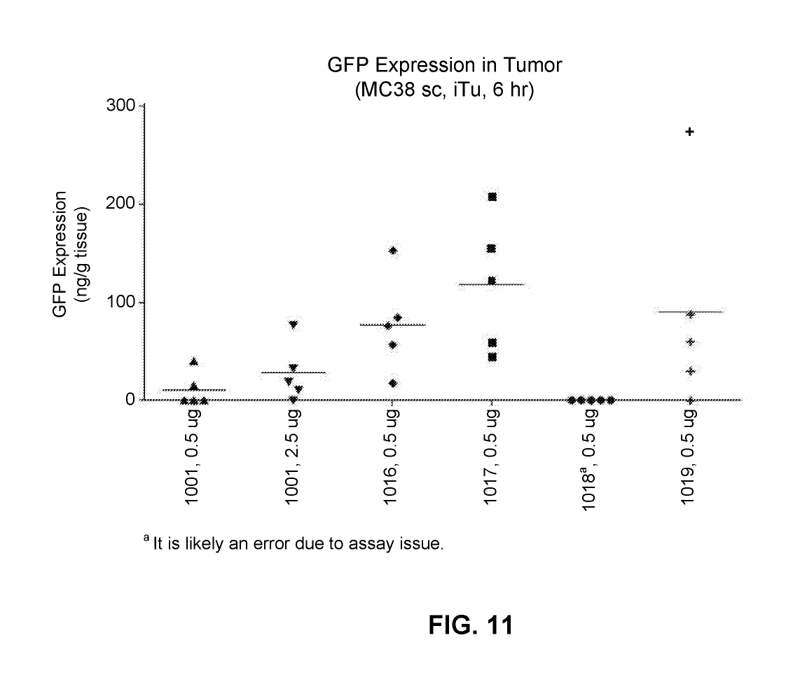

FIG. 11 shows the GFP expression levels in tumor at 6 hours post dosing. Formulations containing Compound 18 showed significantly higher protein expression compared reference Formulation 1001 containing MC3. The highest protein expression level was obtained using formulations containing Compound 18+MSPC, or Compound 18+MSPC+5% DOTAP.

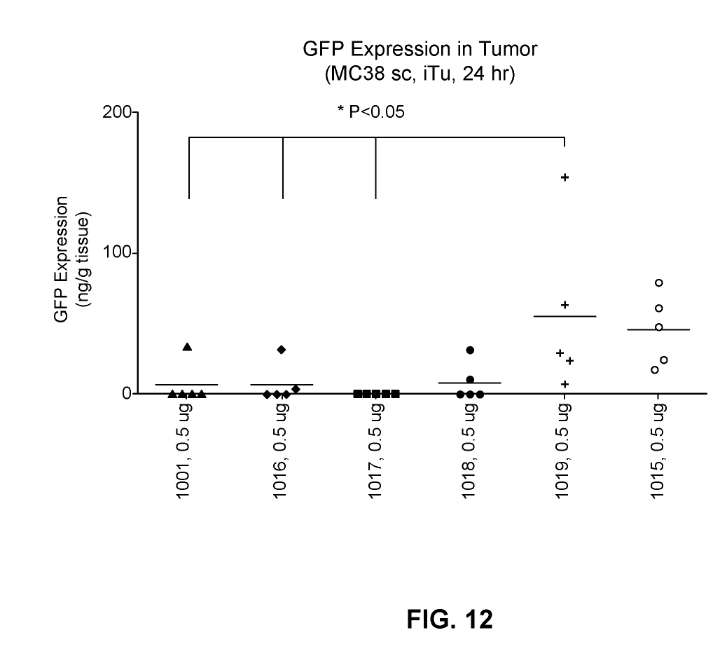

FIG. 12 shows the GFP expression levels in tumor at 24 hours posting dosing for doses of 0.5 μg/mouse. Formulation 1018 showed a similar level of protein expression compared to reference Formulation 1001. Formulations 1019 and 1015—both containing an ionizable amino lipid (Compound 18 and MC 3, respectively), MSPC, and 5% DOTAP—showed a higher level of protein expression compared to reference Formulation 1001.

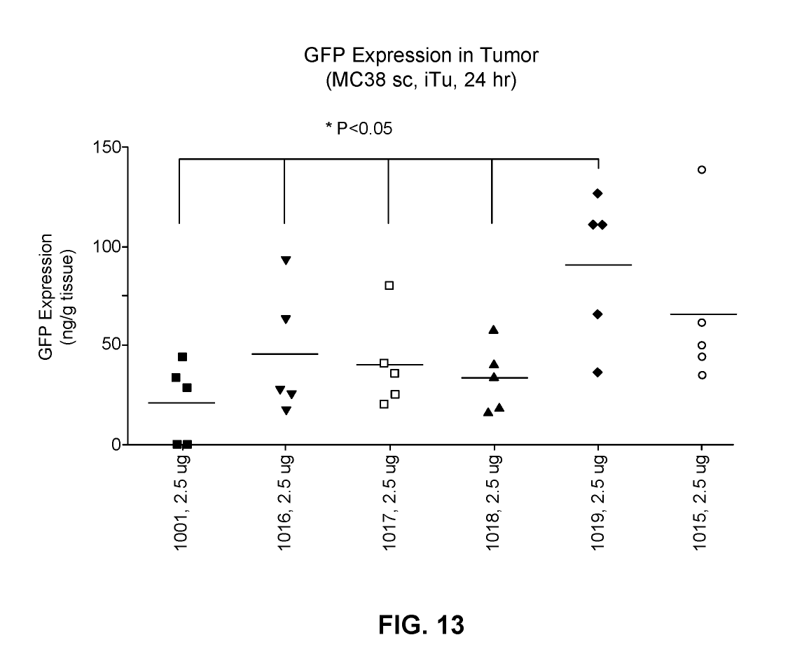

FIG. 13 shows the GFP expression levels in tumor at 24 hours posting dosing for doses of 2.5 μg/mouse. At a dose of 2.5 μg/mouse, Formulations 1019 and 1015 showed a higher level of protein expression compared to reference Formulation 1001. Formulation 1019 was observed to have the highest level of protein expression.

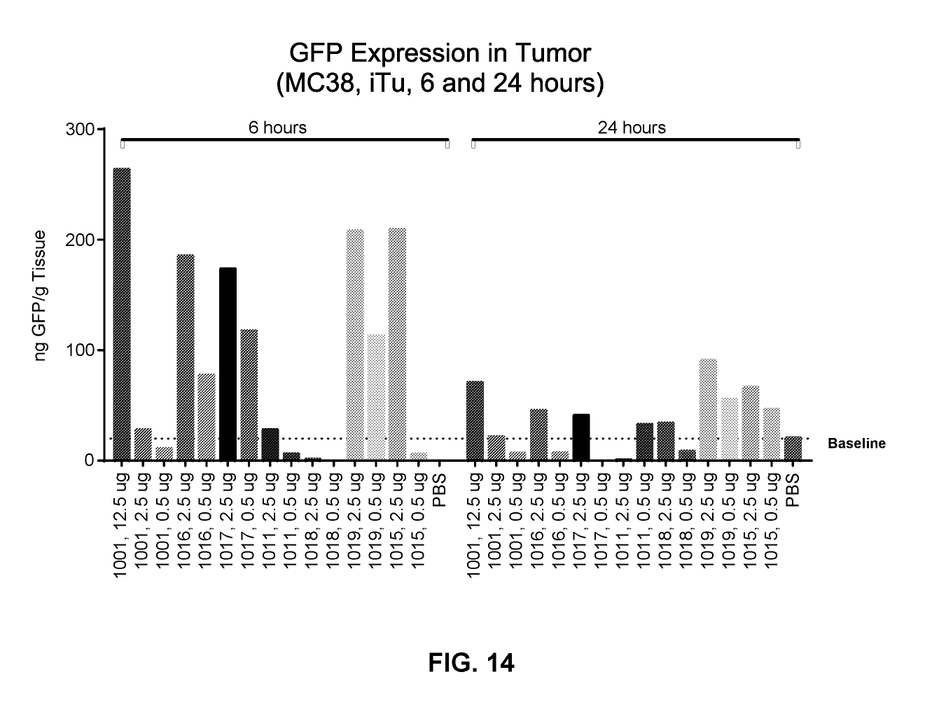

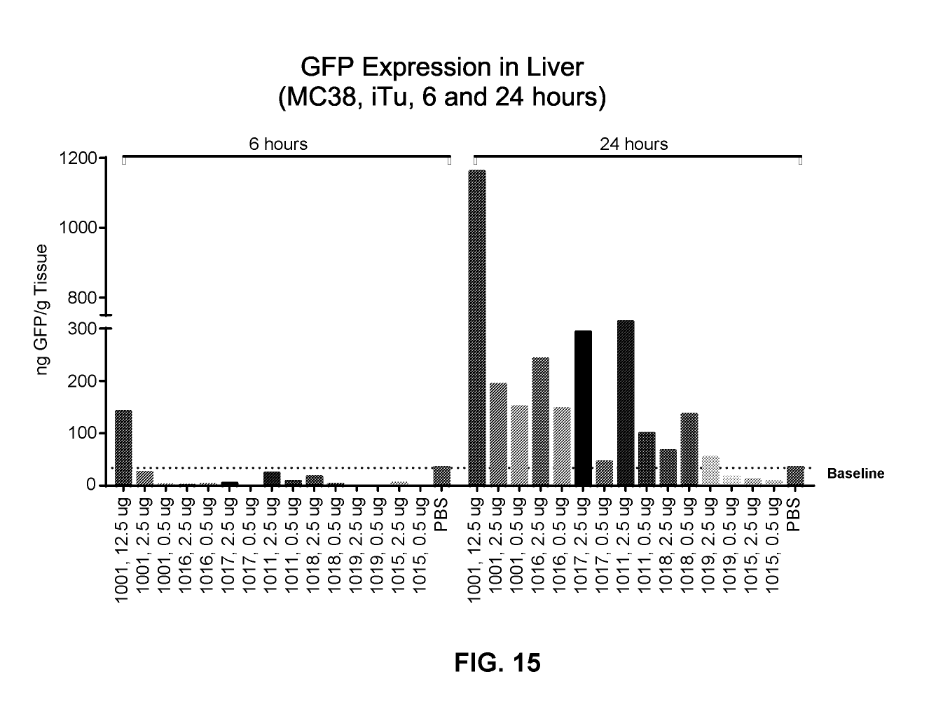

A summary of GFP expression results in tumor and liver with various formulations is shown in FIG. 14 and FIG. 15, respectively. Formulations 1019 and 1015 showed a higher ratio of protein expression in tumor to protein expression in liver 24 hour post administration, compared to all other formulations tested.

### Example 10

### Cytokine Profiles Induced by Intratumoral Administration of Lipid Compositions

The introduction of foreign material into a mammalian body induces an innate immune response that promotes cytokine production. Such immune responses to, for example, nanoparticle compositions including therapeutic and/or prophylactics, are undesirable. The induction of certain cytokines was thus measured to evaluate the efficacy of nanoparticle compositions. These cytokines were interleukin-6 (IL-6), CXCL1 (chemokine (C—X—C motif) ligand 1; formerly known as GROα), interferon-γ (IFNγ), tumor necrosis factor α (TNFα), interferon y-induced protein 10 (IP-10), and granulocyte-colony stimulating factor (G-CSF).

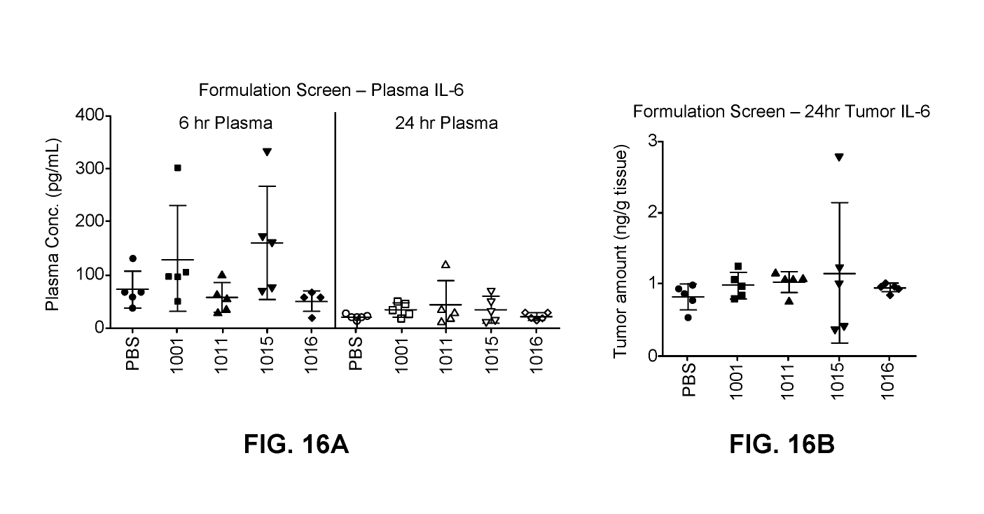

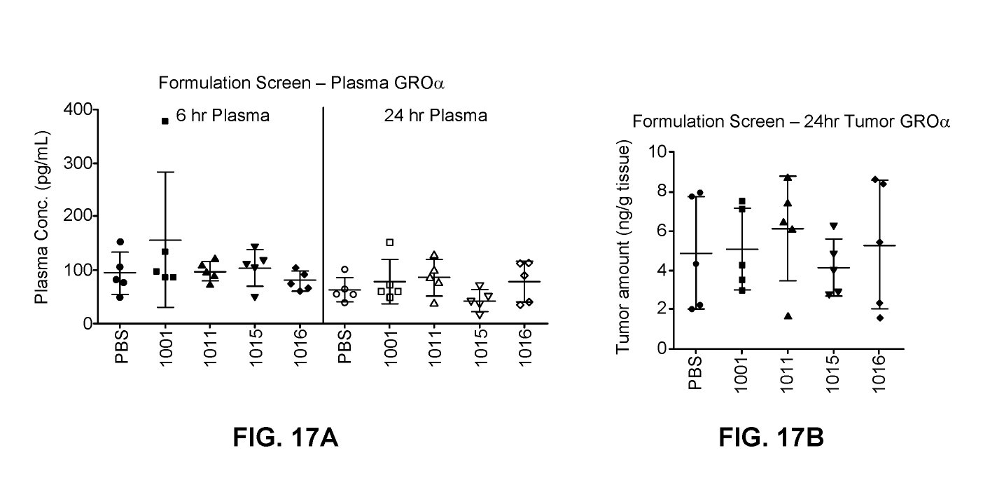

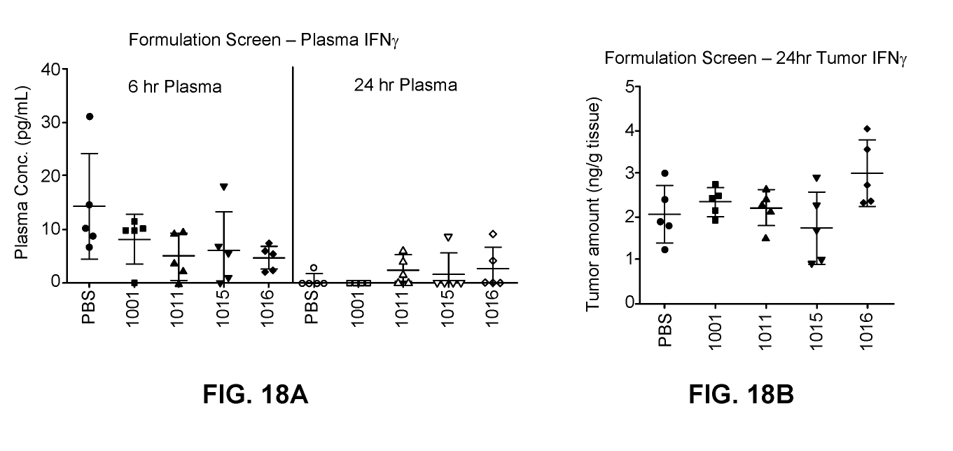

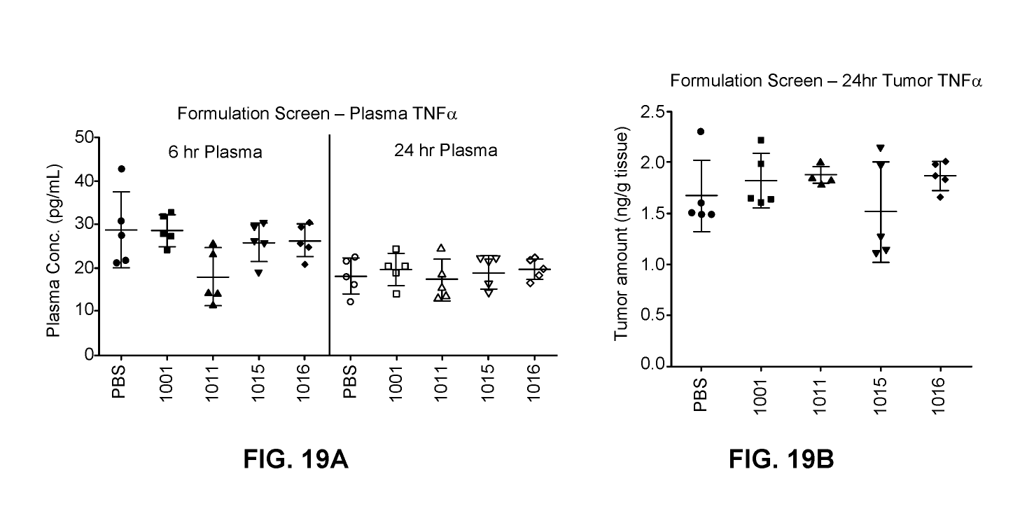

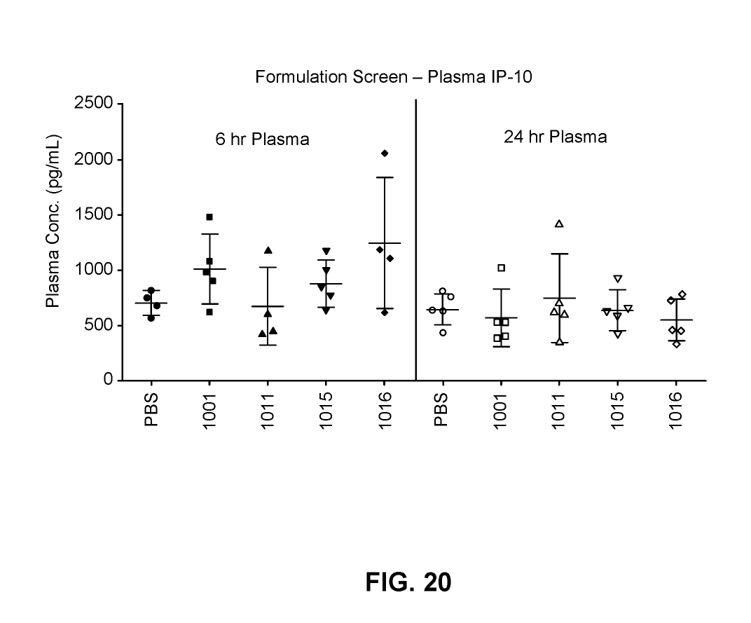

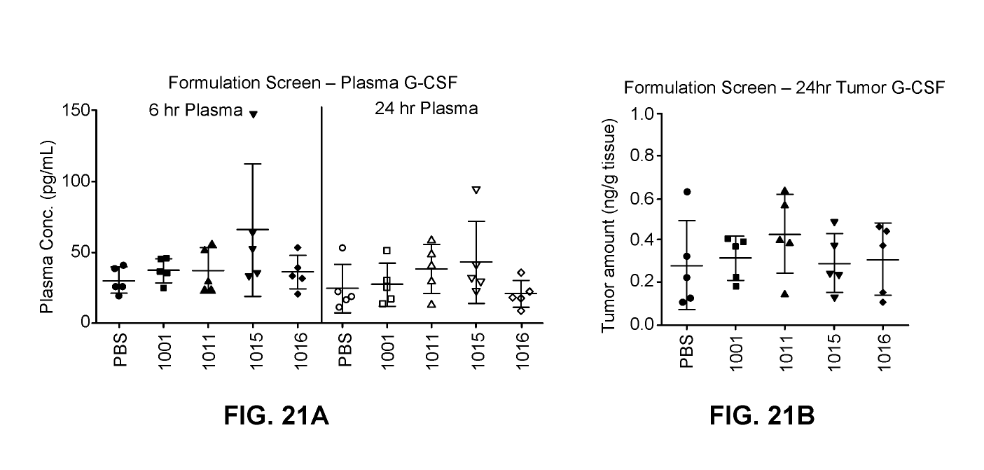

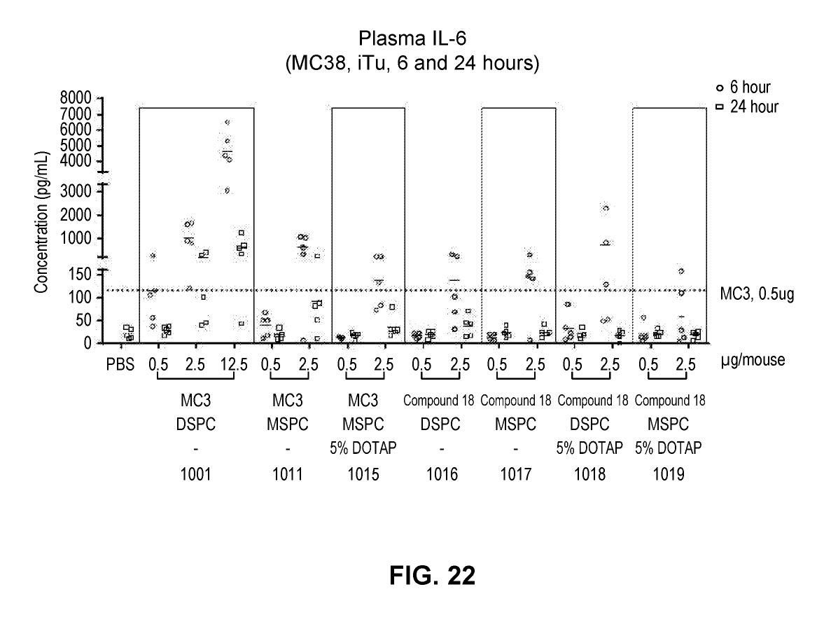

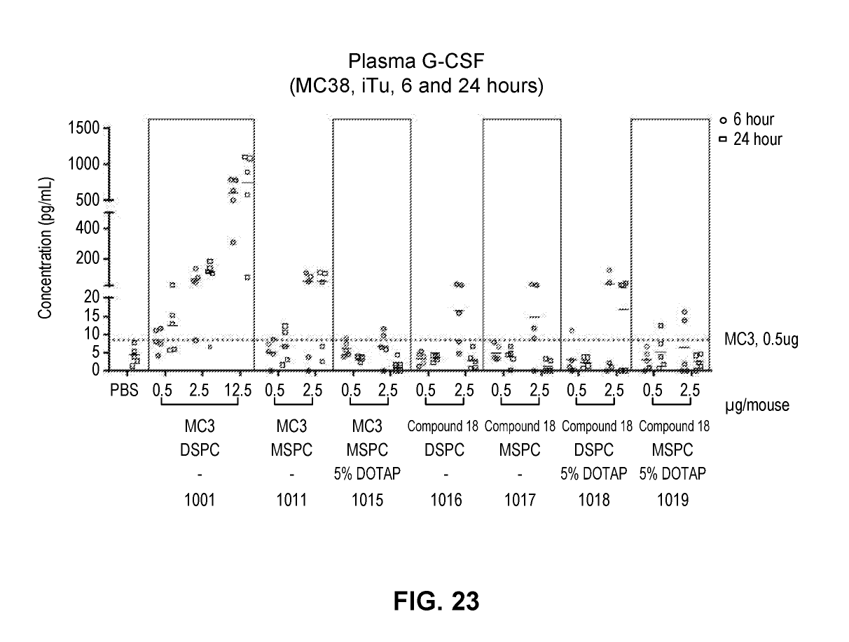

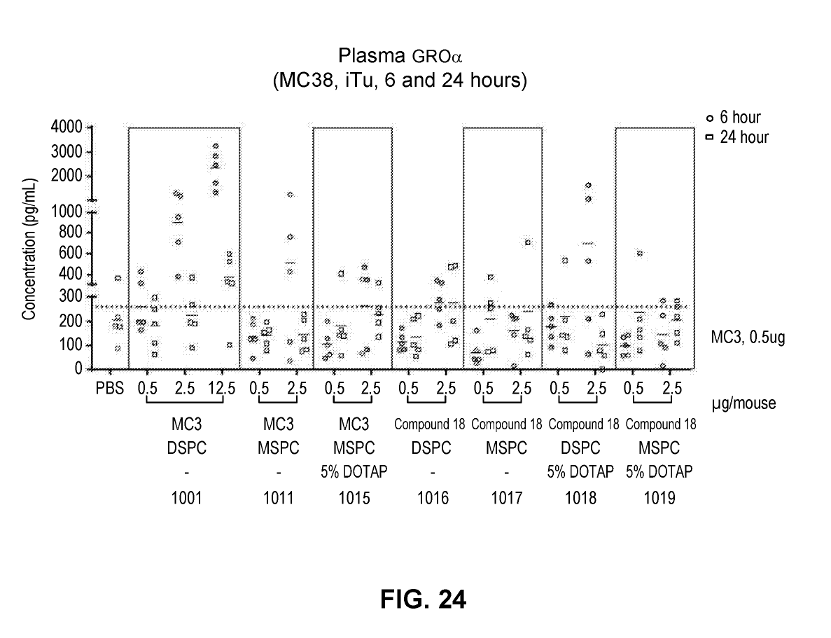

Formulations as discussed above were administered intratumorally to subcutaneous tumor in model mice. The concentrations of the various cytokines after intratumoral administration was measured in plasma samples at 6 hours and 24 hours post-administration and in tumor tissue 24 hours post-administration. A PBS control was also tested. The results are shown in FIGS. 16A, 16B, 17A, 17B, 18A, 18B, 19A, 19B, 20, 21A, 21B, and 22-24.

Overall, the concentrations of the cytokines in plasma at 24 hours post administration for the tested formulations were similar to those for PBS.

### Example 11

### In Vivo Expression of GFP in MC38 Tumors

Similarly to Example 9, Formulations 1016 and 1024-1028 were administered intratumorally at 2.5 μg/mouse doses into sc MC38 tumors. GFP expression levels in tumor and liver were measured 24 hours after administration.

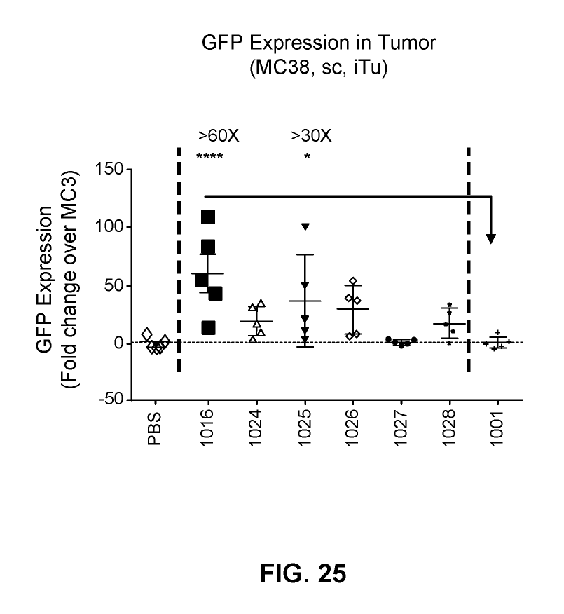

FIG. 25 shows the GFP expression levels in tumor. The highest GFP expression in tumor was observed on Formulation 1016 and 1025. Formulations 1016 and 1025 showed significant improvement over reference Formulation 1001 containing MC3 (approximately 60-fold and 30-fold).

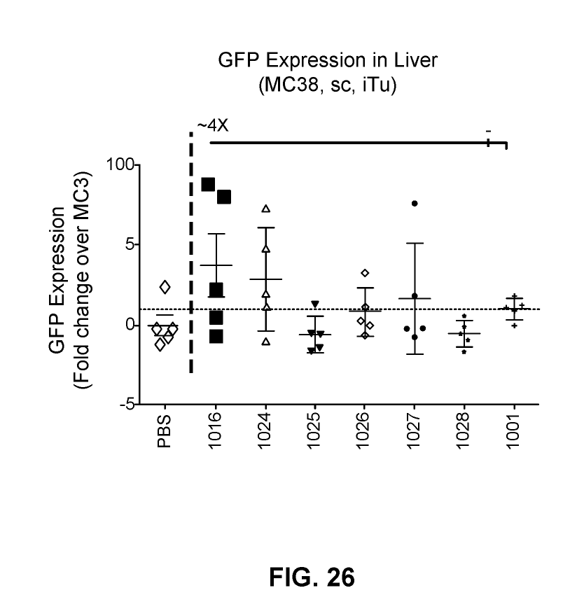

FIG. 26 shows the GFP expression level in liver. A high amount of liver expression was observed on Formulation 1016 and Formulation 1022. Formulation 1025 and Formulation 1028 showed the lowest/no amount of liver expression.

### Example 12

### Interleukin-6 Levels

Induced by Intratumoral Administration of LNP Formulated mRNAs

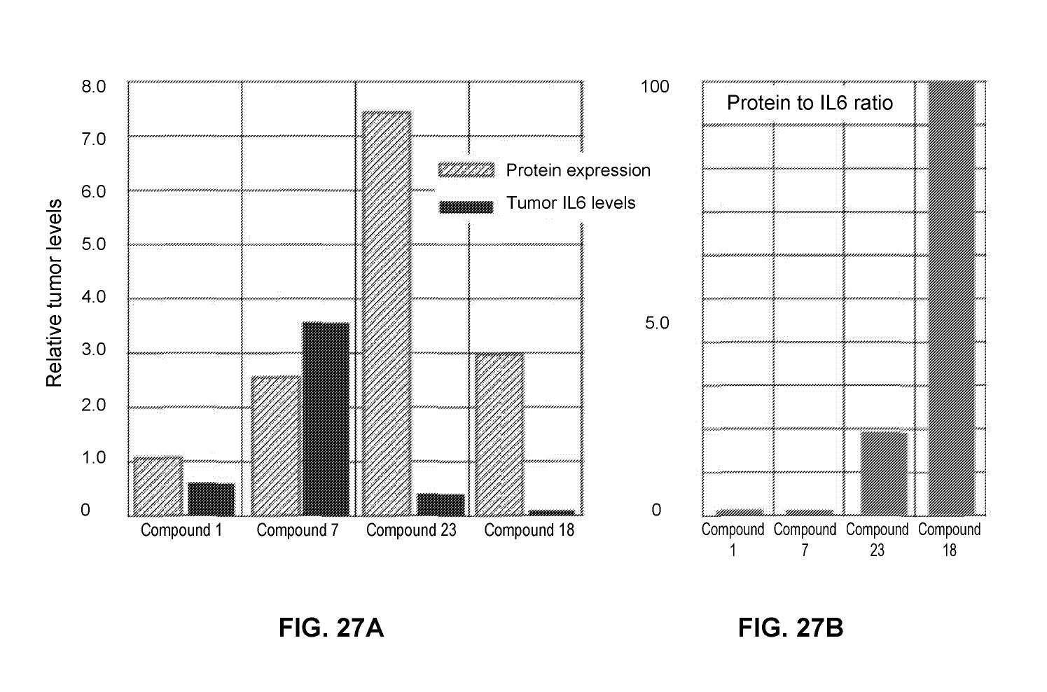

Formulations (LNPs) containing a lipid (e.g., Compound 1, Compound 7, Compound 23, and Compound 18) and an mRNA encoding a protein of interest were administered via injection into tumors. The level of the expressed protein and IL-6 in the tumors were measured and compared. Results are shown in FIGS. 27A and 27B. As the data demonstrate, LNP-formulated mRNAs wherein the LNPs include Compound 18 as the ionizable amino lipid result in significant protein expression in the relative absence of IL6 (cytokine) secretion, making such LNPs very well suited for intratumoral administration of mRNAs.

### Example 13

### Plasma and Liver Pharmacokinetics of LNP Formulated mRNAs

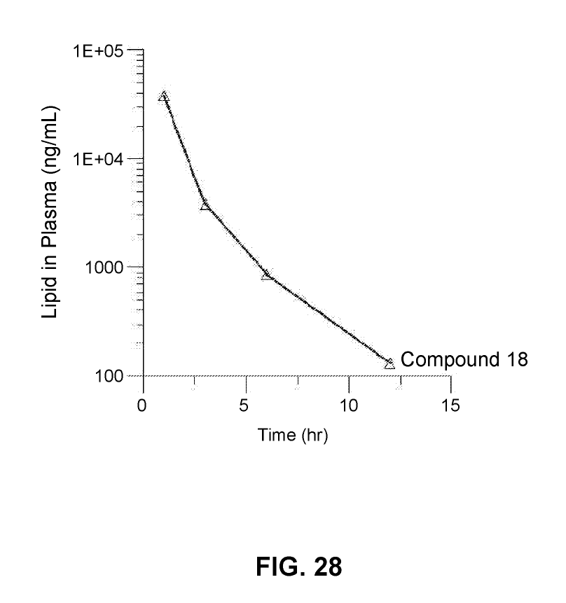

The plasma and liver pharmacokinetics of a lipid formulation (LNP) containing Compound 18 and an mRNA encoding a protein of interest was studied. The concentrations of Compound 18 in plasma after a single IV infusion are shown FIG. 28. The concentration of Compound 18 decreased quickly within about 12 hours.

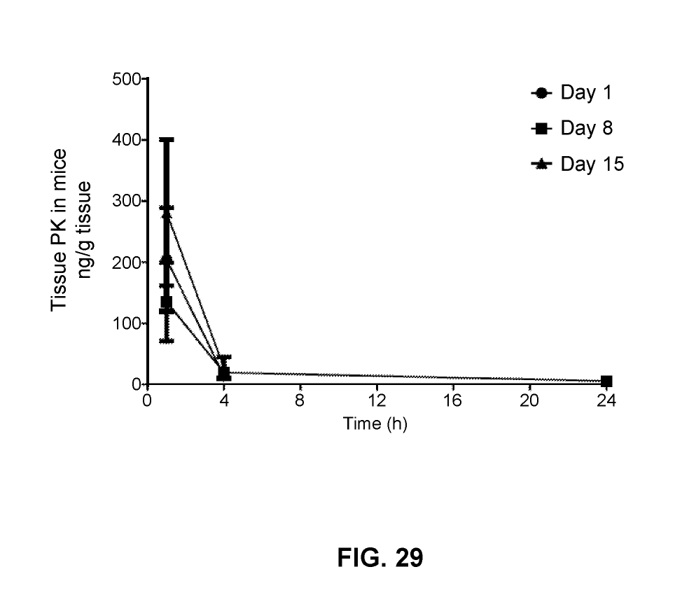

The concentrations of Compound 18 in liver tissues after weekly dosing were measured at day 1, day 8, and day 15. Results are shown in FIG. 29. The liver concentration decreased significantly at four hours after each weekly dosing.

### Example 14

### Tolerability Profile In Vivo

Toxicological studies were conducted in rats and non-human primates for lipid formulations (LNPs) containing Compound 18. These formulations were found to have good tolerability profile in vivo.

The formulation containing Compound 18 has an STD₁₀ of 1 mg RNA/kg, and an HNSTD of 0.3 mg RNA/kg. In addition, the formulations containing Compound 18 are well tolerated after local injection in terms of injection site reactions, systemic inflammation, systemic inflammation induced stress, and secondary findings.

### Example 15

### Levels of Myeloid-Derived Suppressor Cells

Mouse models of B-cell lymphoma using the A20 cell line are useful for analyzing a tumor microenvironment. (Kim et al., Journal of Immunology 122(2):549-554 (1979); Donnou et al., Advances in Hematology 2012:701704 (2012)).

Myeloid-derived suppressor cells (MDSCs) are a heterogeneous population of cells that play a critical role in tumor associated immune suppression. MDSC is believed to consist of two major subsets of Ly6G⁺Ly6C^{low} granulocytic and Ly6G⁻Ly6C^{high} monocytic cells.

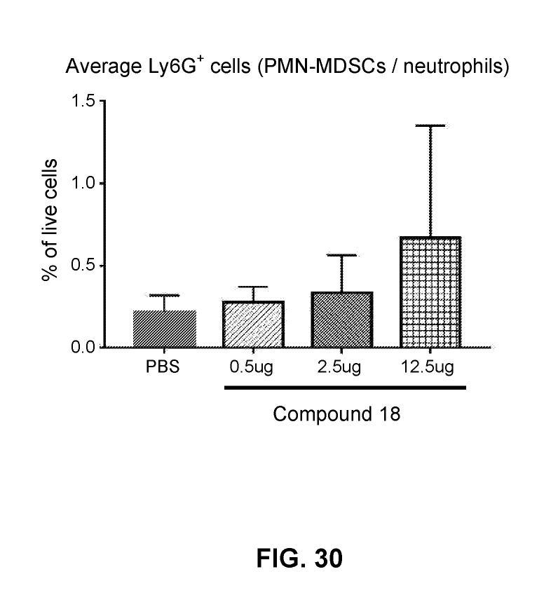

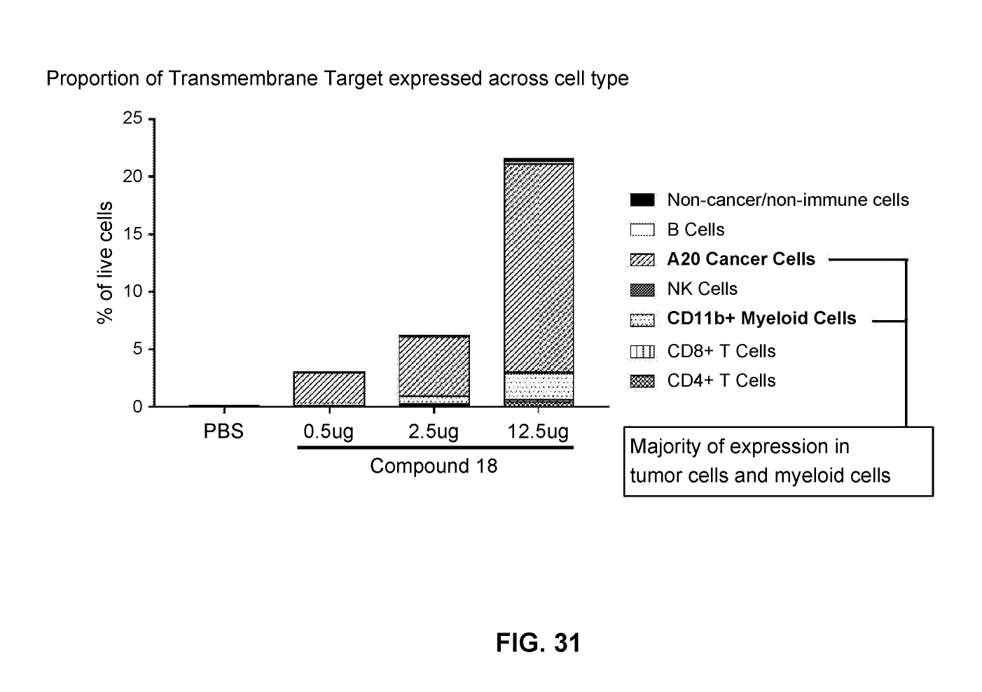

Formulations (LNPs) containing Compound 18 and an mRNA encoding a protein of interest were administered intratumorally into A20 tumors. Live cells from the A20 tumors were analyzed by flow cytometry 24 hours after dosing. The percentage of Ly6G⁺ cells were measured, and the results show minimal increases in MDSCs associated with formulations containing Compound 18 when administered at 0.5, 2.5, and 12.5 μg/mouse doses (FIG. 30). In addition, among the 20-25% of transfected cells, the majority of expression occurred in tumor cells and myeloid cells (FIG. 31).

### Other Embodiments

It is to be understood that the words which have been used are words of description rather than limitation, and that changes can be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

The present application claims benefit to U.S. Application Ser. Nos. 62/321,933, filed Apr. 13, 2016; 62/338,139, filed May 18, 2016; 62/338,126, filed May 18, 2016; and 62/415,395, filed Oct. 31, 2016, all of which are incorporated herein by reference in their entireties.

All publications, patent applications, patents, and other non-patent references mentioned herein are incorporated by reference in their entirety. In case of conflict, the present specification, including definitions, will control. In addition, section headings, the materials, methods, and examples are illustrative only and not intended to be limiting.

1. A composition comprising:
(i) a lipid composition comprising
(1) an ionizable amino lipid; and
(2) a quaternary amine compound; and
(ii) a polynucleotide,
wherein the amount of the quaternary amine compound ranges from about 0.01 to about 20 mole % in the lipid composition, or
wherein the mole ratio of the ionizable amino lipid to the quaternary amine compound is about 100:1 to about 2.5:1.

2. The composition of claim 1, wherein the ionizable amino lipid is selected from the group consisting of DLin-MC3-DMA (MC3), DLin-DMA, DLenDMA, DLin-D-DMA, DLin-K-DMA, DLin-M-C2-DMA, DLin-K-DMA, DLin-KC2-DMA, DLin-KC3-DMA, DLin-KC4-DMA, DLin-C2K-DMA, DLin-MP-DMA, DODMA, 98N12-5, C12-200, DLin-C-DAP, DLin-DAC, DLinDAP, DLinAP, DLin-EG-DMA, DLin-2-DMAP, KL10, KL22, KL25, Octyl-CLinDMA, Octyl-CLinDMA (2R), Octyl-CLinDMA (2S), and any combination thereof, or
wherein the ionizable amino lipid is selected from the group consisting of (13Z,16Z)-N,N-dimethyl-3-nonyldocosa-13,16-dien-1-amine (L608), (20Z,23Z)—N,N-dimethylnonacosa-20,23-dien-10-amine, (17Z,20Z)—N,N-dimemylhexacosa-17,20-dien-9-amine, (16Z,19Z)—N5N-dimethylpentacosa-16,19-dien-8-amine, (13Z,16Z)—N,N-dimethyldocosa-13,16-dien-5-amine, (12Z,15Z)—N,N-dimethylhenicosa-12,15-dien-4-amine, (14Z,17Z)—N,N-dimethyltricosa-14,17-dien-6-amine, (15Z,18Z)—N,N-dimethyltetracosa-15,18-dien-7-amine, (18Z,21Z)—N,N-dimethylheptacosa-18,21-dien-10-amine, (15Z,18Z)—N,N-dimethyltetracosa-15,18-dien-5-amine, (14Z,17Z)—N,N-dimethyltricosa-14,17-dien-4-amine, (19Z,22Z)—N,N-dimeihyloctacosa-19,22-dien-9-amine, (18Z,21Z)—N,N-dimethylheptacosa-18,21-dien-8-amine, (17Z,20Z)—N,N-dimethylhexacosa-17,20-dien-7-amine, (16Z,19Z)—N,N-dimethylpentacosa-16,19-dien-6-amine, (22Z,25Z)—N,N-dimethylhentriaconta-22,25-dien-10-amine, (21Z,24Z)—N,N-dimethyltriaconta-21,24-dien-9-amine, (18Z)—N,N-dimetylheptacos-18-en-10-amine, (17Z)—N,N-dimethylhexacos-17-en-9-amine, (19Z,22Z)—N,N-dimethyloctacosa-19,22-dien-7-amine, N,N-dimethylheptacosan-10-amine, (20Z,23Z)—N-ethyl-N-methylnonacosa-20,23-dien-10-amine, 1-[(11Z,14Z)-1-nonylicosa-11,14-dien-1-yl]pyrrolidine, (20Z)—N,N-dimethylheptacos-20-en-10-amine, (15Z)-N,N-dimethyl eptacos-15-en-10-amine, (14Z)—N,N-dimethylnonacos-14-en-10-amine, (17Z)-N,N-dimethylnonacos-17-en-10-amine, (24Z)—N,N-dimethyltritriacont-24-en-10-amine, (20Z)-N,N-dimethylnonacos-20-en-10-amine, (22Z)—N,N-dimethylhentriacont-22-en-10-amine, (16Z)-N,N-dimethylpentacos-16-en-8-amine, (12Z,15Z)—N,N-dimethyl-2-nonylhenicosa-12,15-dien-1-amine, N,N-dimethyl-1-[(1S,2R)-2-octylcyclopropyl] eptadecan-8-amine, 1-[(1S,2R)-2-hexylcyclopropyl]-N,N-dimethylnonadecan-10-amine, N,N-dimethyl-1-[(1S,2R)-2-octylcyclopropyl]nonadecan-10-amine, N,N-dimethyl-21-[(1S,2R)-2-octylcyclopropyl]henicosan-10-amine, N,N-dimethyl-1-[(1S,2S)-2-{[(1R,2R)-2-pentylcycIopropyl]methyl}cyclopropyl]nonadecan-10-amine, N,N-dimethyl-1-[(1S,2R)-2-octylcyclopropyl]hexadecan-8-amine, N,N-dimethyl-[(1R,2S)-2-undecyIcyclopropyl]tetradecan-5-amine, N,N-dimethyl-3-{7-[(1S,2R)-2-octylcyclopropyl]heptyl}dodecan-1-amine, 1-[(1R,2S)-2-heptylcyclopropyl]-N,N-dimethyloctadecan-9-amine, 1-[(1S,2R)-2-decylcyclopropyl]-N,N-dimethylpentadecan-6-amine, N,N-dimethyl-1-[(1S,2R)-2-octylcyclopropyl]pentadecan-8-amine, R—N,N-dimethyl-1-[(9Z,12Z)-octadeca-9,12-dien-1-yloxy]-3-(octyloxy)propan-2-amine, S—N,N-dimethyl-1-[(9Z,12Z)-octadeca-9,12-dien-1-yloxy]-3-(octyloxy)propan-2-amine, 1-{2-[(9Z,12Z)-octadeca-9,12-dien-1-yloxy]-1-[(octyloxy)methyl]ethyl}pyrrolidine, (2S)—N,N-dimethyl-1-[(9Z,12Z)-octadeca-9,12-dien-1-yloxy]-3-[(5Z)-oct-5-en-1-yloxy]propan-2-amine, 1-{2-[(9Z,12Z)-octadeca-9,12-dien-1-yloxy]-1-[(octyloxy)methyl]ethyl}azetidine, (2S)-1-(hexyloxy)-N,N-dimethyl-3-[(9Z,12Z)-octadeca-9,12-dien-1-yloxy]propan-2-amine, (2S)-1-(heptyloxy)-N,N-dimethyl-3-[(9Z,12Z)-octadeca-9,12-dien-1-yloxy]propan-2-amine, N,N-dimethyl-1-(nonyloxy)-3-[(9Z,12Z)-octadeca-9,12-dien-1-yloxy]propan-2-amine, N,N-dimethyl-1-[(9Z)-octadec-9-en-1-yloxy]-3-(octyloxy)propan-2-amine; (2S)—N,N-dimethyl-1-[(6Z,9Z,12Z)-octadeca-6,9,12-trien-1-yloxy]-3-(octyloxy)propan-2-amine, (2S)-1-[(11Z,14Z)-icosa-11,14-dien-1-yloxy]-N,N-dimethyl-3-(pentyloxy)propan-2-amine, (2S)-1-(hexyloxy)-3-[(11Z,14Z)-icosa-11,14-dien-1-yloxy]-N,N-dimethylpropan-2-amine, 1-[(11Z,14Z)-icosa-11,14-dien-1-yloxy]-N,N-dimethyl-3-(octyloxy)propan-2-amine, 1-[(13Z,16Z)-docosa-13,16-dien-1-yloxy]-N,N-dimethyl-3-(octyloxy)propan-2-amine, (2S)-1-[(13Z,16Z)-docosa-13,16-dien-1-yloxy]-3-(hexyloxy)-N,N-dimethylpropan-2-amine, (2S)-1-[(13Z)-docos-13-en-1-yloxy]-3-(hexyloxy)-N,N-dimethylpropan-2-amine, 1-[(13Z)-docos-13-en-1-yloxy]-N,N-dimethyl-3-(octyloxy)propan-2-amine, 1-[(9Z)-hexadec-9-en-1-yloxy]-N,N-dimethyl-3-(octyloxy)propan-2-amine, (2R)—N,N-dimethyl-H(1-metoyloctyl)oxy]-3-[(9Z,12Z)-octadeca-9,12-dien-1-yloxy]propan-2-amine, (2R)-1-[(3, 7-dimethyloctyl)oxy]-N,N-dimethyl-3-[(9Z,12Z)-octadeca-9,12-dien-1-yloxy]propan-2-amine, N,N-dimethyl-1-(octyloxy)-3-({8-[(1S,2S)-2-{[(1R,2R)-2-pentylcyclopropyl]methyl}cyclopropyl]octyl}oxy)propan-2-amine, N,N-dimethyl-1-{[8-(2-oclylcyclopropyl)octyl]oxy}-3-(octyloxy)propan-2-amine, (11E,20Z,23Z)—N,N-dimethylnonacosa-11,20,2-trien-10-amine, and any combination thereof.

3. The composition of claim 1, wherein the ionizable amino lipid is a compound having the formula (I) 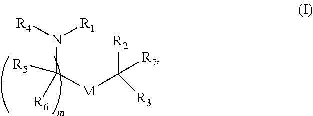
wherein
R₁ is selected from the group consisting of C₅₋₂₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or stereoisomers thereof, wherein alkyl and alkenyl groups may be linear or branched;
provided when R₄ is —(CH₂)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2; or
wherein the ionizable amino lipid is selected from Compound 1 to Compound 147, and salts and stereoisomers thereof.

4. The composition of any one of claims 1 to 3, wherein the quaternary amine compound is selected from the group consisting of 1,2-dioleoyl-3-trimethylammonium-propane (DOTAP), N-[1-(2,3-dioleoyloxy)propyl]-N,N,N-trimethylammonium chloride (DOTMA), 1-[2-(oleoyloxy)ethyl]-2-oleyl-3-(2-hydroxyethyl)imidazolinium chloride (DOTIM), 2,3-dioleyloxy-N-[2(sperminecarboxamido)ethyl]-N,N-dimethyl-1-propanaminium trifluoroacetate (DOSPA), N,N-distearyl-N,N-dimethylammonium bromide (DDAB), N-(1,2-dimyristyloxyprop-3-yl)-N,N-dimethyl-N-hydroxyethyl ammonium bromide (DMRIE), N-(1,2-dioleoyloxyprop-3-yl)-N,N-dimethyl-N-hydroxyethyl ammonium bromide (DORIE), N,N-dioleyl-N,N-dimethylammonium chloride (DODAC), 1,2-dilauroyl-sn-glycero-3-ethylphosphocholine (DLePC), 1,2-distearoyl-3-trimethylammonium-propane (DSTAP), 1,2-dipalmitoyl-3-trimethylammonium-propane (DPTAP), 1,2-dilinoleoyl-3-trimethylammonium-propane (DLTAP), 1,2-dimyristoyl-3-trimethylammonium-propane (DMTAP), 1,2-distearoyl-sn-glycero-3-ethylphosphocholine (DSePC), 1,2-dipalmitoyl-sn-glycero-3-ethylphosphocholine (DPePC), 1,2-dimyristoyl-sn-glycero-3-ethylphosphocholine (DMePC), 1,2-dioleoyl-sn-glycero-3-ethylphosphocholine (DOePC), 1,2-di-(9Z-tetradecenoyl)-sn-glycero-3-ethylphosphocholine (14:1 EPC), 1-palmitoyl-2-oleoyl-sn-glycero-3-ethylphosphocholine (16:0-18:1 EPC), and any combination thereof,
optionally wherein the amount of the quaternary amine compound in the lipid composition ranges from about 0.5 to about 20.0 mole %, from about 0.5 to about 15.0 mole %, from about 0.5 to about 10.0 mole %, from about 1.0 to about 20.0 mole %, from about 1.0 to about 15.0 mole %, from about 1.0 to about 10.0 mole %, from about 2.0 to about 20.0 mole %, from about 2.0 to about 15.0 mole %, from about 2.0 to about 10.0 mole %, from about 3.0 to about 20.0 mole %, from about 3.0 to about 15.0 mole %, from about 3.0 to about 10.0 mole %, from about 4.0 to about 20.0 mole %, from about 4.0 to about 15.0 mole %, from about 4.0 to about 10.0 mole %, from about 5.0 to about 20.0 mole %, from about 5.0 to about 15.0 mole %, from about 5.0 to about 10.0 mole %, from about 6.0 to about 20.0 mole %, from about 6.0 to about 15.0 mole %, from about 6.0 to about 10.0 mole %, from about 7.0 to about 20.0 mole %, from about 7.0 to about 15.0 mole %, from about 7.0 to about 10.0 mole %, from about 8.0 to about 20.0 mole %, from about 8.0 to about 15.0 mole %, from about 8.0 to about 10.0 mole %, from about 9.0 to about 20.0 mole %, from about 9.0 to about 15.0 mole %, from about 9.0 to about 10.0 mole %, about 5.0 mole %, about 10.0 mole %, about 15.0 mole %, or about 20.0 mole %; optionally the amount of the quaternary amine compound in the lipid composition ranges from about 5 to about 10 mole %; and optionally the amount of the quaternary amine compound in the lipid composition is about 5 mole %.

5. The composition of any one of claims 1 to 4, wherein the lipid composition further comprises a phospholipid,
optionally wherein the phospholipid is selected from the group consisting of
1,2-dilinoleoyl-sn-glycero-3-phosphocholine (DLPC),
1,2-dimyristoyl-sn-glycero-phosphocholine (DMPC),
1,2-dioleoyl-sn-glycero-3-phosphocholine (DOPC),
1,2-dipalmitoyl-sn-glycero-3-phosphocholine (DPPC),
1,2-distearoyl-sn-glycero-3-phosphocholine (DSPC),
1,2-diundecanoyl-sn-glycero-phosphocholine (DUPC),
1,2-di-O-octadecenyl-sn-glycero-3-phosphocholine (18:0 Diether PC),
1,2-dilinolenoyl-sn-glycero-3-phosphocholine (DLnPC),
1,2-diarachidonoyl-sn-glycero-3-phosphocholine (DAPC),
1,2-didocosahexaenoyl-sn-glycero-3-phosphocholine (DHAPC),
1,2-dioleoyl-sn-glycero-3-phosphoethanolamine (DOPE),
1,2-diphytanoyl-sn-glycero-3-phosphoethanolamine (4ME 16:0 PE),
1,2-distearoyl-sn-glycero-3-phosphoethanolamine (DSPE),
1,2-dilinoleoyl-sn-glycero-3-phosphoethanolamine (DLPE),
1,2-dilinolenoyl-sn-glycero-3-phosphoethanolamine (DLnPE),
1,2-diarachidonoyl-sn-glycero-3-phosphoethanolamine (DAPE),
1,2-didocosahexaenoyl-sn-glycero-3-phosphoethanolamine (DHAPE),
1,2-dioleoyl-sn-glycero-3-phospho-rac-(1-glycerol)sodium salt (DOPG),
and any combination thereof, and
optionally wherein the phospholipid is selected from the group consisting of
1-myristoyl-2-palmitoyl-sn-glycero-3-phosphocholine (14:0-16:0 PC, MPPC),
1-myristoyl-2-stearoyl-sn-glycero-3-phosphocholine (14:0-18:0 PC, MSPC),
1-palmitoyl-2-myristoyl-sn-glycero-3-phosphocholine (16:0-14:0 PC, PMPC),
1-palmitoyl-2-stearoyl-sn-glycero-3-phosphocholine (16:0-18:0 PC, PSPC),
1-stearoyl-2-myristoyl-sn-glycero-3-phosphocholine (18:0-14:0 PC, SMPC),
1-stearoyl-2-palmitoyl-sn-glycero-3-phosphocholine (18:0-16:0 PC, SPPC),
and any combination thereof.

6. The composition of any one of claims 1 to 5, wherein the lipid composition further comprises a sterol, optionally wherein the sterol is cholesterol,
optionally wherein the lipid composition further comprises a PEG-lipid, and
optionally wherein the net positive charge of the lipid composition is increased compared to the net positive charge of a corresponding lipid composition without the quaternary amine compound.

7. The composition of claim 1, wherein the composition comprising:
(i) a lipid composition comprising
(1) about 50 mole % of MC3 or 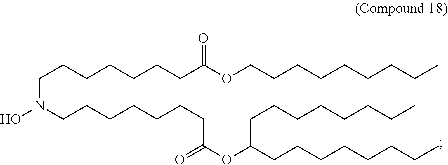 [SMILES: CCCCCCCCCOC(=O)CCCCCCCN(O)CCCCCCCC(=O)OC(CCCCCCCC)CCCCCCCC]
(2) about 10 mole % of DSPC or MSPC;
(3) about 33.5 mole % of cholesterol;
(4) about 1.5 mole % of PEG-DMG;
(5) about 5 mole % of DOTAP; and
(ii) a polynucleotide.

8. The composition of any one of claims 1 to 7, wherein the polynucleotide is selected from a group consisting of plasmid DNA, linear DNA selected from poly and oligo-nucleotides, chromosomal DNA, messenger RNA (mRNA), antisense DNA/RNA, siRNA, microRNA (miRNA), ribosomal RNA, oligonucleotide DNA (ODN) single and double strand, CpG immunostimulating sequence (ISS), locked nucleic acid (LNA), ribozyme, asymmetrical interfering RNA (aiRNA), Dicer-substrate RNA (dsRNA), small hairpin RNA (shRNA), and any combination thereof,
optionally wherein the polynucleotide comprises mRNA,
optionally wherein the mRNA comprises at least one chemically modified nucleobase, and
optionally wherein the nucleobases in the mRNA are chemically modified by at least about 10%, at least about 20%, at least about 30%, at least about 40%, at least about 50%, at least about 60%, at least about 70%, at least about 80%, at least about 90%, at least about 95%, at least about 99%, or about 100%.

9. The composition of any one of claims 1 to 8, wherein the polynucleotide encodes a polypeptide when administered intratumorally to a tumor tissue,
optionally wherein the polypeptide comprises a cytokine, a growth factor, a hormone, a cell surface receptor, an antibody or antigen binding portion thereof, or the polynucleotide encodes a polypeptide which targets a cancer antigen.
optionally wherein the polynucleotide encodes a polypeptide when administered intratumorally to a tumor tissue, and wherein expression of the polypeptide in the tumor tissue is higher than expression of the polypeptide in a non-tumor tissue,
optionally wherein a ratio of the protein expression in the tumor tissue to that in the non-tumor tissue is at least about 200:1, at least about 250:1, at least about 300:1, at least about 350:1, at least about 400:1, at least about 450:1, at least about 500:1, at least about 600:1, at least about 700:1, at least about 800:1, at least about 900:1, or at least about 1000:1, when the protein expression is measured 24 hours post administration, and
optionally wherein a ratio of the protein expression in the tumor tissue to that in the non-tumor tissue is at least about 200:1, at least about 250:1, at least about 300:1, at least about 350:1, at least about 400:1, at least about 450:1, at least about 500:1, at least about 600:1, at least about 700:1, at least about 800:1, at least about 900:1, or at least about 1000:1, when the protein expression is measured 48 hours post administration.

10. The composition of any one of claims 1 to 8, wherein the polynucleotide encodes a polypeptide when administered intratumorally to a tumor tissue, and wherein the composition increases retention of the polynucleotide in the tumor tissue as compared to a corresponding composition without the quaternary amine compound, or
wherein the polynucleotide encodes a polypeptide when administered intratumorally to a tumor tissue, and wherein the composition decreases expression of the polypeptide in a non-tumor tissue as compared to a corresponding composition without the quaternary amine compound.

11. A pharmaceutical composition for intratumoral delivery comprising:
(a) a lipid composition comprising:
(i) a compound of formula (I) 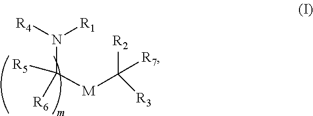
wherein
R₁ is selected from the group consisting of C₅₋₂₀ alkyl, C₅₋₂₀ alkenyl, —R*YR″, —YR″, and —R″M′R′;
R₂ and R₃ are independently selected from the group consisting of H, C₁₋₁₄ alkyl, C₂₋₁₄ alkenyl, —R*YR″, —YR″, and —R*OR″, or R₂ and R₃, together with the atom to which they are attached, form a heterocycle or carbocycle;
R₄ is selected from the group consisting of a C₃₋₆ carbocycle, —(CH₂)ₙQ, —(CH₂)ₙCHQR, —CHQR, —CQ(R)₂, and unsubstituted C₁₋₆ alkyl, where Q is selected from a carbocycle, heterocycle, —OR, —O(CH₂)ₙN(R)₂, —C(O)OR, —OC(O)R, —CX₃, —CX₂H, —CXH₂, —CN, —N(R)₂, —C(O)N(R)₂, —N(R)C(O)R, —N(R)S(O)₂R, —N(R)C(O)N(R)₂, —N(R)C(S)N(R)₂, and —C(R)N(R)₂C(O)OR, and each n is independently selected from 1, 2, 3, 4, and 5;
each R₅ is independently selected from the group consisting of C₁₋₃ alkyl, C₂₋₃ alkenyl, and H;
each R₆ is independently selected from the group consisting of C₁₋₃ alkyl, C₂₋₃ alkenyl, and H;
M and M′ are independently selected from —C(O)O—, —OC(O)—, —C(O)N(R′)—, —N(R′)C(O)—, —C(O)—, —C(S)—, —C(S)S—, —SC(S)—, —CH(OH)—, —P(O)(OR′)O—, —S(O)₂—, an aryl group, and a heteroaryl group;
R₇ is selected from the group consisting of C₁₋₃ alkyl, C₂₋₃ alkenyl, and H;
each R is independently selected from the group consisting of C₁₋₃ alkyl, C₂₋₃ alkenyl, and H;
each R′ is independently selected from the group consisting of C₁₋₁₈ alkyl, C₂₋₁₈ alkenyl, —R*YR″, —YR″, and H;
each R″ is independently selected from the group consisting of C₃₋₁₄ alkyl and C₃₋₁₄ alkenyl;
each R* is independently selected from the group consisting of C₁₋₁₂ alkyl and C₂₋₁₂ alkenyl;
each Y is independently a C₃₋₆ carbocycle;
each X is independently selected from the group consisting of F, Cl, Br, and I; and
m is selected from 5, 6, 7, 8, 9, 10, 11, 12, and 13,
or salts or stereoisomers thereof, wherein alkyl and alkenyl groups may be linear or branched;
provided when R4 is —(CH2)ₙQ, —(CH₂)ₙCHQR, —CHQR, or —CQ(R)₂, then (i) Q is not —N(R)₂ when n is 1, 2, 3, 4 or 5, or (ii) Q is not 5, 6, or 7-membered heterocycloalkyl when n is 1 or 2;
and
(b) a therapeutic agent or a polynucleotide encoding a therapeutic agent.

12. The pharmaceutical composition of claim 11,
(a) wherein the compound of formula (I) is Formula (IA): 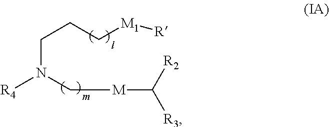
or a salt or stereoisomer thereof, wherein 1 is selected from 1, 2, 3, 4, and 5; m is selected from 5, 6, 7, 8, and 9; M₁ is a bond or M′; R₄ is unsubstituted C₁₋₃ alkyl, or —(CH₂)ₙQ, in which n is 1, 2, 3, 4, or 5 and Q is OH, —NHC(S)N(R)₂, or —NHC(O)N(R)₂; M and M′ are independently selected from —C(O)O—, —OC(O)—, —C(O)N(R′)—, —P(O)(OR′)O—, an aryl group, and a heteroaryl group; and
R₂ and R₃ are independently selected from the group consisting of H, C₁₋₁₄ alkyl, and C₂₋₁₄ alkenyl;
(b) wherein the compound of formula (I) is Formula (II): 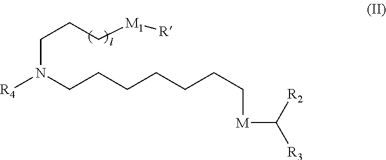
or a salt or stereoisomer thereof, wherein 1 is selected from 1, 2, 3, 4, and 5; M₁ is a bond or M′; R₄ is unsubstituted C₁₋₃ alkyl, or —(CH₂)ₙQ, in which n is 2, 3, or 4, and Q is OH, —NHC(S)N(R)₂, or —NHC(O)N(R)₂; M and M′ are independently selected from —C(O)O—, —OC(O)—, —C(O)N(R′)—, —P(O)(OR′)O—, an aryl group, and a heteroaryl group; and
R₂ and R₃ are independently selected from the group consisting of H, C₁₋₁₄ alkyl, and C₂₋₁₄ alkenyl;
(c) wherein the compound of formula (I) is of the formula (IIa), 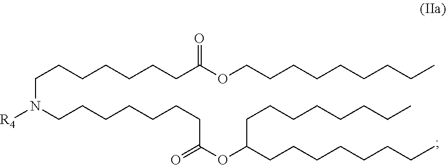
(d) wherein the compound of formula (I) is of the formula (IIb), 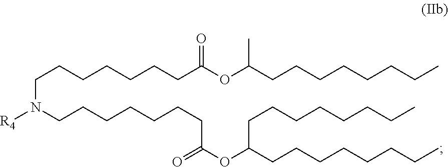
(e) wherein the compound of formula (I) is of the formula (IIc), 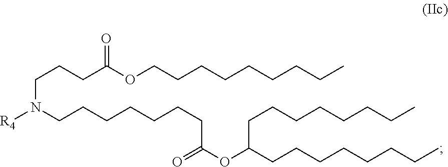
(f) wherein the compound of formula (I) is of the formula (IIe), 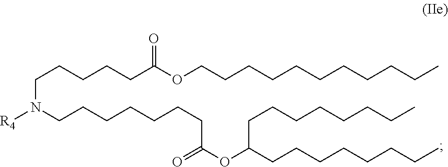
or
(g) wherein the compound of formula (I) is of the formula (IId), 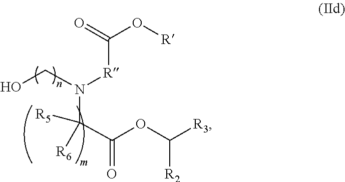
wherein R₂ and R₃ are independently selected from the group consisting of C₅₋₁₄ alkyl and C₅₋₁₄ alkenyl, n is selected from 2, 3, and 4, and R′, R″, R₅, R₆ and m are as defined in claim 11.

13. The pharmaceutical composition of claim 11, wherein the compound of formula (I) is selected from Compound 1 to Compound 147.

14. The pharmaceutical composition of any one of claims 11 to 13, wherein the lipid composition further comprises a phospholipid,
optionally wherein the lipid composition further comprises a quaternary amine compound,
optionally wherein the lipid composition further comprises a structural lipid, and
optionally wherein the lipid composition further comprises a polyethylene glycol (PEG) lipid.

15. The pharmaceutical composition of any one of claims 11 to 13, wherein the lipid composition comprises:
(1) about 50 mole % of the compound of formula (I);
(2) about 10 mole % of DSPC or MSPC;
(3) about 33.5 mole % of cholesterol;
(4) about 1.5 mole % of PEG-DMG; and
(5) about 5 mole % of DOTAP.
